SEQUENCE LISTING (112 sequences)

SEQ ID NO: 1 - DNA, 27 bp, Artificial Sequence
  Description of Artificial Sequence Synthetic primer
atggatttaaccaaactaacgtttgaa

SEQ ID NO: 2 - DNA, 23 bp, Artificial Sequence
  Description of Artificial Sequence Synthetic primer
tcaatccgtgaaatcataggtgt

SEQ ID NO: 3 - protein, 34 aa, Caenorhabditis elegans
PGTTKEEALSNIDQISDKAEELKRSTSDAVQDND

SEQ ID NO: 4 - protein, 385 aa, Caenorhabditis elegans
MDLTKLTFESVFGGSDVPMKPSRSEDNKTPRNRTDLEMFLKKTPLMVLEEAAKAVYQKTPTWGTVELPEGFEMTLILNEITVKGQATSKKAARQKAAVEYLRKVVEKGKHEIFFIPGTTKEEALSNIDQISDKAEELKRSTSDAVQDNDNDDSIPTSAEFPPGISPTENWVGKLQEKSQKSKLQAPIYEDSKNERTERFLVICTMCNQKTRGIRSKKKDAKNLAAWLMWKALEDGIESLESYDMVDVIENLEEAEHLLEIQDQASKIKDKHSALIDILSDKKRFSDYSMDFNVLSVSTMGIHQVLLEISFRRLVSPDPDDLEMGAEHTQTEEIMKATAEKEKLRKKNMPDSGPLVFAGHGSSAEEAKQCACKSAIIHFNTYDFTD

SEQ ID NO: 5 - protein, 9 aa, Caenorhabditis elegans
RIQLKYPHL

SEQ ID NO: 6 - protein, 1002 aa, Caenorhabditis elegans
MSGGPQYLPGVMNSTIQQQPQSATSSFLPSGPISSTSTSSQVVPTSGATQQPPFPSAQAAASTALQNDLEEIFNSPPTQPQTFSDVPQRQAGSLAPGVPIGNTSVSIGEPANTLGGGLPGGAPGQLPGGNQSGIQFQCPRRPNHGVEGRSILLRANHFAVRIPGGTIQHYQVDVTPDKCPRRVNREIISCLISAFSKYFTNIRPVYDGKRNMYTREPLPIGRERMDFDVTLPGDSAVERQFSVSLKWVGQVSLSTLEDAMEGRVRQVPFEAVQAMDVILRHLPSLKYTPVGRSFFSPPVPNASGVMAGSCPPQASGAVAGGAHSAGQYHAESKLGGGREVWFGFHQSVRPSQWKMMLNIDVSATAFYRSMPVIEFIAEVLELPVQALAERRALSDAQRVKFTKEIRGLKIEITHCGQMRRKYRVCNVTRRPAQTQTFPLQLETGQTIECTVAKYFYDKYRIQLKYPHLPCLQVGQEQKHTYLPPEVCNIVPGQRCIKKLTDVQTSTMIKATARSAPEREREISNLVRKAEFSADPFAHEFGITINPAMTEVKGRVLSAPKLLYGGRTRATALPNQGVWDMRGKQFHTGIDVRVWAIACFAQQQHVKENDLRMFTNQLQRISNDAGMPIVGNPCFCKYAVGVEQVEPMFKYLKQNYSGIQLVVVVLPGKTPVYAEVKRVGDTVLGIATQCVQAKNAIRTTPQTLSNLCLKMNVKLGGVNSILLPNVRPRIFNEPVIFFGCDITHPPAGDSRKPSIAAVVGSMDAHPSRYAATVRVQQHRQEIISDLTYMVRELLVQFYRNTRFKPARIVVYRDGVSEGQFFNVLQYELRAIREACMMLERGYQPGITFIAVQKRHHTRLFAVDKKDQVGKAYNIPPGTTVDVGITHPTEFDFYLCSHAGIQGTSRPSHYHVLWDDNNLTADELQQLTYQMCHTYVRCTRSVSIPAPAYYAHLVAFRARYHLVDREHDSGEGSQPSGTSEDTTLSNMARAVQVHPDANNVMYFA

SEQ ID NO: 7 - DNA, 20 bp, Caenorhabditis elegans
tctgagtttggctcgatgtg

SEQ ID NO: 8 - DNA, 20 bp, Caenorhabditis elegans
atgttccttggataccagcg

SEQ ID NO: 9 - DNA, 20 bp, Caenorhabditis elegans
agcccagaactgggaaactt

SEQ ID NO: 10 - DNA, 20 bp, Caenorhabditis elegans
aagtcgaattccgttggatg

SEQ ID NO: 11 - DNA, 20 bp, Artificial Sequence
  Description of Artificial Sequence Synthetic primer
tctaattttccaattttcag

SEQ ID NO: 12 - DNA, 20 bp, Artificial Sequence
  Description of Artificial Sequence Synthetic primer
gatattgttccaggacagcg

SEQ ID NO: 13 - protein, 910 aa, Caenorhabditis elegans
MFPLPVHNGPRLGKLSIFEMPGDSLTSSSFMPDGGAETSSSSQLGGSAHGAIGTKPDAGVQFQCPVRPNHGVEGRSILLRANHFAVRIPGGSVQHYQIDVFPDKCPRRVNREVIGCLISSFSKYFTNIRPVYDGKRNMYTREPLPIGTEPMNFEVTLPGDSAVERKFSVTMKWIGQVCLSALDDAMEGRVRQVPHEAVQSIDVILRHLPSLKYTPVGRSFFTPPGVMKPGMQMHQESKLGGGREVWFGFHQSVRPSQWKMMLNIDVSATAFYRAMPVIEFVAEVLELPVQALAERRALSDAQRVKFTKEIRGLKIEITHCGAVRRKYRVCNVTRRPAQTQTFPLQLETGQTIECTVAKYFFDKYRIQLKYPHLPCLQVGQEQKHTYLPPEVCDIVPGQRCLKKLTDVQTSTMIKATARSAPEREREICKLVSKAELSADPFAHEFGITINPAMTEVKGRVLSAPKLLYGGRHRATTALPNQGVWDMRGKQFHTGMEVRTWAIACFAQQSHVKENDLRMFTTQLQRISTDAGMPIIGTPMFCKYASGVEQVEPMFKYLKQTYSAIQLIVVVLPGKTPIYAEVKRVGDTVLGIATQCVQAKNAIRTTPQTLSNLCLKMNVKLGGVNSILLPNVRPRIFNEPVIFLGCDITHPAAGDTRKPSIAAVVGSMDAHPSRYAATVRVQQHRQEIITDLTYMVRELLVQFYRNTRFKPARIVVYRDGVSEGQLFNVLQYELRAIREACVMLESGYQPGITFIAVQKRHHTRLFAADKADQVGKAFNIPPGTTVDVGITHPTEFDFFLCSHAGIQGTSRPSHYHVLWDDNDLTADELQQLTYQMCHTYVRCTRSVSIPAPAYYAHLVAFRARYHLVDRDHGSGEEGSQPSGTSSEDTTLSSMAKAVQVHPDSNNVMYFA

SEQ ID NO: 14 - protein, 4 aa, Artificial Sequence
  Description of Artificial Sequence Synthetic peptide motif
DEAD

SEQ ID NO: 15 - protein, 4 aa, Artificial Sequence
  Description of Artificial Sequence Synthetic peptide motif
DEAH

SEQ ID NO: 16 - DNA, 27 bp, Artificial Sequence
  Description of Artificial Sequence Synthetic primer
atgaggaaaaagcagtgttcttcaata

SEQ ID NO: 17 - DNA, 27 bp, Artificial Sequence
  Description of Artificial Sequence Synthetic primer
ttatgcttctctgattaaattgactac

SEQ ID NO: 18 - protein, 1037 aa, Caenorhabditis elegans
MRKKQCSSILSLYDKEIILCLEPIYRDPEKGDGFSELLPLGRIDELKIQSENAQEFSKQLYHDLKNSILSNADDERLYKDIMTYLQTYLPKCTVHKLLNCSNREVKLSDFHYILDHFEGFLRFIEPKVVLAYLDSYPQYIDAVAVLRKEIERNEEDNQDSDFIKKLILRTVPLLGEQAVYDIMYTISEKSSNNLDVEAKQFIAKVLRLKNDGFLRFYQIINASRRQLNGRIYICPVHESATEMMVYLGTAALNTNRYRMINIRVDNIVQENSTPRLVIESVRQRIHRQRQLCLRNYQEELCQVALQGKNTIVTAPTGSGKTVIAANIIKEHFESRSSEGKRFKALFMTPNSMILNQQAASISSYLDHVYHTQIIQGSDNVPTRNVIQSKDLIVATPQMIVNLCNEHRNSLDDESRLDQFFLSTFTIIFFDECHNTVKNSPYSNIMREYHYLKNMGNMPEGHSLPQIIGLTASLGTGDKNDCLQVRNYIAGLCASMDVKDLSIVKDNLEELRGYSPIVPDKVLLCERSTDGPIGMFTNRLTLMMQEVEGLIRTALRNEHIGIEQRRQIETTERDFRPDSSFLDPPADKEHAGYQNWVCNQMNLVSGTSFRETGTRTIINEALDVLKECFCTLSYNINFHPEVALNYLKDEMEYRTPNFTVNMIRIWERYHNQLVGTGSAENPMISKTVQYIVEQNLQRADSRTIIFVRTRYEATILNKVLNSNEELLMLGIKSEWMSGLNKSTASSADISASKQKQMEKLKMFADGEIRILVSTSVAEEGLDVPECSLVIKYNYATNEIAHVQRRGRGRALNSECVLITNSIALRDQESNNRDKESLMSETISLIQNSPAEFRKCVDEESNKIWPRILREDTDKAQKIEEQINRNIVYKIICKKCEAILCTSKDIRSRNTQYLVCDPGFWSLVRKTRLTDEQQALIKYNATGSINCRRENCGLKLGQLIEVNTVDLPCLSALSIVLLVEGTDKRIIVKKWKNILDKYFTPTEIRQLDVQTMRDADQARTPMVFEHHANGEVVNLIREA

SEQ ID NO: 19 - DNA, 27 bp, Artificial Sequence
  Description of Artificial Sequence Synthetic primer
atgattgtaaatctttgcaatgagcac

SEQ ID NO: 20 - DNA, 29 bp, Artificial Sequence
  Description of Artificial Sequence Synthetic primer
ttatgcttgtctaattacattgattactt

SEQ ID NO: 21 - protein, 620 aa, Caenorhabditis elegans
MIVNLCNEHRDPLDDEYPPEQFFLSTFTIIFFDECHNTVKNSPYSNVMREYHYLKNMGNMPEGHSFPQIIGLTASLGTGDKKNCMQVRSYIAGLCANMDVKELSIVKDNLEELLDHNPFVTDQVSFCERSNDGPIEMFTKRLKQMMQEVEDLIRTTLKNEPTVKYEIPPTDKEHNRYENWISNQRNCVSLAGSRNKTLIIEVLDVLKDCFYALSYNINFNPEVALKKYLEKELGPERIRNFTDNMNLIWDNCHRELVGIGSAENPMIARTVQFILDQNEQTSDFRAIIFVRTKKEADFLNYVLNDRLHELGIKSDWMSGQKKSTASSADISASKQKQMEKLKMFADGENQILVSTSVAEEGLDIPECSLVIKYNYATNETAHVQRRGRARARNSKCVLITNSIALHVQESNNLAKENLMTETISLIQNSPGEFRQCVDEESNKVWPRIQREDTDKAQRIKEQINRNIVYKIVCMKCDTVLCTNKDIRSKNTQYIVCNPGFWSLVRRIPLPLEQRASNKFNSTGSIECLGERCGSKLGQLIDVNTVNLPCLKVKSILLLIESTNERILVKQWKNILDEHFTPTTLKQRDVQTMKDADYGRAPIEFEHHTANGEVINVIRQA

SEQ ID NO: 22 - protein, 1134 aa, Caenorhabditis elegans
MQPTAIRLEDYDKSKLRLPFESPYFPAYFRLLKWKFLDVCVESTRNNDIGYFKLFESLFPPGKLEEIARMIIDEPTPVSHDPDMIKIRNADLDVKIRKQAETYVTLRHAHQQKVQRRRFSECFLNTVLFDEKGLRIADEVMFNYDKELYGYSHWEDLPDGWLTAETFKNKFYDEEEVTNNPFGYQKLDRVAGAARGMIIMKHLKSNPRCVSETTILAFEVFNKGNHQLSTDLVEDLLTEGPAFELKIENGEEKKYAVKKWSLHKTLTMFLAIIGFKSNDKKEKNEHEEWYYGFIDAMKNDPANRAALYFLDKNWPEELEEREKERDRIRLTLLKSQRTNEEAVGEDVCTTIRPQPKDSGYNPDAVVTELVLRTYQEELVQPALEGKNCVIVAPTGSGKTEVAIYAALKHIEERTSQGKPSRVVLLVPKIPLVGQQKDRFLKYCNGMYEVNGFHGSESSVSGTGRRDEVIATHVSVMTPQILINMLQSVRQNERLYVSDFSMMIFDEVHKAAKNHPYVLINQMVQEWKYEKPQIIGLTASLSVKVDGQKDENQMLNDIYNMLALINAPHLSTITRQSSIDELNEHVGKPDDSVELCLPAKENILRDYIERYLNHAHGKFLEELASMSKSTGRNNTIPPNMINTFKKNQPKNYEYYDSLLQGIIQELNKLNVPEKWNSQTWAKYMKVYLEARGIVDLMPAMVAFKYMEKAIGKLNESHSETVEYSTFIKDHDTLKQTIQSVEPEIVLRLKKYTHQSVPHQFGNYGEQMVGYVLGTNKQGAVQQTSQEQQLTLDKFNNGRLKVIVATSVVEEGLDVTACNLIIKYNCSSGSAIQLVQQRGRARAKNSRSVLLSVKSSINETETNALISEKYMRLCVKKITENGEKQLAAEVKRVAELNAAERKRNLEEQLNLRLRHENKIYKLMCSNCSKEFCKSIYIKKVFSNYMVFDPSVWRFLHVESVETFIKCLKITWKCRIADYQIAEFPNFAFRQLTFRLFLCNFQMFQKRKVSKYLSEDNQPLSDIKCFHCKLDVGRAYKIRGTYLPQLSVKALTFVQESDYSSMTKAKWSDVEQDLFYISEAIEDDFRIMLNALSDTEENIEKKIVLDLDSRQHNKQLEMKRFHIQQEPPTKGVAPEAQ

SEQ ID NO: 23 - protein, 2145 aa, Caenorhabditis elegans
MADELARIQQYEYRQNSNLVLSVDYNLTDRRGREEPTGEVLPITDKEMRKMKMGDRAIKGKAPVQDQKKKRKKKDDEKAQQFGRNVLVDNNELMGAYKPRTQETKQTYEVILSFILDALGDVPREVLCGAADEVLLTLKNDKFRDKEKKKEVEALLGPLTDDRIAVLINLSKKISDFSIEEENKPEGDGDIYENEGVNVQFDSDEEEDDGGMVNEIKGDSEEESEEEEGVDTDYTATLKGDGHLTEDEQKARGILHPRDIDAHWIQRSLAKYFKDPLIAQQKQTEVIGILKNAADDRDAENQLVLLLGFDQFEFIKCLRQNRLMILYCTLLRQANEKERLQIEDDMRSRPELHPILALLQETDEGSVVQVEKSKRDAEKSKKAATAANEAISAGQWQAGRKMLDLNDLTFSQGSHLMSNKRCELPDGSYRRQKKSYEEIHVPALKPRPFAEGEKLVSVSELPKWAQPAFDGYKSLNRIQSRLCDSALRSKEHLLLCAPTGAGKTNVALLTMLQEIGNHLAEDGSVKLDEFKIVYIAPMKSLVQEMVGSFSKRLAPFGITVGEMTGDAQMSKEQFMATQVIVCTPEKYDVVTRKGGERAYNQMVRLLIIDEIHLLHDDRGPVLESIVVRTIRQMEQNHDECRLVGLSATLPNYQDVATFLRVKPEHLHFFDNSYRPVPLEQQYIGVTEKKALKRFQAMNEVVYDKIMEHAGKSQVLVFVHSRKETAKTAKAIRDACLEKDTLSAFMREGSASTEILRTEAEQAKNLDLKDLLPYGFAIHHAGMNRVDRTLVEDLFADRHIQVLFSTATLAWGVNLPAHTVIIKGTQIYNPEKGRWTELGALDIMQMLGRAGRPQYDDRGEGILITNHSELQYYLSLMNQQLPVESQMVSRLTDMLNAEVVLGTVSSVSEATNWLGYTFLFVRMLKNPTLYGITHEQARADPLLEQRRADLIHTACVLLDKAGLIKYDKRSGIIQATELGRIASHFYCTYESMQTYNKLLVETCSDIDLFRIFSMSSEFKLLSVRDEEKLELQKMAEHAPIPIKENLDEASAKTNVLLQAYISQLKLEGFALQADMVFVAQSAGRLFRALFEIVLWRGWAGLAQKVLTLCKMVTQRQWGSLNPLHQFKKIPSEVVRSIDKKNYSFDRLYDLDQHQLGDLIKMPKMGKPLFKFIRQFPKLEMTTLIQPITRTTMRIELTITPDFKWDEKVHGSAEGFWIFIEDTDGEKILHHEFFLLKQKFCSDEHVVKMIVPMFDPMPPLYYVRIVSDRWIGAETVLPISFRHLILPEKYPPPTELLDLQPLPISAVTNKEFQTVFAESGFKVFNPIQTQVFRTVFESNENVIVCAPNGSGKTAIAELAVLRHFENTPEAKAVYITPMEDMATKVYADWKRRLEPAIGHTIVLLTGEQTMDLKLAQRGQLIISTPERWDNISRRWKQRKSVQNVKLFIADDLHMIGASNGAVFEVVCSRTRYISSQLESAVRVVALSSSLTNARDLGMWLGCSASATFNFMPSTRPVPLDLEIKSFNLSHNASRFAAMERPVYQAICRHAGKLEPKPALVFVPVRRQTRPVAVALLTMALADGAPKRFLRLAEHDDTFQALLADIEDESLRESVSCGVGFLHEGTAPKDVHIVQQLFESNAIQVCVVPRGMCYQIEMSAYLVVVMDTQFYNGKYHVYEDYPIADMLHMVGLANRPILDSDAKCVVMCQTSKRAYYKKFLCDPLPVESHLDHCLHDHFNAEIVTKTIENKQDAIDYLTWTLLYRRMTQNPNYYNLQGTTHRHLSDALSELVELTLKDLENSKCIAVKDEMDTVSLNLGMIASYYYISYQTIELFSMSLKEKTKTRALIEIISASSEFGNVPMRHKEDVILRQLAERLPGQLKNQKFTDPHVKVNLLIHAHLSRVKLTAELNKDTELIVLRACRLVQACVDVLSSNGWLSPAIHAMELSQMLTQAMYSNEPYLKQLPHCSAALLERAKAKEVTSVFELLELENDDRSDILQMEGAELADVARFCNHYPSIEVATELENDVVTSNDNLMLAVSLERDNDIDGLAPPVVAPLFPQKRKEEGWWLVIGDSESNALLTIKRLVINEKSSVQLDFAAPRPGHHKFKLFFISDSYLGADQEFDVAFKVEEPGRSNRKRKHEKEED

SEQ ID NO: 24 - protein, 28 aa, Caenorhabditis elegans
VMNIKKDKTAALVEKLGIKLANDEKDLE

SEQ ID NO: 25 - protein, 4 aa, Caenorhabditis elegans
RKRK

SEQ ID NO: 26 - protein, 4 aa, Caenorhabditis elegans
YLDK

SEQ ID NO: 27 - protein, 852 aa, Caenorhabditis elegans
MVNFTVDEIRALMDRKRNIRNMSVIAHVDHGKSTLTDSLVSKAGIIAGSKAGETRFTDTRKDEQERCITIKSTAISLFFELEKKDLEFVKGENQFETVEVDGKKEKYNGFLINLIDSPGHVDFSSEVTAALRVTDGALVVVDCVSGVCVQTETVLRQAIAERIKPVLFMNKMDRALLELQLGAEELFQTFQRIVENINVIIATYGDDDGPMGPIMVDPSIGNVGFGSGLHGWAFTLKQFAEMYAGKFGVQVDKLMKNLWGDRFFDLKTKKWSSTQTDESKRGFCQFVLDPIFMVFDAVMNIKKDKTAALVEKLGIKLANDEKDLEGKPLMKVFMRKWLPAGDTMLQMIAFHLPSPVTAQKYRMEMLYEGPHDDEAAVAIKTCDPNGPLMMYISKMVPTSDKGRFYAFGRVFSGKVATGMKARIQGPNYVPGKKEDLYEKTIQRTILMMGRFIEPIEDIPSGNIAGLVGVDQYLVKGGTITTYKDAHNMRVMKFSVSPVVRVAVEAKNPADLPKLVEGLKRLAKSDPMVQCIFEESGEHIIAGAGELHLEICLKDLEEDHACIPLKKSDPVVSYRETVQSESNQICLSKSPNKHNRLHCTAQPMPDGLADDIEGGTVNARDEFKARAKILAEKYEYDVTEARKIWCFGPDGTGPNLLMDVTKGVQYLNEIKDSVVAGFQWATREGVLSDENMRGVRFNVHDVTLHADAIHRGGGQIIPTARRVFYASVLTAEPRLLEPVYLVEIQCPEAAVGGIYGVLNRRRGHVFEESQVTGTPMFVVKAYLPVNESFGFTADLRSNTGGQAFPQCVFDHWQVLPGDPLEAGTKPNQIVLDTRKRKGLKEGVPALDNYLDKM

SEQ ID NO: 28 - protein, 4 aa, Caenorhabditis elegans
APKK

SEQ ID NO: 29 - protein, 4 aa, Caenorhabditis elegans
PKKK

SEQ ID NO: 30 - protein, 463 aa, Caenorhabditis elegans
MGKEKVHINIVVIGHVDSGKSTTTGHLIYKCGGIDKRTIEKFEKEAQEMGKGSFKYAWVLDKLKAERERGITIDIALWKFETAKYYITIIDAPGHRDFIKNMITGTSQADCAVLVVACGTGEFEAGISKNGQTREHALLAQTLGVKQLIVACNKMDSTEPPFSEARFTEITNEVSGFIKKIGYNPKAVPFVPISGFNGDNMLEVSSNMPWFKGWAVERKEGNASGKTLLEALDSIIPPQRPTDRPLRLPLQDVYKIGGIGTVPVGRVETGIIKPGMVVTFAPQNVTTEVKSVEMHHESLPEAVPGDNVGFNVKNVSVKDIRRGSVCSDSKQDPAKEARTFHAQVIIMNHPGQISNGYTPVLDCHTAHIACKFNELKEKVDRRTGKKVEDFPKFLKSGDAGIVELIPTKPLCVESFTDYAPLGRFAVRDMRQTVAVGVIKSVEKSDGSSGKVTKSAQKAAPKKK

SEQ ID NO: 31 - protein, 709 aa, Caenorhabditis elegans
SWSGDKLAWLQTWRRVISLVDPYTNSSAHVAIDCMSLTIENLLLVNLHPLAHWLACRLVTVPPILLPRCVPALSAILNESTIRRPPPLLSANILLCFIRLMQSKEQLVVPAICGLSAHELSIVAPRALEHLPKMLQAAKSSKDTKVSSNSLKLFSMLASSYPGAEQILLDQLVNTDVSENAVVIVNSLAILIVQKAQIDLVLTALKTIETHQFAMRLIPLFCSSIASLAQFSSTTLLQALLRAASLLRDERTRTEIEWQMVKLCMQWPQPQMPLVIRGILADRHMVLHGELVTLGGQYPVRGFEVQRWSSAGAPPLQGEDKTVYINRQSAIISVSRKDFHAKSPCEITSRTVVGRHIWDLDTHEDVRKPATNVTNWLRKEALKGKRPGRESQGILGAMDDPFDDLPDYPPSRGSPSPVDGAAQFTSMIETSRRQPQPLGTSSAAHDHLPAFTPNAKLLEWRSLSASLGFVPLVSQVHANFPRDLKHLDQTSSREVHKVAVIYVGESQEDRASILSNTTASASAQFDSFTSELGWEVKVGRGHDGYTGGLPVETRAPYFADAEAEVIFHVSTMLNGDVQQKWKHIGNDEVHVVWTENTRKVYTRETIATKFCDVLIVLEQVGDKMVRVRVDTASALEFGPLFDGALVTMSELSQLVRLTVINASRAYRLARVEHSRPLRHREEVFCNEALAHMKPMPLAQSINHLYVPTI

SEQ ID NO: 32 - protein, 526 aa, Caenorhabditis elegans
MVEGDVDESASGTSGTNKKILFTKKPSVWKDFDNWINDEPENRYDLFQVVKSAMLLQSGYTTILMDQVTDNGADELRISLEYSNFIKIVNSTKLVVGKEQCPPSNVFTLLAEIFANTPGNTSEVGRISTWLTSHLGALLHNDVIWKIHFFDPDLFRSVYWQLIFTLKLAPGDTENLEEDENYAKLLFSCFITAVMVALWHDHEMSFNSICPDYLKPETASEYMVMLISSPPFRSLSQFFLFGLHLLGKYQSEGGCVVVREEAYIAEIRQNDEEKRQSIETRTNLISDDMVYDDGEDLLEQIDRVQQLHEAHCIVLLKKGFLKAPDGFKIVQKGGRPRKYPASATKKRKKKTPRSSPKKKMSKESPINHQKEPIDEQKPSTSLPIYSVATLKPRRKVVKTADEVGLCAPIFVMQSELLKKFREEVQRRYAEGSSASDQERVRNMVYEAYDNIYHINRLSANEGPRILTSDQKLVMQQYKTTFRQGPTFAEETESDVEEEEEKKVVEVVTAKVIKGSAKSSKKFKRRY

SEQ ID NO: 33 - protein, 160 aa, Caenorhabditis elegans
MTISKNNKMMAHLNYRMKIILQDGRTFIGFFKAFDKHMNILLAECEEHRQIKPKAGKKTDGEEKRILGLVLVRGEHIVSMTVDGPPPRDDDSVRLAKAGGAGGVGQAKPGGRGMPAMPGMPGMPPGGAPGGLSGAMRGHGGPGMAAMQPGYGGPPGGRPF

SEQ ID NO: 34 - protein, 126 aa, Caenorhabditis elegans
MKLVRFLMKLSHETVNIELKNGTQVSGTIMGVDVAMNTHLRAVSMTVKNKEPVKLDTLSIRGNNIRYIILPDPLALDTLLIDDEPRKKARAARAGASRGRGRGGMRGGRGGRGRGRGGPRGGGPRR

SEQ ID NO: 35 - protein, 261 aa, Caenorhabditis elegans
MPEPRCTAIVNFLNLSHSILISIFSVSVMSNYHHNHNYQHRPRGYERLPGKRLPDRWNIYDNVGRDIDGTRFVPFKTPLDSSFFDGKNMPVELQFGVKTLISLAQQANKQIGLVIDLTNTDRYYKKTEWADHGVKYLKLNCPGHEVNEREDLVQDFINAVKEFVNDKENDGKLIGVHCTHGLNRTGYLICRYMIDVDNYSASDAISMFEYYRGHPMEREHYKKSLYEAERKKKYGKSSGKSSGNSADSTISSEQLHRNNSQ

SEQ ID NO: 36 - DNA, 22 bp, Artificial Sequence
  Description of Artificial Sequence Synthetic primer
atggcgaccatcgtgccatgct

SEQ ID NO: 37 - DNA, 30 bp, Artificial Sequence
  Description of Artificial Sequence Synthetic primer
ctagaagacacggatgcaattgtttccgaa

SEQ ID NO: 38 - protein, 65 aa, Caenorhabditis elegans
TAENEIRAAFDTHVNALEERRKELLKRVETVKNLKLSVLISQAESLQSKQIDLQQAIQTATKLMD

SEQ ID NO: 39 - protein, 1143 aa, Caenorhabditis elegans
MATIVPCSLEKEEGAPSGPRRLQTEIDVDANDSGNELSMGGSSSEGDSMSHHRGEHSPNHHHQDNHLGSGPPPPQFTGSLFDTPPSMIQSPQQQPQFQFNTGFGLGLPQDSFRCSVCSKSSTIGVLPFVCAHKTCQSCYQMTPSSYDRRACKLCGAVSTATANFTSQMYLSPTLPSPPRGALMSDCSTPTMNNHINSSTPLHQPRAFSFSLSGMPGSPSPVMGARMPSSAGGLMMRPIGFPDSDSSLTSWSPLQQPSQLSINNLSSIGGHQQQSPMLMQNVFDSLAVNDDTPVFSPLSPTNTSMHMPPSLMASPDVPKHSATIAPPRNSMCSTPRLQLATPMSSQSQQTFPIPSPLGSQPQQQQPMGPIQCQGCESKISFAYCMQCQEALCIHCVQAHQRVRATKQHAFVELQQLMATLMSRAVQPQQAQQYTQNVGGSVRQALGSVGSGDGHVSGVENDSIGSGESSPRSSSVCGTHDSVIIGICENCPHSVLLCAICVAQHPGKHRVQPLGDIRVAVGEVVNESQLLQWQCEKTGDTIKQIIDGIVTNATTAENEIRAAFDTHVNALEERRKELLKRVETVKNLKLSVLISQAESLQSKQIDLQQAIQTATKLMDSSDCDEMVLRQVFEKLASCQMGNEGTEPNNNILNVLMLACQVNEDDRLKFTAPQDGILLNKARQFGNIESGPCAKNSSIVGDSFKKAIRERQTVIYVQLRDACGDLLSSSIAATQPTSQALLPHQEPHSHLEQAMPTSDVQAFVISPDGSTVEVTMTPRENGIVALSYYPSIEGSYTLNILVKGTPISGCPTTMDIRRGRNYDEIAAKGPILTFGKEGSGDGELCRPWGICVDQRGRVIVADRSNNRVQIFDKDGNFISKFGTSGNRPGQFDRPAGITTNSLNNIVVADKDNHRVQVFDENGMFLLKFGDRGRAVGYFNYPWGVATNSHNAIAVSDTRNHRVQIFTPQGQFVRKCGFDSAYFFKNLDSPRGLCYLPDGQLLITDFNNHRLAVLSPRNMSEMKVYGSEGDGDGMFVRPQGVVIDPEGHILVCDSRNNRVQVFASDDMRFIGSFGLGPVPNSGFQMPQELPAPYSSLGGPFGAPAFSSAPTPLTPSPRQLLDRPTDLAVGPDGRIYVVDFGNNCIRVF

SEQ ID NO: 40 - DNA, 22 bp, Artificial Sequence
  Description of Artificial Sequence Synthetic primer
atggcgaccatcgtgccatgct

SEQ ID NO: 41 - DNA, 30 bp, Artificial Sequence; identical to SEQ ID NO: 37
  Description of Artificial Sequence Synthetic primer

SEQ ID NO: 42 - protein, 65 aa, Caenorhabditis elegans; identical to SEQ ID NO: 38

SEQ ID NO: 43 - protein, 1147 aa, Caenorhabditis elegans
MATIVPCSLEKEEGAPSGPRRLQTEIDVDANDSGNELSMGGSSSEGDSMSHHRGEHSPNHHHQDNHLGSGPPPPQFTGSLFDTPPSMIQSPQQQPQFQFNTGFGLGLPQDSFRCSVCSKSSTIGVLPFVCAHKTCQSCYQMTPSSYDRRACKLCGAVSTATANFTSQMYLSPTLPSPPRGALMSDCSTPTMNNHINSSTPLHQPRAFSFSLSGMPGSPSPVMGARMPSSAGGLMMRPIGFPDSDSSLTSWSPLQQPSQLSINNLSSIGGHQQQSPMLMQNVFDSLAVNDDTPVFSPLSPTNTSMHMPPSLMASPDVPKHSATIAPPRNSMCSTPRLQLATPMSSQSQQTFPIPSPLGSQPQQQQPMGPIQCQGCESKISFAYCMQCQEALCIHCVQAHQRVRATKQHAFVELQQLMATLMSRAVQPQQAQQYTQNVGGSVRQALGSVGSGDVFFSGHVSGVENDSIGSGESSPRSSSVCGTHDSVIIGICENCPHSVLLCAICVAQHPGKHRVQPLGDIRVAVGEVVNESQLLQWQCEKTGDTIKQIIDGIVTNATTAENEIRAAFDTHVNALEERRKELLKRVETVKNLKLSVLISQAESLQSKQIDLQQAIQTATKLMDSSDCDEMVLRQVFEKLASCQMGNEGTEPNNNILNVLMLACQVNEDDRLKFTAPQDGILLNKARQFGNIESGPCAKNSSIVGDSFKKAIRERQTVIYVQLRDACGDLLSSSIAATQPTSQALLPHQEPHSHLEQAMPTSDVQAFVISPDGSTVEVTMTPRENGIVALSYYPSIEGSYTLNILVKGTPISGCPTTMDIRRGRNYDEIAAKGPILTFGKEGSGDGELCRPWGICVDQRGRVIVADRSNNRVQIFDKDGNFISKFGTSGNRPGQFDRPAGITTNSLNNIVVADKDNHRVQVFDENGMFLLKFGDRGRAVGYFNYPWGVATNSHNAIAVSDTRNHRVQIFTPQGQFVRKCGFDSAYFFKNLDSPRGLCYLPDGQLLITDFNNHRLAVLSPRNMSEMKVYGSEGDGDGMFVRPQGVVIDPEGHILVCDSRNNRVQVFASDDMRFIGSFGLGPVPNSGFQMPQELPAPYSSLGGPFGAPAFSSAPTPLTPSPRQLLDRPTDLAVGPDGRIYVVDFGNNCIRVF

SEQ ID NO: 44 - protein, 387 aa, Caenorhabditis elegans
MTAQDYLNGKLAAANGPLADDWKNLKELWEKKLWHQLTVLTRSLVKKPQFVASTDMHEFYRLFVAEWELRVNPLQLVEICISIAQNIATKDKQKSMEFLSKIGNVINKDKIAVARLHTGEIEARLENKDKNGQIIDLKSIRTQIDSTQHEVDSLVGVTEVHAPFYRVSSLYLREVGDFAGYYREALRYLGVEDANNLTTEQKQVHAVLLGFAALLGENVHNFGELLAHPILKSLEGTRERWIVDVLLAFNSGDLTRFFSLEGDWGGWDDLKKQKDFLTAKIRLMAVMELAVSRPTKARSVSFKEIATKCQIPFDEVEFLVMKALSKDLIRGDINQVEQVVYVTWVQPRVLDNPQIMQMATRISAWRNDVNSMEGIVSKEAREILTQN

SEQ ID NO: 45 - protein, 4 aa, Caenorhabditis elegans
KKRH

SEQ ID NO: 46 - protein, 470 aa, Caenorhabditis elegans
MSKTVTIRRPSPTKTSEEPAAHQTPIFTQTAAEMLGITSLNTEAAELLEFLSREKLKEIVRLSAKWTQKSARRRMAVADVEHAIRSTRQCGGLNISSVDTLNLGIQQLQPIQGTSTGIYSFLKSSADVDVDKEDTETFIKIPRDLRVISYPLVNEGQPVQSEYTVNVDEDDGNFFEKIVPEVMTMIPEKNTPSSSTTSSLQMFRDAVKTAKIDQKVGLKPSTIEILTVEQQIFMKDIITVCMGQDDKKRHEALYTLETDAGLQVFLPHLTERICKSISANISQRCLSLIIYAGRVLRSLSHNKACDMTVTLHHVLPALLSCCVGRNMCLRPETDNHWALRDFSAKTLVGLVRDQVDKHDAGRTARRLFDFSHRIFRDTGSSFSMIYGTVHILQEFVAGPKKAAWLLTELGETNARCKSHIESGSRIGASQLSIQEAQKLNQQILKCENSIRNRYNLQQQAPGVPINRRFH

SEQ ID NO: 47 - protein, 298 aa, Caenorhabditis elegans
MLRRERDSKCLKFLKIQFLSSKSFFITIKFYSDKSEYDKEKDLLEIERKKLESQPPSTSTSQSSKDYKSKSSSSKHDKNSSEYERNNKMWARTDLLVRFIDEDFKRGSLYKQKVRIVDVAGYNDVTIEDRGNTHYNIRQSWLETVIPREIGEKLMIVAGKRSGQLALMLGKDKRKEKLTARLVATNDVVTAYFEDVCAVKIRHEEDSPQLYGMTQFLFKILLVCTIINEFDTLNILFYVPTLSHSHISFNTKLAQLLATSGHQVTVLLAQVDRKCIKLEKKPTILPILQLLTLSNLII

SEQ ID NO: 48 - protein, 434 aa, Caenorhabditis elegans
MATSFYTGGGEDGDGFNPRVHARIAEREGFQLASGSEDPRTLFVANLDPAITDEFLATLFNQIGAVMKAKIIFEGLNDPYAFVEFSDHNQATLALQSHNGRELLEKEMHVTWAFEPREPGENRSKPETSRHFHVFVGDLCSEIDSTKLREAFVKFGEVSEAKIIRDNNTNKGKGYGFVSYPRREDAERAIDEMNGAWLGRRTIRTNWATRKPDEDGERGGDRGDRRGGGGGGRDRYHNQSEKTYDEIFNQAAADNTSVYVGNIANLGEDEIRRAFDRFGPINEVRTFKIQGYAFVKFETKESAARAIVQMNNADIGGQIVRCSWGKSGDSGKPSERGSGGGGGSGNYGYGYGNSGGGGGSGGPGNSQFSNFNQRPPPSGNGGSGGGSGGQNNQYWQYYSQYYNNPHLMQQWNNYWQKDGPPPPPAAAASSTGGN

SEQ ID NO: 49 - protein, 562 aa, Caenorhabditis elegans
MQPVLVNSRPLRVKSHESESKLNLIEQEDQFEGANYSSSSGVIICYSNGTGEVITQEAFDDSGIHFIFSKATCIQYPSNFDPIGVGSVVQIFWSRSFERVVRGNHIIVQIEKMEVYKCCAMLREQVFVTFNSPSTAGVAIGVTERNITVAFHPNCSPVIRYETLKAHSIGRTEFEIKDVREFEFSNGKNRHRENTNRMVDVILAAVPFRVEIHGNVDKIPFFVIEKCRNSPGRSGAAVITKIMKNHFMEANFLQNSESIYFDSTSCHSNILEKVSIGSLINVLADPTFATSSYKWYGYDVTLCNNYLAHASTQRSFVLENNEILQNCKKLEKSPEEAETTTKNDLRFVPPQPEKGEVKKNELPEREAKSIINSYFIDRLAEGIKIEKIDKNWRTFGEILPKTPKKYSESLKKSIQNVLEPFGLNKPEKAAETPKIVEYFPKNPKKRVEIVEKPTVDEIRELFGALMDAEGFALNQRVKPHFVLPDTRWKPTERRYIGIYDDVQWTFMSTFCPKIEENSENRPLAGGWWYRRTVPRDHPVEIVQKMETRRNIIKDCTESPFIE

SEQ ID NO: 50 - protein, 37 aa, Caenorhabditis elegans
GMDEMEFTEAESNMNDLVSEYQQYQEATADDEGEFDE

SEQ ID NO: 51 - protein, 444 aa, Caenorhabditis elegans
MREIVHIQAGQCGNQIGAKFWEVISDEHGIDPTGAYNGDSDLQLERINVYYNEASGGKYVPRACLVDLEPGTMDSVRAGPFGQLFRPDNFVFGQSGAGNNWAKGHYTEGAELVDNVLDVVRKEAESCDCLQGFQMTHSLGGGTGSGMGTLLISKIREEYPDRIMMTFSVVPSPKVSDTVVEPYNATLSVHQLVENTDETFCIDNEALYDICFRTLKLTTPTYGDLNHLVSMTMSGVTTCLRFPGQLNADLRKLAVNMVPFPRLHFFMPGFAPLTSRGSQQYRSLTVPELTQQMFDAKNMMAACDPRHGRYLTVAAMFRGRMSMKEVDEQMLNVQNKNSSYFVEWIPNNVKTAVCDIPPRGVKMAATFVGNSTAIQELFKRISEQFTAMFRRKAFLHWYTGEGMDEMEFTEAESNMNDLVSEYQQYQEATADDEGEFDEHDQDVE

SEQ ID NO: 52 - protein, 7 aa, Caenorhabditis elegans
PARKGKA

SEQ ID NO: 53 - protein, 152 aa, Caenorhabditis elegans
MAPARKGKAKEEQAVVSLGPQAKEGELIFGVAHIFASFNDTFVHITDISGRETIVRVTGGMKVKADRDESSPYAAMLAAQDVADRCKQLGINALHIKLRATGGTRTKTPGPGAQSALRALARAGMKIGRIEDVTPIPSDCTRRKGGRRGRRL

SEQ ID NO: 54 - protein, 151 aa, Caenorhabditis elegans
MGRMHNPGKGMAKSAIPYRRSVPSWQKMTAEEVQDQIVKMAKKGLRPSQIGVILRDSHGVGQVRRLAGNKIFRILKSKGMAPELPEDLYHLVKKAVAIRKHLERSRKDIDSKYRLILVESRIHRLARYYKTKRQLPPTWKYESGTAASLVS

SEQ ID NO: 55 - protein, 17 aa, Caenorhabditis elegans
KKGTHGQEQVTRKKTKK

SEQ ID NO: 56 - protein, 33 aa, Caenorhabditis elegans
RRQQREQAAKIAKDANKAVRAAKAAANKEKKAS

SEQ ID NO: 57 - protein, 4 aa, Caenorhabditis elegans
VGGK

SEQ ID NO: 58 - protein, 159 aa, Caenorhabditis elegans
MKVETCVYSGYKIHPGHGKRLVRTDGKVQIFLSGKALKGAKLRRNPRDIRWTVLYRIKNKKGTHGQEQVTRKKTKKSVQVVNRAVAGLSLDAILAKRNQTEDFRRQQREQAAKIAKDANKAVRAAKAAANKEKKASQPKTQQKTAKNVKTAAPRVGGKR

SEQ ID NO: 59 - protein, 17 aa, Caenorhabditis elegans
KKKKNPRKLRFTKAARR

SEQ ID NO: 60 - protein, 17 aa, Caenorhabditis elegans
KKNPRKLRFTKAARRAR

SEQ ID NO: 61 - protein, 162 aa, Caenorhabditis elegans
MRIEKCYFCSSPIYPGHGIQFVRNDSTVFKFCRSRCNKLFKKKKNPRKLRFTKAARRARGKELINDATQLLEQRRDEPVKYERAMFQKTIEAAKTISALKTKRYGNLIRKRLQPGKIVQKKGLLAKVDKKMHLIRAPVANRDGVKTRAAAKEKKTAESMETN

SEQ ID NO: 62 - protein, 17 aa, Caenorhabditis elegans
RRDYLHYIKKYRRYEKR

SEQ ID NO: 63 - protein, 4 aa, Caenorhabditis elegans
KRHK

SEQ ID NO: 64 - protein, 4 aa, Caenorhabditis elegans
GFSK

SEQ ID NO: 65 - protein, 155 aa, Caenorhabditis elegans
MSEQTERAFLKQPTVNLNNKARILAGSKKTPRYIREVGLGFKAPRDAVEGTYIDKKCPWAGNVPIRGMILTGVVLKNKMTRTIVVRRDYLHYIKKYRRYEKRHKNVPAHCSPAFRDIHPGDLVTIGECRPLSKTVRFNVLKVNKSGTSKKGFSKF

SEQ ID NO: 66 - protein, 821 aa, Caenorhabditis elegans
MTTVRKTYRFCVFSSCLSVSCALVTQVHSSSLPIYSSPFVEKVFLHSSIYVRLCGDMYEQWPTLEFSDLNSSILDLFTKATSQSVASSLLYELTRSDADENGGSIRLNNEEHLKWCMQVLNHSLTLSFATSREYETLKGAVRIYLHWLRALCDTPDNNIPTPLLATPEKYFRNIIDALRWIFCRREDDFDTTVGGQVPRGLAIERQSIEIDMVLDSLKYLTRNSSRKYQDEVWARSISFLLNSSDILLSEPNATEEMGTRTCVRVADTLFDMWLNAVLNEHIPSLTYWSSLATLARRWRHNVPIIECWAKKILGLSTLVCRKMYGDDFLKIDIVDESVLPFENVPMTAEEDENEVHLLYRTWFNMLCLFDSPAKILNHDATRNLCLNGNSPRRTTSSISMSNFELASSSAAQGVSFFLAAVTLQRMVDLFYGDSRVKIDLRNYPVPDGKTAPNTRTASVLTDNHSHHTNRTSSTTGDSSRYVSLGGAVGQIIVDDHQVSMSSGSTASGKTSTATGTSSTHTISSEIRRDQRIMSVNDRSRDPSHRTVSVTDSVNISNQSRYSEQTSSTLTYKSAPIPETANENGHGESISQLVSNSTVSAPVGGAGNDLTLKAGVHPSEMKIGRSSGVIGSAQHNNFYADTTSPYRSAQRFVTNFLTANQATMPYVGGKRPKTDRMLNLVGDWLFAIVNSPTNSPRVTGNDHSGHHKKNNDGVSDVSFISHHFVFTLLSAITTEVISIYICVSMISLTGLNKHHLRIGIIDDETVCTSECPFSPFFAKFTITDGVDFLNNEADSKTTPTSFDFDDFDSFHKFRFQHIYTSK

SEQ ID NO: 67 - protein, 159 aa, Caenorhabditis elegans
MSSLCPYTGRPTGLFRDFEDMMPYWAQRHSMLNNFNNIVPQQLNEVENTAQKFCVKLDVAAFKPEELKVNLEGHVLTIEGHHEVKTEHGFSKRSFTRQFTLPKDVDLAHIHTVINKEGQMTIDAPKTGSNTTVRALPIHTSAGHAVTQKPSSTTTTGKH

SEQ ID NO: 68 - protein, 539 aa, Caenorhabditis elegans
MSSIQCLNPKAELARHAAALELNISGARGLQDVMRSNLGPKGTLKMLVSGAGDIKLTKDGNVLLHEMAIQHPTASMIAKASTAQDDVTGDGTTSTVLLIGELLKQAESLVLEGLHPRIVTEGFEWANTKTLELLEKFKKEAPVERDLLVEVCRTALRTKLHQKLADHITECVVDAVLAIRRDGEEPDLHMVEKMEMHHDSDMDTTLVRGLVLDHGARHPDMPRHVKDAYILTCNVSLEYEKTEVNSGLFYKTAKEREALLAAEREFITRRVHKIIELKKKVIDNSPDGKNKGFVVINQKGIDPPSLDLLASEGILALRRAKRRNMERLQLAVGGEAVNSVDDLTPEDLGWAGLVYEHSLGEEKYTFIEECRAPKSVTLLIKGPNKHTITQIKDAIHDGLRAVFNTIVDKAVLPGAAAFEIAAYVMLKKDVENLKGRAKLGAEAFAQALLVIPKTLAVNGGYDAQETLVKLIEEKTAAGPDIAVGLDLETGGAVEPQGIWDNVTVKKNSISSATVLACNLLLVDEVMRAGMTNLKQPQPE

SEQ ID NO: 69 - protein, 4 aa, Caenorhabditis elegans
VEKR

SEQ ID NO: 70 - protein, 429 aa, Caenorhabditis elegans
MSSIQCLNPKAELARHAAALELNISGARGLQDVMRSNLGPKGTLKMLVSGAGDIKLTKDGNVLLHEMAIQHPTASMIAKASTAQDDVTGDGTTSTVLLIGELLKQAESLVLEGLHPRIVTEGFEWANTKTLELLEKFKKEAPVERDLLVEVCRTALRTKLHQKLADHITECVVDAVLAIRRDGEEPDLHMVEKMEMHHDSDMDTTLVRGLVLDHGARHPDMPRHVKDAYILTCNVSLEYEKTEVNSGLFYKTAKEREALLAAEREFITRRVHKIIELKKKVIDNSPDGKNKGFVVINQKGIDPPSLDLLASEGILALRRAKRRNMERLQLAVGGEAVNSVDDLTPEDLGWAGLVYEHSLGEEKYTFIEECRAPKSVTLLIKGPNKHTITQIKDAIHDGLRAVFNTIVDSCSPWSCCFRNCCLRDVEKRC

SEQ ID NO: 71 - protein, 385 aa, Caenorhabditis elegans; identical to SEQ ID NO: 4

SEQ ID NO: 72 - protein, 1119 aa, Caenorhabditis elegans
MQPTAIRLEDYDKSKLRLPFESPYFPAYFRLLKWKFLDVCVESTRNNDIGYFKLFESLFPPGKLEEIARMIIDEPTPVSHDPDMIKIRNADLDVKIRKQAETYVTLRHAHQQKVQRRRFSECFLNTVLFDEKGLRIADEVMFNYDKELYGYSHWEDLPDGWLTAETFKNKFYDEEEVTNNPFGYQKLDRVAGAARGMIIMKHLKSNPRCVSETTILAFEVFNKGNHQLSTDLVEDLLTEGPAFELKIENGEEKKYAVKKWSLHKTLTMFLAIIGFKSNDKKEKNEHEEWYYGFIDAMKNDPANRAALYFLDKNWPEELEEREKERDRIRLTLLKSQRTNEEAVGEDVCTTIRPQPKDSGYNPDAVVTELVLRTYQEELVQPALEGKNCVIVAPTGSGKTEVAIYAALKHIEERTSQGKPSRVVLLVPKIPLVGQQKDRFLKYCNGMYEVNGFHGSESSVSGTGRRDEVIATHVSVMTPQILINMLQSVRQNERLYVSDFSMMIFDEVHKAAKNHPYVLINQMVQEWKYEKPQIIGLTASLSVKVDGQKDENQMLNDIYNMLALINAPHLSTITRQSSIDELNEHVGKPDDSVELCLPAKENILRDYIERYLNHAHGKFLEELASMSKSTGRNNTIPPNMINTFKKNQPKNYEYYDSLLQGIIQELNKLNVPEKWNSQTWAKYMKVYLEARGIVDLMPAMVAFKYMEKAIGKLNESHSETVEYSTFIKDHDTLKQTIQSVEPEIVLRLKNTLTNQFHVAPESRVIIFVTQRSTAQRVSDFLNESKVLDQFGNYGEQMVGYVLGTNKQGAVQQTSQEQQLTLDKFNNGRLKVIVATSVVEEGLDVTACNLIIKYNCSSGSAIQLVQQRGRARAKNSRSVLLSVKSSINETETNALISEKYMRLCVKKITENGEKQLAAEVKRVAELNAAERKRNLEEQLNLRLRHENKIYKLMCSNCSKEFCKSIYIKKVFSNYMVFDPSVWRFLHVESKRKVSKYLSEDNQPLSDIKCFHCKLDVGRAYKIRGTYLPQLSVKALTFVQESDYSSMTKAKWSDVEQDLFYISEAIEDDFRIMLNALSDTEENIEKKIVLDLDSRQHNKQLEMKRFHIQQEPPTKGVAPEAQ

SEQ ID NO: 73 - protein, 562 aa, Caenorhabditis elegans
MLRELVKKQIIENILRPQNYDSKLGHRKFSVLVLDKSAMVVVNSCLSLNEVFEEGVTLVEDLTRNREPMPSMDAIYIISPVAESIDILINDFSRKTKFNPGNSYRSAHIFFLDPCCDELFEKLSKSPAVKWIKTLKELNLNLKPVESQIFTVNSQFRGDMTKTADGIVSLCATLNIHPTLRFQSDFAQSSEICQRVEQKLKEFGNEGMGTDAELVVLDRSFDLVSPLLHEVTLQAMVVDVTAFKDGVYRYTEAGDSKEIVLDEKDQNWLDLRHKLLPEVMKSVNKMVKDFKNTNKTEPENIKNQSSKDFSTTVRTLQPYLKMKAKMAAYISLTEECRSKYFDSLEKIIALEQDMAVEHTPEHVRITDSQAVGRLSTFILPAIPTETRLRLILIFMLTIGKDKDEQYFNRLLHHTDIPESEFQIIKRMLIWRDKTQKSQFQHRRPPPEDERFIASRWDPKIKNLIEEIYERRLDEREFKVAGKKSTSDFRPAASARYGSGLAGKPREKRKIIIFVVGGITYSEMRVAYELSKKTNTTVILGSDEILTPSSFLESLRDRNTVNC

SEQ ID NO: 74 - protein, 1780 aa, Caenorhabditis elegans
MLPFDNDDSSDDATTSVRPKHPRGVPQSQSTFPRGRSNFSSGTLPNRKTECTPVNTLTIGHSNKMLLTTFRMDRNSKSKSEVDVQEQPVHSSSSAFPGNHLNNFSYPVNRGYLRDYLLQSQRPSTSKPVDCSVLKRHSLPSTHILYEKTKHRGGVNIEEQEKLVRMLWAAAEESETVAKTRQFSKKQAIELNFDAKLIGSMNNDCFGYCRAHMENIKDVLKTHLKLSKVDEVNWIKVGMVPRAAYEDKSYVIDAHLVLTPNGEVEDENELFSEFASSFTSRITGMLHDQVFLEVPKMHTLFTKITPQHMDINISAIAIGNCPNSGLFLVRGDFISQENTVCSVKLQSHHNADASRENSSFKVAGSNKYLSYARFEHDKRLAVVYFGVRLAEFADDGLDHAGFRLNLYYNLFVRIVVDMSHETTNSIYIQMKNPPHLWEGIPKNTIFHPSKSKVLNMETCTEWTRVLSWPGDAEGRGVGCTSEAFSQSSWIRLTMRKDDDNDSVSSTQLMDIVTRLSARSKAKVMFGSIFSIRRKLAPSPAFHSLGSFRANYALQALITRGSVFTDQLFDATDENIPSSDNDNDEDDDDDVDDTKKPMELVHEPLFLKLVRRGMKECSQATEETLEQLLNAFDERRQIDVVTAFTTMYQSRKIQYERLLKGESLQDVGLAKPLPKNCVSVAKVIVTPSRILLMAPEVMMVNRVVRRFGPDYALRCVFRDDNLGRLAIRDFSINNIDHMSNIVTEGIYLTLKNGIQVADRVYSFLGWSNSQMRDQGCYLYAPRVNALTGEVTGTVEDIRVWMGDFRDAISVPKMMSRMGQCFTQAQPTVYSSVKNIHIVENIQVRLERHHWIVEPDIEGGVENKYCFSDGCGRISIKLATHISKILQLKEVPACFQVRFKGFKGILVIDPTIDDIINMPKVIFRKSQQKFGEGGGELQDEYLEVVKYAMPSPVCLNRPFITILDQVSEKQSASSHRRITNRVHYYLERELCSLSNMLINENQAAEELVNRTNLAIDWNAASKRAGFELSVDPLIRDMLFSIYRYNIIHHISKAKIFLPPSLGRSMYGVVDETGLLQYGQVFIQYSPSIRQTSNRPILKTGKVLITKNPCHVPGDVRVFDAVWQPALAHLVDVVVFPQHGPRPHPDEMAGSDLDGDEYSIIWDQEMLLDYNEEAMVFPSSSAAEEDKEPTTDDMVEFFLRYLQQDSIGRMSHAHLAYADLHGLFHENCHAIALKCAVAVDFPKSGVPAEPLSSFEQCEMTPDYMMSGGKPMYYSTRLNGQLHRKARKVEEVLEEFETRGSVFEREYDKLICPEDVDVFFGNEIKLVQTLTLRDEYVDRMQQLLDEYGIEDEASVVSGHAASIKRLAGMERDDYSFYHTDKVVELRYEKLYAVFRAKFFEEFGGEEINIENDGKNTRLKCTKAMHEKIRQWYFVAYVQPKINKAGRCIGQSLPWVAWDALCDLRRQLMLDKNDAVLRGKYPIAARLEEEIENSIERQFDKFLKLKDLIESHKDALFLRRYVYFYGDQIIKMLFILKVWLERENVLPSSVLSIWQLGRLLIRLGLGDLLGNPTIDYEKSLLMPTTMFQQWISKKEDADEAPILRNFDMGTMMLEFLRYLASQSFASAESISLRVFYEKDIVEPILTKSAQWMPLHLIAYRTFHSIAVSGRFDALHLDDEDAVDQITESKDPILVNESLFSSRNYNDDYPISRSRILQSLKDWSGVKEIIPREITGTRKSDMIYVTSVGTVLARQRLARLLLLSGETIRDAIANNVVPNEVRDEFL

SEQ ID NO: 75 - protein, 4 aa, Caenorhabditis elegans
KKKK

SEQ ID NO: 76 - protein, 4 aa, Caenorhabditis elegans
ILPK

SEQ ID NO: 77 - protein, 7 aa, Caenorhabditis elegans
PKNPKKR

SEQ ID NO: 78 - protein, 7 aa, Caenorhabditis elegans
PKKRVEI

SEQ ID NO: 79 - protein, 578 aa, Caenorhabditis elegans
MQPVLVNSRPLRVKSHESESKLNLIEQEDQFEGANYSSSSGVIICYSNGTGEVITQEAFDDSGIHFIFSKATCIQYPSNFDPIGVGSVVQIFWSRSFERVVRGNHIIVQIEKMEVYKCCAMLREQVFVTFNSPSTAGVAIGVTERNITVAFHPNCSPVIRYETLKAHSIGRTEFEIKDRHRENTNRMVDVILAAVPFRVEIHGNVDKIPFFVIEKCRNSPGRSGAAVITKIMKNHFMEANFLQNSESIYFDSTSCHSNILEKVSIGSLINVLADPTFATSSYKWYGYDVTLCNNYLAHASTQRSFVLENNEILQNCKKLEKSPEEAETTTKNDLRFVPPQPEKGEVKKKKMTNCLKFNSKSAQFKLRHLILDRCFSELPEREAKSIINSYFIDRLAEGIKIEKIDKNWRTFGEILPKTPKKYSESLKKSIQNVLEPFGLNKPEKAAETPKIVEYFPKNPKKRVEIVEKPTVDEIRELFGALMDAEGFALNQRVKPHFVLPDTRWKPTERRYIGIYDDVQWTFMSTFCPKIEENSENRPLAGGWWYRRTVPRDHPVEIVQKMETRRNIIKDCTESPFIE

SEQ ID NO: 80 - protein, 4 aa, Caenorhabditis elegans
DKDS

SEQ ID NO: 81 - protein, 458 aa, Caenorhabditis elegans
MAMAPLRPRVFARCLILKNLELIEAARIFFIDSAVTANVSWKCLFQIDENLKFHPWQAMHCTLGRLVHLSDSWTDTQCTEFRNIVSKFAKFQITANQCDVDFRSDRPSLLVNLYGLPNGTEIDKKVAIEEICAVSMQNVMVSQFPTNFMVNPKLEELDKEQDHLDVILLEEFRRDLPADWAHEPPADYREDDADWDILQCHVAEWNDTALEQFRRADGSFWAMLEPSCTVSPWEMHVTPILAPEKMSDNEHWIFEQLVKNSENQQKIDDFYSNLKNQRPLEMEEIKFALQTGRTYVMATIKNRQKSSAQWLRCEIIDFLPNANVALRYVDLGTRGILKLKNLHRMHIEHTKIAPACIEIGRFLDDDLSMADSEMEWNTHFWREIVPYDVPIVVGPDMEFLETGKLQFSQIRVAGDEDDENLLDKIPSPSPFFTERSDDLRTQKEDDDDGNVSDDKDSG

SEQ ID NO: 82 - protein, 261 aa, Caenorhabditis elegans; identical to SEQ ID NO: 35

SEQ ID NO: 83 - protein, 346 aa, Caenorhabditis elegans
MQADGEKKKKKTNPERSTHDDTPKSRTRVLFSQYFFLSFSLFFRAIFMLRSLCSIAVRLGGARQPRLLSSAASGDGNDGKGAKDAIDEDLLNAIEGVANNIHPQNGSEKKSLKNTLINRLVANEKASFDAAAASASSEMLDDQALIGLLADVAGDAKVEKKLPPKSAQLRQEKRGLVLLRKEIFYQAVQSGFTTEEARVKSETIVNEAQIKLQEQRKALLNDVREKVEQEEVEETERSEKDQKLFTMALEFMEKIYKDDLISSAVQFPTAHSDQQILSKNKSNGQQKENNGNIQSIMSSKWAMNRMFHSLITYSWRDIYHHWVSRNLVQLLILCIWFVLVYPRIHI

SEQ ID NO: 84 - protein, 1025 aa, Homo sapiens
MSNGYSTDENFRYLISCFRARVKMYIQVEPVLDYLTFLPAEVKEQIQRTVATSGNMQAVELLLSTLEKGVWHLGWTREFVEALRRTGSPLAARYMNPELTDLPSPSFENAHDEYLQLLNLLQPTLVDKLLVRDVLDKCMEEELLTIEDRNRIAAAENNGNESGVRELLKRIVQKENWFSAFLNVLRQTGNNELVQELTGSDCSESNAEIENLSQVDGPQVEEQLLSTTVQPNLEKEVWGMENNSSESSFADSSVVSESDTSLAEGSVSCLDESLGHNSNMGSDSGTMGSDSDEENVAARASPEPELQLRPYQMEVAQPALEGKNIIICLPTGSGKTRVAVYIAKDHLDKKKKASEPGKVIVLVNKVLLVEQLFRKEFQPFLKKWYRVIGLSGDTQLKISFPEVVKSCDIIISTAQILENSLLNLENGEDAGVQLSDFSLIIIDECHHTNKEAVYNNIMRHYLMQKLKNNRLKKENKPVIPLPQILGLTASPGVGGATKQAKAEEHILKLCANLDAFTIKTVKENLDQLKNQIQEPCKKFAIADATREDPFKEKLLEIMTRIQTYCQMSPMSDFGTQPYEQWAIQMEKKAAKEGNRKERVCAEHLRKYNEALQINDTIRMIDAYTHLETFYNEEKDKKFAVIEDDSDEGGDDEYCDGDEDEDDLKKPLKLDETDRFLMTLFFENNKMLKRLAENPEYENEKLTKLRNTIMEQYTRTEESARGIIFTKTRQSAYALSQWITENEKFAEVGVKAHHLIGAGHSSEFKPMTQNEQKEVISKFRTGKINLLIATTVAEEGLDIKECNIVIRYGLVTNEIAMVQARGRARADESTYVLVAHSGSGVIEHETVNDFREKMMYKAIHCVQNMKPEEYAHKILELQMQSIMEKKMKTKRNIAKHYKNNPSLITFLCKNCSVLACSGEDIHVIEKMHHVNMTPEFKELYIVRENKALQKKCADYQINGEIICKCGQAWGTMMVHKGLDLPCLKIRNFVVVFKNNSTKKQYKKWVELPITFPNLDYSECCLFSDED

SEQ ID NO: 85 - protein, 119 aa, Homo sapiens
MKLVRFLMKLSHETVTIELKNGTQVHGTITGVDVSMNTHLKAVKMTLKNREPVQLETLSIRGNNIRYFILPDSLPLDTLLVDVEPKVKSKKREAVAGRGRGRGRGRGRGRGRGRGGPRR

SEQ ID NO: 86 - protein, 8 aa, Homo sapiens
FKVYRYLQ

SEQ ID NO: 87 - protein, 16 aa, Homo sapiens
LILIYSRHLFFYESKC

SEQ ID NO: 88 - protein, 744 aa, Homo sapiens
MASEGTNIPSPVVRQIDKQFLICSICLERYKNPKVLPCLHTFCERCLQNYIPAHSLTLSCPVCRQTSILPEKGVAALQNNFFITNLMDVLQRTPGSNAEESSILETVTAVAAGKPLSCPNHDGNVMEFYCQSCETAMCRECTEGEHAEHPTVPLKDVVEQHKASLQVQLDAVNKRLPEIDSALQFISEIIHQLTNQKASIVDDIHSTFDELQKTLNVRKSVLLMELEVNYGLKHKVLQSQLDTLLQGQESIKSCSNFTAQALNHGTETEVLLVKKQMSEKLNELADQDFPLHPRENDQLDFIVETEGLKKSIHNLGTILTTNAVASETVATGEGLRQTIIGQPMSVTITTKDKDGELCKTGNAYLTAELSTPDGSVADGEILDNKNGTYEFLYTVQKEGDFTLSLRLYDQHIRGSPFKLKVIRSADVSPTTEGVKRRVKSPGSGHVKQKAVKRPASMYSTGKRKENPIEDDLIFRVGTKGRNKGEFTNLQGVAASTNGKILIADSNNQCVQIFSNDGQFKSRFGIRGRSPGQLQRPTGVAVHPSGDIIIADYDNKWVSIFSSDGKFKTKIGSGKLMGPKGVSVDRNGHIIVVDNKACCVFIFQPNGKIVTRFGSRGNGDRQFAGPHFAAVNSNNEIIITDFHNHSVKVFNQEGEFMLKFGSNGEGNGQFNAPTGVAVDSNGNIIVADWGNSRIQVFDGSGSFLSYINTSADPLYGPQGLALTSDGHVVVADSGNHCFKVYRYLQ

SEQ ID NO: 89 - protein, 677 aa, Homo sapiens
MAEEKKLKLSNTVLPSESMKVVAESMGIAQIQEETCQLLTDEVSYRIKEIAQDALKFMHMGKRQKLTTSDIDYALKLKNVEPLYGFHAQEFIPFRFASGGGRELYFYEEKEVDLSDIINTPLPRVPLDVCLKAHWLSIEGCQPAIPENPPPAPKEQQKAEATEPLKSAKPGQEEDGPLKGKGQGATTADGKGKEKKAPPLLEGAPLRLKPRSIHELSVEQQLYYKEITEACVGSCEAKRAEALQSIATDPGLYQMLPRFSTFISEGVRVNVVQNNLALLIYLMRMVKALMDNPTLYLEKYVHELIPAVMTCIVSRQLCLRPDVDNHWALRDFAARLVAQICKHFSTTTNNIQSRITKTFTKSWVDEKTPWTTRYGSIAGLAELGHDVIKTLILPRLQQEGERIRSVLDGPVLSNIDRIGADHVQSLLLKHCAPVLAKLRPPPDNQDAYRAEFGSLGPLLCSQVVKARAQAALQAQQVNRTTLTITQPRPTLTLSQAPQPGPRTPGLLKVPGSIALPVQTLVSARAAAPPQPSPPPTKFIVMSSSSSAPSTQQVLSLSTSAPGSGSTTTSPVTTTVPSVQPIVKLVSTATTAPPSTAPSGPGSVQKYIVVSLPPTGEGKGGPTSHPSPVPPPASSPSPLSGSALCGGKQEAGDSPPPAPGTPKANGSQPNSGSPQPAP

SEQ ID NO: 90 - protein, 403 aa, Homo sapiens
MSHRKFSAPRHGSLGFLPRKRSSRHRGKVKSFPKDDPSKPVHLTAFLGYKAGMTHIVREVDRPGSKVNKKEVVEAVTIVETPPMVVVGIVGYVETPRGLRTFKTVFAEHISDECKRRFYKNWHKSKKKAFTKYCKKWQDEDGKKQLEKDFSSMKKYCQVIRVIAHTQMRLLPLRQKKAHLMEIQVNGGTVAEKLDWARERLEQQVPVNQVFGQDEMIDVIGVTKGKGYKGVTSRWHTKKLPRKTHRGLRKVACIGAWHPARVAFSVARAGQKGYHHRTEINKKIYKIGQGYLIKDGKLIKNNASTDYDLSDKSINPLGGFVHYGEVTNDFVMLKGCVVGTKKRVLTLRKSLLVQTKRRALEKIDLKFIDTTSKFGHGRFQTMEEKKAFMGPLKKDRIAKEEGA

SEQ ID NO: 91 - protein, 349 aa, Homo sapiens
MEDPQSKEPAGEAVALALLESPRPEGGEEPPRPSPEETQQCKFDGQETKGSKFITSSASDFSDPVYKEIAITNGCINRMSKEELRAKLSEFKLETRGVKDVLKKRLKNYYKKQKLMLKESNFADSYYDYICIIDFEATCEEGNPPEFVHEIIEFPVVLLNTHTLEIEDTFQQYVRPEINTQLSDFCISLTGITQDQVDRADTFPQVLKKVIDWMKLKELGTKYKYSLLTDGSWDMSKFLNIQCQLSRLKYPPFAKKWINIRKSYGNFYKVPRSQTKLTIMLEKLGMDYDGRPHCGLDDSKNIARIAVRMLQDGCELRINEKMHAGQLMSVSSSLPIEGTPPPQMPHFRK

SEQ ID NO: 92 - protein, 5 aa, Homo sapiens
KKKKK

SEQ ID NO: 93 - protein, 777 aa, Homo sapiens
MEQYCSIKIVDILEEEVVTFAVEVELPNSGKLLDHVLIEMGYGLKPSGQDSKKENADQSDPEDVGKMTTENNIVVDKSDLIPKVLTLNVGDEFCGVVAHIQTPEDFFCQQLQSGRKLAELQASLSKYCDQLPPRSDFYPAIGDICCAQFSEDDQWYRASVLAYASEESVLVGYVDYGNFEILSLMRLCPIIPKLLELPMQAIKCVLAGVKPSLGIWTPEAICLMKKLVQNKIITVKVVDKLENSSLVELIDKSETPHVSVSKVLLDAGFAVGEQSMVTDKPSDVKETSVPLGVEGKVNPLEWTWVELGVDQTVDVVVCVIYSPGEFYCHVLKEDALKKLNDLNKSLAEHCQQKLPNGFKAEIGQPCCAFFAGDGSWYRALVKEILPNGHVKVHFVDYGNIEEVTADELRMISSTFLNLPFQGIRCQLADIQSRNKHWSEEAITRFQMCVAGIKLQARVVEVTENGIGVELTDLSTCYPRIISDVLIDEHLVLKSASPHKDLPNDRLVNKHELQVHVQGLQATSSAEQWKTIELPVDKTIQANVLEIISPNLFYALPKGMPENQEKLCMLTAELLEYCNAPKSRPPYRPRIGDACCAKYTSDDFWYRAVVLGTSDTDVEVLYADYGNIETLPLCRVQPITSSHLALPFQIIRCSLEGLMELNGSSSQLIIMLLKNFMLNQNVMLSVKGITKNVHTVSVEKCSENGTVDVADKLVTFGLAKNITPQRQSALNTEKMYRTNCCCTELQKQVEKHEHILLFLLNNSTNQNKFIEMKKLVKS

SEQ ID NO: 94 - protein, 330 aa, Homo sapiens
MSQWHHPRSGWGRRRDFSGRSSAKKKGGNHIPERWKDYLPVGQRMPGTRFIAFKVPLQKSFEKKLAPEECFSPLDLFNKIREQNEELGLIIDLTYTQRYYKPEDLPETVPYLKIFTVGHQVPDDETIFKFKHAVNGFLKENKDNDKLIGVHCTHGLNRTGYLICRYLIDVEGVRPDDAIELFNRCRGHCLERQNYIEDLQNGPIRKNWNSSVPRSSDFEDSAHLMQPVHNKPVKQGPRYNLHQIQGHSAPRHFHTQTQSLQQSVRKFSENPHVYQRHHLPPPGPPGEDYSHRRYSWNVKPNASRAAQDRRRWYPYNYSRLSYPACWEWTQ

SEQ ID NO: 95 - protein, 29 aa, Caenorhabditis elegans
PEKLMEQLKKLESAMDSVIETASDLVSLS

SEQ ID NO: 96 - protein, 7 aa, Caenorhabditis elegans
PEMKKIK

SEQ ID NO: 97 - protein, 7 aa, Caenorhabditis elegans
PKRRKFE

SEQ ID NO: 98 - protein, 7 aa, Caenorhabditis elegans
PLNKRKD

SEQ ID NO: 99 - protein, 7 aa, Caenorhabditis elegans
PRRSRTV

SEQ ID NO: 100 - protein, 29 aa, Caenorhabditis elegans
IQQLRDLNQKSIEDQERETRENDKIDDGE

SEQ ID NO: 101 - protein, 36 aa, Caenorhabditis elegans
PKQLTKEEEQFKKLQNDLLKQAKERLEALEMSEDME

SEQ ID NO: 102 - protein, 4 aa, Caenorhabditis elegans
KPRR

SEQ ID NO: 103 - protein, 1845 aa, Caenorhabditis elegans
MVRVRADLQCFNPRDYQVELLDKATKKNTIVQLGTGSGKTFIAVLLLKEYGVQLFAPLDQGGKRAFFVVEKVNLVEQQAIHIEVHTSFKVGQVHGQTSSGLWDSKEQCDQFMKRHHVVVITAQCLLDLIRHAYLKIEDMCVLIFDECHHALGSQHPYRSIMVDYKLLKKDKPVPRVLGLTASLIKAKVAPEKLMEQLKKLESAMDSVIETASDLVSLSKYGAKPYEVVIICKDFEIGCLGIPNFDTVIEIFDETVAFVNTTTEFHPDLDLDPRRPIKDSLKTTRAVFRQLGPWAAWRTAQVWEKELGKIIKSQVLPDKTLRFLNMAKTSMITIKRLLEPEMKKIKSIEALRPYVPQRVIRLFEILETFNPEFQKERMKLEKAEHLSAIIFVDQRYIAYSLLLMMRHIKSWEPKFKFVNPDYVVGASGRNLASSDSQGLHKRQTEVLRRFHRNEINCLIATSVLEEGVDVKQCNLVIKFDRPLDMRSYVQSKGRARRAGSRYVITVEEKDTAAYCSKLPSDIFTRLVPHNQIIPIEENGVTKYCAELLLPINSPIKHAIVLKNPMPNKKTAQMAVALEACRQLHLEGELDDNLLPKGRESIAKLLEHIDEEPDEYAPGIAAKVGSSKRKQLYDKKIARALNESFVEADKECFIYAFELERFREAELTLNPKRRKFEDPFNYEYCFGFLSAKEIPKIPPFPVFLRQGNMKVRLIVAPKKTTVTAAQLQEIQLFHNYLFTQVLQMCKTGNLEFDGTSNAPLNTLIVPLNKRKDDMSYTINMKYVSEVVANMENMPRIPKDEVRRQYKFNAEDYKDAIVMPWYRNLEQPVFYYVAEILPEWRPSSKFPDTHFETFNEYFIKKYKLEIYDQNQSLLDVDFTSTRLNLLQPRIQNQPRRSRTVSNSSTSNIPQASASDSKESNTSVPHSSQRQILVPELMDIHPISATLWNVIAALPSIFYRVNQLLLTDELRETILVKAFGKEKTKLDDNVEWNSLAYATEYEEKQTIIVKKIQQLRDLNQKSIEDQERETRENDKIDDGEELFNIGVWDPEEAVRIGVEISSRDDRMDGEDQDTVGLTQGLHDGNISDEDDELPFVMHDYTARLTSNRNGIGAWSGSESIVPSGWGDWDGPEPDNSPMPFQILGGPGGLNVQALMADVGRVFDPSTASSSLSQTVQESTVSPPKQLTKEEEQFKKLQNDLLKQAKERLEALEMSEDMEKPRRLEDTVNLEDYGDDQENQEDENTPTNFPKTIDEEIEELSIGARKKQEIDDNAAKTDVLERENCEVLPVAINEKSRSFSFEKESKAINGRLIRQRSEEYVSHIDSDIGLGVSPCLLLTALTTSNAADGMSLERFETIGDSFLKFATTDYLYHTLLDQHEGKLSFARSKEVSNCNLYRLGKKLGIPQLIVANKFDAHDSWLPPCYIPTCDFKAPNTDDAEEKDNEIERILDGQVIEEKPENKTGWDIGGDVSKSTTDGIETITFPKQARVGNDDISPLPYNLLTQQHISDKSIADAVEALIGVHLLTLGPNPTLKVMNWMGLKVIQKDQKSDVPSPLLRFIDTPTNPNASLNFLNNLWQQFQFTQLEEKIGYRFKERAYLVQAFTHASYINNRVTGCYQRLEFLGDAVLDYMITRYLFEDSRQYSPGVLTDLRSALVNNTIFASLAVKFEFQKHFIAMCPGLYHMIEKFVKLCSERNFDTNFNAEMYMVTTEEEIDEGQEEDIEVPKAMGDIFESVAGAIYLDSGRNLDTTWQVIFHMMRGTIELCCANPPRSPIRELMEFEQSKVRFSKMERILESGKVRVTVEVVNNMRFTGMGRNYRIAKATAAKRALKYLHQIEQQRRQSPSLTTV

SEQ ID NO: 104 - protein, 1922 aa, Homo sapiens
MKSPALQPLSMAGLQLMTPASSPMGPFFGLPWQQEAIHDNIYTPRKYQVELLEAALDHNTIVCLNTGSGKTFIAVLLTKELSYQIRGDFSRNGKRTVFLVNSANQVAQQVSAVRTHSDLKVGEYSNLEVNASWTKERWNQEFTKHQVLIMTCYVALNVLKNGYLSLSDINLLVFDECHLAILDHPYREIMKLCENCPSCPRILGLTASILNGKCDPEELEEKIQKLEKILKSNAETATDLVVLDRYTSQPCEIVVDCGPFTDRSGLYERLLMELEEALNFINDCNISVHSKERDSTLISKQILSDCRAVLVVLGPWCADKVAGMMVRELQKYIKHEQEELHRKFLLFTDTFLRKIHALCEEHFSPASLDLKFVTPKVIKLLEILRKYKPYERQQFESVEWYNNRNQDNYVSWSDSEDDDEDEEIEEKEKPETNFPSPFTNILCGIIFVERRYTAVVLNRLIKEAGKQDPELAYISSNFITGHGIGKNQPRNKQMEAEFRKQEEVLRKFRAHETNLLIATSIVEEGVDIPKCNLVVRFDLPTEYRSYVQSKGRARAPISNYIMLADTDKIKSFEEDLKTYKAIEKILRNKCSKSVDTGETDIDPVMDDDDVFPPYVLRPDDGGPRVTINTAIGHINRYCARLPSDPFTHLAPKCRTRELPDGTFYSTLYLPINSPLRASIVGPPMSCVRLAERVVALICCEKLHKIGELDDHLMPVGKETVKYEEELDLHDEEETSVPGRPGSTKRRQCYPKAIPECLRDSYPRPDQPCYLYVIGMVLTTPLPDELNFRRRKLYPPEDTTRCFGILTAKPIPQIPHFPVYTRSGEVTISIELKKSGFMLSLQMLELITRLHQYIFSHILRLEKPALEFKPTDADSAYCVLPLNVVNDSSTLDIDFKFMEDIEKSEARIGIPSTKYTKETPFVFKLEDYQDAVIIPRYRNFDQPHRFYVADVYTDLTPLSKFPSPEYETFAEYYKTKYNLDLTNLNQPLLDVDHTSSRLNLLTPRHLNQKGKALPLSSAEKRKAKWESLQNKQILVPELCAIHPIPASLWRKAVCLPSILYRLHCLLTAEELRAQTASDAGVGVRSLPADFRYPNLDFGWKKSIDSKSFISISNSSSAENDNYCKHSTIVPENAAHQGANRTSSLENHDQMSVNCRTLLSESPGKLHVEVSADLTAINGLSYNQNLANGSYDLANRDFCQGNQLNYYKQEIPVQPTTSYSIQNLYSYENQPQPSDECTLLSNKYLDGNANKSTSDGSPVMAVMPGTTDTIQVLKGRMDSEQSPSIGYSSRTLGPNPGLILQALTLSNASDGFNLERLEMLGDSFLKHAITTYLFCTYPDAHEGRLSYMRSKKVSNCNLYRLGKKKGLPSRMVVSIFDPPVNWLPPGYVVNQDKSNTDKWEKDEMTKDCMLANGKLDEDYEEEDEEEESLMWRAPKEEADYEDDFLEYDQEHIRFIDNMLMGSGAFVKKISLSPFSTTDSAYEWKMPKKSSLGSMPFSSDFEDFDYSSWDAMCYLDPSKAVEEDDFVVGFWNPSEENCGVDTGKQSISYDLHTEQCIADKSIADCVEALLGCYLTSCGERAAQLFLCSLGLKVLPVIKRTDREKALCPTRENFNSQQKNLSVSCAAASVASSRSSVLKDSEYGCLKIPPRCMFDHPDADKTLNHLISGFENFEKKINYRFKNKAYLLQAFTHASYHYNTITDCYQRLEFLGDAILDYLITKHLYEDPRQHSPGVLTDLRSALVNNTIFASLAVKYDYHKYFKAVSPELFHVIDDFVQFQLEKNEMQGMDSELRRSEEDEEKEEDIEVPKAMGDIFESLAGAIYMDSGMSLETVWQVYYPMMRPLIEKFSANVPRSPVRELLEMEPETAKFSPAERTYDGKVRVTVEVVGKGKFKGVGRSYRIAKSAAARRALRSLKANQPQVPNS

SEQ ID NO: 105 - protein, 1917 aa, Mus musculus
MNEKPCFAALSMAGLQLMTPASSPMGPFFGLPWQQEAIHDNIYTPRKYQVELLEAALDHNTIVCLNTGSGKTFIAVLLTKELAHQIRGDLNPHAKRTVFLVNSANQVCQQVSAVRTHSDLKVGEYSDLEVNASWTKERWSQEFTKHQVLIMTCYVALTVLKNGYLSLSDINLLVFDECHLAILDHPYREIMKLCESCPSCPRILGLTASILNGKCDPEELEEKIQKLERILRSDAETATDLVVLDRYTSQPCEIVVDCGPFTDRSGLYERLLMELEAALDFINDCNVAVYSKERDSTLISKQILSDCRAVLVVLGPWCADKVAGMMVRELQKYIKHEQEELHRKFLLFTDTLLRKIHALCEEYFSPASLDLKYVTPKVMKLLEILRKYKPYERQQFESVEWYNNRNQDNYVSWSDSEDDDDDEEIEEKEKPETNFPSPFTNILCGIIFVERRYTAVVLNRLIKEAGKQDPELAYISSNFITGHGIGKNQPRSKQMEAEFRKQEEVLRKFRAHETNLLIATSVVEEGVDIPKCNLVVRFDLPTEYRSYVQSKGRARAPISNYVMLADTDKIKSFEEDLKTYKAIEKILRNKCSKSADGAEADVHAGVDDEDAFPPYVLRPDDGGPRVTINTAIGHINRYCARLPSDPFTHLAPKCRTRELPDGTFYSTLYLPINSPLRASIVGPPMDSVRLAERVVALICCEKLHKIGELDEHLMPVGKETVKYEEELDLHDEEETSVPGRPGSTKRRQCYPKAIPECLRDSYPKPDQPCYLYVIGMVLTTPLPDELNFRRRKLYPPEDTTRCFGILTAKPIPQIPHFPVYTRSGEVTISIELKKSGFILSQQMLELITRLHQYIFSHILRLEKPALEFKPTGAESAYCVLPLNVVNDSGTLDIDFKFMEDIEKSEARIGIPSTKYSKETPFVFKLEDYQDAVIIPRYRNFDQPHRFYVADVYTDLTPLSKFPSPEYETFAEYYKTKYNLDLTNLNQPLLDVDHTSSRLNLLTPRHLNQKGKALPLSSAEKRKAKWESLQNKQILVPELCAIHPIPASLWRKAVCLPSILYRLHCLLTAEELRAQTASDAGVGVRSLPVDFRYPNLDFGWKKSIDSKSFISTCNSSLAESDNYCKHSTTVVPEHAAHQGATRPSLENHDQMSVNCKRLPAESPAKLQSEVSTDLTAINGLSYNKNLANGSYDLVNRDFCQGNQLNYFKQEIPVQPTTSYPIQNLYNYENQPKPSNECPLLSNTYLDGNANTSTSDGSPAVSTMPAMMNAVKALKDRMDSEQSPSVGYSSRTLGPNPGLILQALTLSNASDGFNLERLEMLGDSFLKHAITTYLFCTYPDAHEGRLSYMRSKKVSNCNLYRLGKKKGLPSRMVVSIFDPPVNWLPPGYVVNQDKSNSEKWEKDEMTKDCLLANGKLGEACEEEEDLTWRAPKEEAEDEDDFLEYDQEHIQFIDSMLMGSGAFVRKISLSPFSASDSAYEWKMPKKASLGSMPFASGLEDFDYSSWDAMCYLDPSKAVEEDDFVVGFWNPSEENCGVDTGKQSISYDLHTEQCIADKSIADCVEALLGCYLTSCGERAAQLFLCSLGLKVLPVIKRTSREKALDPAQENGSSQQKSLSGSCAAPVGPRSSAGKDLEYGCLKIPPRCMFDHPDAEKTLNHLISGFETFEKKINYRFKNKAYLLQAFTHASYHYNTITDCYQRLEFLGDAILDYLITKHLYEDPRQHSPGVLTDLRSALVNNTIFASLAVKYDYHKYFKAVSPELFHVIDDFVKFQLEKNEMQGMDSELRRSEEDEEKEEDIEVPKAMGDIFESLAGAIYMDSGMSLEVVWQVYYPMMQPLIEKFSANVPRSPVRELLEMEPETAKFSPAERTYDGKVRVTVEVVGKGKFKGVGRSYRIAKSAAARRALRSLKANQPQVPNS

SEQ ID NO: 106 - DNA, 33 bp, Caenorhabditis elegans
tcgccggcgggctcctcgccacttaaaatgtca

SEQ ID NO: 107 - protein, 56 aa, Caenorhabditis elegans
VYRRCRIVDVCRNNELLKVFFIDDAVIAWVQPECLGELDQHYMYYPWQAIQVSMFG

SEQ ID NO: 108 - protein, 53 aa, Caenorhabditis elegans
RCLILKNLELIEAARIFFIDSAVTANVSWKCLFQIDENLKFHPWQAMHCTLGR

SEQ ID NO: 109 - protein, 84 aa, Caenorhabditis elegans
FGNYGEQMVGYVLGTNKQGAVQQTSQEQQLTLDKFNNGRLKVIVATSVVEEGLDVTACNLIIEYNCSSGSAIQLVQQRGRARAK

SEQ ID NO: 110 - protein, 87 aa, Caenorhabditis elegans
ELLMLGIKSEWMSGLNKSTASSADISASKQKQMEKLKMFADGEIRILVSTSVAEEGLDVPECSLVIKYNYATNEIAHVQRRGRGRAL

SEQ ID NO: 111 - protein, 87 aa, Caenorhabditis elegans
ELAYISSNGITGHGIGKNQPRNKQMEAEFRKQEEVLRKFRAHETNLLIATSIVEEGVDIPKCHLVVRFDLPTEYRSYVQSKGRARAP

SEQ ID NO: 112 - protein, 77 aa, Caenorhabditis elegans
VVGASGRNLASSDSQGLHKRQTEVLRRFHRNEINCLIATSVLEEGVDVKQCNLVIKFDRPLDMRSYVQSKGRARRAG

(19) United States
(12) Patent Application Publication
(10) Pub. No.: US 2007/0031417 A2
(43) Pub. Date: Feb. 8, 2007

(54) DICER INTERACTING PROTEINS AND USES THEREFOR

(75) Inventor: Craig MELLO, Shrewsbury, MA (US)

Correspondence Address: LAHIVE & COCKFIELD, LLP, ONE POST OFFICE SQUARE, BOSTON, MA, 02109-2127, US

(73) Assignee: UNIVERSITY OF MASSACHUSETTS, Boston, MA (US)

(21) Appl. No.: 11/107,336

(22) Filed: Apr. 14, 2005

Related U.S. Application Data
(60) Provisional application No. 60/562,420, filed on Apr. 14, 2004.

(51) Int. Cl. A61K 48/00; G01N 33/53; C12Q 1/37; A61K 39/395; C07K 16/40

(52) U.S. Cl. 424/146.1; 435/7.1; 435/23; 514/44; 530/388.26

(57) ABSTRACT

Abstract of the Disclosure

ABSTRACT Dicer (e.g., DCR-1) interactors are disclosed as are methods to positively or negatively modulate Dicer activity. Uses of Dicer interactors as drug targets are featured. Also featured are uses of Dicer interactors and modulators of same to modulate various Dicer functions in vitro, in cell cultures, and in vivo.

### Detailed Description of the Invention

### CROSS REFERENCE TO RELATED APPLICATIONS

This application claims the benefit of prior-filed provisional patent application Serial No. 60/562,420, filed April 14, 2004, entitled "DICERINTERACTING PROTEINS AND USES THEREFOR." The contents of any patents, patent applications, references, and appendices cited throughout this specification are hereby incorporated by reference in their entireties.

### STATEMENT REGARDING FEDERALLY SPONSORED RESEARCH OR DEVELOPMENT

Funding for the work described herein was, at least in part, supported by grants from the National Institutes of Health (R01 GM058800; R21 ES012021-02).

### BACKGROUND OF THE INVENTION

RNA-mediated gene silencing phenomena, known as post-transcriptional gene silencing in plants, quelling in fungi, and RNA interference (RNAi) in animals, are mediated by double-stranded RNA (dsRNA) and mechanistically intersect at the ribonuclease Dicer. Dicer is an RNase III-family enzyme characterized by its ribonuclease activity and dsRNA-binding properties. The enzyme generates nucleotide products from dsRNA of approximately 21-23. Processing of microRNAs, for example the let-7 precursor, by Dicer has also been observed. Dicer includes a dsRNA-binding domain located at the C-terminus of the enzyme.

Given the important role of Dicer in the generation of RNA-mediated gene silencing agents, the identification of proteins that interact with and/or regulate Dicer will help improve our understanding of RNA silencing and other Dicer-related processes. Moreover, Dicer-interacting and/or Dicer-regulating proteins are useful for the identification of a variety of modulatory agents for use in regulating RNA-mediated gene silencing.

### BRIEF SUMMARY OF THE INVENTION

Important in the RNAi pathway of most organisms is the ribonuclease III enzyme Dicer. In particular, Dicer has been shown to play a key role in the processing of RNA precursors triggering the activation of both endogenous and exogenous pathogen responses (i.e., RNAi) and of small RNAs active as developmental regulators called microRNAs. The enzyme and its ancillary components have been poorly characterized to date. The instant invention is based, at least in part, on the identification of numerous interacting components of the enzyme Dicer, in particular, proteins previously unknown to interact with this critical protein. Moreover, the invention provides an assay for the identification of other components of this and related enzymes. Importantly, the invention demonstrates that the identified interactors of Dicer are capable of modulating its function in, for example RNAi. Still further, the identified C. elegans proteins have related homologs in vertebrates, for example, the mouse and humans, and therefore have application in the development of human diagnostic and therapeutic agents. Accordingly, the invention has several advantages, which include, but are not limited to, the following: - providing interacting proteins of Dicer and there use in modulating Dicer function; - methods for identifying further interactors of Dicer and their structural and functional characteristics; - method for regulating Dicer activity though the use of Dicer interactors; - methods for improving the in vitro or in vivo processing of Dicer proteins or for use as targets for pharmaceutical intervention in order to modulate the properties of Dicer in vivo for improved RNAi; and - methods for stabilizing RNAi agents / compositions comprising Dicer by the addition of stabilizing interactor proteins or the same for use in purifying Dicer and other Dicer components.

Other features and advantages of the invention will be apparent from the following detailed description and claims.

### BRIEF DESCRIPTION OF THE DRAWINGS

Figure 1 depicts a schematic of major components of the RNAi pathway, the role of Dicer, and Dicer interacting proteins, which have roles in microRNA maturation, RNAi initiation, and as enhancers of RNAi.

Figures 2A-C depicts biochemical fractionation and immunoprecipitations of DCR-1 from C. elegans embryos, and adults using the coupled HA monoclonal method. dcr-/- 8xHA rescue fractions and IP were realized using a complex array rescued strain of dcr-1(ok247) with a transgene driving a 8xHA fusion.

Figures 3A-C depict the molecular architecture of the eri genes. Figure 3A discloses SEQ ID NO: 106. Figure 3B discloses SEQ ID NOS: 106 and 107, respectively, in order of appearance. Figure 3C discloses SEQ ID NOS: 109-112, respectively, in order of appearance.

Figures 4A-B depict results regarding RNAi sensitivity, enhancement, and developmental defects of the eri genes. A. N2(WT) or eri mutants were fed on unc-73 (rnai) feeding strain for a generation and F1 broods of animals were scored for their exhibition of the associated phenotype: uncoordination, twisted morphology and limited movement (see lower panel). In the upper panel, results are shown for n=15, depicted error bars are shown for a confidence interval p=0.05. B. Brood sizes of the eri mutants at 15°C (blue) and 25°C (purple) are shown (upper panel). WT brood size is restored at 25°C for all the eri mutations when crossing in with N2(wt) males (see lower panel). For all the broods, n=10, depicted error bars are shown for a confidence interval p=0.05.

Figures 5A-E depict small RNA defects in depletions for the DCR-1 interactors. In addition to dcr-1 and drh-3, the k02e2.6 locus also required the eri genes for accumulation, and the siRNAs were also absent from the eri genes RNA preparations from animals grown at 15°C (5A-D). The lack of small RNAs in k02e2.6 in the eri mutants correlated with an upregulation of its mRNA, as quantified by real time PCR (5E). See the Materials and Methods for further detail.

Figure 6 depicts a schematic showing that multiple silencing pathways are initiated by DCR-1, the eri gene products, and DRH-3. Distinct subsets of DCR-1 interactions are responsible for initiation of multiple small RNA silencing pathways. Shown here are the ‘classical' RNAi pathway involving the RDE-1, RDE-4 and the DRH-1/2 proteins, the eri ‘endo' RNAi pathway relying on the eri gene products, and the broader drh-3 dependent endo siRNA pathway.

### DETAILED DESCRIPTION OF THE INVENTION

The present invention is based, at least in part, on the discovery of previously unrecognized activity of several proteins as Dicer-interacting proteins (i.e., Dicer-interactors) and/or Dicer modulatory proteins (e.g., positive and/or negative regulatory proteins), see Tables 1 and 3. The invention features the defining of Dicer (DCR-1) interactions with an array of proteins involved in a variety of functions in C. elegans or other species, and the usage and alteration of these interactors and/or interactions to modulate or modify the different functions or activities of Dicer. The invention also features methods for efficient Dicer purification and identification of further interactors and/or interactions. This invention features methods for more efficient in vitro Dicer processing and materials for use in said methods, e.g., by the addition of a Dicer interacting protein that enhances Dicer activity. Knowledge of these Dicer interactors and/or interactions allows for the development of drug screening and/or targeting strategies or rationales, e.g., screening and/or targeting of Dicer and/or Dicer interactors in C. elegans,as well as in other species having homologous genes, to activate or antagonize Dicer's different functions and activities or to modulate its specificity toward its different proteins.

Accordingly, the present invention features Dicer interactors and methods of use of said interactors. In certain aspects, the invention provides methods for identifying a Dicer modulator, RNAi modulator and/or gene silencing modulator, including contacting a composition comprising, or a cell or organism that expresses Dicer or a bioactive fragment thereof and a Dicer interactor or a bioactive fragment thereof with a test compound and determining the ability of the test compound to modulate interaction (e.g., binding) of Dicer or the Dicer bioactive fragment to the Dicer interactor or the Dicer interactor bioactive fragment, such that the Dicer modulator, RNAi modulator and/or gene silencing modulator is identified.

In other aspects, the present invention provides methods for identifying a Dicer modulator, RNAi modulator and/or gene silencing modulator, including contacting a composition comprising, or a cell or organism that expresses Dicer or a bioactive fragment thereof and a Dicer interactor or a bioactive fragment thereof with a test compound and determining the ability of the test compound to modulate an activity of Dicer or the Dicer bioactive fragment, such that an the modulator is identified.

In certain embodiments, the activity of Dicer or the bioactive fragment thereof may be selected from the group consisting of: (1) processing of miRNA precursors; (2) processing of siRNA precursors; (3) mediating mRNA cleavage; (4) mediating assembly of RISC (e.g., via siRNAs); (5) directing translation repression (e.g., via miRNAs); (6) a ribonuclease activity (e.g., cleavage of dsRNA); and (7) initiation of RNAi.

In other aspects, the invention provides methods for identifying a Dicer modulator, RNAi modulator and/or gene silencing modulator, including contacting a composition comprising, or a cell or organism that expresses Dicer or a bioactive fragment thereof and a Dicer interactor or a bioactive fragment thereof with a test compound and determining the ability of the test compound to modulate an activity of the protein or the protein bioactive fragment, such that the Dicer modulator, RNAi modulator and/or gene silencing modulator is identified. In various embodiments of the preceding aspects the modulator identified may be a positive modulator or a negative modulator.

In various embodiments of the preceding aspects of the invention, the Dicer interactor may be selected from the proteins described in subsections IIIA-IIIMM, infra. In other embodiments, the Dicer is either Dicer1 or Dicer2. A Dicer bioactive fragment is any fragment of Dicer having sufficient size and structure to carry out at least one activity (e.g., biological activity) of the corresponding full-length Dicer protein. Similarly, a Dicer interactor bioactive fragment is any fragment of the Dicer interactor having sufficient size and structure to carry out at least one activity (e.g., biological activity) of the corresponding full-length Dicer interactor protein. Exemplary bioactive fragments include, but are not limited to, enzymatic domains, protein binding and/or interaction domains, and nucleic acid binding domains. Preferred bioactive fragments include regions or domains as described in detail in subsections IIIA-IIIMM, infra. The Dicer, Dicer bioactive fragment, Dicer interactor or the interactor bioactive fragment may be detectably labeled, radioactively labeled, or fluorescently labeled. Furthermore, in other embodiments, the interaction or activity may be compared to an appropriate control. In addition, at least one of the Dicer, Dicer bioactive fragment, Dicer interactor or protein bioactive fragment may be immobilized.

In various embodiments, the activity of the Dicer interactor or protein bioactive fragment is an activity set forth in subsections IIIA-IIIMM, infra. Bioactive fragments and/or fragment activities (and accordingly, Dicer interactor activities) are further described in detail in the references cited throughout subsections IIIA-IIIMM, infra. The entire content of these references is incorporated herein by reference.

In the aspects of the present invention, where the method involves a cell or organism, the cell or organism may overexpress the Dicer interactor or the bioactive fragment thereof, Dicer or the bioactive fragment thereof, or both the Dicer interactor (or protein bioactive fragment) and Dicer (or Dicer bioactive fragment).

In another aspect, the invention provides a modulator as identified by any of the preceding claims. The invention also provides for a pharmaceutical composition including the modulator.

In one aspect, the invention provides a method for identifying a Dicer:Dicer interactor modulator, including contacting a cell or organism expressing, or a composition comprising, Dicer or a bioactive fragment thereof and a Dicer interactor or a bioactive fragment thereof with a test compound and determining the ability of the test compound to affect interaction (e.g., binding) of the Dicer or the bioactive fragment thereof to the Dicer interactor or the bioactive fragment thereof, such that the modulator is identified.

In another aspect, the invention provides a method for identifying a Dicer:Dicer interactor modulator, including contacting a cell or organism expressing, or a composition comprising, Dicer or a bioactive fragment thereof and a Dicer interactor or a bioactive fragment thereof with a test compound and determining the ability of the test compound to affect activity of the Dicer or the bioactive fragment thereof, such that the modulator is identified.

In another aspect, the invention provides a method for identifying a Dicer:Dicer interactor modulator, including contacting a cell or organism expressing, or a composition comprising, Dicer or a bioactive fragment thereof and a Dicer interactor or a bioactive fragment thereof with a test compound and determining the ability of the test compound to affect activity of the Dicer interactor protein or the bioactive fragment thereof, such that the modulator is identified.

In yet another aspect, the invention provides a method for identifying a Dicer:Dicer interactor modulator, including contacting a cell or organism expressing, or a composition comprising, Dicer or a bioactive fragment thereof and a Dicer interactor or a bioactive fragment thereof with a test compound and determining the ability of the test compound to affect the phosphorylation state of the Dicer interactor or the bioactive fragment thereof, such that the modulator is identified.

In certain embodiments of the preceding aspects, the ability of the test compound to affect, for example, an interaction or activity includes the ability of the test compound to either enhance or inhibit such an interaction or activity. The Dicer may be Dicer1 or Dicer2.

In certain embodiments, the present invention provides methods of modulating Dicer, RNAi or gene silencing in a subject including administering to the subject a Dicer modulator, RNAi modulator and/or gene silencing modulator identified according to any of the above methods.

In another aspect, the invention provides an antibody that specifically binds to Dicer, a Dicer-interacting protein, or fragment thereof, wherein the antibody is capable of identifying, altering, or interfering with a Dicer:Dicer interactor interaction. In a related embodiment, the invention provides an antibody capable of binding an epitope within amino acid residue positions 1145 to 1347 of Dicer (DCR-1), or corresponding residues of a homolog thereof. The invention also provides polypeptides comprising Dicer epitopes suitable for raising such antibodies, e.g., for use as immunogens or screening polypeptides. In one embodiment, the epitope is within amino acid residue positions 1145 to 1347 of Dicer (DCR-1), or corresponding residues of a homolog thereof. The invention further provides for a pharmaceutical composition including the antibody.

In another aspect, the present invention provides a pharmaceutical composition including a Dicer-interacting protein. In yet another aspect, the present invention provides a pharmaceutical composition including a Dicer interacting protein domain of a Dicer protein or a Dicer interacting domain of a Dicer interacting protein, wherein either or both domains are capable of interfering with a Dicer:Dicer interacting protein interaction.

In yet another aspect, the invention provides a modulator of Dicer activity suitable for enhancing an RNAi therapy, and pharmaceutical compositions comprising such a modulator.

In other aspects, the present invention provides methods for treating an disease or disorder including administering any of the pharmaceutical compositions described above.

Various aspects of the invention are described in further detail in the following subsections:

I. Definitions

So that the invention may be more readily understood, certain terms are first defined.

As used herein, a "Dicer interacting protein" or "Dicer interactor" includes polypeptides having the amino acid sequences set forth in subsections IV, infra, as well as homologs, paralogs, and/or orthologs of such polypeptides, i.e. polypeptides having sufficient sequence identity to function in the same manner as the described polypeptides. Such polypeptides can interact directly, for example, physically bind with Dicer or a bioactive fragment thereof, and/or interact indirectly, for example, as measured by affecting a change in Dicer activity either in vitro or in vivo.

The term "Dicer" includes polypeptides having the amino acid sequences set forth in subsections III, infra, as well as homologs, paralogs, and/or orthologs of such polypeptides, i.e. polypeptides having sufficient sequence identity to function in the same manner as the described polypeptides.

The term "Dicer activity" includes any of the following properties or functions that can be ascribed to a Dicer protein such as: protein:protein binding activity (e.g., direct association with a Dicer interacting protein), miRNA maturation activity, RNAi initiation activity, RNAi enhancer activity, helicase activity, RISC activity, target recognition activity, and/or target gene cleavage activity.

The term "modulator of Dicer activity" includes agents capable of affecting a change in Dicer activity. Modulator agents include small molecules, nucleic acids (e.g., RNAi agents, siRNAs, shRNAs), peptides, and polypeptides. Dicer interacting proteins can be modulators of Dicer either directly or indirectly, for example, by physically interacting with Dicer or by affecting a change in Dicer activity. Thus, a modulator of a Dicer interacting protein which results in a change in Dicer activity can be considered a modulator of Dicer activity, albeit indirectly.

The term "derived from" includes partial, synthetic, recombinant, or genetically engineered nucleic acids or polypeptides that encode or represent a gene product substantially similar to a gene product from a particular source, for example, a nucleic acid source, a cell, or organismal source, from, for example, a nematode, fruit fly, rat, mouse, primate, or human.

The terms "homolog," "paralog," "ortholog," includes their art recognized meaning. Typically, a homolog of a given gene product is one of functional similarity as well as sequence similarity. If the homolog is derived from a different organism, the homolog may be referred to as the ortholog. If several homologs exist in a given organism, the homolog may be referred to as a paralog. Typically, the sequence similarity/identity between homologs is at least about 40%, 50%, 60%, 70%, 80%, 90%, or more (or a percentage falling within any interval or range of the foregoing). Methods for determining such similarity/identity are described, infra. Motifs conserved between homologs can have a sequence similarity/identity of at least about 70%, 80%, 90%, or more. It is understood that when comparing gene product sequence between diverse organisms, for example, nematodes and humans, sequence similarity between given homologs across the entire protein sequence may be low. However, if functional complementarity exists, and in addition, if conserved motifs exist, e.g., protein; protein interaction motifs, e.g., motifs involved in Dicer activity or Dicer:Dicer interacting protein interactions, then the gene products being compared can be considered homologs and thus selected as compositions for use in the methods of the invention, as described herein.

The phrase "introducing into the cell or organism" includes any art recognized method for introducing genetic information into an cell extract, cell, or organism. Typical modes of such transfer of genetic information include the contacting, transfection, microinjection and/or feeding of nucleic acid agents or expression vectors to an extract, cell, or organism. Other methods include cell fusion, pronuclear injection, genetic crosses/mutagenesis, and the like.

The term "bioactive fragment" includes any portion (e.g., a segment of contiguous amino acids) of a Dicer interactor or Dicer protein sufficient to exhibit or exert at least one Dicer protein- or Dicer- associated activity including, for example, the ability to bind to Dicer or Dicer interactor protein, respectively.

The phrase "encodes a gene product" includes the generation of a RNA molecule from a DNA molecule (i.e., a complementary RNA molecule generated from the DNA molecule by the process of transcription) and/or the generation of a polypeptide or protein molecule from an RNA (i.e., by the processes of transcription and translation).

The term "kit" is any manufacture (e.g. a package or container) comprising at least one reagent or component, e.g. a construct, molecule, and/or compound, the manufacture being promoted, distributed, or sold as a unit for performing the methods of the invention.

The term "target gene" includes a gene intended for downregulation via RNA interference ("RNAi"). The term "target gene product" or "target protein" refers to a gene product, e.g., a nucleic acid or protein, intended for downregulation via RNAi. The term "target RNA" refers to an RNA molecule intended for degradation by RNAi, e.g., by nucleic acid cleavage. An exemplary "target RNA" is a coding RNA molecule (e.g., an RNA molecule encoding a gene product, e.g., an mRNA and protein so encoded therefrom).

The term "expression" of a gene or nucleic acid encompasses not only cellular gene expression, but also the transcription and translation of nucleic acid(s) in cloning systems and in any other context.

The term "RNA interference" or "RNAi", as used herein, refers generally to a sequence-specific or selective process by which a target molecule (e.g., a target gene, protein, or RNA) is downregulated. In specific embodiments, the process of "RNA interference" or "RNAi" features degradation of RNA molecules, e.g., RNA molecules within a cell, the degradation being triggered by an RNAi agent. Degradation is catalyzed by an enzymatic, RNA-induced silencing complex (RISC). RNAi occurs in cells naturally to remove foreign RNAs (e.g., viral RNAs). Natural RNAi proceeds via fragments cleaved from free dsRNA which direct the degradative mechanism to other similar RNA sequences. Alternatively, RNAi can be initiated by the hand of man, for example, to silence the expression of target genes.

The term "RNAi agent", includes an RNA (or analog thereof), comprising a sequence having sufficient complementarity to a target RNA (i.e., the RNA being degraded) to direct RNAi. A sequence having a "sufficiently complementary to a target RNA sequence to direct RNAi" means that the RNAi agent has a sequence sufficient to trigger the destruction of the target RNA by the RNAi machinery (e.g., the RISC complex) or process. The term RNA agent or RNAi agent includes small interfering RNA (siRNA) (also referred to in the art as short interfering RNAs) as well as small hairpin RNA or shRNA.

The term "small interfering RNA," "siRNA," or "short interfering RNAs" includes a double-stranded RNA agent, which is capable of directing or mediating RNA interference. Naturally occurring siRNAs are generated from longer dsRNA molecules (e.g., > 25 nucleotides in length) by a cell's RNAi machinery (e.g., the RISC complex).

The term "small hairpin RNA" or "shRNA" (also referred to in the art as "short hairpin RNA"), includes an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region.

The term "subject", as used herein, includes living organisms at risk for or having a cellular, neurological, e.g. neurodegenerative disease, or disorder. Examples of subjects include humans, monkeys, cows, sheep, goats, dogs, cats, mice, rats, and transgenic species thereof. Administration of the compositions of the present invention to a subject to be treated can be carried out using known procedures, at dosages and for periods of time effective to modulate RNAi in the subject as further described herein.

The term "treatment", as used herein, is defined as the application or administration of a therapeutic agent to a subject, or application or administration of a therapeutic agent to an isolated tissue or cell line from a subject, who has a disease or disorder, a symptom of a disease or disorder, or a predisposition toward a disease or disorder, with the purpose to cure, heal, alleviate, relieve, alter, remedy, ameliorate, improve or affect the disease or disorder, the symptoms of the disease or disorder, or the predisposition toward a disease or disorder. A therapeutic agent includes, but is not limited to, small molecules, peptides, antibodies, ribozymes, antisense oligonucleotides, RNAi agents, chemotherapeutic agents, and radiation.

The term "effective amount", as used here in, is defined as that amount necessary or sufficient to treat or prevent a disorder, e.g. a neurological or a neurodegenerative disease or disorder. The effective amount can vary depending on such factors as the size and weight of the subject, the type of illness, or the particular agent being administered. One of ordinary skill in the art would be able to study the aforementioned factors and make the determination regarding the effective amount of the agent without undue experimentation.

The term "pharmaceutical composition" as used herein, refers to an agent formulated with one or more compatible solid or liquid filler diluents or encapsulating substances, which are suitable for administration to a human or lower animal.

The phrase "a gene involved" in a disorder includes a gene, the normal or aberrant expression or function of which effects or causes a disease or disorder or at least one symptom of said disease or disorder.

The phrase "examining the function of a gene in a cell or organism" refers to examining or studying the expression, activity, function, or phenotype arising therefrom.

A "suitable control" or "appropriate control" refers to any control or standard familiar to one of ordinary skill in the art useful for comparison purposes. In one embodiment, a "suitable control" or "appropriate control" is a value, level, feature, characteristic, property, etc. determined prior to performing an RNAi methodology, as described herein. For example, a Dicer activity, a RISC level of activity or amount, target gene level or target gene degradation level, a transcription rate, mRNA level, translation rate, protein level, biological activity, cellular characteristic or property, genotype, phenotype, etc. can be determined prior to introducing a nucleic acid or test compound of the invention into a cell extract, cell, or organism.

The term "cell" refers to any eukaryotic cell which exhibits RNAi activity and includes, e.g., animal cells (e.g., mammalian cells, e.g., human or murine cells), nematode cells, plant cells, and yeast. The term includes cell lines, e.g., mammalian cell lines such as HeLa cells as well as embryonic cells, e.g., embryonic stem cells and collections of cells in the form of, e.g., a tissue.

The term "cell extract" refers to a lysate or acellular preparation of a cell as defined above and can be a crude extract or partially purified as well as comprise additional agents such as recombinant polypeptides, nucleic acids, and/or buffers or stabilizers.

The term "organism" refers to multicellular organisms such as, e.g., C. elegans, Drosophila, mouse, and human.

The term "vector" refers to a nucleic acid molecule (either DNA or RNA) capable of conferring the expression of a gene product when introduced into a host cell or host cell extract. In one embodiment, the vector allows for temporal or conditional expression of one or more nucleic acids of the invention, e.g., a single strand, RNA agent, siRNA, or shRNA. The vector may be episomal or chromosomally (e.g., transgenically) integrated into a host cell genome.

The terms used herein are not intended to be limiting of the invention.

II. Overview

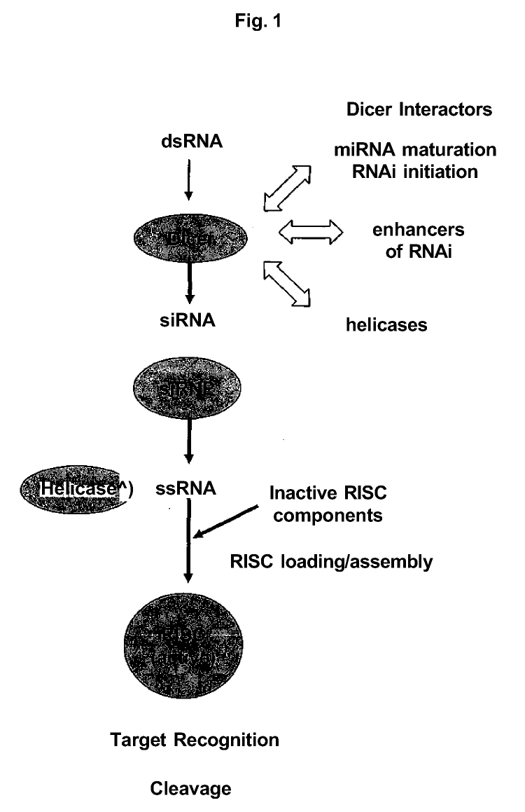

Dicer, a ribonuclease III / DExH-box helicase (DCR-1 in C. elegans) plays a central role in a variety of small RNA-directed gene silencing mechanisms for a large range of organisms (see Figs. 1 & 6).

Its best characterized activity is the processing of double-stranded RNAs into smaller RNA hybrid species of 21 to 25 nucleotides (nt) in length with staggered 2 nucleotides overhangs at the 3' ends of the duplex, and a 5' phosphate group; both of which determinants have been shown to be required for efficient silencing.

This Dicer activity was first shown to act in the initiation phase of two modes of post-transcriptional gene silencing. In RNA interference (RNAi) and in the microRNA-dependent silencing, Dicer recognizes a double-stranded RNA (dsRNA) trigger to direct a potent, and sequence-specific gene silencing response. This process requires the assembly of the small RNA product in a downstream complex called RISC, for which Argonaute proteins are a central component. This complex is responsible for a cognate mRNA search, and for the subsequent silencing of the complementary transcript.

Dicer is responsible for the integration of a variety of RNA signals with distinct biological outcomes. Dicer also initiates other RNA-dependent silencing pathways such as chromosome folding and the like. Therefore a key problem to address is how some specific classes of dsRNAs are recognized and recruited to be processed by Dicer, and how RNA triggers of distinct origins potentiate different silencing responses.

The present invention provides methods and compositions for conducting in vitro and in vivo assays for identifying Dicer interacting proteins, in particular, Dicer interacting proteins that can affect Dicer activity, and modulators thereof.

III.Dicer Interacting Proteins or Dicer Interactors

According to the invention, several proteins have been identified as interacting with and/or regulating Dicer, e.g., Dicer activity. These Dicer interactors are described in detail below under subsections IIIA through IIIMM. Using methods described in the present disclosure, use of any one of these proteins, or cognate orthologs or paralogs, in appropriate screening assays would provide for the identification of Dicer modulators and/or RNAi-modulators, and/or gene silencing modulators.

IIIA.RDE-4 LOCUS NP_499265 385 aa linear DEFINITION RNAi Defective RDE-4, RNA interference promoting factor with double-stranded RNA binding motif (43.4 kD) (rde-4) [Caenorhabditis elegans]. ACCESSION NP_499265 VERSION NP_499265.1 GI:17555186 DBSOURCE REFSEQ: accession NM_066864.2 KEYWORDS . SOURCE Caenorhabditis elegans ORGANISM Caenorhabditis elegans Eukaryota; Metazoa; Nematoda; Chromadorea; Rhabditida; Rhabditoidea; Rhabditidae; Peloderinae; Caenorhabditis. REFERENCE 1 (residues 1 to 385) AUTHORS Walhout,A.J., Reboul,J., Shtanko,O., Bertin,N., Vaglio,P., Ge,H., Lee,H., Doucette-Stamm,L., Gunsalus,K.C., Schetter,A.J., Morton,D.G., Kemphues,K.J., Reinke,V., Kim,S.K., Piano,F. and Vidal,M. TITLE Integrating interactome, phenome, and transcriptome mapping data for the C. elegans germline JOURNAL Curr. Biol. 12 (22), 1952-1958 (2002) MEDLINE 22335532 PUBMED 12445390 REFERENCE 2 (residues 1 to 385) AUTHORS Tabara,H., Yigit,E., Siomi,H. and Mello,C.C. TITLE The dsRNA binding protein RDE-4 interacts with RDE-1, DCR-1, and a DExH-box helicase to direct RNAi in C. elegans JOURNAL Cell 109 (7), 861-871 (2002) MEDLINE 22105477 PUBMED 12110183 REFERENCE 3 (residues 1 to 385) AUTHORS Tijsterman,M., Ketting,R.F., Okihara,K.L., Sijen,T. and Plasterk,R.H. TITLE RNA helicase MUT-14-dependent gene silencing triggered in C. elegans by short antisense RNAs JOURNAL Science 295 (5555), 694-697 (2002) MEDLINE 21669321 PUBMED 11809977 REFERENCE 4 (residues 1 to 385) AUTHORS Parrish,S. and Fire,A. TITLE Distinct roles for RDE-1 and RDE-4 during RNA interference in Caenorhabditis elegans JOURNAL RNA 7 (10), 1397-1402 (2001) MEDLINE 21535543 PUBMED 11680844 REFERENCE 5 (residues 1 to 385) AUTHORS Grishok,A., Tabara,H. and Mello,C.C. TITLE Genetic requirements for inheritance of RNAi in C. elegans JOURNAL Science 287 (5462), 2494-2497 (2000) MEDLINE 20207007 PUBMED 10741970 REFERENCE 6 (residues 1 to 385) AUTHORS Tabara,H., Sarkissian,M., Kelly,W.G., Fleenor,J., Grishok,A., Timmons,L., Fire,A. and Mello,C.C. TITLE The rde-1 gene, RNA interference, and transposon silencing in C. elegans JOURNAL Cell 99 (2), 123-132 (1999) MEDLINE 20004389 PUBMED 10535731 COMMENT REVIEWED REFSEQ: This record has been curated by NCBI staff. This record is derived from an annotated genomic sequence (NC_003281). The reference sequence was derived from AY071926.1. Summary: This gene rde-4, also known as T20G5.11, 3L306 or YK5801, maps at (III; +1.89). Its phenotype is rnai defective. It encodes a RNA interference promoting factor with double-stranded RNA binding motif. From Pfam homology, the product would have double-stranded RNA binding activity and would localize in intracellular. According to the Worm Transcriptome Project, it is well expressed at all stages of development [Kohara cDNAs]. Its sequence is defined by 10 cDNA clones. Phenotype Selected strain available from the CGC. WM49 rde-4(ne301) III [Craig Mello, Tabara/Mello, mut-6] RNAi deficient. RNA interference results: [T.Hyman 2000] No obvious phenotype (by injecting genomic PCR product TH:T20G5.11). [J.Ahringer 2003] No obvious phenotype (by feeding genomic PCR product JA:T20G5.11). [F.Piano 2002] No P0 sterility detected. No postembryonic phenotypes observed among progeny. No obvious phenotype. Expression The expression profile for the gene, derived from the proportion of animals at each stage in each Kohara library is: embryos 47%, L1 or L2 larvae 29%, L3 to adult 25%. In situ hybridisation pictures to all stages of development are available from Kohara NextDB. Pattern germline enriched [Piano, 2002]. This complete mRNA is 1747 bp long. Its sequence exactly matches the genome. The premessenger has 4 exons. It covers 1.89 kb on the WS97 genome. It is transpliced to SL1. It has a very long 3' UTR. The protein (385 aa, 43.4 kDa, pI 5.2) contains 2 Double-stranded RNA binding (DsRBD) domain motifs. It also contains a coil coil stretch [Psort2]. Taxblast (threshold 10^-3) tracks ancestors down to caenorhabditis elegans. COMPLETENESS: full length. Method: conceptual translation. FEATURES Location/Qualifiers source 1..385 /organism="Caenorhabditis elegans" /db_xref="taxon:6239" /chromosome="III" /map="III;+1.89 cM (interpolated genetic position)" /map="III; covering 1888 bp, from base 10218484 to 10216597 on genome release WS97" /clone="Primers to amplify the CDS (3468 bp, Stop included): ATGGATTTAACCAAACTAACGTTTGAA (SEQ ID NO: 1)(T=55.9), TCAATCCGTGAAATCATAGGTGT (SEQ ID NO: 2)(T=56.6). Complete CDS clones: AY071926, yk832c2. Recommended clone (from the Kohara collection): yk832c2. Other clone(s): yk627d6, yk333g4, yk596c11, yk565d11, yk469h7, yk1429h2, yk1706h7, yk1726d4. for edited clone sequences see clone_lib="Kohara embryonic lambda gt11 library: yk627d6, yk333g4, yk596c11, yk565d11, yk469h7; Kohara Sugano L1 larvae cap-selected library: yk832c2; Kohara Sugano L2 larvae cap-selected library: yk1706h7, yk1726d4; Kohara Sugano L4 larvae cap-selected library: yk1429h2; gb: AY071926" Protein 1..385 /product="RNAi DEfective RDE-4, RNA interference promoting factor with double-stranded RNA binding motif (43.4 kD) (rde-4)" Region 41..104 /region_name="[Pfam/InterPro description] double-stranded RNA binding (DsRBD) domain" /db_xref="CDD:pfam00035" Region 116..149 /region_name="[PSORT] coil coil domain: PGTTKEEALSNIDQISDKAEELKRSTSDAVQDND" (SEQ ID NO: 3) Region 170..232 /region_name="[Pfam/InterPro description] double-stranded RNA binding (DsRBD) domain" /db_xref="CDD:pfam00035" CDS 1..385 /gene="rde-4" /locus_tag="3L306" /coded_by="NM_066864.2:1..1158" /db_xref="AceView/WormGenes:rde-4" /db_xref="GeneID:176438" /db_xref="LocusID:176438" /db_xref="WormBase:T20G5.11" ORIGIN 1 mdltkltfes vfggsdvpmk psrsednktp rnrtdlemfl kktplmvlee aakavyqktp 61 twgtvelpeg femtlilnei tvkgqatskk aarqkaavey lrkvvekgkh eiffipgttk 121 eealsnidqi sdkaeelkrs tsdavqdndn ddsiptsaef ppgisptenw vgklqeksqk 181 sklqapiyed sknerterfl victmcnqkt rgirskkkda knlaawlmwk aledgiesle 241 sydmvdvien leeaehllei qdqaskikdk hsalidilsd kkrfsdysmd fnvlsvstmg 301 ihqvlleisf rrlvspdpdd lemgaehtqt eeimkataek eklrkknmpd sgplvfaghg 361 ssaeeakqca cksaiihfnt ydftd (SEQ ID NO: 4)

IIIB.ALG-1

ALG-1 is a homolog of rde-1 that is involved in RNA interference and affects developmental timing along with alg-2 and dcr-1 by regulating expression of the lin-4 and let-7 small temporal RNAs. The ALG-1 protein contains regions of similarity to Pfam domains PF02170 (PAZ domain, Residues 377-514), PF02171 (Piwi domain, Residues 660-961). The protein has been implicated in embryonic development, inferred from mutant phenotype Grishok, A. et al., Cell 2001 106:23-34. Homologs include H. sapiens eukaryotic translation initiation factor 2C 4, C. elegans gene T07D3.7a, M. musculus Argonaute 1 protein (Fragment), R. norvegicus eukaryotic translation initiation factor 2C 2 (eIF2C 2) (eIF-2C 2)s(Golgi ER protein 95 kDa) (GERp95) and D. melanogaster AGO1. LOCUS NP_510322 1002 aa linear DEFINITION argonaute (plant)-Like Gene (110.9 kD) (alg-1) [Caenorhabditis elegans]. ACCESSION NP_510322 VERSION NP_510322.2 GI:25148113 DBSOURCE REFSEQ: accession NM_077921.2 KEYWORDS . SOURCE Caenorhabditis elegans ORGANISM Caenorhabditis elegans Eukaryota; Metazoa; Nematoda; Chromadorea; Rhabditida; Rhabditoidea; Rhabditidae; Peloderinae; Caenorhabditis. REFERENCE 1 (residues 1 to 1002) AUTHORS Kamath,R.S., Fraser,A.G., Dong,Y., Poulin,G., Durbin,R., Gotta,M., Kanapin,A., Le Bot,N., Moreno,S., Sohrmann,M., Welchman,D.P., Zipperlen,P. and Ahringer,J. TITLE Systematic functional analysis of the Caenorhabditis elegans genome using RNAi JOURNAL Nature 421 (6920), 231-237 (2003) MEDLINE 22417569 PUBMED 12529635 REFERENCE 2 (residues 1 to 1002) AUTHORS Morel,J.B., Godon,C., Mourrain,P., Beclin,C., Boutet,S., Feuerbach,F., Proux,F. and Vaucheret,H. TITLE Fertile hypomorphic ARGONAUTE (ago1) mutants impaired in post-transcriptional gene silencing and virus resistance JOURNAL Plant Cell 14 (3), 629-639 (2002) MEDLINE 21907852 PUBMED 11910010 REFERENCE 3 (residues 1 to 1002) AUTHORS Grishok,A., Pasquinelli,A.E., Conte,D., Li,N., Parrish,S., Ha,I., Baillie,D.L., Fire,A., Ruvkun,G. and Mello,C.C. TITLE Genes and mechanisms related to RNA interference regulate expression of the small temporal RNAs that control C. elegans developmental timing JOURNAL Cell 106 (1), 23-34 (2001) MEDLINE 21354308 PUBMED 11461699 COMMENT VALIDATED REFSEQ: This record has undergone preliminary review of the sequence, but has not yet been subject to final review. This record is derived from an annotated genomic sequence (NC_003284). The reference sequence was derived from WormBase CDS:F48F7.1. Summary: This gene alg-1, also known as F48F7.1, XO573 or YK3586, maps at (X; +14.45). Its phenotype is clear, translucent appearance, uncoordinated locomotion, protruding vulva. It encodes an argonaute (plant)-Like Gene. According to the Worm Transcriptome Project, it is well expressed at all stages of development [Kohara cDNAs]. Its sequence is fully supported by 17 cDNA clones. RNA interference results [J.Ahringer 2003] Clear, uncoordinated, protruding vulva (by feeding genomic PCR product JA:F48F7.1). Expression The expression profile for the gene, derived from the proportion of animals at each stage in each Kohara library is: embryos 26%, L1 or L2 larvae 27%, L3 to adult 48%. In situ hybridisation pictures to all stages of development are available from Kohara NextDB. The CDS has 6 exons. It covers 3.42 kb on the WS97 genome. The protein (1002 aa, 110.9 kDa, pI 9.3) contains one Argonaute and Dicer protein, PAZ motif, one stem cell self-renewal protein Piwi motif. It also contains a 2nd peroximal domain [Psort2]. Taxblast (threshold 10^-3) tracks ancestors down to eukaryota. COMPLETENESS: full length. Method: conceptual translation. FEATURES Location/Qualifiers source 1..1002 /organism="Caenorhabditis elegans" /db_xref="taxon:6239" /chromosome="X" /map="X;+14.45 cM (interpolated genetic position)" /map="X; covering 4989 bp, from base 13941769 to 13946759 on genome release WS97" /clone_lib="Kohara embryonic lambda gt11 library: yk60e5, yk403g7, yk142f4, yk375c8, yk481b11, yk245e6; Kohara Sugano L1 larvae cap-selected library: yk759f4, yk889c6, yk1013a7, yk1108b4, yk1164h8; Kohara Sugano L2 larvae cap-selected library: yk1609d8; Kohara Sugano L4 larvae cap-selected library: yk1427e7; Kohara mixed stage library, from him-8 strain, containing 15-30% males: yk100d5, yk545h7, yk369b2; Kohara mixed stage library, from him-8 strain, containing 15-30% males: yk286h2" Protein 1..1002 /product="argonaute (plant)-Like Gene (110.9 kD) (alg-1)" Region 377..514 /region_name="[Pfam/InterPro description] argonaute and Dicer protein, PAZ" /db_xref="CDD:pfam02170" Region 460..468 /region_name="[PSORT] 2nd peroximal domain: RIQLKYPHL" (SEQ ID NO: 5) Region 660..961 /region_name="[Pfam/InterPro description] stem cell self-renewal protein Piwi" /db_xref="CDD:pfam02171" CDS 1..1002 /gene="alg-1" /locus_tag="XO573" /coded_by="NM_077921.2:1..3009" /db_xref="AceView/WormGenes:alg-1" /db_xref="GeneID:181504" /db_xref="LocusID:181504" /db_xref="WormBase:F48F7.1" ORIGIN 1 msggpqylpg vmnstiqqqp qsatssflps gpisststss qvvptsgatq qppfpsaqaa 61 astalqndle eifnspptqp qtfsdvpqrq agslapgvpi gntsvsigep antlggglpg 121 gapgqlpggn qsgiqfqcpr rpnhgvegrs illranhfav ripggtiqhy qvdvtpdkcp 181 rrvnreiisc lisafskyft nirpvydgkr nmytreplpi grermdfdvt lpgdsaverq 241 fsvslkwvgq vslstledam egrvrqvpfe avqamdvilr hlpslkytpv grsffsppvp 301 nasgvmagsc ppqasgavag gahsagqyha esklgggrev wfgfhqsvrp sqwkmmlnid 361 vsatafyrsm pviefiaevl elpvqalaer ralsdaqrvk ftkeirglki eithcgqmrr 421 kyrvcnvtrr paqtqtfplq letgqtiect vakyfydkyr iqlkyphlpc lqvgqeqkht 481 ylppevcniv pgqrcikklt dvqtstmika tarsaperer eisnlvrkae fsadpfahef 541 gitinpamte vkgrvlsapk llyggrtrat alpnqgvwdm rgkqfhtgid vrvwaiacfa 601 qqqhvkendl rmftnqlqri sndagmpivg npcfckyavg veqvepmfky lkqnysgiql 661 vvvvlpgktp vyaevkrvgd tvlgiatqcv qaknairttp qtlsnlclkm nvklggvnsi 721 llpnvrprif nepviffgcd ithppagdsr kpsiaavvgs mdahpsryaa tvrvqqhrqe 781 iisdltymvr ellvqfyrnt rfkparivvy rdgvsegqff nvlqyelrai reacmmlerg 841 yqpgitfiav qkrhhtrlfa vdkkdqvgka ynippgttvd vgithptefd fylcshagiq 901 gtsrpshyhv lwddnnltad elqqltyqmc htyvrctrsv sipapayyah lvafraryhl 961 vdrehdsgeg sqpsgtsedt tlsnmaravq vhpdannvmy fa (SEQ ID NO: 6)IIIC.ALG-2 LOCUS NP_871992 910 aa linear DEFINITION argonaute (plant)-Like Gene (101.6 kD) (alg-2) [Caenorhabditis elegans] ACCESSION NP_871992 VERSION NP_871992.1 GI:32564644 DBSOURCE REFSEQ: accession NM_182192.1 KEYWORDS . SOURCE Caenorhabditis elegans ORGANISM Caenorhabditis elegans Eukaryota; Metazoa; Nematoda; Chromadorea; Rhabditida; Rhabditoidea; Rhabditidae; Peloderinae; Caenorhabditis REFERENCE 1 (residues 1 to 910) AUTHORS Morel,J.B., Godon,C., Mourrain,P., Beclin,C., Boutet,S., Feuerbach,F., Proux,F. and Vaucheret,H. TITLE Fertile hypomorphic ARGONAUTE (ago1) mutants impaired in post-transcriptional gene silencing and virus resistance JOURNAL Plant Cell 14 (3), 629-639 (2002) MEDLINE 21907852 PUBMED 11910010 REFERENCE 2 (residues 1 to 910) AUTHORS Grishok,A., Pasquinelli,A.E., Conte,D., Li,N., Parrish,S., Ha,I., Baillie,D.L., Fire,A., Ruvkun,G. and Mello,C.C. TITLE Genes and mechanisms related to RNA interference regulate expression of the small temporal RNAs that control C. elegans developmental timing JOURNAL Cell 106 (1), 23-34 (2001) MEDLINE 21354308 PUBMED 11461699 REFERENCE 3 (residues 1 to 910) AUTHORS Missotten,M., Nichols,A., Rieger,K. and Sadoul,R. TITLE Alix, a novel mouse protein undergoing calcium-dependent interaction with the apoptosis-linked-gene 2 (ALG-2) protein JOURNAL Cell Death Differ. 6 (2), 124-129 (1999) MEDLINE 99218669 PUBMED 10200558 COMMENT VALIDATED REFSEQ: This record has undergone preliminary review of the sequence, but has not yet been subject to final review. This record is derived from an annotated genomic sequence (NC_003280). The reference sequence was derived from WormBase CDS:T07D3.7a. Summary: This gene alg-2, also known as T07D3.7, 2B167 or YK2467, maps at (II; -13.80). It encodes an argonaute (plant)- Like Gene. According to the Worm Transcriptome Project, it is expressed at high level mainly in embryos and some in L1 larvae [Kohara cDNAs]. Its sequence is fully supported by 29 cDNA clones and produces, by alternative splicing, 2 different transcripts a, b altogether encoding 2 different protein isoforms. Phenotype Knock-out allele, deletion obtained by the Gene Knockout Consortium ok215, ok304 (strain RB574) [R Barstead, Oklahoma MRF, USA] Selected strain available from the CGC. RB574 alg-2(ok304) II [Robert Barstead, OMRF Knockout Group/Barstead, UV/TMP] [Craig Mello description] Homozygous viable, contains an out of frame deletion removing nucleotides encoding amino acids 34-374. [R Barstead] Homozygous. Outer Left Sequence: tctgagtttggctcgatgtg (SEQ ID NO: 7). Outer Right Sequence: atgttccttggataccagcg (SEQ ID NO: 8). Inner Left Sequence: agcccagaactgggaaactt (SEQ ID NO: 9). Inner Right Sequence: aagtcgaattccgttggatg (SEQ ID NO: 10). Inner Primer PCR Product: 3297. Deletion length: 1378 bp. Deletion breakpoints: Flanking positions are T07D3 coordinates 2397/3776. Sequence read at break from ok304 internal left primer: TCTAATTTTCCAATTTTCAG (SEQ ID NO: 11) / GATATTGTTCCAGGACAGCG (SEQ ID NO: 12). Breakpoint data provided by the Vancouver Gene Knockout Lab (URL: RNA interference results: [J.Ahringer 2003] No obvious phenotype (by feeding genomic PCR product JA:T07D3.7). Expression The expression profile for the gene, derived from the proportion of animals at each stage in each Kohara library is: embryos 75%, L1 or L2 larvae 16%, L3 to adult 9%. In situ hybridisation pictures to all stages of development are available from Kohara NextDB. The CDS has 7 exons. It covers 5.26 kb on the WS97 genome. The protein (910 aa, 101.6 kDa, pI 9.2) contains one Argonaute and Dicer protein, PAZ motif, one stem cell self-renewal protein Piwi motif. It also contains a 2nd peroximal domain [Psort2]. It is predicted to localise in the cytoplasm [Psort2]. Taxblast (threshold 10^-3) tracks ancestors down to eukaryota. COMPLETENESS: full length. Method: conceptual translation. FEATURES Location/Qualifiers source 1..910 /organism="Caenorhabditis elegans" /db_xref="taxon:6239" /chromosome="II" /map="II;-13.80 cM (interpolated genetic position)" /map="II; covering 6702 bp, from base 873182 to 879885 on genome release WS97" Protein 1..910 /product="argonaute (plant)-Like Gene (101.6 kD) (alg-2)" Region 282..419 /region_name="[Pfam/InterPro description] argonaute and Dicer protein, PAZ" /db_xref="CDD:pfam02170" Region 365..373 /region_name="[PSORT] 2nd peroximal domain: RIQLKYPHL" (SEQ ID NO: 5) Region 566..867 /region_name="[Pfam/InterPro description] stem cell self-renewal protein Piwi" /db_xref="CDD:pfam02171" CDS 1..910 /gene="alg-2" /locus_tag="2B167" /coded_by="NM_182192.1:1..2733" /db_xref="AceView/WormGenes:alg-2" /db_xref="GeneID:173468" /db_xref="LocusID:173468" /db_xref="WormBase:T07D3.7a" ORIGIN 1 mfplpvhngp rlgklsifem pgdsltsssf mpdggaetss ssqlggsahg aigtkpdagv 61 qfqcpvrpnh gvegrsillr anhfavripg gsvqhyqidv fpdkcprrvn revigcliss 121 fskyftnirp vydgkrnmyt replpigtep mnfevtlpgd saverkfsvt mkwigqvcls 181 alddamegrv rqvpheavqs idvilrhlps lkytpvgrsf ftppgvmkpg mqmhqesklg 241 ggrevwfgfh qsvrpsqwkm mlnidvsata fyrampvief vaevlelpvq alaerralsd 301 aqrvkftkei rglkieithc gavrrkyrvc nvtrrpaqtq tfplqletgq tiectvakyf 361 fdkyriqlky phlpclqvgq eqkhtylppe vcdivpgqrc lkkltdvqts tmikatarsa 421 perereickl vskaelsadp fahefgitin pamtevkgrv lsapkllygg rhrattalpn 481 qgvwdmrgkq fhtgmevrtw aiacfaqqsh vkendlrmft tqlqristda gmpiigtpmf 541 ckyasgveqv epmfkylkqt ysaiqlivvv lpgktpiyae vkrvgdtvlg iatqcvqakn 601 airttpqtls nlclkmnvkl ggvnsillpn vrprifnepv iflgcdithp aagdtrkpsi 661 aavvgsmdah psryaatvrv qqhrqeiitd ltymvrellv qfyrntrfkp arivvyrdgv 721 segqlfnvlq yelraireac vmlesgyqpg itfiavqkrh htrlfaadka dqvgkafnip 781 pgttvdvgit hptefdfflc shagiqgtsr pshyhvlwdd ndltadelqq ltyqmchtyv 841 rctrsvsipa payyahlvaf raryhlvdrd hgsgeegsqp sgtssedttl ssmakavqvh 901 pdsnnvmyfa (SEQ ID NO: 13)IIID.DRH-1 LOCUS NP_501018 1037 aa linear DEFINITION Dicer-Related Helicase, a DExH-box helicase (119.2 kD) (drh-1) [Caenorhabditis elegans]. ACCESSION NP_501018 VERSION NP_501018.1 GI:17539846 DBSOURCE REFSEQ: accession NM_068617.2 KEYWORDS . SOURCE Caenorhabditis elegans ORGANISM Caenorhabditis elegans Eukaryota; Metazoa; Nematoda; Chromadorea; Rhabditida; Rhabditoidea; Rhabditidae; Peloderinae; Caenorhabditis. REFERENCE 1 (residues 1 to 1037) AUTHORS Tabara,H., Yigit,E., Siomi,H. and Mello,C.C. TITLE The dsRNA binding protein RDE-4 interacts with RDE-1, DCR-1, and a DExH-box helicase to direct RNAi in C. elegans JOURNAL Cell 109 (7), 861-871 (2002) MEDLINE 22105477 PUBMED 12110183 REFERENCE 2 (residues 1 to 1037) AUTHORS Marcotte,E.M., Xenarios,I., van Der Bliek,A.M. and Eisenberg,D. TITLE Localizing proteins in the cell from their phylogenetic profiles JOURNAL Proc. Natl. Acad. Sci. U.S.A. 97 (22), 12115-12120 (2000) MEDLINE 20504472 PUBMED 11035803 COMMENT REVIEWED REFSEQ: This record has been curated by NCBI staff. This record is derived from an annotated genomic sequence (NC_003282). The reference sequence was derived from AU205212, AF480439.1 and AU217173. Summary: This gene drh-1, also known as F15B10.2, 4H372 or YK7673, maps at (IV; +3.32). It encodes a Dicer-Related Helicase, a DExH-box helicase. From Pfam homology, the product would have ATP binding, nucleic acid binding, ATP dependent helicase, helicase activities. According to the Worm Transcriptome Project, it is well expressed at all stages of development [Kohara cDNAs]. Its sequence is defined by 19 cDNA clones. RNA interference results: [A.Sugimoto 2000] No obvious phenotype (by injecting cDNA clone SA:yk317d8). [J.Ahringer 2003] No obvious phenotype (by feeding genomic PCR product JA:F15B10.2). Expression The expression profile for the gene, derived from the proportion of animals at each stage in each Kohara library is: embryos 6%, L1 or L2 larvae 19%, L3 to adult 74%. In situ hybridisation pictures to all stages of development are available from Kohara NextDB. Pattern [pm11035803] predicted mitochondrial. This complete mRNA is 3298 bp long. Its sequence exactly matches the genome. The premessenger has 20 exons. It covers 5.98 kb on the WS97 genome. It is transpliced to SL1 or SL2. The protein (1037 aa, 119.2 kDa, pI 6.3) contains one DEAD/DEAH box helicase motif (DEAD disclosed as SEQ ID NO: 14; DEAH disclosed as SEQ ID NO: 15), one helicase, C-terminal motif. Taxblast (threshold 10^-3) tracks ancestors down to archaea and bacteria and eukaryota. COMPLETENESS: full length. Method: conceptual translation. FEATURES Location/Qualifiers source 1..1037 /organism="Caenorhabditis elegans" /db_xref="taxon:6239" /chromosome="IV" /map="IV;+3.32 cM (interpolated genetic position)" /map="IV; covering 5976 bp, from base 6613343 to 6607368 on genome release WS97" /clone="Primers to amplify the CDS (9336 bp, Stop included): ATGAGGAAAAAGCAGTGTTCTTCAATA (SEQ ID NO: 16) (T=57.4), TTATGCTTCTCTGATTAAATTGACTAC (SEQ ID NO: 17) (T=55.9). Complete CDS clones: AF480439, yk850g8, yk1388a5, yk1414c1, yk1627h8. Recommended clone (from the Kohara collection): yk850g8. Other clone(s): yk1716a1, yk447b12, yk296e5, yk6g7, yk317d10, yk354h4, yk240c5, yk134d4, yk606h12, yk317d8, yk225b1, yk207d7, yk219b1, yk1752c2. for edited clone sequences see /clone_lib="Kohara embryonic lambda gt11 library: yk447b12, yk606h12; Kohara Sugano L2 larvae cap- selected library: yk1716a1, yk1388a5, yk1414c1, yk1627h8, yk1752c2; Kohara Sugano L4 larvae cap-selected library: yk850g8; Kohara mixed stage library, from him-8 strain, containing 15-30% males: yk207d7; Kohara mixed stage library, from him-8 strain, containing 15-30% males: yk296e5, yk6g7, yk317d10, yk354h4, yk240c5, yk134d4, yk317d8, yk225b1, yk219b1; gb: AF480439" Protein 1..1037 /product="Dicer-Related Helicase, a DExH-box helicase (119.2 kD) (drh-1)" Region 283..510 /region_name="[Pfam/InterPro description] DEAD/DEAH box (DEAD disclosed as SEQ ID NO: 14; DEAH disclosed as SEQ ID NO: 15) helicase" /db_xref="CDD:pfam00270" Region 723..810 /region_name="[Pfam/InterPro description] helicase, C-terminal" /db_xref="CDD:pfam00271" CDS 1..1037 /gene="drh-1" /locus_tag="4H372" /coded_by="NM_068617.2:6..3119" /db_xref="AceView/WormGenes:drh-1" /db_xref="GeneID:177425" /db_xref="LocusID:177425" /db_xref="WormBase:F15B10.2" ORIGIN 1 mrkkqcssil slydkeiilc lepiyrdpek gdgfsellpl gridelkiqs enaqefskql 61 yhdlknsils nadderlykd imtylqtylp kctvhkllnc snrevklsdf hyildhfegf 121 lrfiepkvvl ayldsypqyi davavlrkei erneednqds dfikklilrt vpllgeqavy 181 dimytiseks snnldveakq fiakvlrlkn dgflrfyqii nasrrqlngr iyicpvhesa 241 temmvylgta alntnryrmi nirvdnivqe nstprlvies vrqrihrqrq lclrnyqeel 301 cqvalqgknt ivtaptgsgk tviaaniike hfesrssegk rfkalfmtpn smilnqqaas 361 issyldhvyh tqiiqgsdnv ptrnviqskd livatpqmiv nlcnehrnsl ddesrldqff 421 lstftiiffd echntvknsp ysnimreyhy lknmgnmpeg hslpqiiglt aslgtgdknd 481 clqvrnyiag lcasmdvkdl sivkdnleel rgyspivpdk vllcerstdg pigmftnrlt 541 lmmqevegli rtalrnehig ieqrrqiett erdfrpdssf ldppadkeha gyqnwvcnqm 601 nlvsgtsfre tgtrtiinea ldvlkecfct lsyninfhpe valnylkdem eyrtpnftvn 661 miriweryhn qlvgtgsaen pmisktvqyi veqnlqrads rtiifvrtry eatilnkvln 721 sneellmlgi ksewmsglnk stassadisa skqkqmeklk mfadgeiril vstsvaeegl 781 dvpecslvik ynyatneiah vqrrgrgral nsecvlitns ialrdqesnn rdkeslmset 841 isliqnspae frkcvdeesn kiwprilred tdkaqkieeq inrnivykii ckkceailct 901 skdirsrntq ylvcdpgfws lvrktrltde qqalikynat gsincrrenc glklgqliev 961 ntvdlpclsa lsivllvegt dkriivkkwk nildkyftpt eirqldvqtm rdadqartpm 1021 vfehhangev vnlirea (SEQ ID NO: 18)IIIE.DRH-2 LOCUS NP_501019 620 aa linear DEFINITION Dicer-Related Helicase (71.3 kD) (drh-2) [Caenorhabditis elegans]. ACCESSION NP_501019 VERSION NP_501019.2 GI:25145329 DBSOURCE REFSEQ: accession NM_068618.2 KEYWORDS . SOURCE Caenorhabditis elegans ORGANISM Caenorhabditis elegans Eukaryota; Metazoa; Nematoda; Chromadorea; Rhabditida; Rhabditoidea; Rhabditidae; Peloderinae; Caenorhabditis. REFERENCE 1 (residues 1 to 620) AUTHORS Tabara,H., Yigit,E., Siomi,H. and Mello,C.C. TITLE The dsRNA binding protein RDE-4 interacts with RDE-1, DCR-1, and a DExH-box helicase to direct RNAi in C. elegans JOURNAL Cell 109 (7), 861-871 (2002) MEDLINE 22105477 PUBMED 12110183 COMMENT REVIEWED REFSEQ: This record has been curated by NCBI staff. This record is derived from an annotated genomic sequence (NC_003282). The reference sequence was derived from AF480440.1 and D33924.1. This gene drh-2, also known as C01B10.1, 4H380 or YK1203, maps at (IV; +3.33). It encodes a Dicer-Related Helicase. From Pfam homology, the product would have ATP binding, nucleic acid binding, helicase activities. According to the Worm Transcriptome Project, it is well expressed mostly from L1 larvae to adult [Kohara cDNAs]. Its sequence is defined by 10 cDNA clones. RNA interference results: [A.Sugimoto 2000] No obvious phenotype (by injecting cDNA clone SA:yk272f7). [J.Ahringer 2003] No obvious phenotype (by feeding genomic PCR product JA:C01B10.1). Expression The expression profile for the gene, derived from the proportion of animals at each stage in each Kohara library is: embryos 2%, L1 or L2 larvae 27%, L3 to adult 70%. In situ hybridisation pictures to all stages of development are available from Kohara NextDB. This complete CDS mRNA is 3277 bp long. Its sequence exactly matches the genome. The premessenger has 19 exons. It covers 4.76 kb on the WS97 genome. It has a very long 5' UTR. The protein (620 aa, 71.3 kDa, pI 6.2) contains one helicase, C- terminal motif. Taxblast (threshold 10^-3) tracks ancestors down to archaea and viruses and bacteria and eukaryota. COMPLETENESS: full length. Method: conceptual translation. FEATURES Location/Qualifiers source 1..620 /organism="Caenorhabditis elegans" /db_xref="taxon:6239" /chromosome="IV" /map="IV;+3.33 cM (interpolated genetic position)" /map="IV; covering 4758 bp, from base 6618488 to 6613731 on genome release WS97" /clone="Primers to amplify the CDS (5583 bp, Stop included): ATGATTGTAAATCTTTGCAATGAGCAC (SEQ ID NO: 19) (T=57.4), TTATGCTTGTCTAATTACATTGATTACTT (SEQ ID NO: 20) (T=55.0). Complete CDS clones: AF480440, yk38c3, yk226c6, yk1564a4, yk1605b6. Recommended clone (from the Kohara collection): yk226c6. Other clone(s): yk315f1, yk1017f9, yk1007f1, yk1080b12, yk272f7. Anomalous clones: yk1080b12 (Suspected internal deletion) for edited clone sequences see /clone_lib="Kohara Sugano L1 larvae cap-selected library: yk1017f9, yk1007f1, yk1080b12; Kohara Sugano L2\ larvae cap-selected library: yk1605b6; Kohara Sugano L4 larvae cap-selected library: yk1564a4; Kohara mixed stage library, from him-8 strain, containing 15-30% males: yk226c6, yk38c3, yk315f1, yk272f7; gb: AF480440" Protein 1..620 /product="Dicer-Related Helicase (71.3 kD) (drh-2)" Region 305..392 /region_name="[Pfam/InterPro description] helicase, C-terminal" /db_xref="CDD:pfam00271" CDS 1..620 /gene="drh-2" /locus_tag="4H380" /coded_by="NM_068618.2:1238..3100" /db_xref="AceView/WormGenes:drh-2" /db_xref="GeneID:177426" /db_xref="LocusID:177426" /db_xref="WormBase:C01B10.1" ORIGIN 1 mivnlcnehr dplddeyppe qfflstftii ffdechntvk nspysnvmre yhylknmgnm 61 peghsfpqii gltaslgtgd kkncmqvrsy iaglcanmdv kelsivkdnl eelldhnpfv 121 tdqvsfcers ndgpiemftk rlkqmmqeve dlirttlkne ptvkyeippt dkehnryenw 181 isnqrncvsl agsrnktlii evldvlkdcf yalsyninfn pevalkkyle kelgperirn 241 ftdnmnliwd nchrelvgig saenpmiart vqfildqneq tsdfraiifv rtkkeadfln 301 yvlndrlhel giksdwmsgq kkstassadi saskqkqmek lkmfadgenq ilvstsvaee 361 gldipecslv ikynyatnet ahvqrrgrar arnskcvlit nsialhvqes nnlakenlmt 421 etisliqnsp gefrqcvdee snkvwpriqr edtdkaqrik eqinrnivyk ivcmkcdtvl 481 ctnkdirskn tqyivcnpgf wslvrriplp leqrasnkfn stgsieclge rcgsklgqli 541 dvntvnlpcl kvksilllie stnerilvkq wknildehft pttlkqrdvq tmkdadygra 601 piefehhtan gevinvirqa (SEQ ID NO: 21)

IIIF.Helicase homologous to DCR-2 (DRH-3) DCR-2 has been officially renamed DRH-3 and is a paralog of DRH-1 and DRH-2 which are essential for RNAi. Importantly, the human ortholog for DRH-3 is melanoma differentiation associated protein-5. MQPTAIRLEDYDKSKLRLPFESPYFPAYFRLLKWKFLDVCVESTRNNDIG YFKLFESLFPPGKLEEIARMIIDEPTPVSHDPDMIKIRNADLDVKIRKQA ETYVTLRHAHQQKVQRRRFSECFLNTVLFDEKGLRIADEVMFNYDKELYG YSHWEDLPDGWLTAETFKNKFYDEEEVTNNPFGYQKLDRVAGAARGMIIM KHLKSNPRCVSETTILAFEVFNKGNHQLSTDLVEDLLTEGPAFELKIENG EEKKYAVKKWSLHKTLTMFLAIIGFKSNDKKEKNEHEEWYYGFIDAMKND PANRAALYFLDKNWPEELEEREKERDRIRLTLLKSQRTNEEAVGEDVCTT IRPQPKDSGYNPDAVVTELVLRTYQEELVQPALEGKNCVIVAPTGSGKTE VAIYAALKHIEERTSQGKPSRVVLLVPKIPLVGQQKDRFLKYCNGMYEVN GFHGSESSVSGTGRRDEVIATHVSVMTPQILINMLQSVRQNERLYVSDFS MMIFDEVHKAAKNHPYVLINQMVQEWKYEKPQIIGLTASLSVKVDGQKDE NQMLNDIYNMLALINAPHLSTITRQSSIDELNEHVGKPDDSVELCLPAKE NILRDYIERYLNHAHGKFLEELASMSKSTGRNNTIPPNMINTFKKNQPKN YEYYDSLLQGIIQELNKLNVPEKWNSQTWAKYMKVYLEARGIVDLMPAMV AFKYMEKAIGKLNESHSETVEYSTFIKDHDTLKQTIQSVEPEIVLRLKKY THQSVPHQFGNYGEQMVGYVLGTNKQGAVQQTSQEQQLTLDKFNNGRLKV IVATSVVEEGLDVTACNLIIKYNCSSGSAIQLVQQRGRARAKNSRSVLLS VKSSINETETNALISEKYMRLCVKKITENGEKQLAAEVKRVAELNAAERK RNLEEQLNLRLRHENKIYKLMCSNCSKEFCKSIYIKKVFSNYMVFDPSVW RFLHVESVETFIKCLKITWKCRIADYQIAEFPNFAFRQLTFRLFLCNFQM FQKRKVSKYLSEDNQPLSDIKCFHCKLDVGRAYKIRGTYLPQLSVKALTF VQESDYSSMTKAKWSDVEQDLFYISEAIEDDFRIMLNALSDTEENIEKKI VLDLDSRQHNKQLEMKRFHIQQEPPTKGVAPEAQ (SEQ ID NO: 22)

Also see the C. elegans Protein Database: Wormpep at Wellcome Trust Sanger Institute, Wellcome Trust Genome Campus, Hinxton, Cambridge, CB10 ISA, UK; Accesion No. ce09069.

IIIG.Double helicase MADELARIQQYEYRQNSNLVLSVDYNLTDRRGREEPTGEVLPITDKEMRK MKMGDRAIKGKAPVQDQKKKRKKKDDEKAQQFGRNVLVDNNELMGAYKPR TQETKQTYEVILSFILDALGDVPREVLCGAADEVLLTLKNDKFRDKEKKK EVEALLGPLTDDRIAVLINLSKKISDFSIEEENKPEGDGDIYENEGVNVQ FDSDEEEDDGGMVNEIKGDSEEESEEEEGVDTDYTATLKGDGHLTEDEQK ARGILHPRDIDAHWIQRSLAKYFKDPLIAQQKQTEVIGILKNAADDRDAE NQLVLLLGFDQFEFIKCLRQNRLMILYCTLLRQANEKERLQIEDDMRSRP ELHPILALLQETDEGSVVQVEKSKRDAEKSKKAATAANEAISAGQWQAGR KMLDLNDLTFSQGSHLMSNKRCELPDGSYRRQKKSYEEIHVPALKPRPFA EGEKLVSVSELPKWAQPAFDGYKSLNRIQSRLCDSALRSKEHLLLCAPTG AGKTNVALLTMLQEIGNHLAEDGSVKLDEFKIVYIAPMKSLVQEMVGSFS KRLAPFGITVGEMTGDAQMSKEQFMATQVIVCTPEKYDVVTRKGGERAYN QMVRLLIIDEIHLLHDDRGPVLESIVVRTIRQMEQNHDECRLVGLSATLP NYQDVATFLRVKPEHLHFFDNSYRPVPLEQQYIGVTEKKALKRFQAMNEV VYDKIMEHAGKSQVLVFVHSRKETAKTAKAIRDACLEKDTLSAFMREGSA STEILRTEAEQAKNLDLKDLLPYGFAIHHAGMNRVDRTLVEDLFADRHIQ VLFSTATLAWGVNLPAHTVIIKGTQIYNPEKGRWTELGALDIMQMLGRAG RPQYDDRGEGILITNHSELQYYLSLMNQQLPVESQMVSRLTDMLNAEVVL GTVSSVSEATNWLGYTFLFVRMLKNPTLYGITHEQARADPLLEQRRADLI HTACVLLDKAGLIKYDKRSGIIQATELGRIASHFYCTYESMQTYNKLLVE TCSDIDLFRIFSMSSEFKLLSVRDEEKLELQKMAEHAPIPIKENLDEASA KTNVLLQAYISQLKLEGFALQADMVFVAQSAGRLFRALFEIVLWRGWAGL AQKVLTLCKMVTQRQWGSLNPLHQFKKIPSEVVRSIDKKNYSFDRLYDLD QHQLGDLIKMPKMGKPLFKFIRQFPKLEMTTLIQPITRTTMRIELTITPD FKWDEKVHGSAEGFWIFIEDTDGEKILHHEFFLLKQKFCSDEHVVKMIVP MFDPMPPLYYVRIVSDRWIGAETVLPISFRHLILPEKYPPPTELLDLQPL PISAVTNKEFQTVFAESGFKVFNPIQTQVFRTVFESNENVIVCAPNGSGK TAIAELAVLRHFENTPEAKAVYITPMEDMATKVYADWKRRLEPAIGHTIV LLTGEQTMDLKLAQRGQLIISTPERWDNISRRWKQRKSVQNVKLFIADDL HMIGASNGAVFEVVCSRTRYISSQLESAVRVVALSSSLTNARDLGMWLGC SASATFNFMPSTRPVPLDLEIKSFNLSHNASRFAAMERPVYQAICRHAGK LEPKPALVFVPVRRQTRPVAVALLTMALADGAPKRFLRLAEHDDTFQALL ADIEDESLRESVSCGVGFLHEGTAPKDVHIVQQLFESNAIQVCVVPRGMC YQIEMSAYLVVVMDTQFYNGKYHVYEDYPIADMLHMVGLANRPILDSDAK CVVMCQTSKRAYYKKFLCDPLPVESHLDHCLHDHFNAEIVTKTIENKQDA IDYLTWTLLYRRMTQNPNYYNLQGTTHRHLSDALSELVELTLKDLENSKC IAVKDEMDTVSLNLGMIASYYYISYQTIELFSMSLKEKTKTRALIEIISA SSEFGNVPMRHKEDVILRQLAERLPGQLKNQKFTDPHVKVNLLIHAHLSR VKLTAELNKDTELIVLRACRLVQACVDVLSSNGWLSPAIHAMELSQMLTQ AMYSNEPYLKQLPHCSAALLERAKAKEVTSVFELLELENDDRSDILQMEG AELADVARFCNHYPSIEVATELENDVVTSNDNLMLAVSLERDNDIDGLAP PVVAPLFPQKRKEEGWWLVIGDSESNALLTIKRLVINEKSSVQLDFAAPR PGHHKFKLFFISDSYLGADQEFDVAFKVEEPGRSNRKRKHEKEED (SEQ ID NO: 23)

Also see the C. elegans Protein Database: Wormpep at Wellcome Trust Sanger Institute, Wellcome Trust Genome Campus, Hinxton, Cambridge, CB10 ISA, UK; Accesion No. ce21971.IIIH.EFT-2, EF-Tu family GTP Binding Protein LOCUS NP_492457 852 aa linear DEFINITION translation Elongation FacTor (94.8 kD) (eft-2) [Caenorhabditis elegans]. ACCESSION NP_492457 VERSION NP_492457.1 GI:17506493 DBSOURCE REFSEQ: accession NM_060056.2 KEYWORDS . SOURCE Caenorhabditis elegans ORGANISM Caenorhabditis elegans Eukaryota; Metazoa; Nematoda; Chromadorea; Rhabditida; Rhabditoidea; Rhabditidae; Peloderinae; Caenorhabditis. REFERENCE 1 (residues 1 to 852) AUTHORS Fraser,A.G., Kamath,R.S., Zipperlen,P., Martinez-Campos,M., Sohrmann,M. and Ahringer,J. TITLE Functional genomic analysis of C. elegans chromosome I by systematic RNA interference JOURNAL Nature 408 (6810), 325-330 (2000) MEDLINE 20548709 PUBMED 11099033 REFERENCE 2 (residues 1 to 852) AUTHORS Ofulue,E.N. and Candido,E.P. TITLE Isolation and characterization of eft-1, an elongation factor 2-like gene on chromosome III of Caenorhabditis elegans JOURNAL DNA Cell Biol. 11 (1), 71-82 (1992) MEDLINE 92153310 PUBMED 1739435 REFERENCE 3 (residues 1 to 852) AUTHORS Ofulue,E.N. and Candido,E.P. TITLE Molecular cloning and characterization of the Caenorhabditis elegans elongation factor 2 gene (eft-2) JOURNAL DNA Cell Biol. 10 (8), 603-611 (1991) MEDLINE 92029622 PUBMED 1930695 COMMENT REVIEWED REFSEQ: This record has been curated by NCBI staff. This record is derived from an annotated genomic sequence (NC_003279). The reference sequence was derived from BJ105642.1, AU205829, M86959 and AU218565. Summary: This essential gene eft-2, also known as F25H5.4, 1J741 or YK6, maps at (I; +3.37). Its phenotype is embryonic lethal, protruding vulva. It encodes a translation Elongation FacTor. From Pfam homology, the product would have GTP binding, translation elongation factor activities, would be involved in translational elongation. According to the Worm Transcriptome Project, it is expressed at very high level at all stages of development except in embryos [Kohara cDNAs]. Its sequence is defined by 1015 cDNA clones. RNA interference results [J.Ahringer 2000] embryonic lethal (100%), protruding vulva (by feeding genomic PCR product JA:F25H5.4). Function Protein properties: [C.elegansII] NMK. Encodes protein with >80% similarity to elongation factor EF-2 from yeast, Drosophila, human. [Ofolue and Candido 1992]. Expression The expression profile for the gene, derived from the proportion of animals at each stage in each Kohara library is: embryos 3%, L1 or L2 larvae 13%, L3 to adult 34%. In situ hybridisation pictures to all stages of development are available from Kohara NextDB. For a detailed expression pattern description, see Wormbase Expr1390. This complete mRNA is 2819 bp long. Its sequence exactly matches the genome. The premessenger has 6 exons. It covers 3.23 kb on the WS97 genome. It is transpliced to SL1. The protein (852 aa, 94.8 kDa, pI 6.1) contains one Elongation factor, GTP-binding motif, one Elongation factor Tu, domain 2 motif, one Elongation factor G, domain IV motif, one Elongation factor G, C-terminal motif. It also contains a coil coil stretch, an ER membrane domain [Psort2]. Taxblast (threshold 10^-3) tracks ancestors down to archaea and bacteria and eukaryota. COMPLETENESS: full length. Method: conceptual translation. FEATURES Location/Qualifiers source 1..852 /organism="Caenorhabditis elegans" /db_xref="taxon:6239" /chromosome="I" /map="I;+3.37 cM (interpolated genetic position)" /map="I; covering 3303 bp, from base 9171586 to 9174890 on genome release WS97" Protein 1..852 /product="translation Elongation FacTor (94.8 kD) (eft-2)" Region 17..356 /region_name="[Pfam/InterPro description] elongation factor, GTP-binding" /db_xref="CDD:pfam00009" Region 176..177 /region_name="[PSORT] dileucine domain: LL" Region 298..325 /region_name="[PSORT] coil coil domain: VMNIKKDKTAALVEKLGIKLANDEKDLE" (SEQ ID NO: 24) Region 401..480 /region_name="[Pfam/InterPro description] elongation factor Tu, domain 2" /db_xref="CDD:pfam03144" Region 614..731 /region_name="[Pfam/InterPro description] elongation factor G, domain IV" /db_xref="CDD:pfam03764" Region 655..656 /region_name="[PSORT] dileucine domain: LL" Region 733..821 /region_name="[Pfam/InterPro description] elongation factor G, C-terminal" /db_xref="CDD:pfam00679" Region 734..735 /region_name="[PSORT] dileucine domain: LL" Region 833..836 /region_name="[PSORT] nuclear localization domain: RKRK" (SEQ ID NO: 25) Region 848..851 /region_name="[PSORT] ER membrane domain: YLDK" (SEQ ID NO: 26) CDS 1..852 /gene="eft-2" /locus_tag="1J741" /coded_by="NM_060056.2:124..2682" /db_xref="AceView/WormGenes:eft-2" /db_xref="GeneID:172743" /db_xref="LocusID:172743" /db_xref="WormBase:F25H5.4" ORIGIN 1 mvnftvdeir almdrkrnir nmsviahvdh gkstltdslv skagiiagsk agetrftdtr 61 kdeqerciti kstaislffe lekkdlefvk genqfetvev dgkkekyngf linlidspgh 121 vdfssevtaa lrvtdgalvv vdcvsgvcvq tetvlrqaia erikpvlfmn kmdrallelq 181 lgaeelfqtf qriveninvi iatygdddgp mgpimvdpsi gnvgfgsglh gwaftlkqfa 241 emyagkfgvq vdklmknlwg drffdlktkk wsstqtdesk rgfcqfvldp ifmvfdavmn 301 ikkdktaalv eklgikland ekdlegkplm kvfmrkwlpa gdtmlqmiaf hlpspvtaqk 361 yrmemlyegp hddeaavaik tcdpngplmm yiskmvptsd kgrfyafgrv fsgkvatgmk 421 ariqgpnyvp gkkedlyekt iqrtilmmgr fiepiedips gniaglvgvd qylvkggtit 481 tykdahnmrv mkfsvspvvr vaveaknpad lpklveglkr laksdpmvqc ifeesgehii 541 agagelhlei clkdleedha ciplkksdpv vsyretvqse snqiclsksp nkhnrlhcta 601 qpmpdgladd ieggtvnard efkarakila ekyeydvtea rkiwcfgpdg tgpnllmdvt 661 kgvqylneik dsvvagfqwa tregvlsden mrgvrfnvhd vtlhadaihr gggqiiptar 721 rvfyasvlta eprllepvyl veiqcpeaav ggiygvlnrr rghvfeesqv tgtpmfvvka 781 ylpvnesfgf tadlrsntgg qafpqcvfdh wqvlpgdple agtkpnqivl dtrkrkglke 841 gvpaldnyld km (SEQ ID NO: 27) III.EFT-4 (eIF1 alpha) LOCUS NP_509323 463 aa linear DEFINITION translation Elongation FacTor (50.7 kD) (eft-4) [Caenorhabditis elegans]. ACCESSION NP_509323 VERSION NP_509323.1 GI:17569207 DBSOURCE REFSEQ: accession NM_076922.1 KEYWORDS . SOURCE Caenorhabditis elegans ORGANISM Caenorhabditis elegans Eukaryota; Metazoa; Nematoda; Chromadorea; Rhabditida; Rhabditoidea; Rhabditidae; Peloderinae; Caenorhabditis. REFERENCE 1 (residues 1 to 463) AUTHORS Kamath,R.S., Fraser,A.G., Dong,Y., Poulin,G., Durbin,R., Gotta,M., Kanapin,A., Le Bot,N., Moreno,S., Sohrmann,M., Welchman,D.P., Zipperlen,P. and Ahringer,J. TITLE Systematic functional analysis of the Caenorhabditis elegans genome using RNAi JOURNAL Nature 421 (6920), 231-237 (2003) MEDLINE 22417569 PUBMED 12529635 COMMENT VALIDATED REFSEQ: This record has undergone preliminary review of the sequence, but has not yet been subject to final review. This record is derived from an annotated genomic sequence (NC_003284). The reference sequence was derived from WormBase CDS:R03G5.1a. Summary: This essential gene eft-4, also known as eln-2, R03G5.1, XI443 or YK211, maps at (X; -0.81). Its phenotype is embryonic lethal, partial, slow growth. It encodes a translation Elongation FacTor. From Pfam homology, the products would have GTP binding, translation elongation factor activities, would be involved in translational elongation. According to the Worm Transcriptome Project, it is expressed at very high level at all stages of development [Kohara cDNAs]. Its sequence is fully supported by 406 cDNA clones and produces, by alternative splicing, 4 different transcripts a, b, c, d altogether encoding 4 different protein isoforms. RNA interference results [J.Ahringer 2003] Embryonic lethal (40%), slow growth (by feeding genomic PCR product JA:R03G5.1). Function Protein properties: [C.elegansII] NMK. Encodes EF1 alpha protein, aa sequence identical to eft-3. [FK]. Expression The expression profile for the gene, derived from the proportion of animals at each stage in each Kohara library is: embryos 6%, L1 or L2 larvae 58%, L3 to adult 37%. In situ hybridisation pictures to all stages of development are available from Kohara NextDB. The CDS has 3 exons. It covers 1.59 kb on the WS97 genome. The protein (463 aa, 50.7 kDa, pI 9.1) contains one Elongation factor, GTP-binding motif, one Elongation factor Tu, domain 2 motif, one Elongation factor Tu, C-terminal motif. It also contains an ER membrane domain [Psort2]. Taxblast (threshold 10^-3) tracks ancestors down to eukaryota. COMPLETENESS: full length. Method: conceptual translation. FEATURES Location/Qualifiers source 1..463 /organism="Caenorhabditis elegans" /db_xref="taxon:6239" /chromosome="X" /map="X;-0.81 cM (interpolated genetic position)" /map="X; covering 2129 bp, from base 7814176 to 7816306 on genome release WS97" Protein 1..463 /product="translation Elongation FacTor (50.7 kD) (eft-4)" Region 5..239 /region_name="[Pfam/InterPro description] elongation factor, GTP-binding" /db_xref="CDD:pfam00009" Region 258..327 /region_name="[Pfam/InterPro description] elongation factor Tu, domain 2" /db_xref="CDD:pfam03144" Region 333..442 /region_name="[Pfam/InterPro description] elongation factor Tu, C-terminal" /db_xref="CDD:pfam03143" Region 459..462 /region_name="[PSORT] ER membrane domain: APKK" (SEQ ID NO: 28) Region 460..463 /region_name="[PSORT] nuclear localization domain: PKKK" (SEQ ID NO: 29) CDS 1..463 /gene="eft-4" /locus_tag="XI443" /coded_by="NM_076922.1:1..1392" /db_xref="AceView/WormGenes:eft-4" /db_xref="GeneID:181044" /db_xref="LocusID:181044" ORIGIN 1 mgkekvhini vvighvdsgk stttghliyk cggidkrtie kfekeaqemg kgsfkyawvl 61 dklkaererg itidialwkf etakyyitii dapghrdfik nmitgtsqad cavlvvacgt 121 gefeagiskn gqtrehalla qtlgvkqliv acnkmdstep pfsearftei tnevsgfikk 181 igynpkavpf vpisgfngdn mlevssnmpw fkgwaverke gnasgktlle aldsiippqr 241 ptdrplrlpl qdvykiggig tvpvgrvetg iikpgmvvtf apqnvttevk svemhheslp 301 eavpgdnvgf nvknvsvkdi rrgsvcsdsk qdpakeartf haqviimnhp gqisngytpv 361 ldchtahiac kfnelkekvd rrtgkkvedf pkflksgdag iveliptkpl cvesftdyap 421 lgrfavrdmr qtvavgviks veksdgssgk vtksaqkaap kkk (SEQ ID NO: 30)IIIJ.GAP/RAN-GAP family SWSGDKLAWLQTWRRVISLVDPYTNSSAHVAIDCMSLTIENLLLVNLHPL AHWLACRLVTVPPILLPRCVPALSAILNESTIRRPPPLLSANILLCFIRL MQSKEQLVVPAICGLSAHELSIVAPRALEHLPKMLQAAKSSKDTKVSSNS LKLFSMLASSYPGAEQILLDQLVNTDVSENAVVIVNSLAILIVQKAQIDL VLTALKTIETHQFAMRLIPLFCSSIASLAQFSSTTLLQALLRAASLLRDE RTRTEIEWQMVKLCMQWPQPQMPLVIRGILADRHMVLHGELVTLGGQYPV RGFEVQRWSSAGAPPLQGEDKTVYINRQSAIISVSRKDFHAKSPCEITSR TVVGRHIWDLDTHEDVRKPATNVTNWLRKEALKGKRPGRESQGILGAMDD PFDDLPDYPPSRGSPSPVDGAAQFTSMIETSRRQPQPLGTSSAAHDHLPA FTPNAKLLEWRSLSASLGFVPLVSQVHANFPRDLKHLDQTSSREVHKVAV IYVGESQEDRASILSNTTASASAQFDSFTSELGWEVKVGRGHDGYTGGLP VETRAPYFADAEAEVIFHVSTMLNGDVQQKWKHIGNDEVHVVWTENTRKV YTRETIATKFCDVLIVLEQVGDKMVRVRVDTASALEFGPLFDGALVTMSE LSQLVRLTVINASRAYRLARVEHSRPLRHREEVFCNEALAHMKPMPLAQS INHLYVPTI (SEQ ID NO: 31)

Also see the C. elegans Protein Database: Wormpep at Wellcome Trust Sanger Institute, Wellcome Trust Genome Campus, Hinxton, Cambridge, CB10 ISA, UK; Accesion No. ce21437.

IIIK.HMG-I/Y DNA Binding Protein MVEGDVDESASGTSGTNKKILFTKKPSVWKDFDNWINDEPENRYDLFQVV KSAMLLQSGYTTILMDQVTDNGADELRISLEYSNFIKIVNSTKLVVGKEQ CPPSNVFTLLAEIFANTPGNTSEVGRISTWLTSHLGALLHNDVIWKIHFF DPDLFRSVYWQLIFTLKLAPGDTENLEEDENYAKLLFSCFITAVMVALWH DHEMSFNSICPDYLKPETASEYMVMLISSPPFRSLSQFFLFGLHLLGKYQ SEGGCVVVREEAYIAEIRQNDEEKRQSIETRTNLISDDMVYDDGEDLLEQ IDRVQQLHEAHCIVLLKKGFLKAPDGFKIVQKGGRPRKYPASATKKRKKK TPRSSPKKKMSKESPINHQKEPIDEQKPSTSLPIYSVATLKPRRKVVKTA DEVGLCAPIFVMQSELLKKFREEVQRRYAEGSSASDQERVRNMVYEAYDN IYHINRLSANEGPRILTSDQKLVMQQYKTTFRQGPTFAEETESDVEEEEE KKVVEVVTAKVIKGSAKSSKKFKRRY (SEQ ID NO: 32)

Also see the C. elegans Protein Database: Wormpep at Wellcome Trust Sanger Institute, Wellcome Trust Genome Campus, Hinxton, Cambridge, CB10 ISA, UK; Accesion No. ce08872.

IIIL.HMG-I/Y DNA binding PB1 domain

Also see the C. elegans Protein Database: Wormpep at Wellcome Trust Sanger Institute, Wellcome Trust Genome Campus, Hinxton, Cambridge, CB10 ISA, UK; Accesion No. ce20336.

IIIM.SNR-2 SM protein

Member of the Small Nuclear Ribonucleoprotein gene class. MTISKNNKMM AHLNYRMKII LQDGRTFIGF FKAFDKHMNI LLAECEEHRQ IKPKAGKKTD GEEKRILGLV LVRGEHIVSM TVDGPPPRDD DSVRLAKAGG AGGVGQAKPG GRGMPAMPGM PGMPPGGAPG GLSGAMRGHG GPGMAAMQPG YGGPPGGRPF (SEQ ID NO: 33)

Also see the C. elegans Protein Database: Wormpep at Wellcome Trust Sanger Institute, Wellcome Trust Genome Campus, Hinxton, Cambridge, CB10 ISA, UK; Accesion No. ce14704.

Homologs include Swiss-Prot. TrEMBL Accession No. Q15182 H. sapiens and TrEMBL Accession No. O70499, M. musculus Small nuclear ribonucleoprotein N.

IIIN.SNR-3 SM protein

The SNR-3 SM protein is a member of the Small Nuclear Ribonucleoprotein SMD1 gene class. A homolog for this gene product is human SMD1.

MKLVRFLMKL SHETVNIELK NGTQVSGTIM GVDVAMNTHL RAVSMTVKNK EPVKLDTLSI RGNNIRYIIL PDPLALDTLL IDDEPRKKAR AARAGASRGR GRGGMRGGRG GRGRGRGGPR GGGPRR (SEQ ID NO: 34)

Also see the C. elegans Protein Database: Wormpep at Wellcome Trust Sanger Institute, Wellcome Trust Genome Campus, Hinxton, Cambridge, CB10 ISA, UK; Accesion No. ce02065.

IIIO.Dual specificity phosphatase MPEPRCTAIV NFLNLSHSIL ISIFSVSVMS NYHHNHNYQH RPRGYERLPG KRLPDRWNIY DNVGRDIDGT RFVPFKTPLD SSFFDGKNMP VELQFGVKTL ISLAQQANKQ IGLVIDLTNT DRYYKKTEWA DHGVKYLKLN CPGHEVNERE DLVQDFINAV KEFVNDKEND GKLIGVHCTH GLNRTGYLIC RYMIDVDNYS ASDAISMFEY YRGHPMEREH YKKSLYEAER KKKYGKSSGK SSGNSADSTI SSEQLHRNNS Q (SEQ ID NO: 35)

Homologs include, for example, Swiss Prot. Accession No. O75319, H. sapiens Dual specificity protein phosphatase 11 and TrEMLB Accession No. Q8BTR4, similar to dual specificity protein phosphatase 11.

Also see the C. elegans Protein Database: Wormpep at Wellcome Trust Sanger Institute, Wellcome Trust Genome Campus, Hinxton, Cambridge, CB10 ISA, UK; Accesion No. ce03706.

IIIP.LIN-41

A homolog of LIN-41 is the human tripartite motif protein 2 (RING finger protein 86) LOCUS NP_492487 1143 aa linear DEFINITION abnormal cell LINeage LIN-41, heterochronic gene; Drosophila dappled/ vertebrate TRipartite Motif protein related; B-box zinc finger, Filamin and NHL repeat containing protein (123.8 kD) (lin-41) [Caenorhabditis elegans]. ACCESSION NP_492487 VERSION NP_492487.2 GI:25149908 DBSOURCE REFSEQ: accession NM_060086.2 KEYWORDS . SOURCE Caenorhabditis elegans ORGANISM Caenorhabditis elegans Eukaryota; Metazoa; Nematoda; Chromadorea; Rhabditida; Rhabditoidea; Rhabditidae; Peloderinae; Caenorhabditis. REFERENCE 1 (residues 1 to 1143) AUTHORS Lin,S.Y., Johnson,S.M., Abraham,M., Vella,M.C., Pasquinelli,A., Gamberi,C., Gottlieb,E. and Slack,F.J. TITLE The C elegans hunchback homolog, hbl-1, controls temporal patterning and is a probable microRNA target JOURNAL Dev. Cell 4 (5), 639-650 (2003) MEDLINE 22623382 PUBMED 12737800 REFERENCE 2 (residues 1 to 1143) AUTHORS Grosshans,H. and Slack,F.J. TITLE Micro-RNAs: small is plentiful JOURNAL J. Cell Biol. 156 (1), 17-21 (2002) MEDLINE 21640444 PUBMED 11781331 REFERENCE 3 (residues 1 to 1143) AUTHORS Ketting,R.F., Fischer,S.E., Bernstein,E., Sijen,T., Hannon,G.J. and Plasterk,R.H. TITLE Dicer functions in RNA interference and in synthesis of small RNA involved in developmental timing in C. elegans JOURNAL Genes Dev. 15 (20), 2654-2659 (2001) MEDLINE 21521222 PUBMED 11641272 REFERENCE 4 (residues 1 to 1143) AUTHORS Sonoda,J. and Wharton,R.P. TITLE Drosophila Brain Tumor is a translational repressor JOURNAL Genes Dev. 15 (6), 762-773 (2001) MEDLINE 21172744 PUBMED 11274060 REFERENCE 5 (residues 1 to 1143) AUTHORS Slack,F.J., Basson,M., Liu,Z., Ambros,V., Horvitz,H.R. and Ruvkun,G. TITLE The lin-41 RBCC gene acts in the C. elegans heterochronic pathway between the let-7 regulatory RNA and the LIN-29 transcription factor JOURNAL Mol. Cell 5 (4), 659-669 (2000) MEDLINE 20337950 PUBMED 10882102 REFERENCE 6 (residues 1 to 1143) AUTHORS Reinhart,B.J., Slack,F.J., Basson,M., Pasquinelli,A.E., Bettinger,J.C., Rougvie,A.E., Horvitz,H.R. and Ruvkun,G. TITLE The 21-nucleotide let-7 RNA regulates developmental timing in Caenorhabditis elegans JOURNAL Nature 403 (6772), 901-906 (2000) MEDLINE 20168806 PUBMED 10706289 COMMENT REVIEWED REFSEQ: This record has been curated by NCBI staff. This record is derived from an annotated genomic sequence (NC_003279). The reference sequence was derived from AF195610. Summary: This gene lin-41, also known as C12C8.3, 1J912 or YK872, maps at (I; +3.53). Its phenotype is abnormal cell lineage, heterochronic. It encodes a heterochronic gene; Drosophila dappled/vertebrate TRipartite Motif protein related; B-box zinc finger, Filamin and NHL repeat containing protein. From Pfam homology, the products would have zinc binding activity and would localize in intracellular. According to the Worm Transcriptome Project, it is well expressed mostly from L1 larvae to adult [Kohara cDNAs]. Its sequence is defined by 11 cDNA clones and produces, by alternative splicing, at least 2 different transcripts b, a altogether encoding 2 different protein isoforms. The transcripts appear to differ by common exons with different boundaries. Phenotype [from C. elegans II book] Allele ma104: heterochronic defect in L4 larvae to adult switch. [Victor Ambros]. Selected strains available from the CGC. CT8 lin-41(ma104) I [Frank Slack, V. Ambros, mutator TR679] Dpy. Precocious heterochronic. Reduced brood size. There may be a linked Dpy mutation in this strain. MT7897 lin-41(n2914)/unc-29(e1072) lin-11(n1281) I [Bob Horvitz, M. Basson, EMS] Heterozygotes are WT and segregate WT, UncVul and lin-41 (Dpy, Scrawny and Sterile). RNA interference results: [J.Ahringer 2000] No obvious phenotype (by feeding genomic PCR product JA:C12C8.3). Expression The expression profile for the gene, derived from the proportion of animals at each stage in each Kohara library is: embryos 3%, L1 or L2 larvae 50%, L3 to adult 47%. In situ hybridisation pictures to all stages of development are available from Kohara NextDB. The report below describes variant a. This complete mRNA is 4797 bp long. It is supported by 2 cDNA clones. Its sequence exactly matches the genome. The premessenger has 16 exons. It covers 7.70 kb on the WS97 genome. It is transpliced to SL1. It has a very long 3' UTR. The protein (1143 aa, 123.8 kDa, pI 6.1) contains 2 Zn-finger, B-box motifs, one Filamin/ABP280 repeat motif, 6 NHL repeat motifs. It also contains a coil coil stretch [Psort2]. Taxblast (threshold 10^-3) tracks ancestors down to archaea and bacteria and eukaryota. COMPLETENESS: full length. Method: conceptual translation. FEATURES Location/Qualifiers source 1..1143 /organism="Caenorhabditis elegans" /db_xref="taxon:6239" /chromosome="I" /map="I;+3.53 cM (interpolated genetic position)" /map="I;+3.75 cM (measured genetic position)" /map="I; covering 7702 bp, from base 9350549 to 9342848 on genome release WS97" /clone="Primers to amplify the CDS (10290 bp, Stop included): ATGGCGACCATCGTGCCATGCT (SEQ ID NO: 36) (T=63.8), CTAGAAGACACGGATGCAATTGTTTCCGAA (SEQ ID NO: 37) (T=63.4). Clone specific of this variant is AF195610. Complete CDS clones: yk1728d7. Recommended clone (from the Kohara collection): yk1728d7. for edited clone sequences see /clone_lib="Kohara Sugano L2 larvae cap-selected library: yk1728d7; gb: AF195610" Protein 1..1143 /product="abnormal cell LINeage LIN-41, heterochronic gene; Drosophila dappled/ vertebrate TRipartite Motif protein related; B-box zinc finger, Filamin and NHL repeat containing protein (123.8 kD) (lin-41)" Region 366..412 /region_name="[Pfam/InterPro description] zn-finger, B-box" /db_xref="CDD:pfam00643" Region 470..512 /region_name="[Pfam/InterPro description] zn-finger, B-box" /db_xref="CDD:pfam00643" Region 553..617 /region_name="[PSORT] coil coil domain: TAENEIRAAFDTHVNALEERRKELLKRVETVKNLKLSVLISQAESLQSKQIDLQQAIQ (SEQ ID NO: 38) TATKLMD" Region 688..810 /region_name="[Pfam/InterPro description] Filamin/ABP280 repeat" /db_xref="CDD:pfam00630" Region 841..868 /region_name="[Pfam/InterPro description] NHL repeat" /db_xref="CDD:pfam01436" Region 888..915 /region_name="[Pfam/InterPro description] NHL repeat" /db_xref="CDD:pfam01436" Region 935..962 /region_name="[Pfam/InterPro description] NHL repeat" /db_xref="CDD:pfam01436" Region 983..1010 /region_name="[Pfam/InterPro description] NHL repeat" /db_xref="CDD:pfam01436" Region 1031..1058 /region_name="[Pfam/InterPro description] NHL repeat" /db_xref="CDD:pfam01436" Region 1116..1143 /region_name="[Pfam/InterPro description] NHL repeat" /db_xref="CDD:pfam01436" CDS 1..1143 /gene="lin-41" /locus_tag="1J912" /coded_by="NM_060086.2:196..3627" /db_xref="AceView/WormGenes:lin-41" /db_xref="GeneID:172760" /db_xref="LocusID:172760" /db_xref="WormBase:C12C8.3a" ORIGIN 1 mativpcsle keegapsgpr rlqteidvda ndsgnelsmg gsssegdsms hhrgehspnh 61 hhqdnhlgsg ppppqftgsl fdtppsmiqs pqqqpqfqfn tgfglglpqd sfrcsvcsks 121 stigvlpfvc ahktcqscyq mtpssydrra cklcgavsta tanftsqmyl sptlpspprg 181 almsdcstpt mnnhinsstp lhqprafsfs lsgmpgspsp vmgarmpssa gglmmrpigf 241 pdsdssltsw splqqpsqls innlssiggh qqqspmlmqn vfdslavndd tpvfsplspt 301 ntsmhmppsl maspdvpkhs atiapprnsm cstprlqlat pmssqsqqtf pipsplgsqp 361 qqqqpmgpiq cqgceskisf aycmqcqeal cihcvqahqr vratkqhafv elqqlmatlm 421 sravqpqqaq qytqnvggsv rqalgsvgsg dghvsgvend sigsgesspr sssvcgthds 481 viigicencp hsvllcaicv aqhpgkhrvq plgdirvavg evvnesqllq wqcektgdti 541 kqiidgivtn attaeneira afdthvnale errkellkrv etvknlklsv lisqaeslqs 601 kqidlqqaiq tatklmdssd cdemvlrqvf eklascqmgn egtepnnnil nvlmlacqvn 661 eddrlkftap qdgillnkar qfgniesgpc aknssivgds fkkairerqt viyvqlrdac 721 gdllsssiaa tqptsqallp hqephshleq amptsdvqaf vispdgstve vtmtprengi 781 valsyypsie gsytlnilvk gtpisgcptt mdirrgrnyd eiaakgpilt fgkegsgdge 841 lcrpwgicvd qrgrvivadr snnrvqifdk dgnfiskfgt sgnrpgqfdr pagittnsln 901 nivvadkdnh rvqvfdengm fllkfgdrgr avgyfnypwg vatnshnaia vsdtrnhrvq 961 iftpqgqfvr kcgfdsayff knldsprglc ylpdgqllit dfnnhrlavl sprnmsemkv 1021 ygsegdgdgm fvrpqgvvid peghilvcds rnnrvqvfas ddmrfigsfg lgpvpnsgfq 1081 mpqelpapys slggpfgapa fssaptpltp sprqlldrpt dlavgpdgri yvvdfgnnci 1141 rvf (SEQ ID NO: 39) LOCUS NP_492488 1147 aa linear DEFINITION abnormal cell LINeage LIN-41, heterochronic gene; Drosophila dappled/ vertebrate TRipartite Motif protein related; B-box zinc finger, Filamin and NHL repeat containing protein (124.2 kD) (lin-41) [Caenorhabditis elegans]. ACCESSION NP_492488 VERSION NP_492488.2 GI:25149913 DBSOURCE REFSEQ: accession NM_060087.2 KEYWORDS . SOURCE Caenorhabditis elegans ORGANISM Caenorhabditis elegans Eukaryota; Metazoa; Nematoda; Chromadorea; Rhabditida; Rhabditoidea; Rhabditidae; Peloderinae; Caenorhabditis. REFERENCE 1 (residues 1 to 1147) AUTHORS Lin,S.Y., Johnson,S.M., Abraham,M., Vella,M.C., Pasquinelli,A., Gamberi,C., Gottlieb,E. and Slack,F.J. TITLE The C elegans hunchback homolog, hbl-1, controls temporal patterning and is a probable microRNA target JOURNAL Dev. Cell 4 (5), 639-650 (2003) MEDLINE 22623382 PUBMED 12737800 REFERENCE 2 (residues 1 to 1147) AUTHORS Grosshans,H. and Slack,F.J. TITLE Micro-RNAs: small is plentiful JOURNAL J. Cell Biol. 156 (1), 17-21 (2002) MEDLINE 21640444 PUBMED 11781331 REFERENCE 3 (residues 1 to 1147) AUTHORS Ketting,R.F., Fischer,S.E., Bernstein,E., Sijen,T., Hannon,G.J. and Plasterk,R.H. TITLE Dicer functions in RNA interference and in synthesis of small RNA involved in developmental timing in C. elegans JOURNAL Genes Dev. 15 (20), 2654-2659 (2001) MEDLINE 21521222 PUBMED 11641272 REFERENCE 4 (residues 1 to 1147) AUTHORS Sonoda,J. and Wharton,R.P. TITLE Drosophila Brain Tumor is a translational repressor JOURNAL Genes Dev. 15 (6), 762-773 (2001) MEDLINE 21172744 PUBMED 11274060 REFERENCE 5 (residues 1 to 1147) AUTHORS Slack,F.J., Basson,M., Liu,Z., Ambros,V., Horvitz,H.R. and Ruvkun,G. TITLE The lin-41 RBCC gene acts in the C. elegans heterochronic pathway between the let-7 regulatory RNA and the LIN-29 transcription factor JOURNAL Mol. Cell 5 (4), 659-669 (2000) MEDLINE 20337950 PUBMED 10882102 REFERENCE 6 (residues 1 to 1147) AUTHORS Reinhart,B.J., Slack,F.J., Basson,M., Pasquinelli,A.E., Bettinger,J.C., Rougvie,A.E., Horvitz,H.R. and Ruvkun,G. TITLE The 21-nucleotide let-7 RNA regulates developmental timing in Caenorhabditis elegans JOURNAL Nature 403 (6772), 901-906 (2000) MEDLINE 20168806 PUBMED 10706289 COMMENT REVIEWED REFSEQ: This record has been curated by NCBI staff. This record is derived from an annotated genomic sequence (NC_003279). The reference sequence was derived from AF195611. Summary: This gene lin-41, also known as C12C8.3, 1J912 or YK872, maps at (I; +3.53). Its phenotype is abnormal cell lineage, heterochronic. It encodes a heterochronic gene; Drosophila dappled/vertebrate TRipartite Motif protein related; B-box zinc finger, Filamin and NHL repeat containing protein. From Pfam homology, the products would have zinc binding activity and would localize in intracellular. According to the Worm Transcriptome Project, it is well expressed mostly from L1 larvae to adult [Kohara cDNAs]. Its sequence is defined by 11 cDNA clones and produces, by alternative splicing, at least 2 different transcripts b, a altogether encoding 2 different protein isoforms. The transcripts appear to differ by common exons with different boundaries. Phenotype [from C. elegans II book] Allele ma104: heterochronic defect in L4 larvae to adult switch. [Victor Ambros]. Selected strains available from the CGC. CT8 lin-41(ma104) I [Frank Slack, V. Ambros, mutator TR679] Dpy. Precocious heterochronic. Reduced brood size. There may be a linked Dpy mutation in this strain. MT7897 lin-41(n2914)/unc-29(e1072) lin-11(n1281) I [Bob Horvitz, M. Basson, EMS] Heterozygotes are WT and segregate WT, UncVul and lin-41 (Dpy, Scrawny and Sterile). RNA interference results: [J.Ahringer 2000] No obvious phenotype (by feeding genomic PCR product JA:C12C8.3). Expression The expression profile for the gene, derived from the proportion of animals at each stage in each Kohara library is: embryos 3%, L1 or L2 larvae 50%, L3 to adult 47%. The expression profile for the gene, derived from the proportion of animals at each stage in each Kohara library is: embryos 3%, L1 or L2 larvae 45%, L3 to adult 51%. In situ hybridisation pictures to all stages of development are available from Kohara NextDB. The report below describes variant b. This complete mRNA is 4809 bp long. It is supported by 8 cDNA clones, 7 of which match only this alternative variant. Its sequence exactly matches the genome. The premessenger has 16 exons. It covers 7.70 kb on the WS97 genome. It is transpliced to SL1. It has a very long 3' UTR. The protein (1147 aa, 124.2 kDa, pI 6.1) contains 2 Zn-finger, B-box motifs, one Filamin/ABP280 repeat motif, 6 NHL repeat motifs. It also contains a coil coil stretch [Psort2]. Taxblast (threshold 10^-3) tracks ancestors down to archaea and bacteria and eukaryota. COMPLETENESS: full length. Method: conceptual translation. FEATURES Location/Qualifiers source 1..1147 /organism="Caenorhabditis elegans" /db_xref="taxon:6239" /chromosome="I" /map="I;+3.53 cM (interpolated genetic position)" /map="I;+3.75 cM (measured genetic position)" /map="I; covering 7702 bp, from base 9350549 to 9342848 on genome release WS97" /clone="Primers to amplify the CDS (10326 bp, Stop included): ATGGCGACCATCGTGCCATGCT (SEQ ID NO: 40) (T=63.8), CTAGAAGACACGGATGCAATTGTTTCCGAA (SEQ ID NO: 41) (T=63.4). Clones specific of this variant are AF195611, yk20b11, yk307c10, yk1100f6, yk1102h6, yk1111g2, yk1223b8. Complete CDS clones: yk1728d7. Recommended clone (from the Kohara collection): yk1728d7. for edited clone sequences /clone_lib="Kohara Sugano L1 larvae cap-selected library: yk1111g2, yk1100f6, yk1102h6, yk1223b8; Kohara Sugano L2 larvae cap-selected library: yk1728d7; Kohara mixed stage library, from him-8 strain, containing 15-30% males: yk307c10; Kohara mixed stage library, from him-8 strain, containing 15-30% males: yk20b11; gb: AF195611" Protein 1..1147 /product="abnormal cell LINeage LIN-41, heterochronic gene; Drosophila dappled/ vertebrate TRipartite Motif protein related; B-box zinc finger, Filamin and NHL repeat containing protein (124.2 kD) (lin-41)" Region 366..412 /region_name="[Pfam/InterPro description] zn-finger, B-box" /db_xref="CDD:pfam00643" Region 474..516 /region_name="[Pfam/InterPro description] zn-finger, B-box" /db_xref="CDD:pfam00643" Region 557..621 /region_name="[PSORT] coil coil domain: TAENEIRAAFDTHVNALEERRKELLKRVETVKNLKLSVLISQAESLQSKQIDLQQAIQ (SEQ ID NO: 42) TATKLMD" Region 692..814 /region_name="[Pfam/InterPro description] Filamin/ABP280 repeat" /db_xref="CDD:pfam00630" Region 845..872 /region_name="[Pfam/InterPro description] NHL repeat" /db_xref="CDD:pfam01436" Region 892..919 /region_name="[Pfam/InterPro description] NHL repeat" /db_xref="CDD:pfam01436" Region 939..966 /region_name="[Pfam/InterPro description] NHL repeat" /db_xref="CDD:pfam01436" Region 987..1014 /region_name="[Pfam/InterPro description] NHL repeat" /db_xref="CDD:pfam01436" Region 1035..1062 /region_name="[Pfam/InterPro description] NHL repeat" /db_xref="CDD:pfam01436" Region 1120..1147 /region_name="[Pfam/InterPro description] NHL repeat" /db_xref="CDD:pfam01436" CDS 1..1147 /gene="lin-41" /locus_tag="1J912" /coded_by="NM_060087.2:196..3639" /db_xref="AceView/WormGenes:lin-41" /db_xref="GeneID:172760" /db_xref="LocusID:172760" ORIGIN 1 mativpcsle keegapsgpr rlqteidvda ndsgnelsmg gsssegdsms hhrgehspnh 61 hhqdnhlgsg ppppqftgsl fdtppsmiqs pqqqpqfqfn tgfglglpqd sfrcsvcsks 121 stigvlpfvc ahktcqscyq mtpssydrra cklcgavsta tanftsqmyl sptlpspprg 181 almsdcstpt mnnhinsstp lhqprafsfs lsgmpgspsp vmgarmpssa gglmmrpigf 241 pdsdssltsw splqqpsqls innlssiggh qqqspmlmqn vfdslavndd tpvfsplspt 301 ntsmhmppsl maspdvpkhs atiapprnsm cstprlqlat pmssqsqqtf pipsplgsqp 361 qqqqpmgpiq cqgceskisf aycmqcqeal cihcvqahqr vratkqhafv elqqlmatlm 421 sravqpqqaq qytqnvggsv rqalgsvgsg dvffsghvsg vendsigsge ssprsssvcg 481 thdsviigic encphsvllc aicvaqhpgk hrvqplgdir vavgevvnes qllqwqcekt 541 gdtikqiidg ivtnattaen eiraafdthv naleerrkel lkrvetvknl klsvlisqae 601 slqskqidlq qaiqtatklm dssdcdemvl rqvfeklasc qmgnegtepn nnilnvlmla 661 cqvneddrlk ftapqdgill nkarqfgnie sgpcaknssi vgdsfkkair erqtviyvql 721 rdacgdllss siaatqptsq allphqephs hleqamptsd vqafvispdg stvevtmtpr 781 engivalsyy psiegsytln ilvkgtpisg cpttmdirrg rnydeiaakg piltfgkegs 841 gdgelcrpwg icvdqrgrvi vadrsnnrvq ifdkdgnfis kfgtsgnrpg qfdrpagitt 901 nslnnivvad kdnhrvqvfd engmfllkfg drgravgyfn ypwgvatnsh naiavsdtrn 961 hrvqiftpqg qfvrkcgfds ayffknldsp rglcylpdgq llitdfnnhr lavlsprnms 1021 emkvygsegd gdgmfvrpqg vvidpeghil vcdsrnnrvq vfasddmrfi gsfglgpvpn 1081 sgfqmpqelp apysslggpf gapafssapt pltpsprqll drptdlavgp dgriyvvdfg 1141 nncirvf (SEQ ID NO: 43) LIN-41 homologs include H. sapiens gi|37550026|ref|XP_067369.3|[37550026], M. musculus gi|38090144|ref|XP_356199.1|[38090144] and R. norvegicus gi|34866457|ref|XP_236676.2|[34866457].

IIIQ.low homology MADS box protein, novel

Also see the C. elegans Protein Database: Wormpep at Wellcome Trust Sanger Institute, Wellcome Trust Genome Campus, Hinxton, Cambridge, CB10 ISA, UK; Accesion No. ce01506.

IIIR.RPN-9 proteasome subunit MTAQDYLNGK LAAANGPLAD DWKNLKELWE KKLWHQLTVL TRSLVKKPQF VASTDMHEFY RLFVAEWELR VNPLQLVEIC ISIAQNIATK DKQKSMEFLS KIGNVINKDK IAVARLHTGE IEARLENKDK NGQIIDLKSI RTQIDSTQHE VDSLVGVTEV HAPFYRVSSL YLREVGDFAG YYREALRYLG VEDANNLTTE QKQVHAVLLG FAALLGENVH NFGELLAHPI LKSLEGTRER WIVDVLLAFN SGDLTRFFSL EGDWGGWDDL KKQKDFLTAK IRLMAVMELA VSRPTKARSV SFKEIATKCQ IPFDEVEFLV MKALSKDLIR GDINQVEQVV YVTWVQPRVL DNPQIMQMAT RISAWRNDVN SMEGIVSKEA REILTQN (SEQ ID NO: 44)

Homologs include, for example, Swiss-Prot. Accession No. Q9WVJ2, M. musculus 26S proteasome non-ATPase regulatory subunit 13 (26S proteasomesregulatory subunit S11) (26S proteasome regulatory subunit p40.5). Swiss-Prot. Q9UNM6, H. sapiens 26S proteasome non-ATPase regulatory subunit and Swiss Prot. Accession No. Q04062, S. cerevisiae Regulatory Particle Non-ATPase.

IIIS.TAF 6.1

The TAF 6.1 is part of an operon with w09b6.3 (an enhancer of RNAi) and expressed as a polypeptide fusion. This protein is well conserved and the human ortholog is Transcription initiation factor TFIID subunit 6. LOCUS NP_493919 470 aa linear DEFINITION TBP-Associated transcription Factor family member (52.7 kD) (taf-6.1) [Caenorhabditis elegans]. ACCESSION NP_493919 VERSION NP_493919.1 GI:17536589 DBSOURCE REFSEQ: accession NM_061518.1 KEYWORDS . SOURCE Caenorhabditis elegans ORGANISM Caenorhabditis elegans Eukaryota; Metazoa; Nematoda; Chromadorea; Rhabditida; Rhabditoidea; Rhabditidae; Peloderinae; Caenorhabditis. COMMENT VALIDATED REFSEQ: This record has undergone preliminary review of the sequence, but has not yet been subject to final review. This record is derived from an annotated genomic sequence (NC_003280). The reference sequence was derived from WormBase CDS:W09B6.2. Summary: This gene taf-6.1, also known as W09B6.2, 2B421 or YK5540, maps at (II; -12.83). It encodes a TBP-Associated transcription Factor family member. According to the Worm Transcriptome Project, it is well expressed mostly in embryos, and some at all stages of development [Kohara cDNAs]. Its sequence is fully supported by 8 cDNA clones. RNA interference results: [J.Ahringer 2003] No obvious phenotype (by feeding genomic PCR product JA:W09B6.2). Warning: this double stranded RNA may also interfere with gene 2B417. Function Protein properties: used to be called taf-3. Expression The expression profile for the gene, derived from the proportion of animals at each stage in each Kohara library is: embryos 56%, L1 or L2 larvae 21%, L3 to adult 24%. In situ hybridisation pictures to all stages of development are available from Kohara NextDB. The CDS has 8 exons. It covers 4.31 kb on the WS97 genome. The protein (470 aa, 52.7 kDa, pI 8.7) contains no Pfam motif. It is predicted to localise in the mitochondria [Psort2]. Taxblast (threshold 10^-3) tracks ancestors down to eukaryota. COMPLETENESS: full length. Method: conceptual translation. FEATURES Location/Qualifiers source 1..470 /organism="Caenorhabditis elegans" /db_xref="taxon:6239" /chromosome="II" /map="II;-12.83 cM (interpolated genetic position)" /map="II; covering 4373 bp, from base 1127981 to 1132355 on genome release WS97" /clone_lib="Kohara embryonic lambda gt11 library: yk314a6, yk502e6, yk649h1, yk650b11, yk670h10; Kohara Sugano L1 larvae cap-selected library: yk1035e11, yk1330h4; Kohara Sugano L4 larvae cap-selected library: yk850e10" Protein 1..470 /product="TBP-Associated transcription Factor family member (52.7 kD) (taf-6.1)" Region 247..250 /region_name="[PSORT] nuclear localization domain: KKRH" (SEQ ID NO: 45) CDS 1..470 /gene="taf-6.1" /locus_tag="2B421" /coded_by="NM_061518.1:1..1413" /db_xref="AceView/WormGenes:taf-6.1" /db_xref="GeneID:173498" /db_xref="LocusID:173498" /db_xref="WormBase:W09B6.2" ORIGIN 1 msktvtirrp sptktseepa ahqtpiftqt aaemlgitsl nteaaellef lsreklkeiv 61 rlsakwtqks arrrmavadv ehairstrqc gglnissvdt lnlgiqqlqp iqgtstgiys 121 flkssadvdv dkedtetfik iprdlrvisy plvnegqpvq seytvnvded dgnffekivp 181 evmtmipekn tpsssttssl qmfrdavkta kidqkvglkp stieiltveq qifmkdiitv 241 cmgqddkkrh ealytletda glqvflphlt ericksisan isqrclslii yagrvlrsls 301 hnkacdmtvt lhhvlpalls ccvgrnmclr petdnhwalr dfsaktlvgl vrdqvdkhda 361 grtarrlfdf shrifrdtgs sfsmiygtvh ilqefvagpk kaawlltelg etnarckshi 421 esgsrigasq lsiqeaqkln qqilkcensi rnrynlqqqa pgvpinrrfh (SEQ ID NO: 46)

IIIT.T54 homology MLRRERDSKCLKFLKIQFLSSKSFFITIKFYSDKSEYDKEKDLLEIERKK LESQPPSTSTSQSSKDYKSKSSSSKHDKNSSEYERNNKMWARTDLLVRFI DEDFKRGSLYKQKVRIVDVAGYNDVTIEDRGNTHYNIRQSWLETVIPREI GEKLMIVAGKRSGQLALMLGKDKRKEKLTARLVATNDVVTAYFEDVCAVK IRHEEDSPQLYGMTQFLFKILLVCTIINEFDTLNILFYVPTLSHSHISFN TKLAQLLATSGHQVTVLLAQVDRKCIKLEKKPTILPILQLLTLSNLII (SEQ ID NO: 47)

Also see the C. elegans Protein Database: Wormpep at Wellcome Trust Sanger Institute, Wellcome Trust Genome Campus, Hinxton, Cambridge, CB10 ISA, UK; Accesion No. ce05915.

IIIU.RRM protein MATSFYTGGGEDGDGFNPRVHARIAEREGFQLASGSEDPRTLFVANLDPA ITDEFLATLFNQIGAVMKAKIIFEGLNDPYAFVEFSDHNQATLALQSHNG RELLEKEMHVTWAFEPREPGENRSKPETSRHFHVFVGDLCSEIDSTKLRE AFVKFGEVSEAKIIRDNNTNKGKGYGFVSYPRREDAERAIDEMNGAWLGR RTIRTNWATRKPDEDGERGGDRGDRRGGGGGGRDRYHNQSEKTYDEIFNQ AAADNTSVYVGNIANLGEDEIRRAFDRFGPINEVRTFKIQGYAFVKFETK ESAARAIVQMNNADIGGQIVRCSWGKSGDSGKPSERGSGGGGGSGNYGYG YGNSGGGGGSGGPGNSQFSNFNQRPPPSGNGGSGGGSGGQNNQYWQYYSQ YYNNPHLMQQWNNYWQKDGPPPPPAAAASSTGGN (SEQ ID NO: 48)

Also see the C. elegans Protein Database: Wormpep at Wellcome Trust Sanger Institute, Wellcome Trust Genome Campus, Hinxton, Cambridge, CB10 ISA, UK; Accesion No. ce21988.

IIIV.Worm unique/Novel MQPVLVNSRPLRVKSHESESKLNLIEQEDQFEGANYSSSSGVIICYSNGT GEVITQEAFDDSGIHFIFSKATCIQYPSNFDPIGVGSVVQIFWSRSFERV VRGNHIIVQIEKMEVYKCCAMLREQVFVTFNSPSTAGVAIGVTERNITVA FHPNCSPVIRYETLKAHSIGRTEFEIKDVREFEFSNGKNRHRENTNRMVD VILAAVPFRVEIHGNVDKIPFFVIEKCRNSPGRSGAAVITKIMKNHFMEA NFLQNSESIYFDSTSCHSNILEKVSIGSLINVLADPTFATSSYKWYGYDV TLCNNYLAHASTQRSFVLENNEILQNCKKLEKSPEEAETTTKNDLRFVPP QPEKGEVKKNELPEREAKSIINSYFIDRLAEGIKIEKIDKNWRTFGEILP KTPKKYSESLKKSIQNVLEPFGLNKPEKAAETPKIVEYFPKNPKKRVEIV EKPTVDEIRELFGALMDAEGFALNQRVKPHFVLPDTRWKPTERRYIGIYD DVQWTFMSTFCPKIEENSENRPLAGGWWYRRTVPRDHPVEIVQKMETRRN IIKDCTESPFIE (SEQ ID NO: 49)

Also see the C. elegans Protein Database: Wormpep at Wellcome Trust Sanger Institute, Wellcome Trust Genome Campus, Hinxton, Cambridge, CB10 ISA, UK; Accesion No. ce27223.

IIIW.TBB-4 LOCUS NP_509585 444 aa linear DEFINITION tubulin, Beta (49.8 kD) (tbb-4) [Caenorhabditis elegans]. ACCESSION NP_509585 VERSION NP_509585.1 GI:17549915 DBSOURCE REFSEQ: accession NM_077184.1 KEYWORDS . SOURCE Caenorhabditis elegans ORGANISM Caenorhabditis elegans Eukaryota; Metazoa; Nematoda; Chromadorea; Rhabditida; Rhabditoidea; Rhabditidae; Peloderinae; Caenorhabditis. REFERENCE 1 (residues 1 to 444) AUTHORS Maeda,I., Kohara,Y., Yamamoto,M. and Sugimoto,A. TITLE Large-scale analysis of gene function in Caenorhabditis elegans by high-throughput RNAi JOURNAL Curr. Biol. 11 (3), 171-176 (2001) MEDLINE 21154836 PUBMED 11231151 COMMENT VALIDATED REFSEQ: This record has undergone preliminary review of the sequence, but has not yet been subject to final review. This record is derived from an annotated genomic sequence (NC_003284). The reference sequence was derived from WormBase CDS:B0272.1. Summary: This essential gene tbb-4, also known as B0272.1, XK54 or YK4801, maps at (X; +1.30). Its phenotype is embryonic lethal, partial. It encodes a tubulin, Beta. According to the Worm Transcriptome Project, it is well expressed mainly in embryos and some in L1 and L2 larvae [Kohara cDNAs]. Its sequence is fully supported by 7 cDNA clones. RNA interference results: [J.Ahringer 2003] No obvious phenotype (by feeding genomic PCR product JA:B0272.1). [A.Sugimoto 2002] Embryonic lethal (20%) (by injecting cDNA clone SA:yk313f12). Expression The expression profile for the gene, derived from the proportion of animals at each stage in each Kohara library is: embryos 82%, L1 or L2 larvae 14%, L3 to adult 4%. In situ hybridisation pictures to all stages of development are available from Kohara NextDB. The CDS has 7 exons. It covers 2.21 kb on the WS97 genome. The protein (444 aa, 49.8 kDa, pI 4.8) contains one Tubulin/FtsZ protein motif, one Tubulin/FtsZ protein motif. It also ontains a coil coil stretch [Psort2]. It is predicted to localise in the cytoskeleton [Psort2]. Taxblast (threshold 10^-3) tracks ancestors down to eukaryota. COMPLETENESS: full length. Method: conceptual translation. FEATURES Location/Qualifiers source 1..444 /organism="Caenorhabditis elegans" /db_xref="taxon:6239" /chromosome="X" /map="X;+1.30 cM (interpolated genetic position)" /map="X; covering 2444 bp, from base 9427407 to 9424964 on genome release WS97" /clone_lib="Kohara embryonic lambda gt11 library: yk230e11, yk313f12, yk646g2, yk671e8, yk674c9, yk80b7; Kohara Sugano L2 larvae cap-selected library: yk1730e2" Protein 1..444 /product="tubulin, Beta (49.8 kD) (tbb-4)" Region 45..244 /region_name="[Pfam/InterPro description] Tubulin/FtsZ protein" /db_xref="CDD:pfam00091" Region 246..383 /region_name="[Pfam/InterPro description] Tubulin/FtsZ protein" /db_xref="CDD:pfam03953" Region 402..438 /region_name="[PSORT] coil coil domain: GMDEMEFTEAESNMNDLVSEYQQYQEATADDEGEFDE" (SEQ ID NO: 50) CDS 1..444 /gene="tbb-4" /locus_tag="XK54" /coded_by="NM_077184.1:1..1335" /db_xref="AceView/WormGenes:tbb-4" /db_xref="GeneID:181170" /db_xref="LocusID:181170" /db_xref="WormBase:B0272.1" ORIGIN 1 mreivhiqag qcgnqigakf wevisdehgi dptgayngds dlqlerinvy yneasggkyv 61 praclvdlep gtmdsvragp fgqlfrpdnf vfgqsgagnn wakghytega elvdnvldvv 121 rkeaescdcl qgfqmthslg ggtgsgmgtl liskireeyp drimmtfsvv pspkvsdtvv 181 epynatlsvh qlventdetf cidnealydi cfrtlklttp tygdlnhlvs mtmsgvttcl 241 rfpgqlnadl rklavnmvpf prlhffmpgf apltsrgsqq yrsltvpelt qqmfdaknmm 301 aacdprhgry ltvaamfrgr msmkevdeqm lnvqnknssy fvewipnnvk tavcdipprg 361 vkmaatfvgn staiqelfkr iseqftamfr rkaflhwytg egmdemefte aesnmndlvs 421 eyqqyqeata ddegefdehd qdve (SEQ ID NO: 51)

IIIX.RPS-14 LOCUS NP_498572 152 aa linear DEFINITION ribosomal Protein, Small subunit (16.2 kD) (rps-14) [Caenorhabditis elegans]. ACCESSION NP_498572 VERSION NP_498572.1 GI:17554776 DBSOURCE REFSEQ: accession NM_066171.1 KEYWORDS . SOURCE Caenorhabditis elegans ORGANISM Caenorhabditis elegans Eukaryota; Metazoa; Nematoda; Chromadorea; Rhabditida; Rhabditoidea; Rhabditidae; Peloderinae; Caenorhabditis. REFERENCE 1 (residues 1 to 152) AUTHORS Kamath,R.S., Fraser,A.G., Dong,Y., Poulin,G., Durbin,R., Gotta,M., Kanapin,A., Le Bot,N., Moreno,S., Sohrmann,M., Welchman,D.P., Zipperlen,P. and Ahringer,J. TITLE Systematic functional analysis of the Caenorhabditis elegans genome using RNAi JOURNAL Nature 421 (6920), 231-237 (2003) MEDLINE 22417569 PUBMED 12529635 REFERENCE 2 (residues 1 to 152) AUTHORS Gonczy,P., Echeverri,C., Oegema,K., Coulson,A., Jones,S.J., Copley,R.R., Duperon,J., Oegema,J., Brehm,M., Cassin,E., Hannak,E., Kirkham,M., Pichler,S., Flohrs,K., Goessen,A., Leidel,S., Alleaume,A.M., Martin,C., Ozlu,N., Bork,P. and Hyman,A.A. TITLE Functional genomic analysis of cell division in C. elegans using RNAi of genes on chromosome III JOURNAL Nature 408 (6810), 331-336 (2000) MEDLINE 20548710 PUBMED 11099034 COMMENT VALIDATED REFSEQ: This record has undergone preliminary review of the sequence, but has not yet been subject to final review. This record is derived from an annotated genomic sequence (NC_003281). The reference sequence was derived from WormBase CDS:F37C12.9. Summary: This essential gene rps-14, also known as F37C12.9, 3I268 or YK9313, maps at (III; -0.77). Phenotypes and affected processes are abnormal cytoplasmic appearance, embryonic lethal, sterile adult, unhealthy, abnormal pseudocleavage. It encodes a ribosomal Protein, Small subunit. The product would be involved in pseudocleavage (sensu Nematoda). According to the Worm Transcriptome Project, it is expressed at very high level at all stages of development [Kohara cDNAs]. Its sequence is fully supported by 144 cDNA clones. RNA interference results [T.Hyman; 2000] All embryos dead. DIC phenotype -- Multiple female pronuclei; irregular cytoplasmic appearance; karyomeres in daughter blastomeres; nuclei in AB are off-center for a while, nuclei in P1 stay close to posterior cortex for a while. Phenotype comment -- Semi-sterile. Phenotype confirmed with independent dsRNA (F37C12.9-RNA2; similar phenotype) (by injecting genomic PCR product TH:330a9). Same description as TH:330a9 (by injecting genomic PCR product TH:340d4). [J.Ahringer 2003] Sterile, sick (by feeding genomic PCR product JA:F37C12.9). Function Protein properties: Orthologous to yeast (S.cerevisiae) ribosomal protein rps14 using blastP. Expression The expression profile for the gene, derived from the proportion of animals at each stage in each Kohara library is: embryos 23%, L1 or L2 larvae 49%, L3 to adult 27%. In situ hybridisation pictures to all stages of development are available from Kohara NextDB. The CDS has 3 exons. It covers 0.55 kb on the WS97 genome. The protein (152 aa, 16.2 kDa, pI 10.4) contains one ribosomal protein S11 motif. It is predicted to localise in the cytoplasm [Psort2]. Taxblast (threshold 10^-3) tracks ancestors down to archaea and bacteria and eukaryota. COMPLETENESS: full length. Method: conceptual translation. FEATURES Location/Qualifiers source 1..152 /organism="Caenorhabditis elegans" /db_xref="taxon:6239" /chromosome="III" /map="III;-0.77 cM (interpolated genetic position)" /map="III; covering 615 bp, from base 7179511 to 7178897 on genome release WS97" Protein 1..152 /product="ribosomal Protein, Small subunit (16.2 kD) (rps-14)" Region 3..9 /region_name="[PSORT] nuclear localization domain: PARKGKA" (SEQ ID NO: 52) Region 30..148 /region_name="[Pfam/InterPro description] ribosomal protein S11" /db_xref="CDD:pfam00411" CDS 1..152 /gene="rps-14" /locus_tag="3I268" /coded_by="NM_066171.1:1..459" /db_xref="AceView/WormGenes:rps-14" /db_xref="GeneID:176006" /db_xref="LocusID:176006" /db_xref="WormBase:F37C12.9" ORIGIN 1 maparkgkak eeqavvslgp qakegelifg vahifasfnd tfvhitdisg retivrvtgg 61 mkvkadrdes spyaamlaaq dvadrckqlg inalhiklra tggtrtktpg pgaqsalral 121 aragmkigri edvtpipsdc trrkggrrgr rl (SEQ ID NO: 53)

IIIZ.RPS-13 LOCUS NP_498393 151 aa linear DEFINITION ribosomal Protein, Small subunit (17.3 kD) (rps-13) [Caenorhabditis elegans]. ACCESSION NP_498393 VERSION NP_498393.1 GI:17554774 DBSOURCE REFSEQ: accession NM_065992.1 KEYWORDS . SOURCE Caenorhabditis elegans ORGANISM Caenorhabditis elegans Eukaryota; Metazoa; Nematoda; Chromadorea; Rhabditida; Rhabditoidea; Rhabditidae; Peloderinae; Caenorhabditis. REFERENCE 1 (residues 1 to 151) AUTHORS Kamath,R.S., Fraser,A.G., Dong,Y., Poulin,G., Durbin,R., Gotta,M., Kanapin,A., Le Bot,N., Moreno,S., Sohrmann,M., Welchman,D.P., Zipperlen,P. and Ahringer,J. TITLE Systematic functional analysis of the Caenorhabditis elegans genome using RNAi JOURNAL Nature 421 (6920), 231-237 (2003) MEDLINE 22417569 PUBMED 12529635 REFERENCE 2 (residues 1 to 151) AUTHORS Gonczy,P., Echeverri,C., Oegema,K., Coulson,A., Jones,S.J., Copley,R.R., Duperon,J., Oegema,J., Brehm,M., Cassin,E., Hannak,E., Kirkham,M., Pichler,S., Flohrs,K., Goessen,A., Leidel,S., Alleaume,A.M., Martin,C., Ozlu,N., Bork,P. and Hyman,A.A. TITLE Functional genomic analysis of cell division in C. elegans using RNAi of genes on chromosome III JOURNAL Nature 408 (6810), 331-336 (2000) MEDLINE 20548710 PUBMED 11099034 COMMENT VALIDATED REFSEQ: This record has undergone preliminary review of the sequence, but has not yet been subject to final review. This record is derived from an annotated genomic sequence (NC_003281). The reference sequence was derived from WormBase CDS:C16A3.9. Summary: This essential gene rps-13, also known as C16A3.9, 3H464 or YK2267, maps at (III; -1.25). Its phenotype is embryonic lethal, sterile adult, abnormal pseudocleavage. It encodes a ribosomal Protein, Small subunit. The product would be involved in pseudocleavage (sensu Nematoda). According to the Worm Transcriptome Project, it is expressed at high level in L3, L4, adult and culminating in embryos [Kohara cDNAs]. Its sequence is fully supported by 34 cDNA clones. RNA interference results [T.Hyman; 2000] All embryos dead. DIC phenotype - Multiple female pronuclei; irregular cytoplasmic appearance; aberrant pseudocleavage stage; karyomeres in daughter blastomeres; nuclei in AB are off-center for a while, nuclei in P1 stay close to posterior cortex for a while (by injecting genomic PCR product TH:309g1). Movies are available on Hyman's site. [J.Ahringer 2003] Sterile (by feeding genomic PCR product JA:C16A3.9). Function Protein properties: Orthologous to yeast (S.cerevisiae) ribosomal protein rps13 using blastP. Expression The expression profile for the gene, derived from the proportion of animals at each stage in each Kohara library is: embryos 81%, L1 or L2 larvae 1%, L3 to adult 17%. In situ hybridisation pictures to all stages of development are available from Kohara NextDB. The CDS has 3 exons. It covers 0.85 kb on the WS97 genome. The protein (151 aa, 17.3 kDa, pI 10.7) contains one Ribosomal protein S15 motif. It is predicted to localise in the cytoplasm [Psort2]. Taxblast (threshold 10^-3) tracks ancestors down to archaea and eukaryota. COMPLETENESS: full length. Method: conceptual translation. FEATURES Location/Qualifiers source 1..151 /organism="Caenorhabditis elegans" /db_xref="taxon:6239" /chromosome="III" /map="III;-1.25 cM (interpolated genetic position)" /map="III; covering 909 bp, from base 6374934 to 6374026 on genome release WS97" /clone_lib="Kohara embryonic lambda gt11 library: yk74d8, yk96e4, yk139e1, yk141e12, yk196b12, yk269e10, yk319c12, yk329h11, yk332c6, yk390g10, yk418c7, yk418e3, yk432h1, yk433b12, yk436g3, yk467c6, yk474h7, yk479b1, yk502h5, yk508h12, yk533f12, yk538g7, yk572a6, yk623c2, yk628b6, yk631e5, yk641b4, yk641h7, yk666c1, yk668a5, yk627h1; mixed stage, Stratagene library [PMID1302005]: CEMSA36, CEMSH68; Kohara mixed stage library, from him-8 strain, containing 15-30% males: yk304b4" Protein 1..151 /product="ribosomal Protein, Small subunit (17.3 kD) (rps-13)" Region 61..151 /region_name="[Pfam/InterPro description] ribosomal protein S15" /db_xref="CDD:pfam00312" CDS 1..151 /gene="rps-13" /locus_tag="3H464" /coded_by="NM_065992.1:1..456" /db_xref="AceView/WormGenes:rps-13" /db_xref="GeneID:175901" /db_xref="LocusID:175901" /db_xref="WormBase:C16A3.9" ORIGIN 1 mgrmhnpgkg maksaipyrr svpswqkmta eevqdqivkm akkglrpsqi gvilrdshgv 61 gqvrrlagnk ifrilkskgm apelpedlyh lvkkavairk hlersrkdid skyrlilves 121 rihrlaryyk tkrqlpptwk yesgtaaslv s (SEQ ID NO: 54)

IIIAA.RPL-24 LOCUS NP_491399 159 aa linear DEFINITION ribosomal Protein, Large subunit (17.8 kD) (rpl-24.1) [Caenorhabditis elegans]. ACCESSION NP_491399 VERSION NP_491399.1 GI:17506331 DBSOURCE REFSEQ: accession NM_058998.1 KEYWORDS . SOURCE Caenorhabditis elegans ORGANISM Caenorhabditis elegans Eukaryota; Metazoa; Nematoda; Chromadorea; Rhabditida; Rhabditoidea; Rhabditidae; Peloderinae; Caenorhabditis. REFERENCE 1 (residues 1 to 159) AUTHORS Fraser,A.G., Kamath,R.S., Zipperlen,P., Martinez-Campos,M., Sohrmann,M. and Ahringer,J. TITLE Functional genomic analysis of C. elegans chromosome I by systematic RNA interference JOURNAL Nature 408 (6810), 325-330 (2000) MEDLINE 20548709 PUBMED 11099033 REFERENCE 2 (residues 1 to 159) AUTHORS Walhout,A.J., Sordella,R., Lu,X., Hartley,J.L., Temple,G.F., Brasch,M.A., Thierry-Mieg,N. and Vidal,M. TITLE Protein interaction mapping in C. elegans using proteins involved in vulval development JOURNAL Science 287 (5450), 116-122 (2000) MEDLINE 20082953 PUBMED 10615043 COMMENT VALIDATED REFSEQ: This record has undergone preliminary review of the sequence, but has not yet been subject to final review. This record is derived from an annotated genomic sequence (NC_003279). The reference sequence was derived from WormBase CDS:D1007.12. Summary: This essential gene rpl-24.1, also known as D1007.12, 1F153 or YK1971, maps at (I; -1.08). Its phenotype is embryonic lethal, sterile adult. It encodes a ribosomal Protein, Large subunit. From Pfam homology, the product would be involved in protein biosynthesis and would localize in intracellular, ribosome. According to the Worm Transcriptome Project, it is expressed at very high level at all stages of development [Kohara cDNAs]. Its sequence is fully supported by 124 cDNA clones. RNA interference results [J.Ahringer 2000] embryonic lethal (100%), larval arrest, sterile (maternal brood size 1 to 5) (by feeding genomic PCR product JA:D1007.12). Expression The expression profile for the gene, derived from the proportion of animals at each stage in each Kohara library is: embryos 24%, L1 or L2 larvae 44%, L3 to adult 31%. In situ hybridisation pictures to all stages of development are available from Kohara NextDB. Interactions The protein encoded by this gene interacts with: protein LIN-15A: [Vidal M, pm10615043] interaction seen in a 2-hybrid screen, with lin-15a as bait. The CDS has 4 exons. It covers 1.00 kb on the WS97 genome. The protein (159 aa, 17.8 kDa, pI 11.3) contains one Ribosomal protein L24E motif. It also contains a coil coil stretch, an ER membrane domain [Psort2]. It is predicted to localise in the nucleus [Psort2]. Taxblast (threshold 10^-3) tracks ancestors down to archaea and eukaryota. COMPLETENESS: full length. Method: conceptual translation. FEATURES Location/Qualifiers source 1..159 /organism="Caenorhabditis elegans" /db_xref="taxon:6239" /chromosome="I" /map="I;-1.08 cM (interpolated genetic position)" /map="I; covering 1100 bp, from base 4585116 to 4586217 on genome release WS97" /clone_lib="Kohara embryonic lambda gt11 library: yk62c11, yk63a1, yk74c6, yk78g2, yk79a4, yk79g4, yk81c9, yk83e5, yk89b7, yk96d9, yk103d8, yk138g11, yk172a2, yk210c12, yk216g11, yk325h1, yk375g5, yk401h10, yk424d6, yk449h5, yk458b1, yk469h3, yk473a2, yk476f10, yk479b3, yk483f11, yk486g10, yk489g9, yk502b6, yk533d12, yk602a11, yk606e6, yk667g3, yk505c9, yk175d3, yk460a5; Kohara Sugano L1 larvae cap-selected library: yk771e9, yk796g4, yk831f10, yk1104c7, yk1310a12, yk874a12, yk877b6, yk878b6, yk1006c9, yk1072h3, yk1087d2, yk1098e12, yk1129g2, yk1149e2, yk1165b8, yk1166d2, yk1168d9, yk1181f2, yk1193a10, yk1204f1, yk1219a9, yk1235a12, yk1272d7, yk1298e3, yk1320g11, yk1352a8, yk890c5, yk1081e12, yk1067g3; Kohara Sugano L2 larvae cap- selected library: yk818c3, yk775h12, yk810g8, yk816b10, yk1377d10, yk1386h10, yk1407b1, yk1418d5, yk1579d7, yk1583a8, yk1590g3, yk1600a2, yk1608h5, yk1670f3, yk1365h1, yk1381b7, yk1386b10, yk1390d12, yk1401g11, yk1420a1, yk1510f8, yk1517c6, yk1518f1, yk1578b5, yk1581a9, yk1587g3, yk1592d5, yk1610e10, yk1638c2, yk1667a11, yk1699g11, yk1719h1, yk1720e12, yk1722a8, yk1742e12, yk1756f2, yk1493f7, yk1360e6; Kohara Sugano L4 larvae cap-selected library: yk1437a7, yk1541g9, yk1685h11; Kohara Sugano mixed stage cap-selected library: yk732a10; mixed stage, Stratagene library [PMID1302005]: CEMSC16; Kohara mixed stage library, from him-8 strain, containing 15-30% males: yk70e3, yk71b8, yk71g7, yk99e8, yk170h5, yk206h1, yk361a7, yk379c9, yk545f5, yk547e1, yk557f9, yk545f11; Marc Vidal 2 hybrid library: mv508, mv1325, mv1525, mv1326" Protein 1..159 /product="ribosomal Protein, Large subunit (17.8 kD) (rpl-24.1)" Region 1..71 /region_name="[Pfam/InterPro description] ribosomal protein L24E" /db_xref="CDD:pfam01246" Region 60..76 /region_name="[PSORT] nuclear localization domain: KKGTHGQEQVTRKKTKK" (SEQ ID NO: 55) Region 104..136 /region_name="[PSORT] coil coil domain: RRQQREQAAKIAKDANKAVRAAKAAANKEKKAS" (SEQ ID NO: 56) Region 155..158 /region_name="[PSORT] ER membrane domain: VGGK" (SEQ ID NO: 57) CDS 1..159 /gene="rpl-24.1" /locus_tag="1F153" /coded_by="NM_058998.1:1..480" /db_xref="AceView/WormGenes:rpl-24.1" /db_xref="GeneID:172062" /db_xref="LocusID:172062" /db_xref="WormBase:D1007.12" ORIGIN 1 mkvetcvysg ykihpghgkr lvrtdgkvqi flsgkalkga klrrnprdir wtvlyriknk 61 kgthgqeqvt rkktkksvqv vnravaglsl dailakrnqt edfrrqqreq aakiakdank 121 avraakaaan kekkasqpkt qqktaknvkt aaprvggkr (SEQ ID NO: 58) LOCUS NP_492572 162 aa linear DEFINITION ribosomal Protein, Large subunit (18.8 kD) (rpl-24.2) [Caenorhabditis elegans]. ACCESSION NP_492572 VERSION NP_492572.1 GI:17505458 DBSOURCE REFSEQ: accession NM_060171.1 KEYWORDS . SOURCE Caenorhabditis elegans ORGANISM Caenorhabditis elegans Eukaryota; Metazoa; Nematoda; Chromadorea; Rhabditida; Rhabditoidea; Rhabditidae; Peloderinae; Caenorhabditis. REFERENCE 1 (residues 1 to 162) AUTHORS Fraser,A.G., Kamath,R.S., Zipperlen,P., Martinez-Campos,M., Sohrmann,M. and Ahringer,J. TITLE Functional genomic analysis of C. elegans chromosome I by systematic RNA interference JOURNAL Nature 408 (6810), 325-330 (2000) MEDLINE 20548709 PUBMED 11099033 COMMENT VALIDATED REFSEQ: This record has undergone preliminary review of the sequence, but has not yet been subject to final review. This record is derived from an annotated genomic sequence (NC_003279) The reference sequence was derived from WormBase CDS:C03D6.8. Summary: This gene rpl-24.2, also known as C03D6.8, 1K245 or YK5780, maps at (I; +3.90). It encodes a ribosomal Protein, Large subunit. From Pfam homology, the product would be involved in protein biosynthesis and would localize in intracellular, ribosome. According to the Worm Transcriptome Project, it is expressed at high level at all stages of development [Kohara cDNAs]. Its sequence is fully supported by 22 cDNA clones. RNA interference results [J.Ahringer 2000] slow growth (by feeding genomic PCR product JA:C03D6.1). Warning: this double stranded RNA may also interfere with gene 1K244. [J.Ahringer 2000] slow growth (by feeding genomic PCR product JA:C03D6.8). Warning: this double stranded RNA may also interfere with gene 1K244. Expression The expression profile for the gene, derived from the proportion of animals at each stage in each Kohara library is: embryos 18%, L1 or L2 larvae 61%, L3 to adult 22%. In situ hybridisation pictures to all stages of development are available from Kohara NextDB. The CDS has 3 exons. It covers 0.59 kb on the WS97 genome. The protein (162 aa, 18.8 kDa, pI 10.6) contains one Ribosomal protein L24E motif. It is predicted to localise in the nucleus [Psort2]. Taxblast (threshold 10^-3) tracks ancestors down to archaea and eukaryota. COMPLETENESS: full length. Method: conceptual translation. FEATURES Location/Qualifiers source 1..162 /organism="Caenorhabditis elegans" /db_xref="taxon:6239" /chromosome="I" /map="I;+3.90 cM (interpolated genetic position)" /map="I; covering 698 bp, from base 9677465 to 9678164 on genome release WS97" /clone_lib="Kohara embryonic lambda gt11 library: yk331a1, yk512c8, yk663g11, yk176h7; Kohara Sugano L1 larvae cap-selected library: yk753c12, yk772h12, yk900d1, yk1127c1, yk1299f7, yk1304b7, yk1057e1, yk1255f6, yk1214c9, yk1159g10, yk1291g4, yk871c5; Kohara Sugano L2 larvae cap-selected library: yk1527g1, yk1569d5, yk1605a7, yk1668g2; Kohara mixed stage library, from him-8 strain, containing 15-30% males: yk361d3, yk582d11" Protein 1..162 /product="ribosomal Protein, Large subunit (18.8 kD) (rpl-24.2)" Region 1..71 /region_name="[Pfam/InterPro description] ribosomal protein L24E" /db_xref="CDD:pfam01246" Region 41..57 /region_name="[PSORT] nuclear localization domain: KKKKNPRKLRFTKAARR" (SEQ ID NO: 59) Region 43..59 /region_name="[PSORT] nuclear localization domain: KKNPRKLRFTKAARRAR" (SEQ ID NO: 60) CDS 1..162 /gene="rpl-24.2" /locus_tag="1K245" /coded_by="NM_060171.1:1..489" /db_xref="AceView/WormGenes:rpl-24.2" /db_xref="GeneID:172815" /db_xref="LocusID:172815" /db_xref="WormBase:C03D6.8" ORIGIN 1 mriekcyfcs spiypghgiq fvrndstvfk fcrsrcnklf kkkknprklr ftkaarrarg 61 kelindatql leqrrdepvk yeramfqkti eaaktisalk tkrygnlirk rlqpgkivqk 121 kgllakvdkk mhlirapvan rdgvktraaa kekktaesme tn (SEQ ID NO: 61)

IIIBB.RPS-11 LOCUS NP_502186 155 aa linear DEFINITION ribosomal Protein, Small subunit (17.7 kD) (rps-11) [Caenorhabditis elegans]. ACCESSION NP_502186 VERSION NP_502186.1 GI:17542016 DBSOURCE REFSEQ: accession NM_069785.1 KEYWORDS SOURCE Caenorhabditis elegans ORGANISM Caenorhabditis elegans Eukaryota; Metazoa; Nematoda; Chromadorea; Rhabditida; Rhabditoidea; Rhabditidae; Peloderinae; Caenorhabditis. REFERENCE 1 (residues 1 to 155) AUTHORS Kamath,R.S., Fraser,A.G., Dong,Y., Poulin,G., Durbin,R., Gotta,M., Kanapin,A., Le Bot,N., Moreno,S., Sohrmann,M., Welchman,D.P., Zipperlen,P. and Ahringer,J. TITLE Systematic functional analysis of the Caenorhabditis elegans genome using RNAi JOURNAL Nature 421 (6920), 231-237 (2003) MEDLINE 22417569 PUBMED 12529635 REFERENCE 2 (residues 1 to 155) AUTHORS Piano,F., Schetter,A.J., Mangone,M., Stein,L. and Kemphues,K.J. TITLE RNAi analysis of genes expressed in the ovary of Caenorhabditis elegans JOURNAL Curr. Biol. 10 (24), 1619-1622 (2000) MEDLINE 21065924 PUBMED 11137018 COMMENT VALIDATED REFSEQ: This record has undergone preliminary review of the sequence, but has not yet been subject to final review\ This record is derived from an annotated genomic sequence (NC_003282) The reference sequence was derived from WormBase CDS:F40F11.1. Summary: This essential gene rps-11, also known as F40F11.1, 4M367 or YK2226, maps at (IV; +5.45). Its phenotype is sterile adult, unhealthy, catastrophic one cell arrest. It encodes a ribosomal Protein, Small subunit. From Pfam homology, the product would be involved in protein biosynthesis and would localize in intracellular, ribosome. According to the Worm Transcriptome Project, it is expressed at very high level at all stages of development [Kohara cDNAs]. Its sequence is fully supported by 87 cDNA clones. RNA interference results [F.Piano 2000] Embryonic lethal; egg production ceases in injected animal; catastrophic one-cell arrest (by injecting cDNA clone FP:SP13H3). [J.Ahringer 2003] Sterile, sick (by feeding genomic PCR product JA:F40F11.1). Function Protein properties: Orthologous to yeast (S.cerevisiae) ribosomal protein rps11 using blastP. Expression The expression profile for the gene, derived from the proportion of animals at each stage in each Kohara library is: embryos 27%, L1 or L2 larvae 44%, L3 to adult 29%. In situ hybridisation pictures to all stages of development are available from Kohara NextDB. Pattern in ovary [F Piano, 2000]. The CDS has 3 exons. It covers 0.57 kb on the WS97 genome. The protein (155 aa, 17.7 kDa, pI 10.5) contains one Ribosomal protein S17 motif. It also contains a peroxisomal domain, an ER membrane domain [Psort2]. It is predicted to localise in the cytoplasm [Psort2]. Taxblast (threshold 10^-3) tracks ancestors down to archaea and bacteria and eukaryota. COMPLETENESS: full length. Method: conceptual translation. FEATURES Location/Qualifiers source 1..155 /organism="Caenorhabditis elegans" /db_xref="taxon:6239" /chromosome="IV" /map="IV;+5.45 cM (interpolated genetic position)" /map="IV; covering 651 bp, from base 11602617 to 11603269 on genome release WS97" /clone_lib="Kohara embryonic lambda gt11 library: yk67b1, yk89c6, yk106b9, yk138a8, yk172e3, yk173e4, yk258c6, yk258d2, yk273e4, yk290h1, yk327b12, yk400d7, yk468h9, yk471b5, yk477h6, yk485b4, yk500e2, yk521e9, yk572f9, yk616b11, yk629c3, yk639h12, yk644g7, yk646g7, yk647f7, yk681e7, yk325e1, yk678h4; Kohara Sugano L1 larvae cap-selected library: yk752e4, yk759c3, yk1292a10, yk753e4, yk1019c9, yk883b2, yk892b7, yk898a5, yk1011f1, yk1028c1, yk1106c9, yk1304c12, yk1326e8, yk1356h8, yk1207c9, yk871f6, yk1169b3, yk1298a10, yk1246f10; Kohara Sugano L2 larvae cap-selected library: yk778e12, yk1636d2, yk1593e1, yk1639a9, yk1576e5, yk1674h10, yk1691b5, yk1359c2, yk1414d7, yk1417e8, yk1417f11, yk1418f7, yk1489g4, yk1520d6, yk1521a3, yk1531b12, yk1567h6, yk1572a9, yk1577g10, yk1639h8, yk1650d4, yk1660f1, yk1671f12, yk1718h2, yk1727a4, yk1741b8, yk1706a1, yk1750b7; Kohara Sugano L4 larvae cap-selected library: yk785e9, yk834a6, yk1439e1, yk1545a1, yk1555g5, yk1442b4, yk1552h12; Kohara mixed stage library, from him-8 strain, containing 15-30% males: yk145g6, yk205e11, yk361b5, yk380g2; Piano ovary library: BE228125" Protein 1..155 /product="ribosomal Protein, Small subunit (17.7 kD) (rps-11)" Region 72..142 /region_name="[Pfam/InterPro description] ribosomal protein S17" /db_xref="CDD:pfam00366" Region 86..102 /region_name="[PSORT] nuclear localization domain: RRDYLHYIKKYRRYEKR" (SEQ ID NO: 62) Region 101..104 /region_name="[PSORT] nuclear localization domain: KRHK" (SEQ ID NO: 63) Region 151..154 /region_name="[PSORT] ER membrane domain: GFSK" (SEQ ID NO: 64) Region 153..155 /region_name="[PSORT] peroxisomal domain: SKF" CDS 1..155 /gene="rps-11" /locus_tag="4M367" /coded_by="NM_069785.1:1..468" /db_xref="AceView/WormGenes:rps-11" /db_xref="GeneID:178083" /db_xref="LocusID:178083" /db_xref="WormBase:F40F11.1" ORIGIN 1 mseqterafl kqptvnlnnk arilagskkt pryirevglg fkaprdaveg tyidkkcpwa 61 gnvpirgmil tgvvlknkmt rtivvrrdyl hyikkyrrye krhknvpahc spafrdihpg 121 dlvtigecrp lsktvrfnvl kvnksgtskk gfskf (SEQ ID NO: 65)

IIICC.Agglutinin MTTVRKTYRFCVFSSCLSVSCALVTQVHSSSLPIYSSPFVEKVFLHSSIY VRLCGDMYEQWPTLEFSDLNSSILDLFTKATSQSVASSLLYELTRSDADE NGGSIRLNNEEHLKWCMQVLNHSLTLSFATSREYETLKGAVRIYLHWLRA LCDTPDNNIPTPLLATPEKYFRNIIDALRWIFCRREDDFDTTVGGQVPRG LAIERQSIEIDMVLDSLKYLTRNSSRKYQDEVWARSISFLLNSSDILLSE PNATEEMGTRTCVRVADTLFDMWLNAVLNEHIPSLTYWSSLATLARRWRH NVPIIECWAKKILGLSTLVCRKMYGDDFLKIDIVDESVLPFENVPMTAEE DENEVHLLYRTWFNMLCLFDSPAKILNHDATRNLCLNGNSPRRTTSSISM SNFELASSSAAQGVSFFLAAVTLQRMVDLFYGDSRVKIDLRNYPVPDGKT APNTRTASVLTDNHSHHTNRTSSTTGDSSRYVSLGGAVGQIIVDDHQVSM SSGSTASGKTSTATGTSSTHTISSEIRRDQRIMSVNDRSRDPSHRTVSVT DSVNISNQSRYSEQTSSTLTYKSAPIPETANENGHGESISQLVSNSTVSA PVGGAGNDLTLKAGVHPSEMKIGRSSGVIGSAQHNNFYADTTSPYRSAQR FVTNFLTANQATMPYVGGKRPKTDRMLNLVGDWLFAIVNSPTNSPRVTGN DHSGHHKKNNDGVSDVSFISHHFVFTLLSAITTEVISIYICVSMISLTGL NKHHLRIGIIDDETVCTSECPFSPFFAKFTITDGVDFLNNEADSKTTPTS FDFDDFDSFHKFRFQHIYTSK (SEQ ID NO: 66)

Also see the C. elegans Protein Database: Wormpep at Wellcome Trust Sanger Institute, Wellcome Trust Genome Campus, Hinxton, Cambridge, CB10 ISA, UK; Accesion No. ce03050.

IIIDD.SIP-1 (hsp20)

Member of the Stress Induced Protein gene class. MSSLCPYTGR PTGLFRDFED MMPYWAQRHS MLNNFNNIVP QQLNEVENTA QKFCVKLDVA AFKPEELKVN LEGHVLTIEG HHEVKTEHGF SKRSFTRQFT LPKDVDLAHI HTVINKEGQM TIDAPKTGSN TTVRALPIHT SAGHAVTQKP SSTTTTGKH (SEQ ID NO: 67)

Homologs include, for example, Swiss-Prot. Accession No.P02511 , H. sapiens Alpha crystallin B chain.

IIIEE.CCT-6 (chaperonin) LOCUS NP_741153 539 aa linear DEFINITION chaperonin Containing TCP-1 (58.9 kD) (cct-6) [Caenorhabditis elegans]. ACCESSION NP_741153 VERSION NP_741153.1 GI:25144678 DBSOURCE REFSEQ: accession NM_171135.1 KEYWORDS SOURCE Caenorhabditis elegans ORGANISM Caenorhabditis elegans Eukaryota; Metazoa; Nematoda; Chromadorea; Rhabditida; Rhabditoidea; Rhabditidae; Peloderinae; Caenorhabditis. REFERENCE 1 (residues 1 to 539) AUTHORS Kamath,R.S., Fraser,A.G., Dong,Y., Poulin,G., Durbin,R., Gotta,M., Kanapin,A., Le Bot,N., Moreno,S., Sohrmann,M., Welchman,D.P., Zipperlen,P. and Ahringer,J. TITLE Systematic functional analysis of the Caenorhabditis elegans genome using RNAi JOURNAL Nature 421 (6920), 231-237 (2003) MEDLINE 22417569 PUBMED 12529635 REFERENCE 2 (residues 1 to 539) AUTHORS Gonczy,P., Echeverri,C., Oegema,K., Coulson,A., Jones,S.J., Copley,R.R., Duperon,J., Oegema,J., Brehm,M., Cassin,E., Hannak,E., Kirkham,M., Pichler,S., Flohrs,K., Goessen,A., Leidel,S., Alleaume,A.M., Martin,C., Ozlu,N., Bork,P. and Hyman,A.A. TITLE Functional genomic analysis of cell division in C. elegans using RNAi of genes on chromosome III JOURNAL Nature 408 (6810), 331-336 (2000) MEDLINE 20548710 PUBMED 11099034 REFERENCE 3 (residues 1 to 539) AUTHORS Leroux,M.R. and Candido,E.P. TITLE Characterization of four new tcp-1-related cct genes from the nematode Caenorhabditis elegans JOURNAL DNA Cell Biol. 14 (11), 951-960 (1995) MEDLINE 96069542 PUBMED 7576182 COMMENT VALIDATED REFSEQ: This record has undergone preliminary review of the sequence, but has not yet been subject to final review. This record is derived from an annotated genomic sequence (NC_003281) The reference sequence was derived from WormBase CDS:F01F1.8a Summary: This essential gene cct-6, also known as F01F1.8, 3G944 or YK828, maps at (III; -1.53). Phenotypes and affected processes are embryonic lethal, sterile adult, unhealthy, clear, translucent appearance, protruding vulva, small embryos, slow embryonic cell division, cytokinesis defect, abnormal cytoplasmic appearance. It encodes a chaperonin Containing TCP-1. According to the Worm Transcriptome Project, it is expressed at very high level at all stages of development [Kohara cDNAs]. Its sequence is fully supported by 122 cDNA clones and produces, by alternative splicing, 2 different transcripts a, b altogether encoding 2 different protein isoforms RNA interference results [T.Hyman; 2000] All embryos dead. DIC phenotype -- Semi- sterile; complex DIC phenotype; many embryos loose structural integrity upon dissection; areas lacking yolk granules; failure in different microtubule-based processes (centration/rotation, spindle assembly, chromosome segregation). DIC phenotype comment -- see also results from C07G2.3. Phenotype comment -- Confirmed with independent dsRNA (F01F1.8-RNA2; similar phenotype) (by injecting genomic PCR product TH:304C1). Movies are available on Hyman's site Same description as TH:304C1 (by injecting genomic PCR product TH:341B5) [J.Ahringer 2003] Embryonic lethal (100%), sterile, sick, clear, protruding vulva (by feeding genomic PCR product JA:F01F1.8) Function Protein properties: [C.elegansII] NMK. Encodes one of 7-9 related subunits of eukaryotic cytosolic chaperonin CCT.Ortholog of mouse Cctz (67% aa sequence identity)[PC] Expression The expression profile for the gene, derived from the proportion of animals at each stage in each Kohara library is: embryos 5%, L1 or L2 larvae 53%, L3 to adult 41%. In situ hybridisation pictures to all stages of development are available from Kohara NextDB For a detailed expression pattern description, see Wormbase Expr2045. The CDS has 6 exons. It covers 1.87 kb on the WS97 genome. The protein (539 aa, 58.9 kDa, pI 5.9) contains one chaperonin Cpn60/TCP-1 motif. It is predicted to localise in the cytoplasm [Psort2]. Taxblast (threshold 10^-3) tracks ancestors down to archaea and eukaryota. COMPLETENESS: full length. Method: conceptual translation. FEATURES Location/Qualifiers source 1..539 /organism="Caenorhabditis elegans" /db_xref="taxon:6239" /chromosome="III" /map="III;-1.53 cM (interpolated genetic position)" /map="III; covering 2163 bp, from base 5855637 to 5853475 on genome release WS97" Protein 1..539 /product="chaperonin Containing TCP-1 (58.9 kD) (cct- 6)" Region 30..530 /region_name="[Pfam/InterPro description] chaperonin Cpn60/TCP-1" /db_xref="CDD:pfam00118" CDS 1..539 /gene="cct-6" /locus_tag="3G944" /coded_by="NM_171135.1:1..1620" /db_xref="AceView/WormGenes:cct-6" /db_xref="GeneID:175819" /db_xref="LocusID:175819" /db_xref="WormBase:F01F1.8a" ORIGIN 1 mssiqclnpk aelarhaaal elnisgargl qdvmrsnlgp kgtlkmlvsg agdikltkdg 61 nvllhemaiq hptasmiaka staqddvtgd gttstvllig ellkqaeslv leglhprivt 121 egfewantkt lellekfkke apverdllve vcrtalrtkl hqkladhite cvvdavlair 181 rdgeepdlhm vekmemhhds dmdttlvrgl vldhgarhpd mprhvkdayi ltcnvsleye 241 ktevnsglfy ktakereall aaerefitrr vhkiielkkk vidnspdgkn kgfvvinqkg 301 idppsldlla segilalrra krrnmerlql avggeavnsv ddltpedlgw aglvyehslg 361 eekytfieec rapksvtlli kgpnkhtitq ikdaihdglr avfntivdka vlpgaaafei 421 aayvmlkkdv enlkgraklg aeafaqallv ipktlavngg ydaqetlvkl ieektaagpd 481 iavgldletg gavepqgiwd nvtvkknsis satvlacnll lvdevmragm tnlkqpqpe (SEQ ID NO: 68) LOCUS NP_741154 429 aa linear DEFINITION chaperonin Containing TCP-1 (cct-6) [Caenorhabditis elegans] ACCESSION NP_741154 VERSION NP_741154.1 GI:25144680 DBSOURCE REFSEQ: accession NM_171136.1 KEYWORDS SOURCE Caenorhabditis elegans ORGANISM Caenorhabditis elegans Eukaryota; Metazoa; Nematoda; Chromadorea; Rhabditida; Rhabditoidea; Rhabditidae; Peloderinae; Caenorhabditis. REFERENCE 1 (residues 1 to 429) AUTHORS Kamath,R.S., Fraser,A.G., Dong,Y., Poulin,G., Durbin,R., Gotta,M., Kanapin,A., Le Bot,N., Moreno,S., Sohrmann,M., Welchman,D.P., Zipperlen,P. and Ahringer,J. TITLE Systematic functional analysis of the Caenorhabditis elegans genome using RNAi JOURNAL Nature 421 (6920), 231-237 (2003) MEDLINE 22417569 PUBMED 12529635 REFERENCE 2 (residues 1 to 429) AUTHORS Gonczy,P., Echeverri,C., Oegema,K., Coulson,A., Jones,S.J., Copley,R.R., Duperon,J., Oegema,J., Brehm,M., Cassin,E., Hannak,E., Kirkham,M., Pichler,S., Flohrs,K., Goessen,A., Leidel,S., Alleaume,A.M., Martin,C., Ozlu,N., Bork,P. and Hyman,A.A. TITLE Functional genomic analysis of cell division in C. elegans using RNAi of genes on chromosome III JOURNAL Nature 408 (6810), 331-336 (2000) MEDLINE 20548710 PUBMED 11099034 REFERENCE 3 (residues 1 to 429) AUTHORS Leroux,M.R. and Candido,E.P. TITLE Characterization of four new tcp-1-related cct genes from the nematode Caenorhabditis elegans JOURNAL DNA Cell Biol. 14 (11), 951-960 (1995) MEDLINE 96069542 PUBMED 7576182 COMMENT PROVISIONAL REFSEQ: This record has not yet been subject to final NCBI review. This record is derived from an annotated genomic sequence (NC_003281). The reference sequence was derived from WormBase CDS:F01F1.8b. Summary: This essential gene cct-6, also known as F01F1.8, 3G944 or YK828, maps at (III; -1.53). Phenotypes and affected processes are embryonic lethal, sterile adult, unhealthy, clear, translucent appearance, protruding vulva, small embryos, slow embryonic cell division, cytokinesis defect, abnormal cytoplasmic appearance. It encodes a chaperonin Containing TCP-1. According to the Worm Transcriptome Project, it is expressed at very high level at all stages of development [Kohara cDNAs]. Its existence, but not its exact sequence, derived here from the genome sequencing consortium annotation, is supported by 122 cDNA clones. It would produce, by alternative splicing, 2 different transcripts a, b altogether encoding 2 different protein isoforms. RNA interference results [T.Hyman; 2000] All embryos dead. DIC phenotype -- Semi- sterile; complex DIC phenotype; many embryos loose structural integrity upon dissection; areas lacking yolk granules; failure in different microtubule-based processes (centration/rotation, spindle assembly, chromosome segregation). DIC phenotype comment -- see also results from C07G2.3. Phenotype comment -- Confirmed with independent dsRNA (F01F1.8-RNA2; similar phenotype) (by injecting genomic PCR product TH:304C1). Movies are available on Hyman's site.| Same description as TH:304C1 (by injecting genomic PCR product TH:341B5). [J.Ahringer 2003] Embryonic lethal (100%), sterile, sick, clear, protruding vulva (by feeding genomic PCR product JA:F01F1.8). Function Protein properties: [C.elegansII] NMK. Encodes one of 7-9 related subunits of eukaryotic cytosolic chaperonin CCT.Ortholog of mouse Cctz (67% aa sequence identity)[PC]. Expression The expression profile for the gene, derived from the proportion of animals at each stage in each Kohara library is: embryos 5%, L1 or L2 larvae 53%, L3 to adult 41%. In situ hybridisation pictures to all stages of development are available from Kohara NextDB. For a detailed expression pattern description, see Wormbase Expr2045. The predicted CDS has 6 exons. It covers 1.54 kb on the WS97 genome. The protein (429 aa, 47.6 kDa, pI 6.3) contains one chaperonin Cpn60/TCP-1 motif. It also contains an ER membrane domain [Psort2]. It is predicted to localise in the cytoplasm [Psort2]. Taxblast (threshold 10^-3) tracks ancestors down to archaea and eukaryota. Method: conceptual translation. FEATURES Location/Qualifiers source 1..429 /organism="Caenorhabditis elegans" /db_xref="taxon:6239" /chromosome="III" /map="III;-1.53 cM (interpolated genetic position)" /map="III; covering 2163 bp, from base 5855637 to 5853475 on genome release WS97" Protein 1..42 /product="chaperonin Containing TCP-1 (cct-6)" Region 30..428 /region_name="[Pfam/InterPro description] chaperonin Cpn60/TCP-1" /db_xref="CDD:pfam00118" Region 425..428 /region_name="[PSORT] ER membrane domain: VEKR" (SEQ ID NO: 69) CDS 1..429 /gene="cct-6" /locus_tag="3G944" /coded_by="NM_171136.1:1..1290" /db_xref="AceView/WormGenes:cct-6" /db_xref="GeneID:175819" /db_xref="LocusID:175819" ORIGIN 1 mssiqclnpk aelarhaaal elnisgargl qdvmrsnlgp kgtlkmlvsg agdikltkdg 61 nvllhemaiq hptasmiaka staqddvtgd gttstvllig ellkqaeslv leglhprivt 121 egfewantkt lellekfkke apverdllve vcrtalrtkl hqkladhite cvvdavlair 181 rdgeepdlhm vekmemhhds dmdttlvrgl vldhgarhpd mprhvkdayi ltcnvsleye 241 ktevnsglfy ktakereall aaerefitrr vhkiielkkk vidnspdgkn kgfvvinqkg 301 idppsldlla segilalrra krrnmerlql avggeavnsv ddltpedlgw aglvyehslg 361 eekytfieec rapksvtlli kgpnkhtitq ikdaihdglr avfntivdsc spwsccfrnc 421 clrdvekrc (SEQ ID NO: 70)IIIFF.RDE-4

The RDE-4 protein is structurally related to drosophila R2D2 and the human TAR binding protein with conservation in the dsRBDs motifs.

LOCUS CAA83012 385 aa linear DEFINITION Hypothetical protein T20G5.11 [Caenorhabditis elegans]. ACCESSION CAA83012 VERSION CAA83012.1 GI:458490 DBSOURCE embl locus CET20G5, accession Z30423.2 KEYWORDS SOURCE Caenorhabditis elegans ORGANISM Caenorhabditis elegans Eukaryota; Metazoa; Nematoda; Chromadorea; Rhabditida; Rhabditoidea; Rhabditidae; Peloderinae; Caenorhabditis. REFERENCE 1 (residues 1 to 385) AUTHORS . CONSRTM WormBase Consortium TITLE Genome sequence of the nematode C. elegans: a platform for investigating biology JOURNAL Science 282 (5396), 2012-2018 (1998) PUBMED 9851916 REFERENCE 2 (residues 1 to 385) AUTHORS Berks,M., Lloyd,C.R. and Smith,A. TITLE Direct Submission JOURNAL Submitted (07-MAR-1994) Nematode Sequencing Project, Sanger Institute, Hinxton, Cambridge CB10 1SA, England and Department of Genetics, Washington University, St. Louis, MO 63110, USA. E-mail: worm@sanger.ac.uk COMMENT Coding sequences below are predicted from computer analysis, using predictions from Genefinder (P. Green, U. Washington), and other available information. Current sequence finishing criteria for the C. elegans genome sequencing consortium are that all bases are either sequenced unambiguously on both strands, or on a single strand with both a dye primer and dye terminator reaction, from distinct subclones. Exceptions are indicated by an explicit note. IMPORTANT: This sequence is NOT necessarily the entire insert of the specified clone. It may be shorter because we only sequence overlapping sections once, or longer because we arrange for a small overlap between neighbouring submissions This sequence is the entire insert of clone T20G5. The start of this sequence (1..100) overlaps with the end of sequence Z30974. The end of this sequence (47996..48095) overlaps with the start of sequence AL032660. For a graphical representation of this sequence and its analysis see:- Wormbase database maintained at Cold Spring Harbor Laboratories, New York, U.S.A. name=ZK1321;class=Sequence. FEATURES Location/Qualifiers source 1..385 /organism="Caenorhabditis elegans" /strain="Bristol N2" /db_xref="taxon:6239" /chromosome="III" /clone="T20G5" Protein 1..385 /product="Hypothetical protein T20G5.11" CDS 1..385 /gene="rde-4" /locus_tag="T20G5.11" /standard_name="T20G5.11" /coded_by="complement(join(Z30423.2:45951..46375, Z30423.2:46424..46544,Z30423.2:46589..46820, Z30423.2:46870..47249))" /note="C. elegans RDE-4 protein; contains similarity to Pfam domain PF00035 (Double-stranded RNA binding motif)" /db_xref="GOA:Q22617" /db_xref="InterPro:IPR001159" /db_xref="UniProt/TrEMBL:Q22617" ORIGIN 1 mdltkltfes vfggsdvpmk psrsednktp rnrtdlemfl kktplmvlee aakavyqktp 61 twgtvelpeg femtlilnei tvkgqatskk aarqkaavey lrkvvekgkh eiffipgttk 121 eealsnidqi sdkaeelkrs tsdavqdndn ddsiptsaef ppgisptenw vgklqeksqk 181 sklqapiyed sknerterfl victmcnqkt rgirskkkda knlaawlmwk aledgiesle 241 sydmvdvien leeaehllei qdqaskikdk hsalidilsd kkrfsdysmd fnvlsvstmg 301 ihqvlleisf rrlvspdpdd lemgaehtqt eeimkataek eklrkknmpd sgplvfaghg 361 ssaeeakqca cksaiihfnt ydftd (SEQ ID NO: 71)

IIIGG.DRH-3 (D2005.5)

The DRH-3 protein now has been officially renamed DRH-3, this protein is a paralog of DRH-1 and DRH-2 which are essential for RNAi and have a human ortholog: melanoma differentiation associated protein-5. LOCUS CAB02082 1119 aa linear DEFINITION Hypothetical protein D2005.5 [Caenorhabditis elegans]. ACCESSION CAB02082 VERSION CAB02082.3 GI:38422755 DBSOURCE embl locus CED2005, accession Z79752.2 KEYWORDS . SOURCE Caenorhabditis elegans ORGANISM Caenorhabditis elegans Eukaryota; Metazoa; Nematoda; Chromadorea; Rhabditida; Rhabditoidea; Rhabditidae; Peloderinae; Caenorhabditis. REFERENCE 1 (residues 1 to 1119) AUTHORS . CONSRTM WormBase Consortium TITLE Genome sequence of the nematode C. elegans: a platform for investigating biology JOURNAL Science 282 (5396), 2012-2018 (1998) PUBMED 9851916 REFERENCE 2 (residues 1 to 1119) AUTHORS Wilkinson,J. TITLE Direct Submission JOURNAL Submitted (04-SEP-1996) Nematode Sequencing Project, Sanger Institute, Hinxton, Cambridge CB10 1SA, England and Department of Genetics, Washington University, St. Louis, MO 63110, USA. E-mail: worm@sanger.ac.uk COMMENT Coding sequences below are predicted from computer analysis, using predictions from Genefinder (P. Green, U. Washington), and other available information. Current sequence finishing criteria for the C. elegans genome sequencing consortium are that all bases are either sequenced unambiguously on both strands, or on a single strand with both a dye primer and dye terminator reaction, from distinct subclones. Exceptions are indicated by an explicit note. IMPORTANT: This sequence is NOT necessarily the entire insert of the specified clone. It may be shorter because we only sequence overlapping sections once, or longer because we arrange for a small overlap between neighbouring submissions. IMPORTANT: This sequence is not the entire insert of clone D2005. It may be shorter because we only sequence overlapping sections once, or longer because we arrange for a small overlap between neighbouring submissions. The true left end of clone D2005 is at 1 in this sequence. The true right end of clone D2005 is at 104 in sequence Z81073. The true left end of clone F30F8 is at 43337 in this sequence. The start of this sequence (1..104) overlaps with the end of sequence AL033124. The end of this sequence (43337..43440) overlaps with the start of sequence Z81073. For a graphical representation of this sequence and its analysis see:- Wormbase database maintained at Cold Spring Harbor Laboratories, New York, U.S.A. name=ZK1321;class=Sequence. FEATURES Location/Qualifiers source 1..1119 /organism="Caenorhabditis elegans" /strain="Bristol N2" /db_xref="taxon:6239" /chromosome="I" /clone="D2005" Protein 1..1119 /product="Hypothetical protein D2005.5" CDS 1..1119 /locus_tag="D2005.5" /standard_name="D2005.5" /coded_by="join(Z79752.2:37322..37419, Z79752.2:37479..37652,Z79752.2:37906..37981, Z79752.2:38029..38110,Z79752.2:38156..38680, Z79752.2:38868..38993,Z79752.2:39040..39221, Z79752.2:39303..39484,Z79752.2:39682..40007, Z79752.2:40444..40724,Z79752.2:40768..41676, Z79752.2:42116..42216,Z79752.2:42273..42411, Z79752.2:42459..42565,Z79752.2:42668..42719)" /note="contains similarity to Pfam domains PF00270 (DEAD (SEQ ID NO: 14) and DEAH (SEQ ID NO: 15) box helicases), PF00271 (Helicases conserved C-terminal domain)" /db_xref="GOA:Q93413" /db_xref="InterPro:IPR001410" /db_xref="InterPro:IPR001650" /db_xref="InterPro:IPR011545" /db_xref="UniProt/TrEMBL:Q93413" ORIGIN 1 mqptairled ydksklrlpf espyfpayfr llkwkfldvc vestrnndig yfklfeslfp 61 pgkleeiarm iideptpvsh dpdmikirna dldvkirkqa etyvtlrhah qqkvqrrrfs 121 ecflntvlfd ekglriadev mfnydkelyg yshwedlpdg wltaetfknk fydeeevtnn 181 pfgyqkldrv agaargmiim khlksnprcv settilafev fnkgnhqlst dlvedllteg 241 pafelkieng eekkyavkkw slhktltmfl aiigfksndk kekneheewy ygfidamknd 301 panraalyfl dknwpeelee rekerdrirl tllksqrtne eavgedvctt irpqpkdsgy 361 npdavvtelv lrtyqeelvq palegkncvi vaptgsgkte vaiyaalkhi eertsqgkps 421 rvvllvpkip lvgqqkdrfl kycngmyevn gfhgsessvs gtgrrdevia thvsvmtpqi 481 linmlqsvrq nerlyvsdfs mmifdevhka aknhpyvlin qmvqewkyek pqiigltasl 541 svkvdgqkde nqmlndiynm lalinaphls titrqsside lnehvgkpdd svelclpake 601 nilrdyiery lnhahgkfle elasmskstg rnntippnmi ntfkknqpkn yeyydsllqg 661 iiqelnklnv pekwnsqtwa kymkvylear givdlmpamv afkymekaig klneshsetv 721 eystfikdhd tlkqtiqsve peivlrlknt ltnqfhvape srviifvtqr staqrvsdfl 781 neskvldqfg nygeqmvgyv lgtnkqgavq qtsqeqqltl dkfnngrlkv ivatsvveeg 841 ldvtacnlii kyncssgsai qlvqqrgrar aknsrsvlls vkssinetet nalisekymr 901 lcvkkiteng ekqlaaevkr vaelnaaerk rnleeqlnlr lrhenkiykl mcsncskefc 961 ksiyikkvfs nymvfdpsvw rflhveskrk vskylsednq plsdikcfhc kldvgrayki 1021 rgtylpqlsv kaltfvqesd yssmtkakws dveqdlfyis eaieddfrim lnalsdteen 1081 iekkivldld srqhnkqlem krfhiqqepp tkgvapeaq (SEQ ID NO: 72)IIIHH.ERI-1

The ERI-1 protein is conserved and enhances RNAi and has a human homolog: AAH35279. LOCUS T32581 562 aa linear DEFINITION hypothetical protein T07A9.10 - Caenorhabditis elegans. ACCESSION T32581 VERSION T32581 GI:7507339 DBSOURCE pir: locus T32581; summary: #length 562 #molecular-weight 64656 #checksum 867; genetic: #gene CESP:T07A9.10 #map_position 4 #introns 9/1; 54/1; 218/1; 258/3; 349/1; 432/3; 516/1; superfamily: vacuolar protein sorting protein VPS45; KEYWORDS SOURCE Caenorhabditis elegans ORGANISM Caenorhabditis elegans Eukaryota; Metazoa; Nematoda; Chromadorea; Rhabditida; Rhabditoidea; Rhabditidae; Peloderinae; Caenorhabditis. REFERENCE 1 (residues 1 to 562) AUTHORS Scheet,P. and Maggi,L. TITLE Direct Submission JOURNAL Submitted (??-DEC-1997) to the EMBL Data Library FEATURES Location/Qualifiers source 1..562 /organism="Caenorhabditis elegans" /db_xref="taxon:6239" Protein 1..562 /product="hypothetical protein T07A9.10" ORIGIN 1 mlrelvkkqi ienilrpqny dsklghrkfs vlvldksamv vvnsclslne vfeegvtlve 61 dltrnrepmp smdaiyiisp vaesidilin dfsrktkfnp gnsyrsahif fldpccdelf 121 eklskspavk wiktlkelnl nlkpvesqif tvnsqfrgdm tktadgivsl catlnihptl 181 rfqsdfaqss eicqrveqkl kefgnegmgt daelvvldrs fdlvspllhe vtlqamvvdv 241 tafkdgvyry teagdskeiv ldekdqnwld lrhkllpevm ksvnkmvkdf kntnktepen 301 iknqsskdfs ttvrtlqpyl kmkakmaayi slteecrsky fdslekiial eqdmavehtp 361 ehvritdsqa vgrlstfilp aiptetrlrl ilifmltigk dkdeqyfnrl lhhtdipese 421 fqiikrmliw rdktqksqfq hrrpppeder fiasrwdpki knlieeiyer rlderefkva 481 gkkstsdfrp aasarygsgl agkprekrki iifvvggity semrvayels kktnttvilg 541 sdeiltpssf leslrdrntv nc (SEQ ID NO: 73)

III.RRF-3

This protein is also conserved in S. pombe and many plants. LOCUS CAA88315 1780 aa linear DEFINITION Hypothetical protein F10B5.7 [Caenorhabditis elegans]. ACCESSION CAA88315 VERSION CAA88315.1 GI:3875716 DBSOURCE embl locus CEF10B5, accession Z48334.1 embl locus CET05C12, accession Z66500.1 KEYWORDS . SOURCE Caenorhabditis elegans ORGANISM Eukaryota; Metazoa; Nematoda; Chromadorea; Rhabditida; Rhabditoidea; Rhabditidae; Peloderinae; Caenorhabditis. REFERENCE 1 (residues 1 to 1780) AUTHORS . CONSRTM C. elegans Sequencing Consortium TITLE Genome sequence of the nematode C. elegans: a platform for investigating biology JOURNAL Science 282 (5396), 2012-2018 (1998) PUBMED 9851916 REFERENCE 2 (residues 1 to 1780) AUTHORS Sims,M.A. TITLE Direct Submission JOURNAL Submitted (16-FEB-1995) Nematode Sequencing Project, Sanger Institute, Hinxton, Cambridge CB10 1SA, England and Department of Genetics, Washington University, St. Louis, MO 63110, USA. E-mail: worm@sanger.ac.uk COMMENT Coding sequences below are predicted from computer analysis, using predictions from Genefinder (P. Green, U. Washington), and other available information. Current sequence finishing criteria for the C. elegans genome sequencing consortium are that all bases are either sequenced unambiguously on both strands, or on a single strand with both a dye primer and dye terminator reaction, from distinct subclones. Exceptions are indicated by an explicit note. IMPORTANT: This sequence is NOT necessarily the entire insert of the specified clone. It may be shorter because we only sequence overlapping sections once, or longer because we arrange for a small overlap between neighbouring submissions. IMPORTANT: This sequence is not the entire insert of clone F10B5. It may be shorter because we only sequence overlapping sections once, or longer because we arrange for a small overlap between neighbouring submissions. The true left end of clone F10B5 is at 1 in this sequence. The true right end of clone F10B5 is at 15182 in sequence Z66500. The true left end of clone T05C12 is at 29032 in this sequence. The true right end of clone C41C4 is at 2219 in this sequence. The start of this sequence (1..99) overlaps with the end of sequence Z48045. The end of this sequence (29032..29132) overlaps with the start of sequence Z66500. For a graphical representation of this sequence and its analysis see:- Wormbase database maintained at Cold Spring Harbor Laboratories, New York, U.S.A. name=ZK1321;class=Sequence. FEATURES Location/Qualifiers source 1..1780 /organism="Caenorhabditis elegans" /strain="Bristol N2" /db_xref="taxon:6239" /chromosome="II" /clone="F10B5" Protein 1..1780 /product="Hypothetical protein F10B5.7" CDS 1..1780 /gene="rrf-3" /locus_tag="F10B5.7" /standard_name="F10B5.7" /coded_by="join(Z48334.1:23435..23502, Z48334.1:23558..24167,Z48334.1:24214..24449, Z48334.1:24497..24610,Z48334.1:24661..25018, Z48334.1:25064..25883,Z48334.1:25931..26489, Z48334.1:26532..26743,Z48334.1:26790..27477, Z48334.1:27526..28249,Z48334.1:28294..28751, Z48334.1:28797..28902,Z48334.1:28954..29132, Z66500.1:102..114,Z66500.1:161..358)" /note="C. elegans RRF-3 protein; contains similarity to Pfam domain PF05183 (RNA dependent RNA polymerase)" /db_xref="InterPro:IPR007855" /db_xref="UniProt/TrEMBL:Q19285" ORIGIN 1 mlpfdnddss ddattsvrpk hprgvpqsqs tfprgrsnfs sgtlpnrkte ctpvntltig 61 hsnkmllttf rmdrnsksks evdvqeqpvh ssssafpgnh lnnfsypvnr gylrdyllqs 121 qrpstskpvd csvlkrhslp sthilyektk hrggvnieeq eklvrmlwaa aeesetvakt 181 rqfskkqaie lnfdakligs mnndcfgycr ahmenikdvl kthlklskvd evnwikvgmv 241 praayedksy vidahlvltp ngevedenel fsefassfts ritgmlhdqv flevpkmhtl 301 ftkitpqhmd inisaiaign cpnsglflvr gdfisqentv csvklqshhn adasrenssf 361 kvagsnkyls yarfehdkrl avvyfgvrla efaddgldha gfrlnlyynl fvrivvdmsh 421 ettnsiyiqm knpphlwegi pkntifhpsk skvlnmetct ewtrvlswpg daegrgvgct 481 seafsqsswi rltmrkdddn dsvsstqlmd ivtrlsarsk akvmfgsifs irrklapspa 541 fhslgsfran yalqalitrg svftdqlfda tdenipssdn dndedddddv ddtkkpmelv 601 heplflklvr rgmkecsqat eetleqllna fderrqidvv tafttmyqsr kiqyerllkg 661 eslqdvglak plpkncvsva kvivtpsril lmapevmmvn rvvrrfgpdy alrcvfrddn 721 lgrlairdfs innidhmsni vtegiyltlk ngiqvadrvy sflgwsnsqm rdqgcylyap 781 rvnaltgevt gtvedirvwm gdfrdaisvp kmmsrmgqcf tqaqptvyss vknihiveni 841 qvrlerhhwi vepdieggve nkycfsdgcg risiklathi skilqlkevp acfqvrfkgf 901 kgilvidpti ddiinmpkvi frksqqkfge gggelqdeyl evvkyampsp vclnrpfiti 961 ldqvsekqsa sshrritnrv hyylerelcs lsnmlinenq aaeelvnrtn laidwnaask 1021 ragfelsvdp lirdmlfsiy ryniihhisk akiflppslg rsmygvvdet gllqygqvfi 1081 qyspsirqts nrpilktgkv litknpchvp gdvrvfdavw qpalahlvdv vvfpqhgprp 1141 hpdemagsdl dgdeysiiwd qemlldynee amvfpsssaa eedkepttdd mvefflrylq 1201 qdsigrmsha hlayadlhgl fhenchaial kcavavdfpk sgvpaeplss feqcemtpdy 1261 mmsggkpmyy strlngqlhr karkveevle efetrgsvfe reydklicpe dvdvffgnei 1321 klvqtltlrd eyvdrmqqll deygiedeas vvsghaasik rlagmerddy sfyhtdkvve 1381 lryeklyavf rakffeefgg eeiniendgk ntrlkctkam hekirqwyfv ayvqpkinka 1441 grcigqslpw vawdalcdlr rqlmldknda vlrgkypiaa rleeeiensi erqfdkflkl 1501 kdlieshkda lflrryvyfy gdqiikmlfi lkvwlerenv lpssvlsiwq lgrllirlgl 1561 gdllgnptid yeksllmptt mfqqwiskke dadeapilrn fdmgtmmlef lrylasqsfa 1621 saesislrvf yekdivepil tksaqwmplh liayrtfhsi avsgrfdalh lddedavdqi 1681 teskdpilvn eslfssrnyn ddypisrsri lqslkdwsgv keiipreitg trksdmiyvt 1741 svgtvlarqr larllllsge tirdaiannv vpnevrdefl (SEQ ID NO: 74)

IIIJJ.ERI-3 (W09B6.3)

This protein is expressed as an operon with TAF-6.1 and expressed as a fusion protein and enhances RNAi when mutated. LOCUS NP_493918 578 aa linear DEFINITION putative protein (66.4 kD) (2B417) [Caenorhabditis elegans]. ACCESSION NP_493918 VERSION NP_493918.2 GI:32565182 DBSOURCE REFSEQ: accession NM_061517.2 KEYWORDS . SOURCE Caenorhabditis elegans ORGANISM Caenorhabditis elegans Eukaryota; Metazoa; Nematoda; Chromadorea; Rhabditida; Rhabditoidea; Rhabditidae; Peloderinae; Caenorhabditis. COMMENT VALIDATED REFSEQ: This record has undergone preliminary review of the sequence, but has not yet been subject to final review. This record is derived from an annotated genomic sequence (NC_003280). The reference sequence was derived from WormBase CDS:W09B6.3. Summary: This gene 2B417, also known as W09B6.3 or YK7122, maps at (II; -12.85). It encodes a putative protein. According to the Worm Transcriptome Project, it is well expressed at all stages of development [Kohara cDNAs]. Its sequence is fully supported by 7 cDNA clones. RNA interference results: [J.Ahringer 2003] No obvious phenotype (by feeding genomic PCR product JA:W09B6.3). [J.Ahringer 2003] No obvious phenotype (by feeding genomic PCR product JA:W09B6.2). Warning: this double stranded RNA may also interfere with gene taf-6.1. Expression The expression profile for the gene, derived from the proportion of animals at each stage in each Kohara library is: embryos 21%, L1 or L2 larvae 31%, L3 to adult (including dauer) 48% In situ hybridisation pictures to all stages of development are available from Kohara NextDB. The CDS has 11 exons. It covers 4.20 kb on the WS97 genome. The protein (578 aa, 66.4 kDa, pI 8.5) contains no Pfam motif. Taxblast (threshold 10^-3) tracks ancestors down to caenorhabditis elegans. COMPLETENESS: full length. Method: conceptual translation. FEATURES Location/Qualifiers source 1..578 /organism="Caenorhabditis elegans" /db_xref="taxon:6239" /chromosome="II" /map="II;-12.85 cM (interpolated genetic position)" /map="II; covering 4252 bp, from base 1123539 to 1127792 on genome release WS97" /clone_lib="Kohara embryonic lambda gt11 library: yk516c2, yk590b10; Kohara Sugano L1 larvae cap- selected library: yk1341c6, yk1341d5; Kohara Sugano L2 larvae cap-selected library: yk1378a11; Kohara Sugano mixed stage cap-selected library: yk724f12; Kohara mixed stage library, from him-8 strain, containing 15-30% males: yk379e7" Protein 1..578 /product="putative protein (66.4 kD) (2B417)" Region 347..350 /region_name="[PSORT] nuclear localization domain: KKKK" (SEQ ID NO: 75) Region 414..417 /region_name="[PSORT] vacuolar domain: ILPK" (SEQ ID NO: 76) Region 456..462 /region_name="[PSORT] nuclear localization domain: PKNPKKR" (SEQ ID NO: 77 Region 459..465 /region_name="[PSORT] nuclear localization domain: PKKRVEI" (SEQ ID NO: 78) CDS 1..578 /gene="2B417" /locus_tag="2B417" /coded_by="NM_061517.2:1..1737" /db_xref="AceView/WormGenes:2B417" /db_xref="GeneID:173497" /db_xref="WormBase:W09B6.3" ORIGIN 1 mqpvlvnsrp lrvksheses klnlieqedq feganyssss gviicysngt gevitqeafd 61 dsgihfifsk atciqypsnf dpigvgsvvq ifwsrsferv vrgnhiivqi ekmevykcca 121 mlreqvfvtf nspstagvai gvternitva fhpncspvir yetlkahsig rtefeikdrh 181 rentnrmvdv ilaavpfrve ihgnvdkipf fviekcrnsp grsgaavitk imknhfmean 241 flqnsesiyf dstschsnil ekvsigslin vladptfats sykwygydvt lcnnylahas 301 tqrsfvlenn eilqnckkle kspeeaettt kndlrfvppq pekgevkkkk mtnclkfnsk 361 saqfklrhli ldrcfselpe reaksiinsy fidrlaegik iekidknwrt fgeilpktpk 421 kyseslkksi qnvlepfgln kpekaaetpk iveyfpknpk krveivekpt vdeirelfga 481 lmdaegfaln qrvkphfvlp dtrwkpterr yigiyddvqw tfmstfcpki eensenrpla 541 ggwwyrrtvp rdhpveivqk metrrniikd ctespfie (SEQ ID NO: 79)

IIIKK.ERI-5 (Y38F2AR.1)

This protein has homologs in multiple species, with conservation found in the TUDOR domain. The paralog f22d6.6 plays a role in other small RNA silencing pathways in C elegans. LOCUS NP_500199 458 aa linear DEFINITION maternal tudor protein (4D159) [Caenorhabditis elegans]. ACCESSION NP_500199 VERSION NP_500199.1 GI:17543178 DBSOURCE REFSEQ: accession NM_067798.1 KEYWORDS . SOURCE Caenorhabditis elegans ORGANISM Caenorhabditis elegans Eukaryota; Metazoa; Nematoda; Chromadorea; Rhabditida; Rhabditoidea; Rhabditidae; Peloderinae; Caenorhabditis. COMMENT PROVISIONAL REFSEQ: This record has not yet been subject to final NCBI review. This record is derived from an annotated genomic sequence (NC_003282). The reference sequence was derived from WormBase CDS:Y38F2AR.1. Summary: This gene 4D159, also known as Y38F2AR.1 or YK7605, maps at (IV; -9.66). It encodes a maternal tudor protein. According to the Worm Transcriptome Project, it is moderately expressed in embryos, L1, L2 and L3 larvae [Kohara cDNAs]. Its existence, but not its exact sequence, derived here from the genome sequencing consortium annotation, is supported by 5 cDNA clones. RNA interference results: [J.Ahringer 2003] No obvious phenotype (by feeding genomic PCR product JA:Y38F2A_6126.j). Expression The expression profile for the gene, derived from the proportion of animals at each stage in each Kohara library is: embryos 16%, L1 or L2 larvae 66%, L3 to adult 18%. In situ hybridisation pictures to all stages of development are available from Kohara NextDB. The predicted CDS has 6 exons. It covers 5.00 kb on the WS97 genome. The protein (458 aa, 53.0 kDa, pI 4.7) contains one maternal tudor protein motif. It also contains an ER membrane domain [Psort2]. Taxblast (threshold 10^-3) tracks ancestors down to caenorhabditis elegans. Method: conceptual translation. FEATURES Location/Qualifiers source 1..458 /organism="Caenorhabditis elegans" /db_xref="taxon:6239" /chromosome="IV" /map="IV;-9.66 cM (interpolated genetic position)" /map="IV; covering 5438 bp, from base 2390825 to 2396264 on genome release WS97" /clone_lib="Kohara embryonic lambda gt11 library: yk592e2; Kohara Sugano L2 larvae cap-selected library: yk818d10, yk1502b5, yk1498b6, yk1503h5" Protein 1..458 /product="maternal tudor protein (4D159)" Region 13..65 /region_name="[Pfam/InterPro description] maternal tudor protein" /db_xref="CDD:pfam00567" Region 454..457 /region_name="[PSORT] ER membrane domain: DKDS" (SEQ ID NO: 80) CDS 1..458 /gene="4D159" /locus_tag="4D159" /coded_by="NM_067798.1:1..1377" /db_xref="AceView/WormGenes:4D159" /db_xref="GeneID:177029" /db_xref="WormBase:Y38F2AR.1" ORIGIN 1 mamaplrprv farclilknl elieaariff idsavtanvs wkclfqiden lkfhpwqamh 61 ctlgrlvhls dswtdtqcte frnivskfak fqitanqcdv dfrsdrpsll vnlyglpngt 121 eidkkvaiee icavsmqnvm vsqfptnfmv npkleeldke qdhldville efrrdlpadw 181 aheppadyre ddadwdilqc hvaewndtal eqfrradgsf wamlepsctv spwemhvtpi 241 lapekmsdne hwifeqlvkn senqqkiddf ysnlknqrpl emeeikfalq tgrtyvmati 301 knrqkssaqw lrceiidflp nanvalryvd lgtrgilklk nlhrmhieht kiapacieig 361 rfldddlsma dsemewnthf wreivpydvp ivvgpdmefl etgklqfsqi rvagdedden 421 lldkipspsp fftersddlr tqkeddddgn vsddkdsg (SEQ ID NO: 81)

IIILL.PIR-1 (T23G7.5)

This gene is an ortholog of the well conserved PIR-1 from human and mouse and required for RNAi in C elegans. An ortholog is the human dual specificity phosphatase 11 (DUSP11). LOCUS CAA92703 261 aa linear DEFINITION Hypothetical protein T23G7.5 [Caenorhabditis elegans]. ACCESSION CAA92703 VERSION CAA92703.1 GI:3880145 DBSOURCE embl locus CET23G7, accession Z68319.1 KEYWORDS . SOURCE Caenorhabditis elegans ORGANISM Caenorhabditis elegans Eukaryota; Metazoa; Nematoda; Chromadorea; Rhabditida; Rhabditoidea; Rhabditidae; Peloderinae; Caenorhabditis. REFERENCE 1 (residues 1 to 261) AUTHORS . CONSRTM WormBase Consortium TITLE Genome sequence of the nematode C. elegans: a platform for investigating biology JOURNAL Science 282 (5396), 2012-2018 (1998) PUBMED 9851916 REFERENCE 2 (residues 1 to 261) AUTHORS Barlow,K. TITLE Direct Submission JOURNAL Submitted (22-DEC-1995) Nematode Sequencing Project, Sanger Institute, Hinxton, Cambridge CB10 1SA, England and Department of Genetics, Washington University, St. Louis, MO 63110, USA. E-mail: worm@sanger.ac.uk COMMENT Coding sequences below are predicted from computer analysis, using predictions from Genefinder (P. Green, U. Washington), and other available information. Current sequence finishing criteria for the C. elegans genome sequencing consortium are that all bases are either sequenced unambiguously on both strands, or on a single strand with both a dye primer and dye terminator reaction, from distinct subclones. Exceptions are indicated by an explicit note. IMPORTANT: This sequence is NOT necessarily the entire insert of the specified clone. It may be shorter because we only sequence overlapping sections once, or longer because we arrange for a small overlap between neighbouring submissions. 951009: yk82b3.3 delimits the 3' end of ZK1067.6 960305: T23G7 deleted in union with ZK1067.6 IMPORTANT: This sequence is not the entire insert of clone T23G7. It may be shorter because we only sequence overlapping sections once, or longer because we arrange for a small overlap between neighbouring submissions. The true left end of clone T23G7 is at 1 in this sequence. The true right end of clone T23G7 is at 16033 in sequence Z70038. The true left end of clone ZK1067 is at 19833 in this sequence. The true right end of clone W07A12 is at 6609 in this sequence. The start of this sequence (1..104) overlaps ith the end of sequence Z68320. The end of this sequence (19833..19934) overlaps with the start of sequence Z70038. For a graphical representation of this sequence and its Analysis see:-Wormbase database maintained at Cold Spring Harbor Laboratories, New York, U.S.A. name=ZK1321;class=Sequence. FEATURES Location/Qualifiers source 1..261 /organism="Caenorhabditis elegans" /strain="Bristol N2" /db_xref="taxon:6239" /chromosome="II" /clone="T23G7" Protein 1..261 /product="Hypothetical protein T23G7.5" CDS 1..261 /locus_tag="T23G7.5" /standard_name="T23G7.5" /coded_by="join(Z68319.1:12488..12654, Z68319.1:12851..13093,Z68319.1:13144..13241, Z68319.1:13297..13407,Z68319.1:13455..13621)" /note="contains similarity to Pfam domain PF00782 (Dual specificity phosphatase, catalytic domain)" /db_xref="GOA:Q22707" /db_xref="InterPro:IPR000340" /db_xref="InterPro:IPR000387" /db_xref="UniProt/TrEMBL:Q22707" ORIGIN 1 mpeprctaiv nflnlshsil isifsvsvms nyhhnhnyqh rprgyerlpg krlpdrwniy 61 dnvgrdidgt rfvpfktpld ssffdgknmp velqfgvktl islaqqankq iglvidltnt 121 dryykktewa dhgvkylkln cpghevnere dlvqdfinav kefvndkend gkligvhcth 181 glnrtgylic rymidvdnys asdaismfey yrghpmereh ykkslyeaer kkkygkssgk 241 ssgnsadsti sseqlhrnns q (SEQ ID NO: 82)

IIIMM.C32A3.2 LOCUS CAA88285 346 aa linear DEFINITION Hypothetical protein C32A3.2 [Caenorhabditis elegans]. ACCESSION CAA88285 VERSION CAA88285.1 GI:3874617 DBSOURCE embl locus CEC32A3, accession Z48241.1 KEYWORDS . SOURCE Caenorhabditis elegans ORGANISM Caenorhabditis elegans Eukaryota; Metazoa; Nematoda; Chromadorea; Rhabditida; Rhabditoidea; Rhabditidae; Peloderinae; Caenorhabditis. REFERENCE 1 (residues 1 to 346) AUTHORS . CONSRTM WormBase Consortium TITLE Genome sequence of the nematode C. elegans: a platform for investigating biology JOURNAL Science 282 (5396), 2012-2018 (1998) PUBMED 9851916 REFERENCE 2 (residues 1 to 346) AUTHORS Thomas,K. TITLE Direct Submission JOURNAL Submitted (14-FEB-1995) Nematode Sequencing Project, Sanger Institute, Hinxton, Cambridge CB10 1SA, England and Department of Genetics, Washington University, St. Louis, MO 63110, USA. E-mail: worm@sanger.ac.uk COMMENT Coding sequences below are predicted from computer analysis, using predictions from Genefinder (P. Green, U. Washington), and other available information. Current sequence finishing criteria for the C. elegans genome sequencing consortium are that all bases are either sequenced unambiguously on both strands, or on a single strand with both a dye primer and dye terminator reaction, from distinct subclones. Exceptions are indicated by an explicit note. IMPORTANT: This sequence is NOT necessarily the entire insert of the specified clone. It may be shorter because we only sequence overlapping sections once, or longer because we arrange for a small overlap between neighbouring submissions IMPORTANT: This sequence is not the entire insert of clone C32A3. It may be shorter because we only sequence overlapping sections once, or longer because we arrange for a small overlap between neighbouring submissions. The true left end of clone C32A3 is at 1 in this sequence. The true right end of clone C32A3 is at 44660 in this sequence. The true left end of clone C46F11 is at 45409 in this sequence. The true right end of clone C48D5 is at 4074 in this sequence. The start of this sequence (1..102) overlaps with the end of sequence Z36237. The end of this sequence (45409..45510) overlaps with the start of sequence Z81449. For a graphical representation of this sequence and its analysis see:- Wormbase database maintained at Cold Spring Harbor Laboratories, New York, U.S.A. name=ZK1321;class=Sequence. FEATURES Location/Qualifiers source 1..346 /organism="Caenorhabditis elegans" /strain="Bristol N2" /db_xref="taxon:6239" /chromosome="III" /clone="C32A3" Protein 1..346 /product="Hypothetical protein C32A3.2" CDS 1..346 /locus_tag="C32A3.2" /standard_name="C32A3.2" /coded_by="complement(join(Z48241.1:31596..31840, Z48241.1:32812..33113,Z48241.1:33160..33321, Z48241.1:33366..33460,Z48241.1:33508..33603, Z48241.1:33657..33797))" /note="contains similarity to Homo sapiens Kinesin-like protein KIF14; ENSEMBL:ENSP00000236917" /db_xref="UniProt/Swiss-Prot:Q09261" ORIGIN 1 mqadgekkkk ktnpersthd dtpksrtrvl fsqyfflsfs lffraifmlr slcsiavrlg 61 garqprllss aasgdgndgk gakdaidedl lnaiegvann ihpqngsekk slkntlinrl 121 vanekasfda aaasasseml ddqaliglla dvagdakvek klppksaqlr qekrglvllr 181 keifyqavqs gftteearvk setivneaqi klqeqrkall ndvrekveqe eveetersek 241 dqklftmale fmekiykddl issavqfpta hsdqqilskn ksngqqkenn gniqsimssk 301 wamnrmfhsl ityswrdiyh hwvsrnlvql lilciwfvlv yprihi (SEQ ID NO: 83

Selected Human Homologs

Under this subsection, selected human homologs referred to above, are described in further detail.

Human melanoma differentiation associated protein-5 LOCUS NP_071451 1025 aa linear PRI 02-MAR-2005 DEFINITION melanoma differentiation associated protein-5 [Homo sapiens]. ACCESSION NP_071451 VERSION NP_071451.2 GI:27886568 DBSOURCE REFSEQ: accession NM_022168.2 KEYWORDS . SOURCE Homo sapiens (human) ORGANISM Homo sapiens Eukaryota; Metazoa; Chordata; Craniata; Vertebrata; Euteleostomi; Mammalia; Eutheria; Euarchontoglires; Primates; Catarrhini; Hominidae; Homo. REFERENCE 1 (residues 1 to 1025) AUTHORS Andrejeva,J., Childs,K.S., Young,D.F., Carlos,T.S., Stock,N., Goodbourn,S. and Randall,R.E. TITLE The V proteins of paramyxoviruses bind the IFN-inducible RNA helicase, mda-5, and inhibit its activation of the IFN-beta promoter JOURNAL Proc. Natl. Acad. Sci. U.S.A. 101 (49), 17264-17269 (2004) PUBMED 15563593 REMARK GeneRIF: mda-5 plays a central role in an intracellular signal transduction pathway that can lead to the activation of the IFN-beta promoter, and that the V proteins of paramyxoviruses interact with mda-5 to block its activity. REFERENCE 2 (residues 1 to 1025) AUTHORS Kang,D.C., Gopalkrishnan,R.V., Lin,L., Randolph,A., Valerie,K., Pestka,S. and Fisher,P.B. TITLE Expression analysis and genomic characterization of human melanoma differentiation associated gene-5, mda-5: a novel type I interferon-responsive apoptosis-inducing gene JOURNAL Oncogene 23 (9), 1789-1800 (2004) PUBMED 14676839 REMARK GeneRIF: mda-5 is a novel type I IFN-inducible gene, which may contribute to apoptosis induction during terminal differentiation and during IFN treatment REFERENCE 3 (residues 1 to 1025) AUTHORS Kang,D.C., Gopalkrishnan,R.V., Wu,Q., Jankowsky,E., Pyle,A.M. and Fisher,P.B. TITLE mda-5: An interferon-inducible putative RNA helicase with double-stranded RNA-dependent ATPase activity and melanoma growth-suppressive properties JOURNAL Proc. Natl. Acad. Sci. U.S.A. 99 (2), 637-642 (2002) PUBMED 11805321 REMARK GeneRIF: mda-5: An interferon-inducible putative RNA helicase with double-stranded RNA-dependent ATPase activity and melanoma growth-suppressive properties COMMENT REVIEWED REFSEQ: This record has been curated by NCBI staff. The reference sequence was derived from AF095844.1 and BU902097.1. Summary: DEAD box proteins (SEQ ID NO: 14), characterized by the conserved motif Asp-Glu-Ala-Asp (DEAD) (SEQ ID NO: 14), are putative RNA helicases. They are implicated in a number of cellular processes involving alteration of RNA secondary structure such as translation initiation, nuclear and mitochondrial splicing, and ribosome and spliceosome assembly. Based on their distribution patterns, some members of this family are believed to be involved in embryogenesis, spermatogenesis, and cellular growth and division. This gene encodes a DEAD box protein (SEQ ID NO: 14) that is upregulated in response to treatment with beta- interferon (IFNB) and a protein kinase C-activating compound, mezerein (MEZ). Irreversible reprogramming of melanomas can be achieved by treatment with both these agents; treatment with either agent alone only achieves reversible differentiation. FEATURES Location/Qualifiers source 1..1025 /organism="Homo sapiens" /db_xref="taxon:9606" /chromosome="2" /map="2p24.3-q24.3" Protein 1..1025 /product="melanoma differentiation associated protein-5" /note="DEAD/H (Asp-Glu-Ala-Asp/His) box polypeptide"(DEAD disclosed as SEQ ID NO: 14; DEAH disclosed as SEQ ID NO: 15) CDS 1..1025 /gene="IFIH1" /coded_by="NM_022168.2:223..3300" /db_xref="CCDS:CCDS2217.1" /db_xref="GeneID:64135" /db_xref="MIM:606951" ORIGIN 1 msngystden fryliscfra rvkmyiqvep vldyltflpa evkeqiqrtv atsgnmqave 61 lllstlekgv whlgwtrefv ealrrtgspl aarymnpelt dlpspsfena hdeylqllnl 121 lqptlvdkll vrdvldkcme eelltiedrn riaaaenngn esgvrellkr ivqkenwfsa 181 flnvlrqtgn nelvqeltgs dcsesnaeie nlsqvdgpqv eeqllsttvq pnlekevwgm 241 ennssessfa dssvvsesdt slaegsvscl deslghnsnm gsdsgtmgsd sdeenvaara 301 spepelqlrp yqmevaqpal egkniiiclp tgsgktrvav yiakdhldkk kkasepgkvi 361 vlvnkvllve qlfrkefqpf lkkwyrvigl sgdtqlkisf pevvkscdii istaqilens 421 llnlengeda gvqlsdfsli iidechhtnk eavynnimrh ylmqklknnr lkkenkpvip 481 lpqilgltas pgvggatkqa kaeehilklc anldaftikt vkenldqlkn qiqepckkfa 541 iadatredpf keklleimtr iqtycqmspm sdfgtqpyeq waiqmekkaa kegnrkervc 601 aehlrkynea lqindtirmi daythletfy neekdkkfav ieddsdeggd deycdgdede 661 ddlkkplkld etdrflmtlf fennkmlkrl aenpeyenek ltklrntime qytrteesar 721 giiftktrqs ayalsqwite nekfaevgvk ahhligaghs sefkpmtqne qkeviskfrt 781 gkinlliatt vaeegldike cniviryglv tneiamvqar graradesty vlvahsgsgv 841 iehetvndfr ekmmykaihc vqnmkpeeya hkilelqmqs imekkmktkr niakhyknnp 901 slitflcknc svlacsgedi hviekmhhvn mtpefkelyi vrenkalqkk cadyqingei 961 ickcgqawgt mmvhkgldlp clkirnfvvv fknnstkkqy kkwvelpitf pnldyseccl 1021 fsded (SEQ ID NO: 84)

Human SMD1 LOCUS NP_008869 119 aa linear DEFINITION small nuclear ribonucleoprotein D1 polypeptide 16kDa [Homo sapiens]. ACCESSION NP_008869 VERSION NP_008869.1 GI:5902102 DBSOURCE REFSEQ: accession NM_006938.2 KEYWORDS . SOURCE Homo sapiens (human) ORGANISM Homo sapiens Eukaryota; Metazoa; Chordata; Craniata; Vertebrata; Euteleostomi; Mammalia; Eutheria; Euarchontoglires; Primates; Catarrhini; Hominidae; Homo. REFERENCE 1 (residues 1 to 119) AUTHORS Fong,Y.W. and Zhou,Q. TITLE Stimulatory effect of splicing factors on transcriptional elongation JOURNAL Nature 414 (6866), 929-933 (2001) PUBMED 11780068 REFERENCE 2 (residues 1 to 119) AUTHORS Sun,D., Ou,Y.C. and Hoch,S.O. TITLE Analysis of genes for human snRNP Sm-D1 protein and identification of the promoter sequence which shows segmental homology to the promoters of Sm-E and U1 snRNA genes JOURNAL Gene 189 (2), 245-254 (1997) PUBMED 9168134 REFERENCE 3 (residues 1 to 119) AUTHORS Lehmeier,T., Raker,V., Hermann,H. and Luhrmann,R. TITLE cDNA cloning of the Sm proteins D2 and D3 from human small nuclear ribonucleoproteins: evidence for a direct D1-D2 interaction JOURNAL Proc. Natl. Acad. Sci. U.S.A. 91 (25), 12317-12321 (1994) PUBMED 7527560 REFERENCE 4 (residues 1 to 119) AUTHORS Lehmeier,T., Foulaki,K. and Luhrmann,R. TITLE Evidence for three distinct D proteins, which react differentially with anti-Sm autoantibodies, in the cores of the major snRNPs U1, U2, U4/U6 and U5 JOURNAL Nucleic Acids Res. 18 (22), 6475-6484 (1990) PUBMED 1701240 REFERENCE 5 (residues 1 to 119) AUTHORS Rokeach,L.A., Haselby,J.A. and Hoch,S.O. TITLE Molecular cloning of a cDNA encoding the human Sm-D autoantigen JOURNAL Proc. Natl. Acad. Sci. U.S.A. 85 (13), 4832-4836 (1988) PUBMED 3260384 COMMENT REVIEWED REFSEQ: This record has been curated by NCBI staff. The reference sequence was derived from J03798.1. Summary: This gene encodes a small nuclear ribonucleoprotein that belongs to the SNRNP core protein family. The protein may act as a charged protein scaffold to promote SNRNP assembly or strengthen SNRNP-SNRNP interactions through nonspecific electrostatic contacts with RNA. FEATURES Location/Qualifiers source 1..119 /organism="Homo sapiens" /db_xref="taxon:9606" /chromosome="18" /map="18q11.2" Protein 1..119 /product="small nuclear ribonucleoprotein D1 polypeptide 16kDa" /note="snRNP core protein D1; Sm-D autoantigen; small nuclear ribonucleoprotein D1 polypeptide (16kD)" CDS 1..119 /gene="SNRPD1" /coded_by="NM_006938.2:132..491" /db_xref="GeneID:6632" /db_xref="MIM:601063" ORIGIN 1 mklvrflmkl shetvtielk ngtqvhgtit gvdvsmnthl kavkmtlknr epvqletlsi 61 rgnniryfil pdslpldtll vdvepkvksk kreavagrgr grgrgrgrgr grgrggprr (SEQ ID NO: 85)

Human Tripartite Motif Protein 2 (RING finger protein 86) LOCUS Q9C040 744 aa linear DEFINITION Tripartite motif protein 2 (RING finger protein 86) ACCESSION Q9C040 VERSION Q9C040 GI:21363034 DBSOURCE swissprot: locus TRIM2_HUMAN, accession Q9C040; class: standard. extra accessions:O60272,Q9BSI9,Q9UFZ1,created: Feb 28, 2003. sequence updated: Feb 28, 2003. annotation updated: May 1, 2005. xrefs: AF220018.1, AAG53472.1, AB011089.1, BAA25443.1, BC005016.1, AAH05016.1, BC011052.1, AAH11052.1, AL110234.1, CAB53687.2, T00082 xrefs (non-sequence databases): HSSPP28990, EnsemblENSG00000109654, GenewHGNC:15974, H-InvDBHIX0004577, GO0005737, GO0017022, GO0008270, InterProIPR011044, InterProIPR003649, InterProIPR001298, InterProIPR001258, InterProIPR000315, InterProIPR001841, PfamPF00630, PfamPF01436, PfamPF00643, PfamPF00097, PRINTSPR01406, SMARTSM00502, SMARTSM00336, SMARTSM00557, SMARTSM00184, PROSITEPS50194, PROSITEPS50119, PROSITEPS00518, PROSITEPS50089 KEYWORDS Metal-binding; Repeat; Zinc; Zinc-finger. SOURCE Homo sapiens (human) ORGANISM Homo sapiens Eukaryota; Metazoa; Chordata; Craniata; Vertebrata; Euteleostomi; Mammalia; Eutheria; Euarchontoglires; Primates; Catarrhini; Hominidae; Homo. REFERENCE 1 (residues 1 to 744) AUTHORS Reymond,A., Meroni,G., Fantozzi,A., Merla,G., Cairo,S., Luzi,L., Riganelli,D., Zanaria,E., Messali,S., Cainarca,S., Guffanti,A., Minucci,S., Pelicci,P.G. and Ballabio,A. TITLE The tripartite motif family identifies cell compartments JOURNAL EMBO J. 20 (9), 2140-2151 (2001) PUBMED 11331580 REMARK NUCLEOTIDE SEQUENCE. REFERENCE 2 (residues 1 to 744) AUTHORS Nagase,T., Ishikawa,K., Miyajima,N., Tanaka,A., Kotani,H., Nomura,N. and Ohara,O. TITLE Prediction of the coding sequences of unidentified human genes.

IX. The complete sequences of 100 new cDNA clones from brain which can code for large proteins in vitro JOURNAL DNA Res. 5 (1), 31-39 (1998) PUBMED 9628581 REMARK NUCLEOTIDE SEQUENCE [LARGE SCALE MRNA]. TISSUE=Brain REFERENCE 3 (residues 1 to 744) AUTHORS Strausberg,R.L., Feingold,E.A., Grouse,L.H., Derge,J.G., Klausner,R.D., Collins,F.S., Wagner,L., Shenmen,C.M., Schuler,G.D., Altschul,S.F., Zeeberg,B., Buetow,K.H., Schaefer,C.F., Bhat,N.K., Hopkins,R.F., Jordan,H., Moore,T., Max,S.I., Wang,J., Hsieh,F., Diatchenko,L., Marusina,K., Farmer,A.A., Rubin,G.M., Hong,L., Stapleton,M., Soares,M.B., Bonaldo,M.F., Casavant,T.L., Scheetz,T.E., Brownstein,M.J., Usdin,T.B., Toshiyuki,S., Carninci,P., Prange,C., Raha,S.S., Loquellano,N.A., Peters,G.J., Abramson,R.D., Mullahy,S.J., Bosak,S.A., McEwan,P.J., McKernan,K.J., Malek,J.A., Gunaratne,P.H., Richards,S., Worley,K.C., Hale,S., Garcia,A.M., Gay,L.J., Hulyk,S.W., Villalon,D.K., Muzny,D.M., Sodergren,E.J., Lu,X., Gibbs,R.A., Fahey,J., Helton,E., Ketteman,M., Madan,A., Rodrigues,S., Sanchez,A., Whiting,M., Madan,A., Young,A.C., Shevchenko,Y., Bouffard,G.G., Blakesley,R.W., Touchman,J.W., Green,E.D., Dickson,M.C., Rodriguez,A.C., Grimwood,J., Schmutz,J., Myers,R.M., Butterfield,Y.S., Krzywinski,M.I., Skalska,U., Smailus,D.E., Schnerch,A., Schein,J.E., Jones,S.J. and Marra,M.A. CONSRTM Mammalian Gene Collection Program Team TITLE Generation and initial analysis of more than 15,000 full- length human and mouse cDNA sequences JOURNAL Proc. Natl. Acad. Sci. U.S.A. 99 (26), 16899-16903 (2002) PUBMED 12477932 REMARK NUCLEOTIDE SEQUENCE [LARGE SCALE MRNA]. TISSUE=Brain, and Placenta REFERENCE 4 (residues 1 to 744) AUTHORS . CONSRTM The German cDNA consortium TITLE Direct Submission JOURNAL Submitted (??-AUG-1999) REMARK NUCLEOTIDE SEQUENCE [LARGE SCALE MRNA] OF 515-744. TISSUE=Kidney COMMENT [FUNCTION] May contribute to the alteration of neural cellular mechanisms (By similarity). [SUBUNIT] Interacts with myosin V (By similarity). [SUBCELLULAR LOCATION] Cytoplasmic (By similarity). [DOMAIN] The interaction with myosin V is dependent upon its NHL repeats, which form a beta-propeller (NHL) domain containing six blades (By similarity). [SIMILARITY] Belongs to the TRIM/RBCC family. [SIMILARITY] Contains 1 B box-type zinc finger. [SIMILARITY] Contains 1 filamin repeat. [SIMILARITY] Contains 6 NHL repeats. [SIMILARITY] Contains 1 RING-type zinc finger. FEATURES Location/Qualifiers source 1..744 /organism="Homo sapiens" /db_xref="taxon:9606" gene 1..744 /gene="TRIM2" /note="synonyms: KIAA0517, RNF86" Protein 1..744 /gene="TRIM2" /product="Tripartite motif protein 2" Region 23..64 /gene="TRIM2" /region_name="Zinc finger region" /note="RING-type." /evidence=experimental Region 113..154 /gene="TRIM2" /region_name="Zinc finger region" /note="B box-type." /evidence=experimental Region 320..421 /gene="TRIM2" /region_name="Repetitive region" /note="Filamin." /evidence=experimental Region 486..513 /gene="TRIM2" /region_name="Repetitive region" /note="NHL 1." /evidence=experimental Region 515 /gene="TRIM2" /region_name="Conflict" /note="N -> G (in Ref. 4)." /evidence=experimental Region 533..560 /gene="TRIM2" /region_name="Repetitive region" /note="NHL 2." /evidence=experimental Region 575..602 /gene="TRIM2" /region_name="Repetitive region" /note="NHL 3." /evidence=experimenta Region 622..649 /gene="TRIM2" /region_name="Repetitive region" /note="NHL 4." /evidence=experimental Region 669..696 /gene="TRIM2" /region_name="Repetitive region" /note="NHL 5." /evidence=experimental Region 713..740 /gene="TRIM2" /region_name="Repetitive region" /note="NHL 6." /evidence=experimental Region 737..744 /gene="TRIM2" /region_name="Conflict" /note="FKVYRYLQ (SEQ ID NO: 86) -> LILIYSRHLFFYESKC (SEQ ID NO: 87) (in Ref. 3; AAH05016)." /evidence=experimental ORIGIN 1 masegtnips pvvrqidkqf licsiclery knpkvlpclh tfcerclqny ipahsltlsc 61 pvcrqtsilp ekgvaalqnn ffitnlmdvl qrtpgsnaee ssiletvtav aagkplscpn 121 hdgnvmefyc qscetamcre ctegehaehp tvplkdvveq hkaslqvqld avnkrlpeid 181 salqfiseii hqltnqkasi vddihstfde lqktlnvrks vllmelevny glkhkvlqsq 241 ldtllqgqes ikscsnftaq alnhgtetev llvkkqmsek lneladqdfp lhprendqld 301 fiveteglkk sihnlgtilt tnavasetva tgeglrqtii gqpmsvtitt kdkdgelckt 361 gnayltaels tpdgsvadge ildnkngtye flytvqkegd ftlslrlydq hirgspfklk 421 virsadvspt tegvkrrvks pgsghvkqka vkrpasmyst gkrkenpied dlifrvgtkg 481 rnkgeftnlq gvaastngki liadsnnqcv qifsndgqfk srfgirgrsp gqlqrptgva 541 vhpsgdiiia dydnkwvsif ssdgkfktki gsgklmgpkg vsvdrnghii vvdnkaccvf 601 ifqpngkivt rfgsrgngdr qfagphfaav nsnneiiitd fhnhsvkvfn qegefmlkfg 661 sngegngqfn aptgvavdsn gniivadwgn sriqvfdgsg sflsyintsa dplygpqgla 721 ltsdghvvva dsgnhcfkvy rylq (SEQ ID NO: 88)

Human TFIID Subunit 6 LOCUS P49848 677 aa linear DEFINITION Transcription initiation factor TFIID subunit 6 (Transcription initiation factor TFIID 70 kDa subunit) (TAF(II)70) (TAFII-70) (TAFII-80) (TAFII80). ACCESSION P49848 VERSION P49848 GI:1729810 DBSOURCE swissprot: locus TAF6_HUMAN, accession P49848; class: standard. created: Oct 1, 1996. sequence updated: Oct 1, 1996. annotation updated: May 1, 2005. xrefs: L25444.1, AAA63643.1, U31659.1, AAA84390.1, AY149894.1, AAN10295.1, BC018115.1, AAH18115.1 xrefs (non-sequence databases): HSSPP49847, TRANSFACT00783, TRANSFACT02208, GenewHGNC:11540, H-InvDBHIX0006909, ReactomeP49848, MIM 602955, GO0005669, GO0005673, GO0016251, GO0005515, InterProIPR007124, InterProIPR009072, InterProIPR004823, PfamPF02969 KEYWORDS Direct protein sequencing; Nuclear protein; Polymorphism; Transcription; Transcription regulation. SOURCE Homo sapiens (human) ORGANISM Homo sapiens Eukaryota; Metazoa; Chordata; Craniata; Vertebrata; Euteleostomi; Mammalia; Eutheria; Euarchontoglires; Primates; Catarrhini; Hominidae; Homo. REFERENCE 1 (residues 1 to 677) AUTHORS Weinzierl,R.O., Ruppert,S., Dynlacht,B.D., Tanese,N. and Tjian,R. TITLE Cloning and expression of Drosophila TAFII60 and human TAFII70 reveal conserved interactions with other subunits of TFIID JOURNAL EMBO J. 12 (13), 5303-5309 (1993) PUBMED 8262073 REMARK NUCLEOTIDE SEQUENCE, AND PARTIAL PROTEIN SEQUENCE. REFERENCE 2 (residues 1 to 677) AUTHORS Hisatake,K., Ohta,T., Takada,R., Guermah,M., Horikoshi,M., Nakatani,Y. and Roeder,R.G. TITLE Evolutionary conservation of human TATA-binding-polypeptide-associated factors TAFII31 and TAFII80 and interactions of TAFII80 with other TAFs and with general transcription factors JOURNAL Proc. Natl. Acad. Sci. U.S.A. 92 (18), 8195-8199 (1995) PUBMED 7667268 REMARK NUCLEOTIDE SEQUENCE. TISSUE=Placenta REFERENCE 3 (residues 1 to 677) AUTHORS Rieder,M.J., Livingston,R.J., Daniels,M.R., Montoya,M.A., Chung,M.-W., Miyamoto,K.E., Nguyen,C.P., Nguyen,D.A., Poel,C.L., Robertson,P.D., Schackwitz,W.S., Sherwood,J.K., Witrak,L.A. and Nickerson,D.A. TITLE Direct Submission JOURNAL Submitted (??-SEP-2002) REMARK NUCLEOTIDE SEQUENCE, AND VARIANT SER-36. EFERENCE 4 (residues 1 to 677) AUTHORS Strausberg,R.L., Feingold,E.A., Grouse,L.H., Derge,J.G., Klausner,R.D., Collins,F.S., Wagner,L., Shenmen,C.M., Schuler,G.D., Altschul,S.F., Zeeberg,B., Buetow,K.H., Schaefer,C.F., Bhat,N.K., Hopkins,R.F., Jordan,H., Moore,T., Max,S.I., Wang,J., Hsieh,F., Diatchenko,L., Marusina,K., Farmer,A.A., Rubin,G.M., Hong,L., Stapleton,M., Soares,M.B., Bonaldo,M.F., Casavant,T.L., Scheetz,T.E., Brownstein,M.J., Usdin,T.B., Toshiyuki,S., Carninci,P., Prange,C., Raha,S.S., Loquellano,N.A., Peters,G.J., Abramson,R.D., Mullahy,S.J., Bosak,S.A., McEwan,P.J., McKernan,K.J., Malek,J.A., Gunaratne,P.H., Richards,S., Worley,K.C., Hale,S., Garcia,A.M., Gay,L.J., Hulyk,S.W., Villalon,D.K., Muzny,D.M., Sodergren,E.J., Lu,X., Gibbs,R.A., Fahey,J., Helton,E., Ketteman,M., Madan,A., Rodrigues,S., Sanchez,A., Whiting,M., Madan,A., Young,A.C., Shevchenko,Y., Bouffard,G.G., Blakesley,R.W., Touchman,J.W., Green,E.D., Dickson,M.C., Rodriguez,A.C., Grimwood,J., Schmutz,J., Myers,R.M., Butterfield,Y.S., Krzywinski,M.I., Skalska,U., Smailus,D.E., Schnerch,A., Schein,J.E., Jones,S.J. and Marra,M.A. CONSRTM Mammalian Gene Collection Program Team TITLE Generation and initial analysis of more than 15,000 full- length human and mouse cDNA sequences JOURNAL Proc. Natl. Acad. Sci. U.S.A. 99 (26), 16899-16903 (2002) PUBMED 12477932 REMARK NUCLEOTIDE SEQUENCE [LARGE SCALE MRNA]. TISSUE=Pancreas COMMENT [FUNCTION] TAFs are components of the transcription factor IID (TFIID) complex, PCAF histone acetylase complex and TBP-free TAFII complex (TFTC). TIIFD is multimeric protein complex that plays a central role in mediating promoter responses to various activators and repressors [SUBUNIT] TFIID and PCAF are composed of TATA binding protein (TBP) and a number of TBP-associated factors (TAFs). TBP is not part of TFTC. Binds tightly to TAFII-250 and also directly interacts with TAFII-40. [SUBCELLULAR LOCATION] Nuclear [SIMILARITY] Belongs to the TAF6 famiLY. FEATURES Location/Qualifiers source 1..677 /organism="Homo sapiens" /db_xref="taxon:9606" gene 1..677 /gene="TAF6" /note="synonyms: TAF2E, TAFII70" Protein 1..677 /gene="TAF6" /product="Transcription initiation factor TFIID subunit 6" Region 36 /gene="TAF6" /region_name="Variant" /note="C -> S. /FTId=VAR_014349." /evidence=experimental ORIGIN 1 maeekklkls ntvlpsesmk vvaesmgiaq iqeetcqllt devsyrikei aqdalkfmhm 61 gkrqklttsd idyalklknv eplygfhaqe fipfrfasgg grelyfyeek evdlsdiint 121 plprvpldvc lkahwlsieg cqpaipenpp papkeqqkae ateplksakp gqeedgplkg 181 kgqgattadg kgkekkappl legaplrlkp rsihelsveq qlyykeitea cvgsceakra 241 ealqsiatdp glyqmlprfs tfisegvrvn vvqnnlalli ylmrmvkalm dnptlyleky 301 vhelipavmt civsrqlclr pdvdnhwalr dfaarlvaqi ckhfstttnn iqsritktft 361 kswvdektpw ttrygsiagl aelghdvikt lilprlqqeg erirsvldgp vlsnidriga 421 dhvqslllkh capvlaklrp ppdnqdayra efgslgpllc sqvvkaraqa alqaqqvnrt 481 tltitqprpt ltlsqapqpg prtpgllkvp gsialpvqtl vsaraaappq psppptkfiv 541 msssssapst qqvlslstsa pgsgstttsp vtttvpsvqp ivklvstatt appstapsgp 601 gsvqkyivvs lpptgegkgg ptshpspvpp passpsplsg salcggkqea gdspppapgt 661 pkangsqpns gspqpap (SEQ ID NO: 89)

Human TAR-binding protein LOCUS NP_000958 403 aa linear DEFINITION ribosomal protein L3 [Homo sapiens]. ACCESSION NP_000958 VERSION NP_000958.1 GI:4506649 DBSOURCE REFSEQ: accession NM_000967.2 KEYWORDS SOURCE Homo sapiens (human) ORGANISM Homo sapiens Eukaryota; Metazoa; Chordata; Craniata; Vertebrata; Euteleostomi; Mammalia; Eutheria; Euarchontoglires; Primates; Catarrhini; Hominidae; Homo. REFERENCE 1 (residues 1 to 403) AUTHORS Collins,J.E., Wright,C.L., Edwards,C.A., Davis,M.P., Grinham,J.A., Cole,C.G., Goward,M.E., Aguado,B., Mallya,M., Mokrab,Y., Huckle,E.J., Beare,D.M. and Dunham,I. TITLE A genome annotation-driven approach to cloning the human ORFeome JOURNAL Genome Biol. 5 (10), R84 (2004) PUBMED 15461802 REFERENCE 2 (residues 1 to 403) AUTHORS Uechi,T., Tanaka,T. and Kenmochi,N. TITLE A complete map of the human ribosomal protein genes: assignment of 80 genes to the cytogenetic map and implications for human disorders JOURNAL Genomics 72 (3), 223-230 (2001) PUBMED 11401437 REFERENCE 3 (residues 1 to 403) AUTHORS Duga,S., Asselta,R., Malcovati,M., Tenchini,M.L., Ronchi,S. and Simonic,T. TITLE The intron-containing L3 ribosomal protein gene (RPL3): sequence analysis and identification of U43 and of two novel intronic small nucleolar RNAs JOURNAL Biochim. Biophys. Acta 1490 (3), 225-236 (2000) PUBMED 10684968 REFERENCE 4 (residues 1 to 403) AUTHORS Kenmochi,N., Kawaguchi,T., Rozen,S., Davis,E., Goodman,N., Hudson,T.J., Tanaka,T. and Page,D.C. TITLE A map of 75 human ribosomal protein genes JOURNAL Genome Res. 8 (5), 509-523 (1998) PUBMED 9582194 REFERENCE 5 (residues 1 to 403) AUTHORS Wool,I.G., Chan,Y.L. and Gluck,A. TITLE Structure and evolution of mammalian ribosomal proteins JOURNAL Biochem. Cell Biol. 73 (11-12), 933-947 (1995) PUBMED 8722009 REMARK Review article REFERENCE 6 (residues 1 to 403) AUTHORS Reddy,T.R., Suhasini,M., Rappaport,J., Looney,D.J., Kraus,G. and Wong-Staal,F. TITLE Molecular cloning and characterization of a TAR-binding nuclear factor from T cells JOURNAL AIDS Res. Hum. Retroviruses 11 (6), 663-669 (1995) PUBMED 7576925 REFERENCE 7 (residues 1 to 403) AUTHORS Matoba,R., Okubo,K., Hori,N., Fukushima,A. and Matsubara,K. TITLE The addition of 5'-coding information to a 3'-directed Cdna library improves analysis of gene expression JOURNAL Gene 146 (2), 199-207 (1994) PUBMED 8076819 COMMENT REVIEWED REFSEQ: This record has been curated by NCBI staff. The reference sequence was derived from BC012146.1 and BC008492.1. Summary: Ribosomes, the organelles that catalyze protein synthesis, consist of a small 40S subunit and a large 60S subunit. Together these subunits are composed of 4 RNA species and approximately 80 structurally distinct proteins. This gene encodes a ribosomal protein that is a component of the 60S subunit. The protein belongs to the L3P family of ribosomal proteins. It is located in the cytoplasm. The protein can bind to the HIV-1 TAR mRNA, and it has been suggested that the protein contributes to tat-mediated transactivation. This gene is co-transcribed with the small nucleolar RNA genes U43, U86, U83a, and U83b, which are located in its first, third, fifth, and seventh introns, respectively. As is typical for genes encoding ribosomal proteins, there are multiple processed pseudogenes of this gene dispersed through the genome FEATURES Location/Qualifiers source 1..403 /organism="Homo sapiens" /db_xref="taxon:9606" /chromosome="22" /map="22q13" Protein 1..403 /product="ribosomal protein L3" /note="60S ribosomal protein L3; HIV-1 TAR RNA- binding protein B" CDS 1..403 /gene="RPL3" /coded_by="NM_000967.2:27..1238" /db_xref="CCDS:CCDS13988.1" /db_xref="GeneID:6122" /db_xref="MIM:604163" ORIGIN 1 mshrkfsapr hgslgflprk rssrhrgkvk sfpkddpskp vhltaflgyk agmthivrev 61 drpgskvnkk evveavtive tppmvvvgiv gyvetprglr tfktvfaehi sdeckrrfyk 121 nwhkskkkaf tkyckkwqde dgkkqlekdf ssmkkycqvi rviahtqmrl lplrqkkahl 181 meiqvnggtv aekldwarer leqqvpvnqv fgqdemidvi gvtkgkgykg vtsrwhtkkl 241 prkthrglrk vacigawhpa rvafsvarag qkgyhhrtei nkkiykigqg ylikdgklik 301 nnastdydls dksinplggf vhygevtndf vmlkgcvvgt kkrvltlrks llvqtkrral 361 ekidlkfidt tskfghgrfq tmeekkafmg plkkdriake ega (SEQ ID NO: 90)

Human ERI-1 (AAH35279) LOCUS AAH35279 349 aa linear DEFINITION Histone mRNA 3' end-specific exonuclease [Homo sapiens] ACCESSION AAH35279 VERSION AAH35279.1 GI:23271401 DBSOURCE accession BC035279.1 KEYWORDS MGC. SOURCE Homo sapiens (human) ORGANISM Homo sapiens Eukaryota; Metazoa; Chordata; Craniata; Vertebrata; Euteleostomi; Mammalia; Eutheria; Euarchontoglires; Primates; Catarrhini; Hominidae; Homo. REFERENCE 1 (residues 1 to 349) AUTHORS Strausberg,R.L., Feingold,E.A., Grouse,L.H., Derge,J.G., Klausner,R.D., Collins,F.S., Wagner,L., Shenmen,C.M., Schuler,G.D., Altschul,S.F., Zeeberg,B., Buetow,K.H., Schaefer,C.F., Bhat,N.K., Hopkins,R.F., Jordan,H., Moore,T., Max,S.I., Wang,J., Hsieh,F., Diatchenko,L., Marusina,K., Farmer,A.A., Rubin,G.M., Hong,L., Stapleton,M., Soares,M.B., Bonaldo,M.F., Casavant,T.L., Scheetz,T.E., Brownstein,M.J., Usdin,T.B., Toshiyuki,S., Carninci,P., Prange,C., Raha,S.S., Loquellano,N.A., Peters,G.J., Abramson,R.D., Mullahy,S.J., Bosak,S.A., McEwan,P.J., McKernan,K.J., Malek,J.A., Gunaratne,P.H., Richards,S., Worley,K.C., Hale,S., Garcia,A.M., Gay,L.J., Hulyk,S.W., Villalon,D.K., Muzny,D.M., Sodergren,E.J., Lu,X., Gibbs,R.A., Fahey,J., Helton,E., Ketteman,M., Madan,A., Rodrigues,S., Sanchez,A., Whiting,M., Madan,A., Young,A.C., Shevchenko,Y., Bouffard,G.G., Blakesley,R.W., Touchman,J.W., Green,E.D., Dickson,M.C., Rodriguez,A.C., Grimwood,J., Schmutz,J., Myers,R.M., Butterfield,Y.S., Krzywinski,M.I., Skalska,U., Smailus,D.E., Schnerch,A., Schein,J.E., Jones,S.J. and Marra,M.A. CONSRTM Mammalian Gene Collection Program Team TITLE Generation and initial analysis of more than 15,000 full- length human and mouse cDNA sequences JOURNAL Proc. Natl. Acad. Sci. U.S.A. 99 (26), 16899-16903 (2002) PUBMED 12477932 REFERENCE 2 (residues 1 to 349) AUTHORS . CONSRTM NIH MGC Project TITLE Direct Submission JOURNAL Submitted (31-JUL-2002) National Institutes of Health, Mammalian Gene Collection (MGC), Bethesda, MD 20892-2590, USA REMARK NIH-MGC Email: cgapbs-r@mail.nih.gov Tissue Procurement: Life Technologies, Inc. cDNA Library Preparation: Life Technologies, Inc. cDNA Library Arrayed by: The I.M.A.G.E. Consortium (LLNL DNA Sequencing by: Baylor College of Medicine Human Genome Sequencing Center Center code: BCM-HGSC Contact: amg@bcm.tmc.edu Gunaratne, P.H., Garcia, A.M., Lu, X., Hulyk, S.W., Loulseged, H., Kowis, C.R., Sneed, A.J., Martin, R.G., Muzny, D.M., Nanavati, A.N., Gibbs, R.A. Clone distribution: MGC clone distribution information can be found through the I.M.A.G.E. Consortium/LLNL at: National Center for Biotechnology Information, National Library of Medicine, Building 38A, Bethesda, MD 20894 Series: IRAK Plate: 50 Row: g Column: 1 This clone was selected for full length sequencing because it passed the following selection criteria: matched mRNA gi: 31543183. Method: conceptual translation. FEATURES Location/Qualifiers source 1..349 /organism="Homo sapiens" /db_xref="taxon:9606" /clone="MGC:35395 IMAGE:5186320" /tissue_type="Colon, Kidney, Stomach, adult, whole pooled" /clone_lib="NIH_MGC_116" /lab_host="DH10B" /note="Vector: pCMV-SPORT6" Protein 1..349 /product="histone mRNA 3' end-specific exonuclease" CDS 1..349 /gene="3'HEXO" /coded_by="BC035279.1:125..1174" /db_xref="GeneID:90459"

ORIGIN 1 medpqskepa geavalalle sprpeggeep prpspeetqq ckfdgqetkg skfitssasd 61 fsdpvykeia itngcinrms keelraklse fkletrgvkd vlkkrlknyy kkqklmlkes 121 nfadsyydyi ciidfeatce egnppefvhe iiefpvvlln thtleiedtf qqyvrpeint 181 qlsdfcislt gitqdqvdra dtfpqvlkkv idwmklkelg tkykyslltd gswdmskfln 241 iqcqlsrlky ppfakkwini rksygnfykv prsqtkltim leklgmdydg rphcglddsk 301 niariavrml qdgcelrine kmhagqlmsv ssslpiegtp ppqmphfrk SEQ ID NO: 91)

Human TUDOR protein LOCUS Q9BXT4 777 aa linear DEFINITION Tudor domain containing protein 1. ACCESSION Q9BXT4 VERSION Q9BXT4 GI:17368689 DBSOURCE swissprot: locus TDRD1_HUMAN, accession Q9BXT4; class: standard. extra accessions:Q9H7B3,created: Feb 28, 2003. sequence updated: Feb 28, 2003. annotation updated: May 1, 2005. xrefs: AF285606.1, AAK31985.1, AK024735.1, BAB14982.1 xrefs (non-sequence databases): GenewHGNC:11712, MIM 605796, InterProIPR008191, InterProIPR002999, PfamPF00567, SMARTSM00333, PROSITEPS50304 KEYWORDS Repeat. SOURCE Homo sapiens (human) ORGANISM Homo sapiens Eukaryota; Metazoa; Chordata; Craniata; Vertebrata; Euteleostomi; Mammalia; Eutheria; Euarchontoglires; Primates; Catarrhini; Hominidae; Homo. REFERENCE 1 (residues 1 to 777) AUTHORS Wang,P.J., McCarrey,J.R., Yang,F. and Page,D.C. TITLE An abundance of X-linked genes expressed in spermatogonia JOURNAL Nat. Genet. 27 (4), 422-426 (2001) PUBMED 11279525 REMARK NUCLEOTIDE SEQUENCE. TISSUE=Testis REFERENCE 2 (residues 1 to 777) AUTHORS Ota,T., Suzuki,Y., Nishikawa,T., Otsuki,T., Sugiyama,T., Irie,R., Wakamatsu,A., Hayashi,K., Sato,H., Nagai,K., Kimura,K., Makita,H., Sekine,M., Obayashi,M., Nishi,T., Shibahara,T., Tanaka,T., Ishii,S., Yamamoto,J., Saito,K., Kawai,Y., Isono,Y., Nakamura,Y., Nagahari,K., Murakami,K., Yasuda,T., Iwayanagi,T., Wagatsuma,M., Shiratori,A., Sudo,H., Hosoiri,T., Kaku,Y., Kodaira,H., Kondo,H., Sugawara,M., Takahashi,M., Kanda,K., Yokoi,T., Furuya,T., Kikkawa,E., Omura,Y., Abe,K., Kamihara,K., Katsuta,N., Sato,K., Tanikawa,M., Yamazaki,M., Ninomiya,K., Ishibashi,T., Yamashita,H., Murakawa,K., Fujimori,K., Tanai,H., Kimata,M., Watanabe,M., Hiraoka,S., Chiba,Y., Ishida,S., Ono,Y., Takiguchi,S., Watanabe,S., Yosida,M., Hotuta,T., Kusano,J., Kanehori,K., Takahashi- Fujii,A., Hara,H., Tanase,T.O., Nomura,Y., Togiya,S., Komai,F., Hara,R., Takeuchi,K., Arita,M., Imose,N., Musashino,K., Yuuki,H., Oshima,A., Sasaki,N., Aotsuka,S., Yoshikawa,Y., Matsunawa,H., Ichihara,T., Shiohata,N., Sano,S., Moriya,S., Momiyama,H., Satoh,N., Takami,S., Terashima,Y., Suzuki,O., Nakagawa,S., Senoh,A., Mizoguchi,H., Goto,Y., Shimizu,F., Wakebe,H., Hishigaki,H., Watanabe,T., Sugiyama,A., Takemoto,M., Kawakami,B., Yamazaki,M., Watanabe,K., Kumagai,A., Itakura,S., Fukuzumi,Y., Fujimori,Y., Komiyama,M., Tashiro,H., Tanigami,A., Fujiwara,T., Ono,T., Yamada,K., Fujii,Y., Ozaki,K., Hirao,M., Ohmori,Y., Kawabata,A., Hikiji,T., Kobatake,N., Inagaki,H., Ikema,Y., Okamoto,S., Okitani,R., Kawakami,T., Noguchi,S., Itoh,T., Shigeta,K., Senba,T., Matsumura,K., Nakajima,Y., Mizuno,T., Morinaga,M., Sasaki,M., Togashi,T., Oyama,M., Hata,H., Watanabe,M., Komatsu,T., Mizushima-Sugano,J., Satoh,T., Shirai,Y., Takahashi,Y., Nakagawa,K., Okumura,K., Nagase,T., Nomura,N., Kikuchi,H., Masuho,Y., Yamashita,R., Nakai,K., Yada,T., Nakamura,Y., Ohara,O., Isogai,T. and Sugano,S. TITLE Complete sequencing and characterization of 21,243 full-length human cDNAs JOURNAL Nat. Genet. 36 (1), 40-45 (2004) PUBMED 14702039 REMARK NUCLEOTIDE SEQUENCE [LARGE SCALE MRNA] OF 67-777. COMMENT [TISSUE SPECIFICITY] Testis and ovary specific. [SIMILARITY] Contains 3 Tudor domains. FEATURES Location/Qualifiers source 1..777 /organism="Homo sapiens" /db_xref="taxon:9606" gene 1..777 /gene="TDRD1" Protein 1..777 /gene="TDRD1" /product="Tudor domain containing protein 1" Region 138..197 /gene="TDRD1" /region_name="Domain" /note="Tudor 1." /evidence=experimental Region 359..418 /gene="TDRD1" /region_name="Domain" /note="Tudor 2." /evidence=experimental Region 587..645 /gene="TDRD1" /region_name="Domain" /note="Tudor 3." /evidence=experimental Region 737 /gene="TDRD1" /region_name="Conflict" /note="T -> M (in Ref. 2)." /evidence=experimental Region 775..777 /gene="TDRD1" /region_name="Conflict" /note="VKS -> KKKKK (SEQ ID NO: 92)(in Ref. 2)." /evidence=experimental

ORIGIN 1 meqycsikiv dileeevvtf avevelpnsg klldhvliem gyglkpsgqd skkenadqsd 61 pedvgkmtte nnivvdksdl ipkvltlnvg defcgvvahi qtpedffcqq lqsgrklael 121 qaslskycdq lpprsdfypa igdiccaqfs eddqwyrasv layaseesvl vgyvdygnfe 181 ilslmrlcpi ipkllelpmq aikcvlagvk pslgiwtpea iclmkklvqn kiitvkvvdk 241 lensslveli dksetphvsv skvlldagfa vgeqsmvtdk psdvketsvp lgvegkvnpl 301 ewtwvelgvd qtvdvvvcvi yspgefychv lkedalkkln dlnkslaehc qqklpngfka 361 eigqpccaff agdgswyral vkeilpnghv kvhfvdygni eevtadelrm isstflnlpf 421 qgircqladi qsrnkhwsee aitrfqmcva giklqarvve vtengigvel tdlstcypri 481 isdvlidehl vlksasphkd lpndrlvnkh elqvhvqglq atssaeqwkt ielpvdktiq 541 anvleiispn lfyalpkgmp enqeklcmlt aelleycnap ksrppyrpri gdaccakyts 601 ddfwyravvl gtsdtdvevl yadygnietl plcrvqpits shlalpfqii rcsleglmel 661 ngsssqliim llknfmlnqn vmlsvkgitk nvhtvsvekc sengtvdvad klvtfglakn 721 itpqrqsaln tekmyrtncc ctelqkqvek hehillflln nstnqnkfie mkklvks (SEQ ID NO: 93)

Human Dual Specificity Phosphatase II (DUSPII) LOCUS NP_003575 330 aa linear DEFINITION dual specificity phosphatase 11 [Homo sapiens]. ACCESSION NP_003575 VERSION NP_003575.1 GI:4503415 DBSOURCE REFSEQ: accession NM_003584.1 KEYWORDS . SOURCE Homo sapiens (human) ORGANISM Homo sapiens Eukaryota; Metazoa; Chordata; Craniata; Vertebrata; Euteleostomi; Mammalia; Eutheria; Euarchontoglires; Primates; Catarrhini; Hominidae; Homo. REFERENCE 1 (residues 1 to 330) AUTHORS Yuan,Y., Li,D.M. and Sun,H. TITLE PIR1, a novel phosphatase that exhibits high affinity to RNA. ribonucleoprotein complexes JOURNAL J. Biol. Chem. 273 (32), 20347-20353 (1998) PUBMED 9685386 COMMENT REVIEWED REFSEQ: This record has been curated by NCBI staff. The reference sequence was derived from AF023917.1. Summary: The protein encoded by this gene is a member of the dual specificity protein phosphatase subfamily. These phosphatases inactivate their target kinases by dephosphorylating both the phosphoserine/threonine and phosphotyrosine residues. They negatively regulate members of the mitogen-activated protein (MAP) kinase superfamily (MAPK/ERK, SAPK/JNK, p38), which is associated with cellular proliferation and differentiation. Different members of the family of dual specificity phosphatases show distinct substrate specificities for various MAP kinases, different tissue distribution and subcellular localization, and different modes of inducibility of their expression by extracellular stimuli. This gene product is localized to the nucleus, and is novel in that it binds directly to RNA and splicing factors, and thus suggested to participate in nuclear mRNA metabolism. FEATURES Location/Qualifiers source 1..330 /organism="Homo sapiens" /db_xref="taxon:9606" /chromosome="2" /map="2p13.1" Protein 1..330 /product="dual specificity phosphatase 11" /EC_number="3.1.3.16" /EC_number="3.1.3.48" /note="serine/threonine specific protein phosphatase; RNA/RNP complex-interacting phosphatase" CDS 1..330 /gene="DUSP11" /coded_by="NM_003584.1:125..1117" /note="go_component: nucleus [goid 0005634] [evidence TAS] [pmid 9685386]; go_function: RNA binding [goid 0003723] [evidence TAS] [pmid 9685386]; go_function: hydrolase activity [goid 0016787] [evidence IEA]; go_function: protein tyrosine phosphatase activity [goid 0004725] [evidence TAS] [pmid 9685386]; go_process: RNA processing [goid 0006396] [evidence TAS] [pmid 9685386]; go_process: protein amino acid dephosphorylation [goid 0006470] [evidence IEA]" /db_xref="CCDS:CCDS1928.1" /db_xref="GeneID:8446" /db_xref="MIM:603092"ORIGIN 1 msqwhhprsg wgrrrdfsgr ssakkkggnh iperwkdylp vgqrmpgtrf iafkvplqks 61 fekklapeec fspldlfnki reqneelgli idltytqryy kpedlpetvp ylkiftvghq 121 vpddetifkf khavngflke nkdndkligv hcthglnrtg ylicrylidv egvrpddaie 181 lfnrcrghcl erqnyiedlq ngpirknwns svprssdfed sahlmqpvhn kpvkqgpryn 241 lhqiqghsap rhfhtqtqsl qqsvrkfsen phvyqrhhlp ppgppgedys hrryswnvkp 301 nasraaqdrr rwypynysrl sypacwewtq (SEQ ID NO: 94)

IV.Dicer

Dicer proteins for use in the present invention can be from any suitable source. Preferred sources include C. elegans, H. sapeins and M. musculus, as depicted infra, although the skilled artisan will appreciate that other sources can readily be used based on the significant conservation exhibited between Dicer homologs. For example, Dicer homologs from D. melanogaster, Rattus norvegicus, and primate are useful (see, e.g., Accession Nos. gi:51316117; gi:34867687; and gi:55641327, respectively). LOCUS NP_498761 1845 aa linear DEFINITION DiCer Related, LEThal LET-740 (dcr-1) [Caenorhabditis elegans]. ACCESSION NP_498761 VERSION NP_498761.1 GI:17552834 DBSOURCE REFSEQ: accession NM_066360.1 KEYWORDS . SOURCE Caenorhabditis elegans ORGANISM Caenorhabditis elegans Eukaryota; Metazoa; Nematoda; Chromadorea; Rhabditida; Rhabditoidea; Rhabditidae; Peloderinae; Caenorhabditis. REFERENCE 1 (residues 1 to 1845) AUTHORS Dillin,A., Hsu,A.L., Arantes-Oliveira,N., Lehrer-Graiwer,J., Hsin,H., Fraser,A.G., Kamath,R.S., Ahringer,J. and Kenyon,C. TITLE Rates of behavior and aging specified by mitochondrial function during development JOURNAL Science 298 (5602), 2398-2401 (2002) MEDLINE 22382053 PUBMED 12471266 REFERENCE 2 (residues 1 to 1845) AUTHORS Piano,F., Schetter,A.J., Morton,D.G., Gunsalus,K.C., Reinke,V., Kim,S.K. and Kemphues,K.J. TITLE Gene clustering based on RNAi phenotypes of ovary-enriched genes in C. elegans JOURNAL Curr. Biol. 12 (22), 1959-1964 (2002) MEDLINE 22335533 PUBMED 12445391 REFERENCE 3 (residues 1 to 1845) AUTHORS Walhout,A.J., Reboul,J., Shtanko,O., Bertin,N., Vaglio,P Schetter,A.J., Morton,D.G., Kemphues,K.J., Reinke,V., Kim,S.K., Piano,F. and Vidal,M. TITLE Integrating interactome, phenome, and transcriptome mapping data for the C. elegans germline JOURNAL Curr. Biol. 12 (22), 1952-1958 (2002) MEDLINE 22335532 PUBMED 12445390 REFERENCE 4 (residues 1 to 1845) AUTHORS Tabara,H., Yigit,E., Siomi,H. and Mello,C.C. TITLE The dsRNA binding protein RDE-4 interacts with RDE-1, DCR-1, and a DExH-box helicase to direct RNAi in C. elegans JOURNAL Cell 109 (7), 861-871 (2002) MEDLINE 22105477 PUBMED 12110183 REFERENCE 5 (residues 1 to 1845) AUTHORS Banerjee,D. and Slack,F. TITLE Control of developmental timing by small temporal RNAs: a paradigm for RNA-mediated regulation of gene expression JOURNAL Bioessays 24 (2), 119-129 (2002) MEDLINE 21823375 PUBMED 11835276 REFERENCE 6 (residues 1 to 1845) AUTHORS Ketting,R.F., Fischer,S.E., Bernstein,E., Sijen,T., Hannon,G.J. and Plasterk,R.H. TITLE Dicer functions in RNA interference and in synthesis of small RNA involved in developmental timing in C. elegans JOURNAL Genes Dev. 15 (20), 2654-2659 (2001) MEDLINE 21521222 PUBMED 11641272 REFERENCE 7 (residues 1 to 1845) AUTHORS Knight,S.W. and Bass,B.L. TITLE A role for the RNase III enzyme DCR-1 in RNA interference and germ line development in Caenorhabditis elegans JOURNAL Science 293 (5538), 2269-2271 (2001) MEDLINE 21451181 PUBMED 11486053 REFERENCE 8 (residues 1 to 1845) AUTHORS Jones,S.J., Riddle,D.L., Pouzyrev,A.T., Velculescu,V.E., Hillier,L., Eddy,S.R., Stricklin,S.L., Baillie,D.L., Waterston,R. and Marra,M.A. TITLE Changes in gene expression associated with developmental arrest and longevity in Caenorhabditis elegans JOURNAL Genome Res. 11 (8), 1346-1352 (2001) MEDLINE 21376140 PUBMED 11483575 REFERENCE 9 (residues 1 to 1845) AUTHORS Grishok,A., Pasquinelli,A.E., Conte,D., Li,N., Parrish,S., Ha,I., Baillie,D.L., Fire,A., Ruvkun,G. and Mello,C.C. TITLE Genes and mechanisms related to RNA interference regulate expression of the small temporal RNAs that control C. elegans developmental timing JOURNAL Cell 106 (1), 23-34 (2001) MEDLINE 21354308 PUBMED 11461699 REFERENCE 10 (residues 1 to 1845) AUTHORS Stewart,H.I., O'Neil,N.J., Janke,D.L., Franz,N.W., Chamberlin,H.M., Howell,A.M., Gilchrist,E.J., Ha,T.T., Kuervers,L.M., Vatcher,G.P., Danielson,J.L. and Baillie,D.L. TITLE Lethal mutations defining 112 complementation groups in a 4.5 Mb sequenced region of Caenorhabditis elegans chromosome III JOURNAL Mol. Gen. Genet. 260 (2-3), 280-288 (1998) MEDLINE 99077298 PUBMED 9862482 COMMENT PROVISIONAL REFSEQ: This record has not yet been subject to final NCBI review. This record is derived from an annotated genomic sequence (NC_003281). The reference sequence was derived from WormBase CDS:K12H4.8. Summary: This essential gene dcr-1, also known as let-740, K12H4.8, 3J162 or YK334, maps at (III; -0.30). Phenotypes and affected processes are required for RNA interference, required for synthesis of microrna, sterile adult, lethal. It encodes a DiCer Related. From Pfam homology, the product would have ATP binding, nucleic acid binding, ATP dependent helicase, helicase, RNA binding, double-stranded RNA binding, ribonuclease III activities, would be involved in RNA processing and would localize in intracellular. According to the Worm Transcriptome Project, it is expressed at high level at all stages of development [Kohara cDNAs], except dauers [SAGE]. Its existence, but not its exact sequence, derived here from the genome sequencing consortium annotation, is supported by 26 cDNA clones. Phenotype [WormBase] dcr-1 is required both for RNA interference and for synthesis of small developmental RNAs. Fertilization of dcr-1 oocytes does not occur. While this fertilization defect can be rescued by a dcr-1(+) transgene, fertilized eggs fail to hatch, and mothers are defective in egg-laying. Whereas wild- type oocytes normally do not undergo cell division in the gonad, dcr-1(pk1531) oocytes undergo such division frequently dcr-1 mutations also cause postembryonic defects: alae are absent in 60%, and a burst vulva is observed in 80%, of dcr- 1(pk1531) homozygotes. The postembryonic defects are consistent with the hypothesis that dcr-1 mutants hyperactivate lin-41 in vivo because they are unable to form active let-7 stRNA; in vitro assays of DCR-1 protein confirm that it can generate let-7 stRNA from a double- stranded let-7 precursor. [Ann Rose, 1998, pm9862482] let-704 homozygous s2624 and s2795 each develop into sterile adults. Knock-out allele, deletion obtained by the Gene Knockout Consortium ok247 (strain BB1) [R Barstead, Oklahoma MRF, USA] Selected strains available from the CGC. BC4825 [David Baillie]. NL687 dcr-1(pk1351)/+ III [Ronald Plasterk, Fischer/Thijssen, UV/TMP] Heterozygotes are WT and segregate WT and animals with protruding vulvas (dcr-1 homozygotes). PD8753 dcr-1(ok247) III/hT2[qIs48] (I;III) [Andrew Fire, Barstead/Moulder] [Brenda Bass description] Heterozygotes are WT and segregate WT, Uncs, and Steriles. [B Barstead] dcr-1 homozygotes are completely sterile. qIs48 is an insertion of ccEx9747 with markers: myo-2::GFP expressed brightly in the pharynx throughout development, pes-10::GFP expressed in embryos, and a gut promoter driving GFP in the intestine. Segregates WT glowing hets, non-glowing steriles , very rare homozygous hT2 glowing animals, and dead eggs. BB1. RNA interference results: [T.Hyman 2000] No obvious phenotype (by injecting genomic PCR product TH:K12H4.8). [J.Ahringer 2003] No obvious phenotype (by feeding genomic PCR product JA:K12H4.8). [F.Piano 2002] No P0 sterility detected. Pleiotropic phenotypes (may include abnormal translucence,Dpy,Egl,Gon,Muv,Pvl,Sma) observed in <10% of progeny. No obvious phenotype. Function Protein properties: [Wormbase] bidentate ribonuclease, contains a helicase domain, a PAZ domain, two RNAse III domains, and a double-stranded RNA-binding domain. Expression The expression profile for the gene, derived from the proportion of animals at each stage in each Kohara library is: embryos 7%, L1 or L2 larvae 19%, L3 to adult 75%. In situ hybridisation pictures to all stages of development are available from Kohara NextDB. Pattern [pm11483575] From SAGE comparative analysis of dauer and mixed stages, this gene is one of 533 whose expression is lowered in dauer larvae, a facultative developmentally arrested and long lived stage in C.elegans life cycle. germline enriched [Piano, 2002]. The predicted CDS has 26 exons. It covers 8.17 kb on the WS97 genome. The protein (1845 aa, 210.9 kDa, pI 5.6) contains one DEAD/DEAH box helicase motif(DEAD disclosed as SEQ ID NO: 14; DEAH disclosed as SEQ ID NO: 15), one helicase, C-terminal motif, one Protein of unknown function DUF283 motif, one Argonaute and Dicer protein, PAZ motif, 2 Ribonuclease III family motifs, one Double-stranded RNA binding (DsRBD) domain motif. It also contains 3 coil coil stretch [Psort2]. It is predicted to localise in the cytoplasm [Psort2]. Taxblast (threshold 10^-3) tracks ancestors down to archaea and viruses and bacteria and eukaryota. Method: conceptual translation. FEATURES Location/Qualifiers source 1..1845 /organism="Caenorhabditis elegans" /db_xref="taxon:6239" /chromosome="III" /map="III;-0.30 cM (interpolated genetic position)" /map="III; covering 6084 bp, from base 8077912 to 8071829 on genome release WS97" /clone_lib="Kohara embryonic lambda gt11 library: yk571d8, yk675c6; Kohara Sugano L1 larvae cap-selected library: yk1080g6, yk1084b3, yk1086f1, yk1249b10, yk1271d8; Kohara Sugano L2 larvae cap-selected library: yk1627e3, yk1734b12; Kohara Sugano L4 larvae cap-selected library: yk1448b2, yk1548a2, yk1554a2; Kohara mixed stage library, from him-8 strain, containing 15-30% males: yk11h10, yk18g7, yk24e10, yk86c11, yk181d7, yk192e1, yk243c2, yk249e11, yk318d2, yk355e9, yk355h8, yk419h11, yk154a11; early embryos, Stratagene library [PMID1302005]: T02268" Protein 1..1845 /product="DiCer Related, LEThal LET-740 (dcr-1)" Region 3..218 /region_name="[Pfam/InterPro description] DEAD/DEAH box(DEAD disclosed as SEQ ID NO: 14; DEAH disclosed as SEQ ID NO: 15) helicase" /db_xref="CDD:pfam00270" Region 190..218 /region_name="[PSORT] coil coil 4: PEKLMEQLKKLESAMDSVIETASDLVSLS" (SEQ ID NO: 95) Region 339..345 /region_name="[PSORT] nuclear localization domain: PEMKKIK" (SEQ ID NO: 96) Region 427..498 /region_name="[Pfam/InterPro description] helicase, C-terminal" /db_xref="CDD:pfam00271" Region 503..602 /region_name="[Pfam/InterPro description] protein of unknown function DUF283" /db_xref="CDD:pfam03368" Region 669..675 /region_name="[PSORT] nuclear localization domain: PKRRKFE" (SEQ ID NO: 97) Region 764..770 /region_name="[PSORT] nuclear localization domain: PLNKRKD" (SEQ ID NO: 98) Region 782..961 /region_name="[Pfam/InterPro description] argonaute and Dicer protein, PAZ" /db_xref="CDD:pfam02170" Region 891..897 /region_name="[PSORT] nuclear localization domain: PRRSRTV" (SEQ ID NO: 99) Region 1008..1036 /region_name="[PSORT] coil coil 4: IQQLRDLNQKSIEDQERETRENDKIDDGE" (SEQ ID NO: 100) Region 1179..1214 /region_name="[PSORT] coil coil 4: PKQLTKEEEQFKKLQNDLLKQAKERLEALEMSEDME" (SEQ ID NO: 101) Region 1215..1218 /region_name="[PSORT] nuclear localization domain: KPRR" (SEQ ID NO: 102) Region 1348..1524 /region_name="[Pfam/InterPro description] ribonuclease III family" /db_xref="CDD:pfam00636" Region 1614..1740 /region_name="[Pfam/InterPro description] ribonuclease III family" /db_xref="CDD:pfam00636" Region 1769..1829 /region_name="[Pfam/InterPro description] double- stranded RNA binding (DsRBD) domain" /db_xref="CDD:pfam00035" CDS 1..1845 /gene="dcr-1" /locus_tag="3J162" /coded_by="NM_066360.1:1..5538" /db_xref="AceView/WormGenes:dcr-1" /db_xref="GeneID:176138" /db_xref="LocusID:176138" /db_xref="WormBase:K12H4.8" ORIGIN 1 mvrvradlqc fnprdyqvel ldkatkknti vqlgtgsgkt fiavlllkey gvqlfapldq 61 ggkraffvve kvnlveqqai hievhtsfkv gqvhgqtssg lwdskeqcdq fmkrhhvvvi 121 taqclldlir haylkiedmc vlifdechha lgsqhpyrsi mvdykllkkd kpvprvlglt 181 aslikakvap eklmeqlkkl esamdsviet asdlvslsky gakpyevvii ckdfeigclg 241 ipnfdtviei fdetvafvnt ttefhpdldl dprrpikdsl kttravfrql gpwaawrtaq 301 vwekelgkii ksqvlpdktl rflnmaktsm itikrllepe mkkiksieal rpyvpqrvir 361 lfeiletfnp efqkermkle kaehlsaiif vdqryiaysl llmmrhiksw epkfkfvnpd 421 yvvgasgrnl assdsqglhk rqtevlrrfh rneincliat svleegvdvk qcnlvikfdr 481 pldmrsyvqs kgrarragsr yvitveekdt aaycsklpsd iftrlvphnq iipieengvt 541 kycaelllpi nspikhaivl knpmpnkkta qmavaleacr qlhlegeldd nllpkgresi 601 akllehidee pdeyapgiaa kvgsskrkql ydkkiaraln esfveadkec fiyafelerf 661 reaeltlnpk rrkfedpfny eycfgflsak eipkippfpv flrqgnmkvr livapkkttv 721 taaqlqeiql fhnylftqvl qmcktgnlef dgtsnaplnt livplnkrkd dmsytinmky 781 vsevvanmen mpripkdevr rqykfnaedy kdaivmpwyr nleqpvfyyv aeilpewrps 841 skfpdthfet fneyfikkyk leiydqnqsl ldvdftstrl nllqpriqnq prrsrtvsns 901 stsnipqasa sdskesntsv phssqrqilv pelmdihpis atlwnviaal psifyrvnql 961 lltdelreti lvkafgkekt klddnvewns layateyeek qtiivkkiqq lrdlnqksie 1021 dqeretrend kiddgeelfn igvwdpeeav rigveissrd drmdgedqdt vgltqglhdg 1081 nisdeddelp fvmhdytarl tsnrngigaw sgsesivpsg wgdwdgpepd nspmpfqilg 1141 gpgglnvqal madvgrvfdp stassslsqt vqestvsppk qltkeeeqfk klqndllkqa 1201 kerlealems edmekprrle dtvnledygd dqenqedent ptnfpktide eieelsigar 1261 kkqeiddnaa ktdvlerenc evlpvainek srsfsfekes kaingrlirq rseeyvshid 1321 sdiglgvspc llltalttsn aadgmslerf etigdsflkf attdylyhtl ldqhegklsf 1381 arskevsncn lyrlgkklgi pqlivankfd ahdswlppcy iptcdfkapn tddaeekdne 1441 ierildgqvi eekpenktgw diggdvskst tdgietitfp kqarvgnddi splpynlltq 1501 qhisdksiad avealigvhl ltlgpnptlk vmnwmglkvi qkdqksdvps pllrfidtpt 1561 npnaslnfln nlwqqfqftq leekigyrfk eraylvqaft hasyinnrvt gcyqrleflg 1621 davldymitr ylfedsrqys pgvltdlrsa lvnntifasl avkfefqkhf iamcpglyhm 1681 iekfvklcse rnfdtnfnae mymvtteeei degqeediev pkamgdifes vagaiyldsg 1741 rnldttwqvi fhmmrgtiel ccanpprspi relmefeqsk vrfskmeril esgkvrvtve 1801 vvnnmrftgm grnyriakat aakralkylh qieqqrrqsp slttv (SEQ ID NO: 103) LOCUS NP_803187 1922 aa linear DEFINITION dicer1; helicase-moi; K12H4.8-LIKE; helicase with RNAse motif [Homo sapiens]. ACCESSION NP_803187 VERSION NP_803187.1 GI:29294651 DBSOURCE REFSEQ: accession NM_177438.1 KEYWORDS SOURCE Homo sapiens (human) ORGANISM Homo sapiens Eukaryota; Metazoa; Chordata; Craniata; Vertebrata; Euteleostomi; Mammalia; Eutheria; Primates; Catarrhini; Hominidae; Homo. REFERENCE 1 (residues 1 to 1922) AUTHORS Handa,V., Saha,T. and Usdin,K. TITLE The fragile X syndrome repeats form RNA hairpins that do not activate the interferon-inducible protein kinase, PKR, but are cut by Dicer JOURNAL Nucleic Acids Res. 31 (21), 6243-6248 (2003) PUBMED 14576312 REMARK GeneRIF: fragile X syndrome CGG repeats readily form RNA hairpins and is digested by the human Dicer enzyme, a step central to the RNA interference effect on gene expression REFERENCE 2 (residues 1 to 1922) AUTHORS Kawasaki,H., Suyama,E., Iyo,M. and Taira,K. TITLE siRNAs generated by recombinant human Dicer induce specific and significant but target site-independent gene silencing in human cells JOURNAL Nucleic Acids Res. 31 (3), 981-987 (2003) PUBMED 12560494 REFERENCE 3 (residues 1 to 1922) AUTHORS Doi,N., Zenno,S., Ueda,R., Ohki-Hamazaki,H., Ui-Tei,K. and Saigo,K. TITLE Short-interfering-RNA-mediated gene silencing in mammalian cells requires Dicer and eIF2C translation initiation factors JOURNAL Curr. Biol. 13 (1), 41-46 (2003) PUBMED 12526743 REFERENCE 4 (residues 1 to 1922) AUTHORS Zhang,H., Kolb,F.A., Brondani,V., Billy,E. and Filipowicz,W. TITLE Human Dicer preferentially cleaves dsRNAs at their termini without a requirement for ATP JOURNAL EMBO J. 21 (21), 5875-5885 (2002) PUBMED 12411505 REMARK GeneRIF: purification and properties of a recombinant human Dicer REFERENCE 5 (residues 1 to 1922) AUTHORS Provost,P., Dishart,D., Doucet,J., Frendewey,D., Samuelsson,B. and Radmark,O. TITLE Ribonuclease activity and RNA binding of recombinant huma Dicer JOURNAL EMBO J. 21 (21), 5864-5874 (2002) PUBMED 12411504 REMARK GeneRIF: cloning and expression of the 218 kDa human Dicer, and characterization of its ribonuclease activity and dsRNA- binding properties REFERENCE 6 (residues 1 to 1922) AUTHORS Matsuda,S., Ichigotani,Y., Okuda,T., Irimura,T., Nakatsugawa,S. and Hamaguchi,M. TITLE Molecular cloning and characterization of a novel human gene (HERNA) which encodes a putative RNA-helicase JOURNAL Biochim. Biophys. Acta 1490 (1-2), 163-169 (2000) PUBMED 10786632 REFERENCE 7 (residues 1 to 1922) AUTHORS Provost,P., Samuelsson,B. and Radmark,O. TITLE Interaction of 5-lipoxygenase with cellular proteins JOURNAL Proc. Natl. Acad. Sci. U.S.A. 96 (5), 1881-1885 (1999) PUBMED 10051563 COMMENT REVIEWED REFSEQ: This record has been curated by NCBI staff. The reference sequence was derived from AB023145.2, AB028449.1, AK091094.1, AW297296.1, BI913232.1 and BQ937506.1. Summary: This gene encodes a protein possessing an RNA helicase motif containing a DEXH box in its amino terminus and an RNA motif in the carboxy terminus. The encoded protein functions as a ribonuclease and is required by the RNA interference and small temporal RNA (stRNA) pathways to produce the active small RNA component that represses gene expression. Two transcript variants encoding the same protein have been identified for this gene. Transcript Variant: This variant (1) represents the longer transcript. Variants 1 and 2 encode the same isoform. FEATURES Location/Qualifiers source 1..1922 /organism="Homo sapiens" /db_xref="taxon:9606" /chromosome="14" /map="14q32.2" Protein 1..1922 /product="dicer1" /EC_number="3.1.26.-" /note="helicase-moi; K12H4.8-LIKE; helicase with RNAse motif" Region 37..>208 /region_name="ERCC4-like helicases [DNA replication, recombination, and repair]" /note="MPH1" /db_xref="CDD:10833" Region 40..211 /region_name="DEAD-like helicases superfamily(DEAD disclosed as SEQ ID NO: 14)" /note="DEXDc" /db_xref="CDD:22813" Region 107..1899 /region_name="dsRNA-specific nuclease Dicer and related ribonucleases [RNA processing and modification]" /note="KOG0701" /db_xref="CDD:18495" variation 257 /replace="*" /replace="C" /db_xref="dbSNP:12432511" Region <499..553 /region_name="Helicase conserved C-terminal domain" /note="helicase_C" /db_xref="CDD:22962" variation 499 /replace="R" /replace="T" /db_xref="dbSNP:4566088" Region 625..722 /region_name="Domain of unknown function" /note="DUF283" /db_xref="CDD:5126" Region 895..1064 /region_name="PAZ domain" /note="PAZ" /db_xref="CDD:17101" Region 1296..>1387 /region_name="Ribonuclease III family" /note="RIBOc" /db_xref="CDD:22830" Region 1682..1846 /region_name="Ribonuclease III family" /note="RIBOc" /db_xref="CDD:22830" CDS 1..1922 /gene="DICER1" /coded_by="NM_177438.1:239..6007" /note="go_component: intracellular [goid 0005622] [evidence NAS] [pmid 12560494]; go_function: double-stranded RNA binding [goid 0003725] [evidence IDA] [pmid 12411504]; go_function: endonuclease activity [goid 0004519] [evidence IEA]; go_function: ATP binding [goid 0005524] [evidence IEA]; go_function: ribonuclease III activity [goid 0004525] [evidence IDA] [pmid 12560494]; go_function: ATP dependent helicase activity [goid 0008026] [evidence IEA]; go_function: hydrolase activity [goid 0016787] [evidence IEA]; go_process: RNA processing [goid 0006396] [evidence IEA]; go_process: RNA interference, targeting of mRNA for destruction [goid 0030423] [evidence IEP] [pmid 12560494]" /db_xref="GeneID:23405" /db_xref="LocusID:23405" /db_xref="MIM:606241" ORIGIN 1 mkspalqpls maglqlmtpa sspmgpffgl pwqqeaihdn iytprkyqve lleaaldhnt 61 ivclntgsgk tfiavlltke lsyqirgdfs rngkrtvflv nsanqvaqqv savrthsdlk 121 vgeysnlevn aswtkerwnq eftkhqvlim tcyvalnvlk ngylslsdin llvfdechla 181 ildhpyreim klcencpscp rilgltasil ngkcdpeele ekiqklekil ksnaetatdl 241 vvldrytsqp ceivvdcgpf tdrsglyerl lmeleealnf indcnisvhs kerdstlisk 301 qilsdcravl vvlgpwcadk vagmmvrelq kyikheqeel hrkfllftdt flrkihalce 361 ehfspasldl kfvtpkvikl leilrkykpy erqqfesvew ynnrnqdnyv swsdseddde 421 deeieekekp etnfpspftn ilcgiifver rytavvlnrl ikeagkqdpe layissnfit 481 ghgigknqpr nkqmeaefrk qeevlrkfra hetnlliats iveegvdipk cnlvvrfdlp 541 teyrsyvqsk grarapisny imladtdkik sfeedlktyk aiekilrnkc sksvdtgetd 601 idpvmddddv fppyvlrpdd ggprvtinta ighinrycar lpsdpfthla pkcrtrelpd 661 gtfystlylp insplrasiv gppmscvrla ervvalicce klhkigeldd hlmpvgketv 721 kyeeeldlhd eeetsvpgrp gstkrrqcyp kaipeclrds yprpdqpcyl yvigmvlttp 781 lpdelnfrrr klyppedttr cfgiltakpi pqiphfpvyt rsgevtisie lkksgfmlsl 841 qmlelitrlh qyifshilrl ekpalefkpt dadsaycvlp lnvvndsstl didfkfmedi 901 eksearigip stkytketpf vfkledyqda viipryrnfd qphrfyvadv ytdltplskf 961 pspeyetfae yyktkynldl tnlnqplldv dhtssrlnll tprhlnqkgk alplssaekr 1021 kakweslqnk qilvpelcai hpipaslwrk avclpsilyr lhclltaeel raqtasdagv 1081 gvrslpadfr ypnldfgwkk sidsksfisi snsssaendn yckhstivpe naahqganrt 1141 sslenhdqms vncrtllses pgklhvevsa dltainglsy nqnlangsyd lanrdfcqgn 1201 qlnyykqeip vqpttsysiq nlysyenqpq psdectllsn kyldgnanks tsdgspvmav 1261 mpgttdtiqv lkgrmdseqs psigyssrtl gpnpglilqa ltlsnasdgf nlerlemlgd 1321 sflkhaitty lfctypdahe grlsymrskk vsncnlyrlg kkkglpsrmv vsifdppvnw 1381 lppgyvvnqd ksntdkwekd emtkdcmlan gkldedyeee deeeeslmwr apkeeadyed 1441 dfleydqehi rfidnmlmgs gafvkkisls pfsttdsaye wkmpkksslg smpfssdfed 1501 fdysswdamc yldpskavee ddfvvgfwnp seencgvdtg kqsisydlht eqciadksia 1561 dcveallgcy ltscgeraaq lflcslglkv lpvikrtdre kalcptrenf nsqqknlsvs 1621 caaasvassr ssvlkdseyg clkipprcmf dhpdadktln hlisgfenfe kkinyrfknk 1681 ayllqaftha syhyntitdc yqrleflgda ildylitkhl yedprqhspg vltdlrsalv 1741 nntifaslav kydyhkyfka vspelfhvid dfvqfqlekn emqgmdselr rseedeekee 1801 dievpkamgd ifeslagaiy mdsgmsletv wqvyypmmrp liekfsanvp rspvrellem 1861 epetakfspa ertydgkvrv tvevvgkgkf kgvgrsyria ksaaarralr slkanqpqvp 1921 ns (SEQ ID NO: 104) LOCUS NP_683750 1917 aa linear DEFINITION dicer1; endoribonuclease Dicer [Mus musculus]. ACCESSION NP_683750 VERSION NP_683750.1 GI:22507359 DBSOURCE REFSEQ: accession NM_148948.1 KEYWORDS . SOURCE Mus musculus (house mouse) ORGANISM Mus musculus Eukaryota; Metazoa; Chordata; Craniata; Vertebrata; Euteleostomi; Mammalia; Eutheria; Rodentia; Sciurognathi; Muridae; Murinae; Mus. REFERENCE 1 (residues 1 to 1917) AUTHORS Bernstein,E., Kim,S.Y., Carmell,M.A., Murchison,E.P., Alcorn,H., Li,M.Z., Mills,A.A., Elledge,S.J., Anderson,K.V. and Hannon,G.J. TITLE Dicer is essential for mouse development JOURNAL Nat. Genet. 35 (3), 215-217 (2003) PUBMED 14528307 REMARK GeneRIF: role in lethality early in development REFERENCE 2 (residues 1 to 1917) AUTHORS Okazaki,N., Kikuno,R., Ohara,R., Inamoto,S., Koseki,H., Hiraoka,S., Saga,Y., Nagase,T., Ohara,O. and Koga,H. TITLE Prediction of the coding sequences of mouse homologues of KIAA gene: III. the complete nucleotide sequences of 500 mouse KIAA-homologous cDNAs identified by screening of terminal sequences of cDNA clones randomly sampled from size- fractionated libraries JOURNAL DNA Res. 10 (4), 167-180 (2003) PUBMED 14621295 REFERENCE 3 (residues 1 to 1917) AUTHORS Doi,N., Zenno,S., Ueda,R., Ohki-Hamazaki,H., Ui-Tei,K. and Saigo,K. TITLE Short-interfering-RNA-mediated gene silencing in mammalian cells requires Dicer and eIF2C translation initiation factors JOURNAL Curr. Biol. 13 (1), 41-46 (2003) PUBMED 12526743 REFERENCE 4 (residues 1 to 1917) AUTHORS Nicholson,R.H. and Nicholson,A.W. TITLE Molecular characterization of a mouse cDNA encoding Dicer, a ribonuclease III ortholog involved in RNA interference JOURNAL Mamm. Genome 13 (2), 67-73 (2002) PUBMED 11889553 COMMENT PROVISIONAL REFSEQ: This record has not yet been subject to final NCBI review. The reference sequence was derived from AF430845.1. FEATURES Location/Qualifiers source 1..1917 /organism="Mus musculus" /strain="CZECHII" /db_xref="taxon:10090" /chromosome="12" /map="12F1" Protein 1..1917 /product="dicer1" /note="endoribonuclease Dicer" Region 38..>226 /region_name="ERCC4-like helicases [DNA replication, recombination, and repair]" /note="MPH1" /db_xref="CDD:10833" Region 41..242 /region_name="DEAD-like helicases superfamily"(DEAD disclosed as SEQ ID NO: 14) /note="DEXDc" /db_xref="CDD:24291" Region 109..1894 /region_name="dsRNA-specific nuclease Dicer and related ribonucleases [RNA processing and modification]" /note="KOG0701" /db_xref="CDD:18495" Region <500..554 /region_name="Helicase conserved C-terminal domain" /note="Helicase_C" /db_xref="CDD:24402" Region 631..723 /region_name="Domain of unknown function" /note="DUF283" /db_xref="CDD:26059" Region <926..>1039 /region_name="Germ-line stem cell division protein Hiwi/Piwi" /note="KOG1042" /db_xref="CDD:18835" Region 1297..>1388 /region_name="Ribonuclease III family" /note="RIBOc" /db_xref="CDD:22830" Region 1677..1841 /region_name="Ribonuclease III family" /note="RIBOc" /db_xref="CDD:22830" CDS 1..1917 /gene="Dicer1" /coded_by="NM_148948.1:255..6008" /note="go_component: cellular_component unknown [goid 0008372] [evidence ND]; go_component: intracellular [goid 0005622] [evidence ISS] [pmid 12466851]; go_function: ribonuclease III activity [goid 0004525] [evidence IDA] [pmid 14528307]; go_function: nuclease activity [goid 0004518] [evidence IEA]; go_function: RNA binding [goid 0003723] [evidence IEA]; go_function: helicase activity [goid 0004386] [evidence IEA]; go_function: endonuclease activity [goid 0004519] [evidence IEA]; go_function: ATP binding [goid 0005524] [evidence IEA]; go_function: ATP-dependent helicase activity [goid 0008026] [evidence IEA]; go_function: hydrolase activity [goid 0016787] [evidence IEA]; go_function: nucleic acid binding [goid 0003676] [evidence IEA]; go_function: double-stranded RNA binding [goid 0003725] [evidence ISS] [pmid 12466851]; go_process: biological_process unknown [goid 0000004] [evidence ND]; go_process: RNA processing [goid 0006396] [evidence IDA] [pmid 14528307]; go_process: stem cell maintenance [goid 0019827] [evidence IMP] [pmid 14528307]; go_process: RNA interference, production of guide RNAs [goid 0030422] [evidence IDA] [pmid 14528307]" /db_xref="GeneID:192119" /db_xref="LocusID:192119" /db_xref="MGI:2177178" ORIGIN 1 mnekpcfaal smaglqlmtp asspmgpffg lpwqqeaihd niytprkyqv elleaaldhn 61 tivclntgsg ktfiavlltk elahqirgdl nphakrtvfl vnsanqvcqq vsavrthsdl 121 kvgeysdlev naswtkerws qeftkhqvli mtcyvaltvl kngylslsdi nllvfdechl 181 aildhpyrei mklcescpsc prilgltasi lngkcdpeel eekiqkleri lrsdaetatd 241 lvvldrytsq pceivvdcgp ftdrsglyer llmeleaald findcnvavy skerdstlis 301 kqilsdcrav lvvlgpwcad kvagmmvrel qkyikheqee lhrkfllftd tllrkihalc 361 eeyfspasld lkyvtpkvmk lleilrkykp yerqqfesve wynnrnqdny vswsdseddd 421 ddeeieekek petnfpspft nilcgiifve rrytavvlnr likeagkqdp elayissnfi 481 tghgigknqp rskqmeaefr kqeevlrkfr ahetnlliat svveegvdip kcnlvvrfdl 541 pteyrsyvqs kgrarapisn yvmladtdki ksfeedlkty kaiekilrnk csksadgaea 601 dvhagvdded afppyvlrpd dggprvtint aighinryca rlpsdpfthl apkcrtrelp 661 dgtfystlyl pinsplrasi vgppmdsvrl aervvalicc eklhkigeld ehlmpvgket 721 vkyeeeldlh deeetsvpgr pgstkrrqcy pkaipeclrd sypkpdqpcy lyvigmvltt 781 plpdelnfrr rklyppedtt rcfgiltakp ipqiphfpvy trsgevtisi elkksgfils 841 qqmlelitrl hqyifshilr lekpalefkp tgaesaycvl plnvvndsgt ldidfkfmed 901 ieksearigi pstkysketp fvfkledyqd aviipryrnf dqphrfyvad vytdltplsk 961 fpspeyetfa eyyktkynld ltnlnqplld vdhtssrlnl ltprhlnqkg kalplssaek 1021 rkakweslqn kqilvpelca ihpipaslwr kavclpsily rlhclltaee lraqtasdag 1081 vgvrslpvdf rypnldfgwk ksidsksfis tcnsslaesd nyckhsttvv pehaahqgat 1141 rpslenhdqm svnckrlpae spaklqsevs tdltaingls ynknlangsy dlvnrdfcqg 1201 nqlnyfkqei pvqpttsypi qnlynyenqp kpsnecplls ntyldgnant stsdgspavs 1261 tmpammnavk alkdrmdseq spsvgyssrt lgpnpglilq altlsnasdg fnlerlemlg 1321 dsflkhaitt ylfctypdah egrlsymrsk kvsncnlyrl gkkkglpsrm vvsifdppvn 1381 wlppgyvvnq dksnsekwek demtkdclla ngklgeacee eedltwrapk eeaededdfl 1441 eydqehiqfi dsmlmgsgaf vrkislspfs asdsayewkm pkkaslgsmp fasgledfdy 1501 sswdamcyld pskaveeddf vvgfwnpsee ncgvdtgkqs isydlhteqc iadksiadcv 1561 eallgcylts cgeraaqlfl cslglkvlpv ikrtsrekal dpaqengssq qkslsgscaa 1621 pvgprssagk dleygclkip prcmfdhpda ektlnhlisg fetfekkiny rfknkayllq 1681 afthasyhyn titdcyqrle flgdaildyl itkhlyedpr qhspgvltdl rsalvnntif 1741 aslavkydyh kyfkavspel fhviddfvkf qleknemqgm dselrrseed eekeedievp 1801 kamgdifesl agaiymdsgm slevvwqvyy pmmqpliekf sanvprspvr ellemepeta 1861 kfspaertyd gkvrvtvevv gkgkfkgvgr syriaksaaa rralrslkan qpqvpns (SEQ ID NO: 105)

V.Screening Assays

According to the invention, the following assays may be used to identify compounds that modulate interaction (e.g., binding) of Dicer or bioactive fragments thereof with Dicer interactors or bioactive fragments thereof, or modulate a Dicer activity or Dicer interactor activity, and hence, modulators of gene silencing or RNAi. Such modulators are particularly useful in regulation of (1) processing of miRNA precursors; (2) processing of siRNA precursors; (3) mediating mRNA cleavage; (4) mediating assembly of RISC (e.g., via siRNAs); (5) directing translation repression (e.g., via miRNAs); (6) a ribonuclease activity (e.g., cleavage of dsRNA); and (7) initiation of RNAi. The assays feature identifying modulators of the activity of Dicer interactors or bioactive fragments thereof, including, but not limited to, those activities identified in supra.

The assays of the present invention are used to identify modulators of the activity of Dicer or bioactive fragments thereof or Dicer interactors or bioactive fragments thereof. The modulators of the present invention are particularly useful in modulating Dicer and/or RNAi related activities but can also affect non-RNAi related activities.

VA.Cell Free Assays

In one embodiment, an assay of the present invention is a cell-free assay in which a composition comprising assay reagents (e.g., a Dicer interactor polypeptide, Dicer polypeptide or biologically active portions thereof), is contacted with a test compound and the ability of the test compound to modulate binding of the Dicer interactor polypeptide to the Dicer polypeptide (or bioactive fragments thereof) is determined. Binding of the Dicer interactor or Dicer (or bioactive fragments thereof) can be accomplished, for example, by coupling the polypeptide or fragment with a radioisotope or enzymatic label such that binding of polypeptide reagents can be determined by detecting the labeled compound or polypeptide in a complex. For example, test compounds or polypeptides can be labeled with ¹²⁵I, ³⁵S, ³³P,³²P,¹⁴C, or ³H, either directly or indirectly, and the radioisotope detected by direct counting of radioemmission or by scintillation counting. Alternatively, polypeptides can be enzymatically labeled with, for example, horseradish peroxidase, alkaline phosphatase, or luciferase, and the enzymatic label detected by determination of conversion of an appropriate protein to product.

Determination of binding of reagents can also be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705. As used herein, "BIA" is a technology for studying biospecific interactions in real time, without labeling any of the interactants (e.g., BIAcore™). Changes in the optical phenomenon of surface plasmon resonance (SPR) can be used as an indication of real-time reactions between biological molecules.

In a preferred embodiment, the assay includes contacting Dicer polypeptide or biologically active portion thereof with a Dicer target molecule, e.g., a Dicer interactor or a bioactive fragment thereof to form an assay mixture, contacting the assay mixture with a test compound, and determining the ability of the test compound to interact with the Dicer polypeptide, wherein determining the ability of the test compound to interact with the Dicer polypeptide comprises determining the ability of the test compound to preferentially bind to Dicer or the bioactive portion thereof as compared to the Dicer target molecule (e.g., a Dicer protein). In another embodiment, the assay includes contacting the Dicer interactor polypeptide or biologically active portion thereof with a Dicer interactor target molecule, e.g., Dicer or a bioactive fragment thereof to form an assay mixture, contacting the assay mixture with a test compound, and determining the ability of the test compound to modulate binding between the Dicer interactor polypeptide and the Dicer polypeptide.

In another embodiment, the assay is a cell-free assay in which a composition comprising a Dicer polypeptide and a Dicer interactor polypeptide (or bioactive portions thereof) is contacted with a test compound and the ability of the test compound to modulate (e.g., stimulate or inhibit) the activity of the Dicer polypeptide or Dicer interactor polypeptide (or biologically active portions thereof) is determined.

Determining the ability of the test compound to modulate the activity of a Dicer or a Dicer interactor polypeptide can be accomplished, for example, by determining the ability of the Dicer interactor polypeptide to modulate the activity of a downstream binding partner or target molecule by one of the methods described herein for cell or organism-based assays. For example, the catalytic/enzymatic activity of the target molecule on an appropriate downstream protein can be determined as previously described.

In yet another embodiment, the cell-free assay involves contacting a Dicer interactor polypeptide or biologically active portion thereof with a Dicer interactor target molecule that binds the Dicer interactor polypeptide to form an assay mixture, contacting the assay mixture with a test compound, and determining the ability of the test compound (e.g., Dicer) to preferentially modulate the activity of a Dicer interactor binding partner or target molecule, as compared to the Dicer protein.

In more than one embodiment of the above assay methods of the present invention, it may be desirable to immobilize either the Dicer interactor or Dicer (or target molecules) to facilitate separation of complexed from uncomplexed forms of one or both of the proteins, as well as to accommodate automation of the assay. The ability of a test compound to modulate Dicer interactor polypeptide activity, Dicer polypeptide activity, interaction of a Dicer interactor polypeptide with a Dicer polypeptide (or target interaction or activity) in the presence and absence of a candidate compound, can be accomplished in any vessel suitable for containing the reactants. Examples of such vessels include microtitre plates, test tubes, and micro-centrifuge tubes. In one embodiment, a fusion protein can be provided so as to add a domain that allows one or both of the proteins to be bound to a matrix. For example, glutathione-S-transferase/Dicer interactor fusion proteins, glutathione-S-transferase/Dicer fusion proteins, or target fusion proteins can be adsorbed onto glutathione sepharose beads (Sigma Chemical, St. Louis, MO) or glutathione derivatized microtitre plates, which are then combined with the test compound or the test compound and either the non-adsorbed Dicer polypeptide or Dicer interactor polypeptide (or target polypeptide), and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of Dicer interactor binding or activity or Dicer binding or activity (or target binding or activity) determined using standard techniques.

Additional exemplary Dicer and/or Dicer interactor fusion proteins (or target fusion proteins) include, but are not limited to, chitin binding domain (CBD) fusion proteins, hemagglutinin epitope tagged (HA)-fusion proteins, His fusion proteins (e.g., His₆ tagged proteins), FLAG tagged fusion proteins, AU1 tagged proteins, and the like.

Other techniques for immobilizing proteins on matrices can also be used in the screening assays of the invention. For example, either a Dicer polypeptide, a Dicer interactor polypeptide or target polypeptide can be immobilized utilizing conjugation of biotin and streptavidin. Biotinylated Dicer polypeptide, Dicer interactor polypeptide or target polypeptide can be prepared from biotin-NHS (N-hydroxy-succinimide) using techniques well known in the art (e.g., biotinylation kit, Pierce Chemicals, Rockford, IL), and immobilized in the wells of streptavidin-coated 96 well plates (Pierce Chemical). Alternatively, antibodies reactive with Dicer polypeptide, Dicer interactor polypeptide or target polypeptide but which do not interfere with binding of the Dicer interactor polypeptide to Dicer polypeptide (or protein to target binding) can be derivatized to the wells of the plate, and unbound Dicer or Dicer interactor polypeptide (or target) trapped in the wells by antibody conjugation. Methods for detecting such complexes, in addition to those described above for the GST-immobilized complexes, include immunodetection of complexes using antibodies reactive with the Dicer interactor polypeptide, Dicer polypeptide or target polypeptide, as well as enzyme-linked assays which rely on detecting an enzymatic activity associated with the Dicer interactor polypeptide, Dicer polypeptide or target polypeptide.

In one aspect of the invention, the Dicer interactor or Dicer polypeptides can be used as "bait proteins" in a two-hybrid assay or three-hybrid assay (see, e.g., U.S. Patent No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with Dicer interactor or Dicer ("binding proteins" or "target molecules") and are involved in Dicer interactor or Dicer activity. Such target molecules are also likely to be involved in the regulation of cellular activities modulated by the Dicer interactor polypeptides or Dicer polypeptides.

At least one exemplary two-hybrid system is based on the modular nature of most transcription factors, which consist of separable DNA-binding and activation domains. Briefly, the assay utilizes two different DNA constructs. In one construct, the gene that codes for a first polypeptide (the "bait" polypeptide, e.g., Dicer or Dicer protein) is fused to a gene encoding the DNA binding domain of a known transcription factor (e.g., GAL-4). In the other construct, a DNA sequence, from a library of DNA sequences, that encodes an unidentified protein ("prey" or "sample") is fused to a gene that codes for the activation domain of the known transcription factor. If the "bait" and the "prey" proteins are able to interact, in vivo, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene that encodes the protein that interacts with the bait polypeptide.

Another exemplary two-hybrid system, referred to in the art as the CytoTrap™ system, is based in the modular nature of molecules of the Ras signal transduction cascade. Briefly, the assay features a fusion protein comprising the "bait" protein and Son-of-Sevenless (SOS) and the cDNAs for unidentified proteins (the "prey") in a vector that encodes myristylated target proteins. Expression of an appropriate bait-prey combination results in translocation of SOS to the cell membrane where it activates Ras. Cytoplasmic reconstitution of the Ras signaling pathway allows identification of proteins that interact with the bait protein of interest, for example, a Dicer or Dicer interactor protein. Additional mammalian two hybrid systems are also known in the art and can be utilized to identify Dicer or Dicer interactor interacting proteins. Moreover, at least one of the above-described assays can be utilized to identify Dicer-interacting domains or regions of the Dicer interactor protein or alternatively, to identify Dicer protein-interacting domain or regions of the Dicer protein.

VB.Cell or Organism Based Assays

In one embodiment, an assay is a cell or organism-based assay in which a cell or organism capable of expressing a Dicer interactor polypeptide, or biologically active portion thereof, is contacted with a test compound and the ability of the test compound to modulate the expression of the Dicer interactor polypeptide, or biologically active portion thereof, determined. In another embodiment, an assay is a cell or organism-based assay in which a cell or organism which expresses a Dicer interactor polypeptide or Dicer polypeptide (or biologically active portions thereof) is contacted with a test compound and the ability of the test compound to modulate the activity of the Dicer interactor polypeptide or Dicer polypeptide (or biologically active portions thereof) determined. The cell, for example, can be of mammalian origin or a yeast cell. The organism can be a nematode, for example, C. elegans or C. briggsae or D. melanogaster. The polypeptides, for example, can be expressed heterologously or native to the cell or organism. Determining the ability of the test compound to modulate the activity of a Dicer interactor or Dicer polypeptide (or biologically active portions thereof) can be accomplished by assaying for any of the activities of a Dicer interactor or Dicer polypeptide described herein. Determining the ability of the test compound to modulate the activity of a Dicer interactor polypeptide or Dicer polypeptide (or biologically active portions thereof) can also be accomplished by assaying for the activity of a Dicer downstream molecule. In one embodiment, determining the ability of the test compound to modulate the activity of a Dicer interactor polypeptide, or biologically active portion thereof, is accomplished by assaying for the ability to bind Dicer or a bioactive portion thereof. In another embodiment, determining the ability of the test compound to modulate the activity of a Dicer interactor polypeptide, or biologically active portion thereof, is accomplished by assaying for the activity of the Dicer interactor polypeptide. In a preferred embodiment, the cell or organism overexpresses the Dicer interactor polypeptide, or biologically active portion thereof, and optionally, overexpresses Dicer, or biologically active portion thereof. In another preferred embodiment, the cell or organism expresses Dicer, or biologically active portion thereof. In yet another preferred example, the cell or organism is contacted with a compound that stimulates a Dicer protein-associated activity or Dicer-associated activity and the ability of a test compound to modulate the Dicer protein-associated activity is determined.

As used herein, the term "bioactive" fragment includes any portion (e.g., a segment of contiguous amino acids) of a Dicer interactor or Dicer protein sufficient to exhibit or exert at least one Dicer protein- or Dicer- associated activity including, for example, the ability to bind to Dicer or Dicer protein, respectively. In various embodiments, the Dicer may be one of two isoforms, Dicer1 or Dicer2. In another embodiment, the bioactive peptide is derived from the amino acid sequence of Dicer. In another embodiment, the bioactive peptide corresponds to a fragment or domain as set forth in subsections IA-IEE, supra or a smaller bioactive fragment thereof. In another embodiment, the bioactive peptide is derived from a Dicer interactor and can include, for example, amino acid residues sufficient to effect enzymatic or nucleic acid-binding activity.

According to the cell or organism-based assays of the present invention, determining the ability of the test compound to modulate the activity of the Dicer polypeptide or biologically active portion thereof, can be determined by assaying for any of the native activities of a Dicer polypeptide as described herein. Moreover, the activity of Dicer, can be determined by assaying for an indirect activity which is coincident to the activity of Dicer. Furthermore, determining the ability of the test compound to modulate the activity of the Dicer and/or Dicer interactor polypeptide or biologically active portion thereof, can be determined by assaying for an activity which is not native to the Dicer interactor or Dicer polypeptide, but for which the cell or organism has been recombinantly engineered. For example, the cell or organism can be engineered to express a target molecule which is a recombinant protein comprising a bioactive portion of Dicer operatively linked to a non-Dicer polypeptide or a bioactive portion of a Dicer interactor operatively linked to a non-Dicer interactor polypeptide. It is also intended that in preferred embodiments, the cell or organism-based assays of the present invention comprise a final step of identifying the compound as a modulator of Dicer interactor activity or Dicer activity.

VI.Assay Reagents

VIA.Test Compounds

The test compounds of the present invention can be obtained using any of the numerous approaches in combinatorial library methods known in the art, including: biological libraries; spatially addressable parallel solid phase or solution phase libraries; synthetic library methods requiring deconvolution; the 'one-bead one-compound' library method; and synthetic library methods using affinity chromatography selection. The biological library approach is limited to peptide libraries, while the other four approaches are applicable to peptide, non-peptide oligomer or small molecule libraries of compounds (Lam, K.S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner USP 5,223,409), spores (Ladner USP '409), plasmids (Cull et al. (1992) Proc Natl Acad Sci USA 89:1865-1869) or on phage (Scott and Smith (1990) Science 249:386-390); (Devlin (1990) Science 249:404-406); (Cwirlaet al. (1990) Proc. Natl. Acad. Sci. 87:6378-6382); (Felici (1991) J. Mol. Biol. 222:301-310); (Ladner supra.).

In a preferred embodiment, the library is a natural product library.

VIB.Antibodies, Bioactive Fragments and Fusion Proteins

Preferred aspects of the invention feature Dicer polypeptides, Dicer interactor polypeptides and biologically active portions (i.e., bioactive fragments) of Dicer polypeptides or Dicer interactor polypeptides, including polypeptide fragments suitable for use in making Dicer interactor or Dicer fusion proteins. In one embodiment, Dicer polypeptides or Dicer interactor polypeptides can be isolated from cells or tissue sources by an appropriate purification scheme using standard protein purification techniques. Dicer polypeptide or Dicer interactor polypeptides can be further derived from said isolated polypeptides using standard enzymatic techniques. In another embodiment, Dicer interactor polypeptides, Dicer polypeptides or bioactive fragments thereof are produced by recombinant DNA techniques. Alternative to recombinant expression, Dicer interactor polypeptides, Dicer polypeptides or bioactive fragments thereof can be synthesized chemically using standard peptide synthesis techniques.

Polypeptides of the invention are preferably "isolated" or "purified". The terms "isolated" and "purified" are used interchangeably herein. "Isolated" or "purified" means that the protein or polypeptide is substantially free of cellular material or other contaminating proteins from the cell or tissue source from which the polypeptide is derived, substantially free of other protein fragments , for example, non-desired fragments in a digestion mixture, or substantially free from chemical precursors or other chemicals when chemically synthesized. The language "substantially free of cellular material" includes preparations in which the polypeptide is separated from other components of the cells from which it is isolated or recombinantly produced. In one embodiment, the language "substantially free of cellular material" includes preparations of polypeptide having less than about 30% (by dry weight) of non-Dicer interactor or non-Dicer polypeptide (also referred to herein as a "contaminating protein"), more preferably less than about 20% of non-Dicer interactor or non-Dicer polypeptide, still more preferably less than about 10% of non-Dicer interactor or non-Dicer polypeptide, and most preferably less than about 5% non-Dicer interactor or non-Dicer polypeptide. When the polypeptide or protein is recombinantly produced, it is also preferably substantially free of culture medium, i.e., culture medium represents less than about 20%, more preferably less than about 10%, and most preferably less than about 5% of the volume of the polypeptide preparation. When the polypeptide or protein is produced by, for example, chemical or enzymatic processing from isolated or purified Dicer interactor or Dicer protein, the preparation is preferably free of enzyme reaction components or chemical reaction components and is free of non-desired Dicer interactor or Dicer fragments, i.e., the desired polypeptide represents at least 75% (by dry weight) of the preparation, preferably at least 80%, more preferably at least 85%, and even more preferably at least 90%, 95%, 99% or more or the preparation.

The language "substantially free of chemical precursors or other chemicals" includes preparations of polypeptide in which the polypeptide is separated from chemical precursors or other chemicals which are involved in the synthesis of the polypeptide. In one embodiment, the language "substantially free of chemical precursors or other chemicals" includes preparations having less than about 30% (by dry weight) of chemical precursors or reagents, more preferably less than about 20% chemical precursors or reagents, still more preferably less than about 10% chemical precursors or reagents, and most preferably less than about 5% chemical precursors or reagents.

Bioactive fragments of Dicer interactor or Dicer include polypeptides comprising amino acid sequences sufficiently identical to or derived from the amino acid sequence of the Dicer interactor protein or the Dicer protein, respectively, which include less amino acids than the full length protein, and exhibit at least one biological activity of the full-length protein. Typically, biologically active portions comprise a domain or motif with at least one activity of the full-length protein. A biologically active portion of a Dicer interactor or Dicer polypeptide can be a polypeptide which is, for example, 10, 20, 30, 40, 50, 100, 150, 200, 250, 300, 350, 400, 450, 500, 550, 600, 650, 700, 750, 800, 850, 900, 950, 1000 or more amino acids in length. In a preferred embodiment, a bioactive portion of a Dicer protein comprises a portion comprising a Dicer interactor interacting domain. Moreover, other biologically active portions, in which other regions of the protein are deleted, can be prepared by recombinant techniques and evaluated for one or more of the functional activities of a native Dicer interactor or Dicer protein. Mutants of Dicer and/or Dicer interactors can also be utilized as assay reagents, for example, mutants having reduced, enhanced or otherwise altered biological properties identified according to one of the activity assays described herein.

As defined herein, a Dicer polypeptide or Dicer interactor polypeptide of the invention includes polypeptides having the amino acid sequences set forth in subsections IA-IMM or II, infra, as well as homologs an/or orthologs of said polypeptides, i.e. polypeptides having sufficient sequence identity to function in the same manner as the described polypeptides. To determine the percent identity of two amino acid sequences (or of two nucleotide or amino acid sequences), the sequences are aligned for optimal comparison purposes (e.g., gaps can be introduced in the first sequence or second sequence for optimal alignment). The amino acid residues or nucleotides at corresponding amino acid positions or nucleotide positions are then compared. When a position in the first sequence is occupied by the same residue as the corresponding position in the second sequence, then the molecules are identical at that position. The percent identity between the two sequences is a function of the number of identical positions shared by the sequences (i.e., % homology = # of identical positions/total # of positions x 100), optionally penalizing the score for the number of gaps introduced and/or length of gaps introduced.

The comparison of sequences and determination of percent identity between two sequences can be accomplished using a mathematical algorithm. In one embodiment, the alignment generated over a certain portion of the sequence aligned having sufficient identity but not over portions having low degree of identity (i.e., a local alignment). A preferred, non-limiting example of a local alignment algorithm utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the BLAST programs (version 2.0) of Altschul, et al. (1990) J. Mol. Biol. 215:403-10. BLAST alignments can be generated and percent identity calculated using BLAST protein searches (e.g., the XBLAST program) using Dicer protein, Dicer or a portion thereof as a query, score = 50, wordlength = 3.

In another embodiment, the alignment is optimized by introducing appropriate gaps and percent identity is determined over the length of the aligned sequences (i.e., a gapped alignment). To obtain gapped alignments for comparison purposes, Gapped BLAST can be utilized as described in Altschul et al., (1997) Nucleic Acids Research 25(17):3389-3402. In another embodiment, the alignment is optimized by introducing appropriate gaps and percent identity is determined over the entire length of the sequences aligned (i.e., a global alignment). A preferred, non-limiting example of a mathematical algorithm utilized for the global comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The invention also provides Dicer interactorsand Dicer chimeric or fusion proteins. As used herein, a Dicer interactor or Dicer "chimeric protein" or "fusion protein" comprises a Dicer interactor or Dicer polypeptide operatively linked to a non-Dicer interactor polypeptide or non-Dicer polypeptide, respectively. A "Dicer interactor polypeptide" or "Dicer polypeptide" refers to a polypeptide having an amino acid sequence corresponding to the Dicer interactor or Dicer protein, respectively, whereas a "non-Dicer interactor polypeptide" or "non-Dicer polypeptide" refers to a polypeptide having an amino acid sequence corresponding to a protein which is not substantially identical to the Dicer interactor protein or Dicer protein. Within a fusion protein the Dicer interactor or Dicer polypeptide can correspond to all or a portion of a Dicer interactor or Dicer protein. In a preferred embodiment, a Dicer interactor or Dicer fusion protein comprises at least one biologically active portion of a Dicer interactor or Dicer protein, respectively. In another preferred embodiment, a Dicer interactor or Dicer fusion protein comprises at least two biologically active portions of a Dicer interactor or Dicer protein, respectively. In yet another preferred embodiment, a fusion protein can comprise Dicer protein, or a bioactive portion thereof, operatively linked to Dicer, or a bioactive portion thereof, such that Dicer interactor and Dicer, or their respective bioactive portions are brought into close proximity. Within the fusion protein, the term "operatively linked" is intended to indicate that the Dicer interactor or Dicer polypeptide and the non-Dicer interactor polypeptide or non-Dicer polypeptide are fused in-frame to each other. The non-Dicer interactor polypeptide or non-Dicer polypeptide can be fused to the N-terminus or C-terminus of the Dicer interactor polypeptide or Dicer polypeptide, respectively.

For example, in one embodiment, the fusion protein is a GST-fusion protein in which the Dicer interactor or Dicer sequences are fused to the C-terminus of the GST sequences. In another embodiment, the fusion protein is a chitin binding domain (CBD) fusion protein in which the Dicer interactor or Dicer sequences are fused to the N-terminus of chitin binding domain (CBD) sequences. Such fusion proteins can facilitate the purification of recombinant Dicer interactor or Dicer.

Preferably, a chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety. A Dicer protein- or Dicer-encoding nucleic acid can be cloned into such an expression vector such that the fusion moiety is linked in-frame to the Dicer interactor or Dicer polypeptide.

A Dicer interactor polypeptide or Dicer polypeptide, or a portion or fragment of Dicer interactor or Dicer, can also be used as an immunogen to generate antibodies that bind Dicer interactor or Dicer or that block Dicer protein/Dicer binding using standard techniques for polyclonal and monoclonal antibody preparation. A full-length polypeptide can be used or, alternatively, the invention provides antigenic peptide fragments for use as immunogens. Preferably, an antigenic fragment comprises at least 8 amino acid residues of the amino acid sequence of a Dicer interactor or Dicer and encompasses an epitope of Dicer interactor or Dicer such that an antibody raised against the peptide forms a specific immune complex with Dicer interactor or Dicer, respectively. Preferably, the antigenic peptide comprises at least 10 amino acid residues, more preferably at least 15 amino acid residues, even more preferably at least 20 amino acid residues, and most preferably at least 30 amino acid residues. Preferred epitopes encompassed by the antigenic peptide are regions of Dicer interactor or Dicer that are located on the surface of the protein, e.g., hydrophilic regions. Antigenic determinants at the termini of Dicer interactor are preferred for the development of antibodies that do not interfere with the Dicer protein:Dicer interaction. Alternatively, interfering antibodies can be generated towards antigenic determinants located within the Dicer interacting domain of Dicer protein. The latter are preferred for therapeutic purposes.

A Dicer interactor or Dicer immunogen typically is used to prepare antibodies by immunizing a suitable subject, (e.g., rabbit, goat, mouse or other mammal) with the immunogen. An appropriate immunogenic preparation can contain, for example, recombinantly expressed Dicer interactor or Dicer polypeptide or a chemically synthesized Dicer interactor or Dicer polypeptide. The preparation can further include an adjuvant, such as Freund's complete or incomplete adjuvant, or similar immunostimulatory agent. Immunization of a suitable subject with an immunogenic Dicer interactor or Dicer preparation induces apolyclonal anti-Dicer interactor or anti-Dicer antibody response, respectively.

Accordingly, another aspect of the invention pertains to anti-Dicer interactor or anti-Dicer antibodies. The term "antibody" as used herein refers to immunoglobulin molecules and immunologically active portions of immunoglobulin molecules, i.e., molecules that contain an antigen binding site which specifically binds (immunoreacts with) an antigen, such as Dicer interactor or Dicer. Examples of immunologically active portions of immunoglobulin molecules include F(ab) and F(ab')₂ fragments which can be generated by treating the antibody with an enzyme such as pepsin. The invention provides polyclonal and monoclonal antibodies that bind Dicer protein. The term "monoclonal antibody" or "monoclonal antibody composition", as used herein, refers to a population of antibody molecules that contain only one species of an antigen binding site capable of immunoreacting with a particular epitope of Dicer interactor or Dicer. A monoclonal antibody composition thus typically displays a single binding affinity for a particular Dicer interactor or Dicer polypeptide with which it immunoreacts.

Polyclonal anti-Dicer interactor or anti-Dicer antibodies can be prepared as described above by immunizing a suitable subject with a Dicer interactor or Dicer immunogen, respectively. The antibody titer in the immunized subject can be monitored over time by standard techniques, such as with an enzyme linked immunosorbent assay (ELISA) using immobilized Dicer interactor or Dicer. If desired, the antibody molecules can be isolated from the mammal (e.g., from the blood) and further purified by well known techniques, such as protein A chromatography to obtain the IgG fraction. At an appropriate time after immunization, e.g., when the anti-Dicer interactor or anti-Dicer antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem .255:4980-83; Yeh et al. (1976) PNAS 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, New York (1980); E. A. Lerner (1981) Yale J. Biol. Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with a Dicer interactor or Dicer immunogen as described above, and the culture supernatants of the resulting hybridoma cells are screened to identify a hybridoma producing a monoclonal antibody that binds Dicer interactor or Dicer, respectively.

Any of the many well known protocols used for fusing lymphocytes and immortalized cell lines can be applied for the purpose of generating an anti-Dicer interactor monoclonal antibody (see, e.g., G. Galfre et al. (1977) Nature 266:55052; Gefter et al.Somatic Cell Genet., cited supra; Lerner, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine ("HAT medium"). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol ("PEG"). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind Dicer interactor or Dicer, e.g., using a standard ELISA assay.

Alternative to preparing monoclonal antibody-secreting hybridomas, a monoclonal anti-Dicer interactor or anti-Dicer antibody can be identified and isolated by screening a recombinant combinatorial immunoglobulin library (e.g., an antibody phage display library) with Dicer interactor or Dicer to thereby isolate immunoglobulin library members that bind Dicer interactor or Dicer, respectively. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Patent No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J 12:725-734; Hawkins et al. (1992) J. Mol. Biol. 226:889-896; Clarkson et al. (1991) Nature 352:624-628; Gram et al. (1992) PNAS 89:3576-3580; Garrad et al. (1991) Bio/Technology 9:1373-1377; Hoogenboom et al. (1991) Nuc. Acid Res. 19:4133-4137; Barbas et al. (1991) PNAS 88:7978-7982; and McCafferty et al.Nature (1990) 348:552-554.

An anti-Dicer interactor or anti-Dicer antibody (e.g., monoclonal antibody) can be used to isolate Dicer interactor or Dicer, bioactive portions thereof, or fusion proteins by standard techniques, such as affinity chromatography or immunoprecipitation. Anti-Dicer antibodies or anti-Dicer interactor antibodies made according to any of the above-described techniques can be used to detect protein levels in donor or acceptor fractions as part of certain assay methodologies described herein. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, -galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

VIC.Recombinant Expression Vectors and Assay Cells or Organisms

Another aspect of the invention pertains to vectors, preferably expression vectors, for producing the proteins reagents of the instant invention. As used herein, the term "vector" refers to a nucleic acid molecule capable of transporting another nucleic acid to which it has been linked. A preferred vector is a "plasmid", which refers to a circular double stranded DNA loop into which additional DNA segments can be ligated. In the present specification, "plasmid" and "vector" can be used interchangeably as the plasmid is the most commonly used form of vector.

The recombinant expression vectors of the invention comprise a nucleic acid that encodes, for example protein or Dicer or a bioactive fragment or Dicer interactor or bioactive fragment, in a form suitable for expression of the nucleic acid in a host cell or organism, which means that the recombinant expression vectors include one or more regulatory sequences, selected on the basis of the host cells or organisms to be used for expression, which is operatively linked to the nucleic acid sequence to be expressed. Within a recombinant expression vector, "operably linked" is intended to mean that the nucleotide sequence of interest is linked to the regulatory sequence(s) in a manner which allows for expression of the nucleotide sequence (e.g., in an in vitro transcription/translation system or in a host cell or organism when the vector is introduced into the host cell or organism). The term "regulatory sequence" is intended to includes promoters, enhancers and other expression control elements (e.g., polyadenylation signals). The expression vectors can be introduced into host cell or organisms to thereby produce proteins,including fusion proteins or peptides. Alternatively, retroviral expression vectors and/or adenoviral expression vectors can be utilized to express the proteins of the present invention.

The recombinant expression vectors of the invention can be designed for expression of Dicer interactor or Dicer polypeptides in prokaryotic or eukaryotic cells. For example, Dicer interactor or Dicer polypeptides can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, CA (1990).

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Purified fusion proteins are particularly useful in the cell-free assay methodologies of the present invention.

In yet another embodiment, a protein or Dicer-encoding or Dicer-protein-encoding nucleic acid is expressed in mammalian cells, for example, for use in the cell or organism-based assays described herein. When used in mammalian cells, the expression vector's control functions are often provided by viral regulatory elements. In another embodiment, the recombinant mammalian expression vector is capable of directing expression of the nucleic acid preferentially in a particular cell type (e.g., tissue-specific regulatory elements are used to express the nucleic acid).

Another aspect of the invention pertains to assay cells into which a recombinant expression vector has been introduced. An assay cell can be prokaryotic or eukaryotic, but preferably is eukaryotic. Cell lines are cultured according to art-recognized techniques. Vector DNA can be introduced into prokaryotic or eukaryotic cells via conventional transformation or transfection techniques. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor,NY, 1989), and other laboratory manuals. An assay cell of the invention, can be contacted with a test compound and assayed for any Dicer interactor and/or Dicer biological activity in order to identify the compound as a modulator. Biological activities that can further be assayed as part of the methodologies of the present invention include, but are not limited to, (1) processing of miRNA precursors; (2) processing of siRNA precursors; (3) mediating mRNA cleavage; (4) mediating assembly of RISC (e.g., via siRNAs); (5) directing translation repression (e.g., via miRNAs); (6) a ribonuclease activity (e.g., cleavage of dsRNA); and (7) initiation of RNAi. In addition, other biological activities which may be assayed for include those listed in Table 1 and/or subsections IA-IMM and II, supra.

VII. Pharmaceutical Compositions

This invention further pertains to modulators identified by the above-described screening assays. Modulators identified by the above-described screening assays can be tested in an appropriate animal model. For example, a Dicer modulator, RNAi modulator and/or gene silencing modulator identified as described herein can be used in an animal model to determine the efficacy, toxicity, or side effects of treatment with such a modulator. Alternatively, a modulator identified as described herein can be used in an animal model to determine the mechanism of action of such an agent. Furthermore, this invention pertains to uses of modulators identified by the above-described screening assays for therapeutic treatments as described infra.

Accordingly, the modulators of the present invention can be incorporated into pharmaceutical compositions suitable for administration. Such compositions typically comprise the nucleic acid molecule, protein, antibody, or modulatory compound and a pharmaceutically acceptable carrier. As used herein the language "pharmaceutically acceptable carrier" is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Patent No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The pharmaceutical compositions can be included in a container, pack, or dispenser together with instructions for administration.

VIII.Methods of Treatment

The present invention also features methods of treatment or therapeutic methods. In one embodiment, the invention features a method of treating a subject (e.g., a human subject in need thereof) with a modulatory compound identified according to the present invention, such that a desired therapeutic effect is achieved. In another embodiment, the method involves administering to an isolated tissue or cell line from the subject a modulatory compound identified according to the methodology described herein, such that a desired therapeutic effect is achieved. In a preferred embodiment, the invention features a method of treating a subject having a disease or disorder characterized by overexpression or aberrant expression of a particular protein. For example, positive modulators of Dicer and/or RNAi can be used to enhance RNAi of deleterious proteins. Likewise, negative modulators of Dicer and/or RNAi can be used to alleviate symptoms resulting from the RNAi pathway. Desired therapeutic effects include a modulation of any Dicer protein-, Dicer- or Dicer protein/Dicer-associated activity, as described herein. Desired therapeutic effects also include, but are not limited to curing or healing the subject, alleviating, relieving, altering or ameliorating a disease or disorder in the subject or at least one symptom of said disease or disorder in the subject, or otherwise improving or affecting the health of the subject. A preferred aspect of the invention pertains to methods of modulating Dicer protein/Dicer interactions for therapeutic purposes.

The modulators identified by the methods disclosed herein may be used in a subject to modulate (1) processing of miRNA precursors; (2) processing of siRNA precursors; (3) mediating mRNA cleavage; (4) mediating assembly of RISC (e.g., via siRNAs); (5) directing translation repression (e.g., via miRNAs); (6) a ribonuclease activity (e.g., cleavage of dsRNA); and/or (7) initiation of RNAi.

The effectiveness of treatment of a subject with a Dicer modulator, RNAi modulator and/or gene silencing modulator can be accomplished by (i) detecting the level of activity in the subject prior to treating with an appropriate modulator; (ii) detecting the level of activity in the subject post treatment with the modulator; (iii) comparing the levels pre-administration and post administration; and (iv) altering the administration of the modulator to the subject accordingly. For example, increased administration of the modulator may be desirable if the subject continues to demonstrate undesireable symptoms of the disease or disorder being treated.

IX.Diagnostic Assays

The present invention also features diagnostic assays, for determining aberrant Dicer protein:Dicer interaction, expression or activity, in the context of a biological sample (e.g., blood, serum, cells, tissue) to thereby determine whether an individual is afflicted with a disease or disorder associated with said aberrancy or is at risk of developing such a disorder. The invention also provides for prognostic (or predictive) assays for determining whether an individual is at risk of developing such a disorder (e.g., a disorder associated with aberrant Dicer interactor expression or activity). Such assays can be used for prognostic or predictive purpose to thereby prophylactically treat an individual prior to the onset of a disease or disorder. A preferred agent for detecting a Dicer interactor or Dicer protein is an antibody capable of binding to protein or Dicer, respectively, preferably an antibody with a detectable label. The term "biological sample" is intended to include tissues, cells and biological fluids isolated from a subject, as well as tissues, cells and fluids present within a subject. The invention also encompasses kits for the detection of aberrant Dicer protein:Dicer interaction, expression or activity in a biological sample. For example, the kit can comprise a labeled compound or agent capable of detecting Dicer interactor and/or Dicer in a biological sample; means for determining the amount of Dicer interactor and/or Dicer in the sample; and/or means for comparing the amount of Dicer interactor in the sample with a standard. The compound or agent can be packaged in a suitable container. The kit can further comprise instructions for using the kit.

X. Uses

The invention has several further advantageous uses which include, but are not limited to, the following: providing interacting proteins of Dicer and there use in modulating Dicer function; methods for identifying further interactors of Dicer and their structural and functional characteristics; method for regulating Dicer activity though the use of Dicer interactors; methods for improving the in vitro or in vivo processing of Dicer proteins or for as targets for pharmaceutical intervention in order to modulate the properties of Dicer in vivo for improved RNAi; and methods for stabilizing RNAi agents / compositions comprising Dicer by the addition of stabilizing interactor proteins or the same for use in purifying Dicer and other Dicer components.

This invention is further illustrated by the following examples which should not be construed as limiting. The contents of all references, patents and published patent applications cited throughout this application are hereby incorporated by reference.

Exemplification

Throughout the examples, the following materials and methods were used unless otherwise stated.

Materials and Methods

In general, the practice of the present invention employs, unless otherwise indicated, conventional techniques of nucleic acid chemistry, recombinant DNA technology, molecular biology, biochemistry, cell biology and transgenic animal biology. See, e.g., DNA Cloning, Vols. 1 and 2, (D.N. Glover, Ed. 1985); Oligonucleotide Synthesis (M.J. Gait, Ed. 1984); Oxford Handbook of Nucleic Acid Structure, Neidle, Ed., Oxford Univ Press (1999); RNA Interference: The Nuts & Bolts of siRNA Technology, by D. Engelke, DNA Press, (2003); Gene Silencing by RNA Interference: Technology and Application, by M. Sohail, CRC Press (2004); Sambrook, Fritsch and Maniatis, Molecular Cloning: Cold Spring Harbor Laboratory Press (1989); and Current Protocols in Molecular Biology, eds. Ausubel et al., John Wiley & Sons (1992), which are incorporated in their entireties by reference herein.

C. elegans Strains

Typical C. elegans strains for carrying out the invention as described herein include, for example, N2; alg-2 strain (ok304); dcr-1 for rescue; dcr-1 counterselectable; f20 counterselectable; drh-3 counterselectable; bn-2 (glp-4); rde-4 ne337; eri-1 (mg366); rrf-3 (pk); eri-3 (tm); and eri-5 (mg, tm).

Antibody Development and Purification

Antisera against C. elegans Dicer, i.e., DCR-1, were raised in rabbits as described by Capralogics services (Capralogics, Hardwick, MA, USA). The antisera used for the somatic purifications, and for the immunoblot analyses were developed using a fragment encoded by residues 1145 to 1347 of the protein fused to the pCal-KC (Stratagene) encoded fusion. For their affinity purification, another fragment encoding residues 966 to 1347 was expressed as a pET-42a (Novagen) fusion, purified under denaturing conditions, using Guanidine HCl (Ultra grade, FLUKA) 6M / 150mM NaCl/ HEPES 25mM pH 8.0 as a lysis, binding and washing buffer. The purified fusion was eluted in Guanidine HCl 6M/ 150mM NaCl/ MES 25mM pH4.8 and added directly to the Affigel 10 (Biorad) and allowed to rock O/N for covalent coupling of the fusion.

The matrix was then washed in coupling buffer (5 column volumes) in Tris (10 column volumes) and remaining active sites were blocked using triethanolamine/HCl for 2h at 4 degrees. The matrix was then rinsed extensively in PBS and used for affinity-purification of the antisera. The sera (4ml per batch) were diluted 1:5 in PBS, filtered sterile and loaded directly on the prepared affinity matrixes. After batch binding, the beads were washed extensively with PBS in a column and, the antibodies were eluted (8 column volumes) using glycine 0.2M pH2.2, while harvesting the fractions if 3:7 volumes of Potassium Phosphate solution at pH10, to neutralize the fractions. Consecutive purifications (3) were realized with the same serum batch with similar antibody recovery.

Fractions were then examined by SDS-PAGE, and quantified by comparison with BSA standards. The fractions containing the antibodies were dialyzed against PBS/5% glycerol, and concentrated to ~1 microgram per microliter using the Centricon 10 centrifuge dialysis system (Millipore). The concentrated antibodies were frozen at -80 until used.

Dicer (Dcr-1) Transgenic Rescue

A fragment encoding the 3' portion of the Dicer (dcr-1) gene was cloned into Bluescript SK (Stratagene) and a Not I site was inserted prior to its stop codon. A NotI cassette encoding 8 copies of HA, and the yeast sup4o gene embedded in an artificial C. elegans intron was then inserted in frame at the 3' portion of the recombination cassette. This fusion was then prepared by PCR and used to transform a yeast strain bearing the YAC Y97B3. The strain was then selected on URA-/LYS- for YAC recombinants.

Confirmed recombinants, were screened by PCR and a genomic preparation of the strain was realized. A C. elegans strain bearing dcr-1 (ok247), and the dpy-13 lesions balanced by the sDp-3 free duplication was used for rescue. The genomic DNA was injected in the balanced animals germline at 200ng per microliter with an additional 50ng per microliter of sur-5::gfp expressing vector, as a secondary marker for transformation. Mosaics (F1) were selected on the basis of their GFP signal, singled out, and transmitting lines were identified with regard of the GFP signal of the F2s.

Genomic DNA was then prepared from 2-5 animals of such lines, and examined for the presence of the recombinant YAC DNA by PCR. 4 out of 22 independent transgenic lines had the YAC, and of such, 2 of the strains consistently produced dpy animals with fertile progeny. Single picks from such animals led to dpy populations, in which only GFP+ animals were fertile thereby indicating that the rescue was due to the recombinant YAC.

Fractionation and Immunoblot Analysis

For the somatic purifications, and the RNA analysis, the C. elegans strains were grown in standard conditions, as synchronous populations and harvested as adults with a single row of embryos, or allowed to grow 12h after the L4 to young adult transition, in the case of sterile animals. Animals were rinsed in M9 twice and floated on sucrose if gravid adults were used. Animals were allowed to rock in M9 for 30 min at RT to allow digestion of the gut bacterial load. For embryonic preparations, gravid adults were hypochlorided as previously described, rinsed in M9 three times, and further rinsed in cold water. The animals were then pelleted using a table-top falcon centrifuge, and frozen at -80°C as a compacted pellet after all the supernatant was drained.

Preparations where further processed using one volume of hypotonic buffer, 10mM HEPES KOAc pH 7.5; 10mM K(OAc); 2mM Mg(OAC)₂ ; 1mM DTT with 4x concentration complete protease inhibitors and RNase inhibitors. The suspension was then transferred to a cold Dounce homogenizer, and stroked 20-30 times, on ice. The resulting slurry was then transferred to an Eppendorf, and the recovered volume was adjusted to 110mM KCl (yields Isotonic buffer), vortexed and allowed to sit on ice for 10 minutes.

The nuclear fraction was prepared in the following manner. The slurry was first centrifugation at 1500xg for 30 sec at 4°. The supernatant was recovered and adjusted to 10% glycerol, 0.01% Triton X-100, and vortexed and allowed to sit on ice for 10 min. The slurry was then loaded on a sucrose cushion (10mM HEPES pH7.5; 10mM KCl; 1M sucrose; 10% glycerol; 1mM EDTA), and centrifuged at 20000xg for 10 min at 4°. The pellet yields the nuclear fraction.

The S100 and P100 fractions were prepared as follows. The supernatant from a short 1500xg centrifugation was further centrifuged at 10000xg for 10min at 4°, and the supernatant was recovered (S10 fraction). This fraction was then loaded in a Beckman microfuge polyallomer tube and further spun 1h 4°C in a TLA100.3 to yield the S100 and P100 fractions. Equivalent volumes of each fraction was precipitated in 2 volumes acetone and resuspended in 1x SDS-PAGE buffer for the fraction analyses.

Immunoblots were realized using PBS/0.1%tween/5% milk for blocking and blotting, and PBS/0.1%tween for washings. Primary antibodies were incubated at RT for 1h, and the corresponding HRP-coupled secondaries were used at 1:5000 for 1h before 3x 5min washes and ECL development (Pierce).

Purifications and Immunoprecipitations

Immunoprecipitation matrixes were prepared by DimethylPimeliimidate (DMP) (Sigma) covalent coupling to rProtA-agarose beads (Pierce) in sodium borate pH9.0 buffer. The beads were then stripped and blocked in 0.2M glycine pH2.2, rinsed extensively in PBS and kept until use at 4°C with thymerosal as antibacterial agent. For typical preparations, 1mg purified polyclonal antibodies were covalently coupled to 200ul rProtA beads. In the case of embryonic purifications, agarose coupled matrixes from both antibody clones were used.

For the Dicer (DCR-1) purifications, the S100 fraction was further quantified and diluted to 3mg per ml concentration in 1% Triton X-100 supplemented Isotonic buffer before the suitable buffer-equilibrated matrixes (30ul bead volume per 2ml IP) were added to the mixtures. Immunoprecipitations were carried out at 4°C for 1h, and beads were then washed 3 times in the immunoprecipitation buffer.

Immunoprecipitates were then treated with 20ug per ml RNaseA for 30 minutes on ice in the same buffer, then washed three more times. The beads were washed one more time in cold PBS, and all the supernatant was drained. Bound proteins were eluted in 8M urea/ 50mM HEPES pH7.5, and acetone-precipitated. 1/5^{th} the elution volume was kept and monitored on silver stain and/or by immunoblot for a qualitative evaluation of the immunoprecipitation process.

RNA Interferences

Feeding and microinjection RNAi was carried out as previously reported by Conte Jr. D. and Mello, C.C. 2003. RNA interference in Caenorhabditis Elegans. In Current Protocols in Molecular Biology.

Northern Blotting and Real Time PCR

Small RNAs were prepared from N2(wt), bn2(glp4), which lack a germline tissue, and mutants for rde-4(ne337), rrf-3(pk1426), eri-1(mg366), eri-3(tm1361), and eri-5(mg370), all at 25°C. Homozygous dcr-1(ok247), f20d12.1, and drh-3(tm1217) sterile adult animals were isolated using the counterselectable genetic balancer method. For alg-1 and alg-2 depleted preparations, alg-2(ok204) L1 animals were exposed to an alg-1(rnai) feeding strain, and the bursting young adults were harvested and used for small RNA preparations. The isolated small RNA preparations were typically examined by Northern blotting for a variety of endogenous small RNAs as well as miR58, tncR7 ,and the X chromosome locus contig of small RNAs described in the art. Real time PCR was performed with primer pairs having efficiencies validated for a multiple10 fold dilution range around the N2(wt) level, and fold changes were calculated using the delta delta Ct method.

Imaging and Video Microscopy

DAPI staining of intact animals was done as described in the art. Endomitotic (Emo) phenotype was scored by intense and irregular DAPI staining or expression of histoneH2::gfp in germ cell nuclei. Nematode gonads were dissected as described in the art with slight modifications. Briefly, young adult worms were placed in a drop of PBS containing 0.15mM of levamisole on a glass slide for gonad extrusion. The dissected gonads were then fixed in 4% paraformaldehyde in PBS for 5 minutes, followed by three washes of PBS before staining with DAPI for 5 minutes. Gonads were then mounted for imaging after 3 washes with PBS. DIC or fluorescence images were collected by a Hamamatsu Ocre-ER digital camera mounted on a Zeiss Axioplan 2 under the control of Openlab 3.0 software. In time-lapse video microscopy experiments, young adult animals expressing a histoneH2::gfp fusion in the germline (AZ212) were cut open in M9 solution and embryos were mounted on 2% agarose pads in M9 solution for recording by a Leica TCS SP2 confocal microscope system. Movies were processed on a Macintosh computer using the public domain Image J 10.2 program (developed at the U.S. National Institutes of Health.

Multi Dimensional Protein Identification Technology

The MudPIT assays were performed essentially as described in Graumann et al., Mol Cell Proteomics, 3(3):226-37 (2004) and Liu et al., Biotechniques. 32(4):898, 900, 902 passim (2002).

Example 1.Methods for Identifying Dicer Interacting Proteins

To identify Dicer interacting proteins a protenomic approach was employed. In particular, a combined and comparative proteomic approach was designed and used to identify novel factors implicated in molecular interactions with Dicer (DCR-1) in the nematode C. elegans. The approach featured a combined transgenic and immuno-biochemical purification scheme with an innovative Mass Spectrometry technology called MudPit (Multi-dimensional protein identification technology) in order to identify proteins interacting with DCR-1 in the embryo and in the adult of the animal and compared with the interactors identified in parallel as being interactors of the RDE-4 and RDE-1 proteins. The MudPit technology has been previously described (see, e.g., Graumann et al., Mol Cell Proteomics, 3(3):226-37 (2004); Liu et al., Biotechniques. 32(4):898, 900, 902 passim (2002)).

Using this approach, several interactions were identified which have important significance as to how DCR-1 can be up- or down-regulated and how DCR-1 is implicated in different functions. Table 1 lists the DCR-1 interactors identified using the above approach. Table 2 shows the corresponding protein interaction data obtained for each interactor. Many of these interactors are widely conserved and have homologs in other species such as human or mouse. These interactors are implicated as activators or inhibitors of the DCR-1 activity, specificity, and/or stability and can be utilized for improved in vitro processing of a variety of DCR-1 proteins. The interactors can also be used as part of a rationale or also as targets for pharmaceutical intervention in order to modulate the properties of DCR-1 in vivo.

Table 1. List of Dicer Interacting Proteins Identified in Pilot Scale

| | | | |
|---|---|---|---|
| § | Protein CE# | Description | Phenotype |
| IIA | | RDE-4 | RDE |
| IIB | | ALG-1 | EXP |
| IIC | | ALG-2 | EXP |
| IID | | DRH-1 | RDE |
| IIE | | DRH-2 | RDE |
| IIF | ce09069 | helicase homologous to | ND |
| | | DCR-2 | |
| IIG | ce21971 | Double helicase | EMB |
| IIH | | EFT-2 EF-Tu family GTP | EMB; Pvl; Ste; Lva |
| | | binding | |
| III | | EFT-4 (elF1 alpha) | EMB; Gro; Lva; Unc; |
| | | | Ste |
| IIJ | ce21437 | GAP/RAN-GAP family | ND |
| IIK | ce08872 | HMG-I/Y DNA binding | WT |
| IIL | ce20336 | HMG-I/Y DNA binding | Dpy; EMB; Lvl; Ste; |
| | | PB1 domain | Unc; Lva |
| IIM | ce14704 | SNR-2 SM protein | EMB; Ste; EXP; Lva |
| IIN | ce02065 | SNR-3 SM protein | EMB; Clr; Sck; Lva |
| IIO | ce03706 | Dual specificity | EMB; EXP |
| | | phosphatase | |
| IIP | | LIN-41 | |
| IIQ | ce001506 | low homology MADS box, | GRO |
| | | novel | |
| IIR | | RPN-9 proteasome subunits | EMB; BMD, Unc, |
| | | | Gro, Lva |
| IIS | ce14736 | TAF 6.1 | WT (ND) |
| IIT | ce05915 | T54 homology | Unc Stp Gro |
| IIU | ce21988 | RRM protein (3 domains) | ND |
| IIV | ce27223 | Worm unique/Novel | WT (ND) |
| IIW | ce00850 | TBB-4 | EMB |
| IIX | | RPS-14 | |
| IIZ | | RPS-13 | |
| IIAA | | RPL-24 | |
| IIBB | | RPS-11 | |
| IICC | ce03050 | Agglutinin | |
| IIDD | | SIP-1 (hsp20) | WT |
| IIEE | | CCT-6 (chaperonin) | |

Table 2. List of Dicer:Dicer Interacting Protein Interaction Results

| | | | | | | | | |
|---|---|---|---|---|---|---|---|---|
| | | IP | | | | | IP | |
| | | Con- | IP | IP | IP | IP | 1001(2)- | R4 (2)- |
| § | description | trols | 1 | 2 | 3 | 4 | ctls | ctrl |
| IIA | RDE-4 | NP | P | P | P | P | P | P |
| IIB | ALG-1 | NP | | P | P | P | P | |
| IIC | ALG-2 | NP | | P | P | P | P | |
| IID | DRH-1 | NP | P | P | P | P | P | p |
| IIE | DRH-2 | NP | P | P | P | P | P | P |
| IIF | helicase | NP | | | P | P | P | |
| | homologous to | | | | | | | |
| | DCR-2 | | | | | | | |
| IIG | Double helicase | NP | P | P | P | P | | |
| IIH | EFT-2 EF-Tu | NP | | P | P | P | P | P |
| | family GTP | | | | | | | |
| | binding | | | | | | | |
| III | EFT-4 (elF1 | NP | | P | P | P | P | |
| | alpha) | | | | | | | |
| IIJ | GAP/RAN-GAP | NP | | P | P | P | | |
| | family | | | | | | | |
| IIK | HMG-I/Y dna | NP | | P | P | P | | |
| | binding | | | | | | | |
| IIL | HMG-I/Y dna | NP | | | P | P | | |
| | binding PB1 | | | | | | | |
| | domain | | | | | | | |
| IIM | SNR-2 SM | NP | | | P | P | | |
| | protein | | | | | | | |
| IIN | SNR-3 SM | NP | | | P | P | P | |
| | protein | | | | | | | |
| IIO | Dual specificity | NP | | P | P | P | P | |
| | phosphatase | | | | | | | |
| IIP | LIN-41 | NP | | | P | P | P | |
| IIQ | low homology | NP | P | P | P | P | P | |
| | MADS box, | | | | | | | |
| | novel | | | | | | | |
| IIR | RPN-9 | NP | | P | P | P | | |
| | proteasome | | | | | | | |
| | subunits | | | | | | | |
| IIS | TAF 6.1 | NP | | | P | P | P | |
| IIT | T54 homology | NP | | | P | P | | |
| IIU | RRM protein | NP | | | P | P | | |
| | (3 domains) | | | | | | | |
| IIV | Worm | NP | | | P | P | P | |
| | unique/Novel | | | | | | | |
| IIW | TBB-4 | NP | P | P | P | P | | P |
| IIX | RPS-14 | NP | | P | P | P | | |
| IIZ | RPS-13 | NP | P | P | P | P | | |
| IIAA | RPL-24 | NP | | | P | P | | |
| IIBB | RPS-11 | NP | P | P | P | P | | |
| IICC | Agglutinin | NP | | | P | P | | |
| IIDD | SIP-1 (hsp20) | NP | | P | P | P | | |
| IIEE | CCT-6 | NP | | P | P | P | | |
| | (chaperonin) | | | | | | | |

NP = not present; P = present; IP = immunoprecipitation; 1 to 4 corresponds to four independent purifications with that affinity matrix; 1001 = second antibody matrix; R4 (2) -ctls = interactors found in two independent purifications of RDE-4, absent from the controls, and also present in the DCR-1 purifications.

Example 2.Methods for Conducting a Whole Organism Search for Dicer Interactions

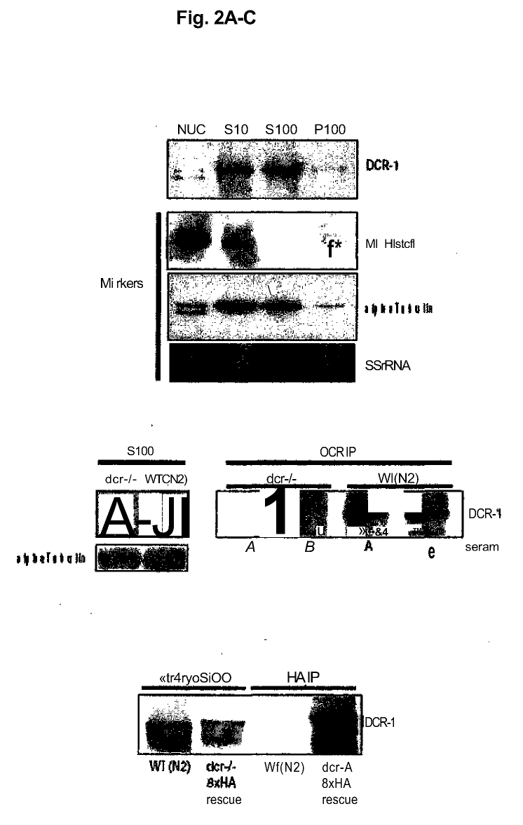

In order to identify Dicer interacting proteins in a whole organism, strategies to affinity-purify Dicer (DCR-1) by multiple independent matrixes, both from embryos and gravid adults C. elegans, were developed. Fractionation analysis showed that most, if not all the C. elegans Dicer protein can be found in the S100 fraction (Figure 2).

For the adult purification, rabbit polyclonal sera having efficient immunoprecipitation capacity for the Dicer protein were identified. The antisera were affinity-purified against their respective antigen, coupled covalently to agarose matrixes, and used for batch immuno-affinity purifications. Controls included preparations from extracts genetically null for any Dicer expression (dcr-1 deletion allele (ok247)), or mock purification comprising neutralized affinity beads.

For the embryonic purifications, a transgenic dcr-1::8xHA genomic fusion driven by its own promoter, was used. The transgene allowed the sterility phenotype of dcr-1-/- to be rescued, and a robust expression in young embryos indicating it can support the functions of DCR-1 in the germline. Purification of DCR-1::8xHA fusion protein was carried out using two distinct monoclonal HA-directed affinity matrixes, and used the non-transgenic WT (N2) embryos as a control.

The purified proteins were eluted and analyzed using the Multi-Dimensional Protein Identification Technology (MudPIT). Interacting proteins were identified by comparison of the detected peptides with both the predicted and confirmed ORF library of the C. elegans genome. Protein candidates were not investigated further if they were also found in the depleted control, or in the mock purification (uncoupled matrix only). Chaperones, and two structural proteins, which were found in multiple non-related purifications, and known to be common non-specific interactions, were intentionally excluded. A high confidence set of interactions for proteins that could be detected in multiple purifications, with at least two independent matrixes, was defined. Using this strategy, 16 proteins were shown to interact with DCR-1. Table 3 depicts the interactions that were detected using this criteria.

Table 3. List of Dicer Interacting Proteins

(DEAD disclosed as SEQ ID NO: 14; DEAH disclosed as SEQ ID NO: 15)

| | | DCR-1 | | |
|---|---|---|---|---|
| | gene | purifi- | | |
| | name | cation | structural description | Phenotype |
| 1FF | RDE-1 | E*W | Piwi/PAZ domain | Rde |
| 1A | RDE-4 | EA**W | dsRBD | Rde |
| 1D | DRH-1 | EAW | DEAH/D box | Rde |
| 1E | DRH-2 | EAW | DEAH/D box | Rde |
| 1GG | D2005.5 | EA | DEAH/D box | early |
| | (DRH-3) | | | embryonic |
| | | | | arrest, |
| | | | | sterile |
| 1HH | ERI-1 | E | SAP domain, Exonuclease | ts sterile, |
| | | | | eri |
| 1II | RRF-3 | E | rdrp | ts sterile, |
| | | | | eri |
| 1JJ | W09B6.3 | EA | Novel (operon and fusion with | ts sterile, |
| | (eri-3) | | TAF-6.1) | eri |
| 1KK | Y38F2AR.1 | EA | TUDOR domain | ts sterile, |
| | (eri-5) | | | eri |
| 1S | TAF-6.1 | EA | TATA box binding protein | ND; eri |
| | | | associated factor (operon and | |
| | | | fusion with eri-3) | |
| 1B | ALG-1 | AW | Piwi/PAZ domain | hetero- |
| | | | | chronic |
| 1C | ALG-2 | AW | Piwi/PAZ domain | hetero- |
| | | | | chronic |
| 1P | LIN-41 | A | RBCC (NHL family) | hetero- |
| | | | | chronic, |
| | | | | pleiotropic |
| 1LL | T23G7.5 | EA | Phosphatase | rde, L4 |
| | (PIR-1) | | | develop- |
| | | | | mental |
| | | | | arrest |
| 1H | EFT-2 | A | EFT-2 family GTPases | lethal, |
| | | | | pleiotropic |
| 1N | SNR-3 | A | SM domain | lethal, |
| | | | | pleiotropic |
| 1MM | C32A3.2 | A | Novel | WT |

Abbreviations are as follows: E : embryonic purification, A : gravid adult purification, W: western detection, rde:required for RNAi; eri:enhancer of RNAi; * weak peptide coverage only, ** due to the robust interaction, weak peptide coverage of RDE-4 is also detected in the dcr counterselected allele, likely due to interaction with the maternal load.

Proteins known to be involved in the initiation step of RNAi were found in all the DCR-1 purifications. The double-stranded binding protein RDE-4 was shown to interact with DCR-1. RDE-4 was also shown to interact with the argonaute family protein RDE-1, and the helicase DRH. In addition, RDE-4, DRH-1 and DRH-2 proteins were detected as interactors when pulling down with DCR-1.

In addition to the proteins involved in initiation of RNAi, other proteins having characterized functions that relate to small RNAs, were detected. Two argonaute proteins, ALG-1 and ALG-2, were also detected in the adult DCR-1 purifications. These paralog proteins are required for the efficient processing of a variety of miRNA precursors, but were heretofore unknown to interact physically with DCR-1.

Interactions with the rdrp RRF-3, and the SAP domain ERI-1 nuclease were also detected in the embryonic purifications. Interestingly when the genes coding for these proteins were inactivated, an enhancement of the classical RNAi response is observed (eri phenotype) indicating that rrf-3 and eri-1 encode negative regulators of RNAi.

In addition, the protein D2005.5 was detected which did not have a characterized small RNA-related function, but is a paralog of the dicer-related helicases drh-1 and drh-2.

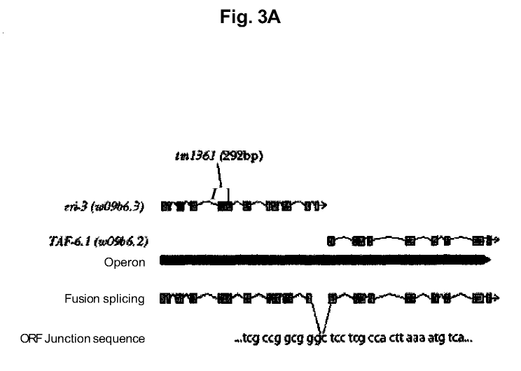

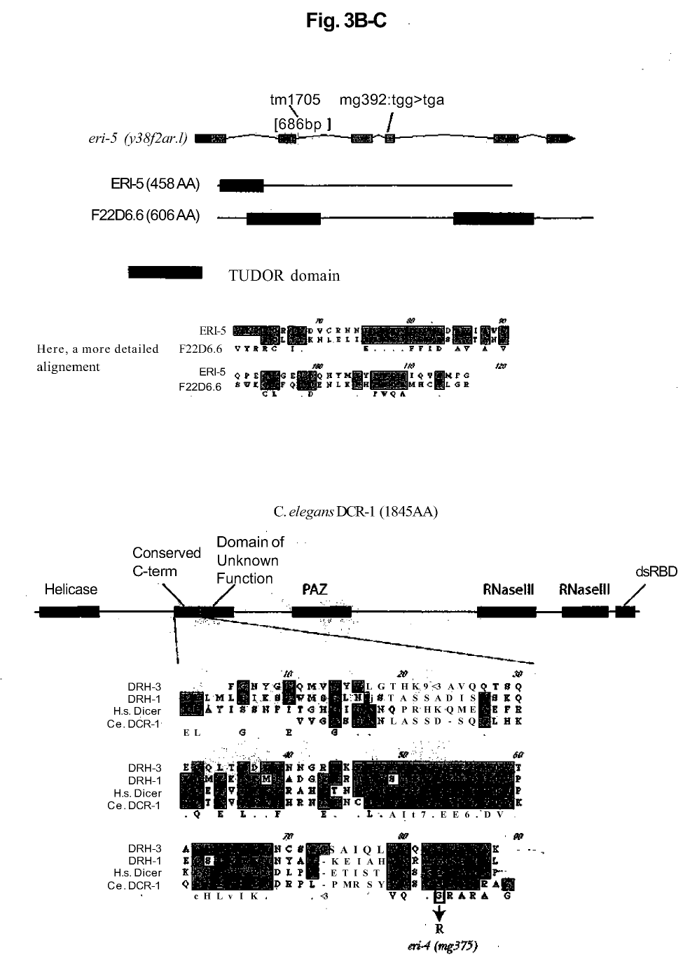

For eight other proteins, no previous link with DCR-1 functions, or with small RNA-mediated silencing was known. Four detected proteins have known functions: snRNP core protein D1 (SNR-3), the translation elongation factor 2 (EFT-2), the NHL family ring finger-B box-Coiled coil translational repressor LIN-41, and subunit TAF6 of the transcription initiation factor TFIID (TAF-6.1). Finally four others have unknown functions. This subgroup includes T23G7.1 (an ortholog of the mammalian PIR-1), the novel proteins C32A3.2, W09B6.3 (expressed as an operon with TAF-6.1), and the TUDOR domain protein Y38F2AR.1 (Fig. 3)

Example 3.Methods for Determining In Vivo Activity of Dicer Interacting Proteins

To address the possible functions of these proteins in DCR-1-related mechanisms, the phenotypes of the rnai knock down for their corresponding genes (Table 3) was examined. The genes snr-3 and eft-2 (rnai) demonstrate pleiotropic phenotypes and growth defects. For the remaining Dicer interacting proteins, deletion alleles for d2005.5 (tm1217), t23g7.5 (tm1496), c32a3.2(tm1314), w09b6.3(tm1361) and y38f2ar.1(tm1705) were generated.

In addition, because two interacting protein were encoded by eri genes, the location of the genes encoding the interacting proteins with the mapping intervals of alleles generated in a screen for mutants that increase sensitivity of a neuronal gfp reporter to gfprnai, were generated. Using this strategy three genes, TAF-6.1, w09b6.3 (part of a common operon), and y38f2ar.1 were mapped within the intervals of the eri-3 and eri-5 mutations, respectively. These three gene sequences were found to comprise nonsense point mutations. In addition, another enhancer (eri-4 (mg375)) mapped in proximity to DCR-1, and a point mutation (glycine 351 to arginine) was discovered in the C-terminus extremity of the conserved C-terminus sub-domain of its helicase domain.

The potency of the RNAi activity in whole animals, either for enhancement or deficiency (Table 2), was examined. First, their response in a high sensitivity unc-22 (rnai) somatic (Po) assay, was determined. This assay revealed that the interactor T23G7.5 allele exhibited a drastically reduced sensitivity to rnai when assayed in the soma, both for endogenous unc-22 (rnai) silencing and for gfp (rnai) silencing of a transgenic reporter. Possibly due to the maternal load the effect on RNAi was important, but partial. The mutant on itself also presents developmental defects: the homozygous null grows normally and suddenly arrests at the L4 stage, never reaching adulthood. A generalized loss of gene expression could not be responsible for the lack of RNAi response, as the arrested animals could still transcribe and translate a reporter de novo to a WT level. This protein encodes a conserved RNA phosphatase with homology to a family of capping enzymes, and associates with RNP particles in mammalian culture cells. Its enzymatic activity was shown to have specificity toward the removal of the β- and γ-phosphate residues on the 5' end of triphosphate RNA substrates. This interaction was consistently detected both in the adult and embryonic purifications of DCR-1, and indicating its role in RNAi mechanisms. Thus, T23G7.5 was determined to be essential for development and RNAi

Example 4.Methods for Determining the In Vivo RNAi Enhancer Activity of Dicer Interacting Proteins

The Dicer interacting proteins, w09b6.3 and y38f2ar.1 were determined enhancers of RNAi. Briefly, mutants using rnai targets, which do not exhibit a phenotype, or exhibit a very low penetrance in the WT (N2) genetic background, were assayed to test the possibility that these genes encode enhancers of rnai (eri). As observed, unc-73 (rnai) does not usually exhibit a strong penetrance when wt (N2) animals are exposed (4+-4%). As previously observed, eri-1(mg366) and rrf-3(pk1426) gave a very penetrant effect when exposed to unc-73 E. coli feeding strain (98+-2%). Similarly, a drastically higher penetrance was observed in the w09b6.3 (tm1361), y38f2ar.1 (mg392), and DCR-1(mg375) alleles (100%, 82.5+-11%, and 100%, respectively). Injection assays for lin-1 (rnai), and feeding assays for dpy-13 (rnai), hmr-1(rnai), or gfp(rnai) also supported these observations (not shown). Thus, it was concluded that the w09b6.3, and y38f2ar.1 mutations are enhancers of rnai (eri).

Example 5.Methods for Determining the In Vivo Developmental Effects of Dicer Interacting Proteins

The Dicer interacting proteins eri-3, eri-4 and eri-5 were determined to have similar developmental defects. In addition to the similar effect on rnai, the rrf-3 and eri-1 genes were previously shown to have indistinguishable developmental defects and to act in the same genetic pathway. Known developmental defects include a strong sterility phenotype at 25°C, which is rescued at 15°C, or by crossing with WT males, suggestive of sperm defects. Mutant animals also exhibit spontaneous silencing of some simple transgenic arrays in the soma and a low incidence of X chromosome non-disjunction, visible by a High Incidence of Males (HIM) phenotype.

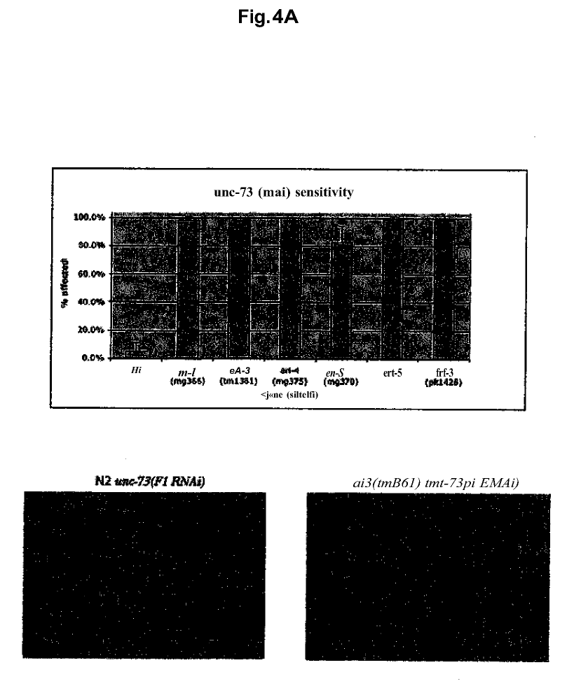

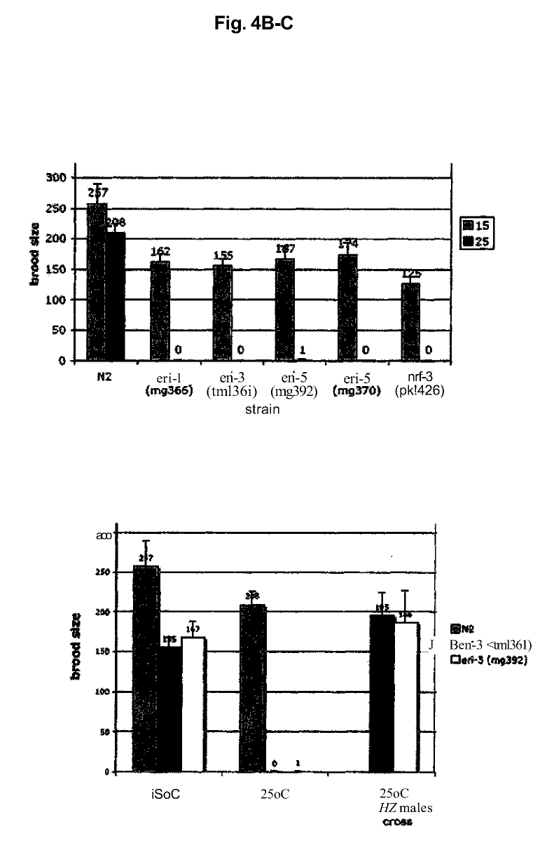

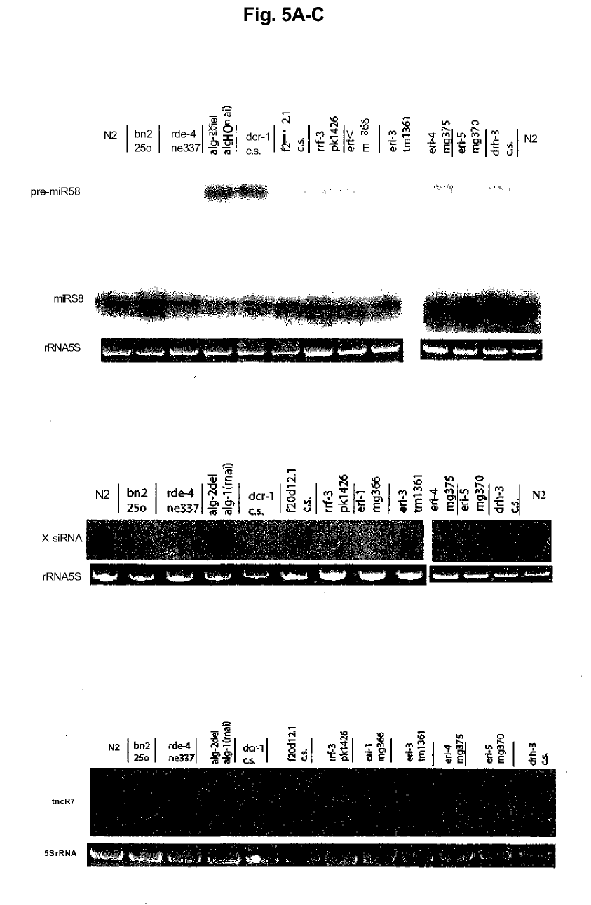

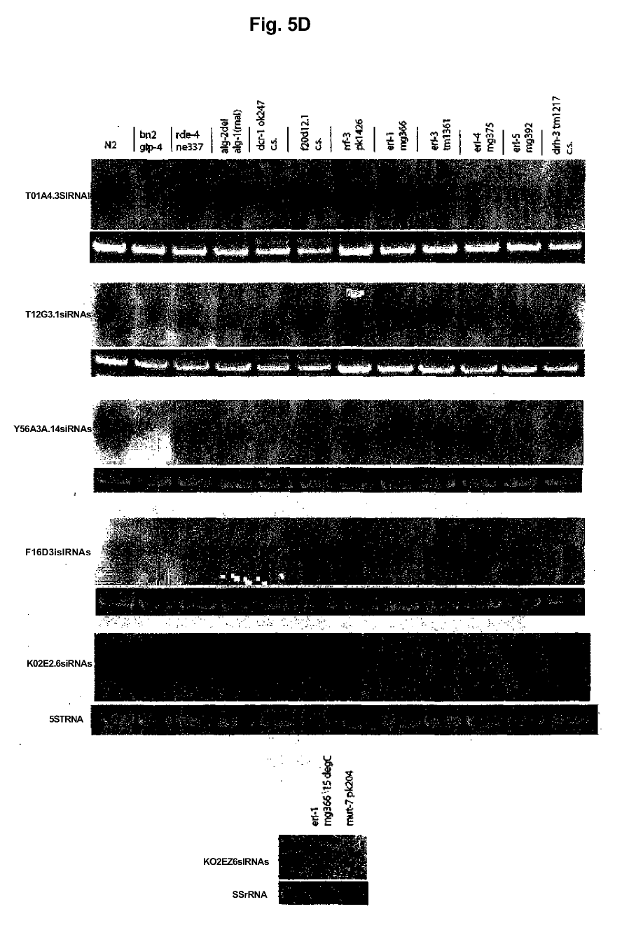

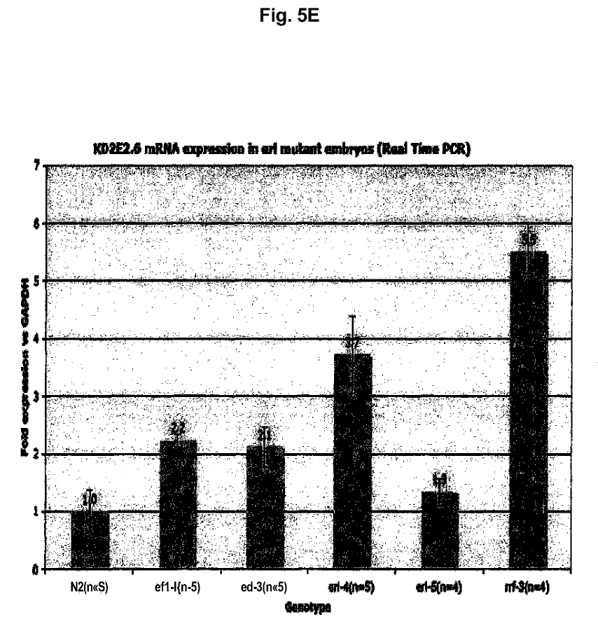

To test the idea that eri-3, eri-4 and eri-5 were acting on the same developmental mechanism, the defects associated with their corresponding alleles, were examined. Akin to alleles of rrf-3 and eri-1, mutations in these two genes led to mean brood sizes of 0+/-1 for w09b6.3 and of 1+/-1 for y38f2ar.1 -/- animals at 25°C. In contrast, at 15°C the same alleles gave mean brood sizes of 155+/-12 for w09b6.3 and 167+/-20 for y38f2ar.1. Interestingly, as what was observed for rrf-3 and eri-1 alleles, the temperature sensitive sterility phenotype of w09b6.3 and y38f2ar.1 can be rescued by crossing with wt males, and therefore believed to be defective in sperm function. Additionally, a 3 to 5 fold increase in incidence of males was also observed in the corresponding mutants, compared to the WT(N2) spontaneous incidence (~0.1%). Altogether, the unique, and specific combination of defects observed in the eri-3, eri-4 and eri-5 mutants indicates their involvement in a common pathway with eri-1 and rrf-3 (Fig. 4)

Example 6.Methods for Determining the In Vivo Helicase/Chromosomal Effects of Dicer Interacting Proteins

The Dicer interacting protein DRH-3, when depleted, was determined to cause sterility and chromosome segregation defects. Mutations in the gene encoding the DCR-1-interacting protein D2005.5 also led to dramatic fertility defects. Because it encodes a paralog of the DEAX/D box helicase drh-1 and drh-2, this Dicer interacting protein was renamed drh-3. In contrast, despite the close homology, drh-3 is not required for initiation of the classical RNAi pathway, at least in the soma where it could be examined (see Table 3). Instead, while the drh-3(tm1217) allele animals were sterile as homozygous and examination of a pie-1::his-3::gfp transgenic strain revealed abnormally shaped oocytes with proximal mitosis, and occasional occurrence of multinucleation, rnai depletion led to a slow onset, but penetrant early embryonic arrest (Table 3). Although the terminal phenotype of this arrest was variable, the observed embryonic arrest was progressively earlier in embryos laid in periods extending two or three days after the adult injection. Interestingly and consistently with the phenotype exhibited in the deletion mutant, earlier injection of Po animals (L3 or L4 animals) also led to sterility. The earliest defects in the affected embryos using time-lapse videomicroscopy were also characterized. As observed, the first cell division was abnormal, and chromosomes lagging on the mitotic spindle could be observed at metaphase. Chromosome segregation later resulted in abnormally shaped nuclei.

Thus, it was noted that this initial developmental defect resembled the observed defects described in S. pombe for mutants in the RNAi machinery.

Example 7.Methods for Determining the In Vivo Effects of Dicer Interacting Proteins on the Accumulation of Endogenous Small RNAs

The Dicer interacting protein drh-3 and the eri are required for the accumulation of classes of endogenous small RNAs.

Because divergent phenotypes were observed in many genes encoding the different DCR-1 interacting proteins, different phenotypic groups would be reflected by defects in accumulation and/or processing of different classes of small RNA. To test this hypothesis, the status of 5 classes of small RNAs known to require dcr-1 for efficient production, in the mutants generated, was examined. Sensitivity to exogenous dsRNA-triggering was used as a functional output for involvement in the classical RNAi pathway. Also examined, was the processing of miRNA precursors, the accumulation of the tncR, and small RNA-derived from an X chromosome-derived contig. Finally, accumulation of endogenous siRNAs (endo siRNAs) for a variety of loci previously shown to naturally produce these small RNAs, was examined

Because dcr-1 -/-, and drh-3-/- are sterile, a counter-selectable balancer strategy to select for nulls within large populations of animals, was employed. The maternal load of the two gene products was sufficient to lead the animals through early development and sterile adults could be studied. To look for an alg-1 / alg-2 depletion effect on small RNAs, animals depleted of alg-1 by rnai in an alg-2-/- (ok304) animal background, were used.

A variety of miRNA were examined and no defects in the mature form accumulation nor in precursor processing was observed in the rde-4, the eri, nor in the drh-3 mutants. In contrast, a moderate to strong miRNA precursor accumulation was visible in alg-1/2, and dcr-1 depleted animals. These results indicate that these two proteins are crucial to most, if not all the miRNA maturation. However, the effect observed here on the precursor accumulation depends on the timing of the miRNA transcription and export relative to the depletion of ALG-1 protein by RNAi or the turnover of the maternal load of DCR-1 in the counterseleted F1 nulls.

Examination of the small RNA populations in the drh-3 counter-selectable nulls revealed that, while this protein is dispensable for normal accumulation of miRNAs, X-derived small RNAs, or the examined tncRs, it is required for the accumulation of all the examined ORF-derived endo-siRNA. Acting as controls, alg-depleted animals, and another counterselectable sterile mutant f20d12.1 did not show such defects in accumulation of these small RNAs. While most of the endo-siRNAs detected were only detected in the germline, drh-3 was also required for the production of soma-derived endo-siRNA k02e2.6. This result implicates the DCR-1-interacting protein DRH-3 specifically in the production and/or stabilization of a broad range of the ORF-derived endogenous-siRNAs.

The five eri mutants exhibited very consistent molecular defects in the accumulation of the mature small RNAs. While they enhanced the classical RNAi response (when triggered from exogenous sources of dsRNA), their mutation prevented accumulation of the examined tncR, and of the X locus-derived small RNAs. Interestingly, rde-4 was also required for the accumulation of the X locus-derived small RNAs, but dispensable for the tncRs or the ORF-derived endo-siRNAs, showing the modulatory nature of the contribution of the DCR-1 interactors for production of diverse small RNA classes.

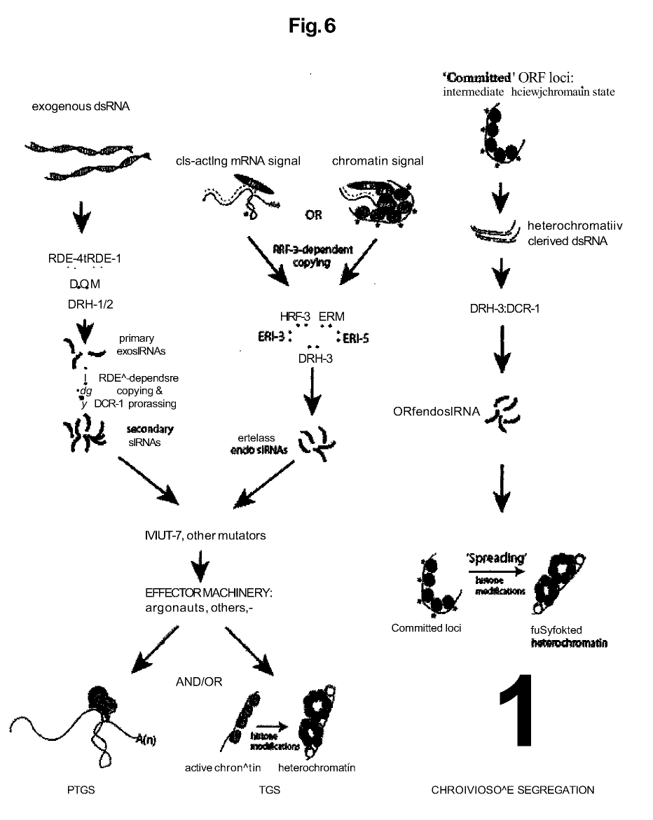

The eri mutations did not affect the accumulation of most of the endo-siRNAs. However, surprisingly, the eri mutants also failed to accumulate endo-siRNAs from a very restricted number of genes. Interestingly, the eri genes also exhibited this defect at the permissive temperature in gravid adults, showing that the developmental process involving the eri genes, and not their function in endo-siRNAs accumulation is a temperature-sensitive process. This observation, and the presence of these endo-siRNAs in germline-less animals (Figure 6, bn2(glp-4)) rules out the idea that the eri genes fail to show these endo-siRNAs because they lack the tissue where they are produced.

These results support the idea that different combinations of DCR-1-interacting proteins are required for efficient accumulation of distinct classes of endogenous small RNAs. These results support a function for the DCR-1-interacting ERIproteins in the initiation of a variety of distinct endogenous small RNA-mediated silencing mechanisms (Fig. 6).

Equivalents

Those skilled in the art will recognize, or be able to ascertain using no more than routine experimentation, many equivalents to the specific embodiments of the invention described herein.

What is Claimed is:

1. A method of identifying a Dicer interacting protein comprising,
contacting a composition having a candidate Dicer interacting protein or bioactive fragment thereof with a Dicer protein or a bioactive fragment thereof, and
determining the presence of one or more proteins capable of interacting with Dicer, as compared to an appropriate control, such that a Dicer interacting protein, is identified.

2. A method of identifying a modulator of Dicer activity comprising,
contacting a composition comprising Dicer or a bioactive fragment thereof and a Dicer interacting protein or a bioactive fragment thereof with a test compound, and
determining the ability of the test compound to modulate activity between Dicer or bioactive fragment thereof and the Dicer interacting protein or bioactive fragment thereof, such that a modulator of Dicer activity, is identified.

3. The method of claims 1 or 2, wherein Dicer or bioactive fragment thereof is derived from an organism selected from the group consisting of nematode, fruit fly, mouse, rat, and human.

4. The method of claims 1 or 2, wherein the Dicer interacting protein or bioactive fragment thereof is derived from an organism selected from the group consisting of nematode, fruit fly, mouse, rat, primate, and human.

5. The method of claim 2, wherein the Dicer activity is selected from the group consisting of protein:protein binding activity, miRNA maturation activity, RNAi initiation activity, RNAi enhancer activity, helicase activity, RISC activity, target recognition activity, and target gene cleavage activity.

6. The method of claims 1 or 2, wherein the composition comprises an extract selected from the group consisting of a cellular extract, nuclear extract, cytoplasmic extract, protein extract, S100 fraction, partially purified protein extract, and purified protein extract.

7. The method of claims 1 or 2, wherein the Dicer interacting protein is selected from the group consisting of RDE-4, ALG-1, ALG-2, DRH-1, DRH-2, helicase homologous to DCR-2 (DRH-3), double helicase, EFT-2 EF-Tu family GTP binding, EFT-4 (eIF1 alpha), GAP/RAN-GAP family, HMG-I/Y DNA binding, HMG-I/Y DNA, binding PB1 domain, SNR-2 SM protein, SNR-3 SM protein, Dual specificity phosphatase, LIN-41, low homology MADS box, novel, RPN-9 proteasome subunits, TAF 6.1, T54 homology, RRM protein (3 domains), Worm unique/Novel (ce27223), TBB-4, RPS-14, RPS-13, RPL-24, RPS-11, Agglutinin, SIP-1 (hsp20), CCT-6 (chaperonin), RDE-1, DRH-3, ERI-1, RRF-3, ERI-3, ERI-5, PIR-1, C32A3.2, and orthologs, paralogs, or bioactive fragments thereof

8. The method of claims 1 or 2, wherein the Dicer interacting proteins are further subjected to a multidimensional protein interaction technology (MudPIT).

9. A method of identifying a modulator of Dicer activity, comprising contacting a cell or cell extract having Dicer or a bioactive fragment and a Dicer interacting protein or bioactive fragment thereof, with a test compound and determining the ability of the test compound to modulate an activity selected from the group consisting of protein:protein binding activity, miRNA maturation activity, RNAi initiation activity, RNAi enhancer activity, helicase activity, RISC activity, target recognition activity, and target gene cleavage activity.

10. The method of claim 9, wherein said cell or cell extract comprises recombinantly expressed Dicer.

11. The method of claim 9, wherein said cell or cell extract comprises a Dicer interacting protein selected from the group consisting of RDE-4, ALG-1, ALG-2, DRH-1, DRH-2, helicase homologous to DCR-2 (DRH-3), double helicase, EFT-2 EF-Tu family GTP binding, EFT-4 (eIF1 alpha), GAP/RAN-GAP family, HMG-I/Y DNA binding, HMG-I/Y DNA, binding PB1 domain, SNR-2 SM protein, SNR-3 SM protein, Dual specificity phosphatase, LIN-41, low homology MADS box, novel, RPN-9 proteasome subunits, TAF 6.1, T54 homology, RRM protein (3 domains), Worm unique/Novel (ce27223), TBB-4, RPS-14, RPS-13, RPL-24, RPS-11, Agglutinin, SIP-1 (hsp20), CCT-6 (chaperonin), RDE-1, DRH-3, ERI-1, RRF-3, ERI-3, ERI-5, PIR-1, and C32A3.2.

12. The method of claim 9, wherein the ability of the test compound to modulate enhancers of RNAi is determined.

13. The method of claim 9, wherein the Dicer or bioactive fragment thereof complexed with a Dicer interacting protein is isolated by immunoaffinity chromatography.

14. The method of claim 13, wherein the Dicer interacting protein or bioactive fragment thereof is subjected to MudPIT.

15. The method of claim 9, wherein a detectable label is associated with a component selected from the group consisting of Dicer, a Dicer interacting protein, and test compound.

16. A modulator identified by any one of the preceding claims.

17. A method of identifying a modulator of Dicer comprising,
contacting a S100 fraction comprising Dicer or a bioactive fragment thereof and a Dicer interacting protein with a test compound, and
determining the activity of Dicer or bioactive fragment in the presence of the test compound as compared to an appropriate control, wherein the test compound is a potential modulator of Dicer based on its ability to affect the activity of Dicer or a bioactive fragment thereof.

18. An antibody that specifically binds to a component selected from the group consisting of Dicer, Dicer interacting protein, and Dicer:Dicer interacting protein complex.

19. The antibody of claim 18, wherein the Dicer interacting protein is selected from the group consisting of RDE-4, ALG-1, ALG-2, DRH-1, DRH-2, helicase homologous to DCR-2 (DRH-3), double helicases, EFT-2 EF-Tu family GTP binding, EFT-4 (eIF1 alpha), GAP/RAN-GAP family, HMG-I/Y DNA binding, HMG-I/Y DNA, binding PB1 domain, SNR-2 SM protein, SNR-3 SM protein, Dual specificity phosphatase, LIN-41, low homology MADS box, novel, RPN-9 proteasome subunits, TAF 6.1, T54 homology, RRM protein (3 domains), Worm unique/Novel (ce27223), TBB-4, RPS-14, RPS-13, RPL-24, RPS-11, Agglutinin, SIP-1 (hsp20), CCT-6 (chaperonin), RDE-1, DRH-3, ERI-1, RRF-3, ERI-3, ERI-5, PIR-1, and C32A3.2.

20. A pharmaceutical composition comprising the modulator of claim 17.

21. A method of activating target-specific RNA interference (RNAi) in an organism comprising,
administering to the organism an RNAi agent and a modulator of Dicer activity, wherein the modulator is administered in an amount sufficient for enhancing the activity of the RNAi agent, thereby achieving degradation of a target mRNA in the organism.

22. The method of claim 21, wherein the target mRNA encodes a gene product involved or predicted to be involved in a human disease or disorder.

23. A method of treating a disease or disorder associated with the activity of a gene product encoded by a target mRNA in a subject comprising,
administering to the subject an RNAi agent, and a modulator of Dicer activity, wherein said modulator is administered in an amount sufficient for enhancing the activity of the RNAi agent, thereby treating the disease or disorder associated with a gene product encoded by the target mRNA.

24. A method for deriving information about the function of a gene in a cell or organism comprising,
introducing into the cell or organism a Dicer interacting protein or an RNAi agent specific therefore, and
maintaining the cell or organism under target-specific RNAi conditions,
determining a characteristic or property of the cell or organism, and
comparing the characteristic or property to a suitable control, the comparison yielding information about the function of the gene.

25. A method of deriving information about the function of a Dicer interacting protein in a extract, cell, or organism comprising,
exposing an extract, cell, or organism capable of expressing Dicer and a Dicer interacting protein to an RNAi agent, maintaining the lysate, cell, or organism under conditions such that target-specific RNAi can occur,
determining a characteristic or property of the extract, cell, or organism, and
comparing the characteristic or property to a suitable control, the comparison yielding information about the function of the gene.

26. A method of validating a candidate Dicer interacting protein as a suitable target for drug discovery comprising,
introducing into a cell or organism a Dicer interacting protein or an RNAi agent specific therefore, and
maintaining the cell or organism under target-specific RNAi conditions,
determining a characteristic or property of the cell or organism, and
comparing the characteristic or property to a suitable control, the comparison yielding information about whether the candidate protein is a suitable target for drug discovery.

27. The method of claims 24, 25, or 26, wherein the organism is selected from the group consisting of nematode, fruit fly, mouse, rat, primate, and human.

28. A method of treating a disease or disorder associated with the activity of a gene product encoded by a target RNA in a subject comprising,
administering to the subject an agent sufficient to modulate Dicer activity in one or more cells and
administering an RNAi agent in an amount sufficient for degradation of the target RNA to occur, thereby treating the disease or disorder associated with the gene product encoded by the target gene.

29. The method of claim 28, wherein the subject is a human patient.

30. A kit comprising a reagent for activating target-specific RNA interference (RNAi) in a cell or organism, the kit comprising:
a component selected from the group consisting of a molecule encoding a Dicer interacting protein, an RNAi agent specific for a Dicer interacting protein, a modulator of Dicer activity, and a modulator of a Dicer interacting protein, and
instructions for use.
